(19) United States
(12) Patent Application Publication
(10) Pub. No.: US 2025/0296912 A1
(43) Pub. Date: Sep. 25, 2025

(54) AFFINITY ILLUDOFULVENE CONJUGATES

(71) Applicant: Illudent, Inc., San Diego, CA (US)

(72) Inventor: MICHAEL KELNER, San Diego, CA (US)

(73) Assignee: Illudent, Inc., San Diego, CA (US)

(21) Appl. No.: 19/084,695

(22) Filed: Mar. 19, 2025

Related U.S. Application Data
(63) Continuation of application No. 18/175,495, filed on Feb. 27, 2023, now Pat. No. 12,269,793.
Application No. 18/175,495 is a continuation of application No. 17/103,780, filed on Nov. 24, 2020, now Pat. No. 11,591,295.
(60) Provisional application No. 62/940,096, filed on Nov. 25, 2019.
(60) Provisional application No. 63/116,772, filed on Nov. 20, 2020.

(30) Foreign Application Priority Data
Nov. 24, 2020 (EP) 20 209 541.0

(51) Int. Cl. C07C 317/44; A61K 31/215; A61K 31/255; A61K 31/265; A61K 31/27; A61K 31/4015; A61K 38/05; A61K 38/06; A61K 47/54; C07C 205/42; C07C 225/14; C07C 271/34; C07C 309/72; C07C 311/16; C07D 207/444; C07K 5/062; C07K 5/083

(52) U.S. Cl. CPC C07C 317/44; A61K 31/215; A61K 31/255; A61K 31/265; A61K 31/27; A61K 31/4015; A61K 38/05; A61K 38/06; A61K 47/554; C07C 205/42; C07C 225/14; C07C 271/34; C07C 309/72; C07C 311/16; C07D 207/444; C07K 5/06026; C07K 5/06052; C07K 5/0806; C07C 2603/94

(57) ABSTRACT

In an embodiment of the invention, a composition for treating a cell population comprises a medicant. The medicant moiety can be an illudofulvene analog. In an embodiment of the invention, a composition for treating a cell population comprises an Affinity Medicant Conjugate (AMC). The affinity moiety can be an antibody, an antibody fragment, a receptor protein, a peptidic growth factor, an anti-angiogenic protein, a specific binding peptide, protease cleavable peptide, a glycopeptide, a peptide, a peptidic toxin, a protein toxin and an oligonucleotide. The affinity moiety can be covalently bound to the medicant via a linker.

### PRIORITY CLAIM

This application is a continuation of and claims priority to (i) U.S. Utility application Ser. No. 18/175,495 entitled “AFFINITY ILLUDOFULVENE CONJUGATES”, inventor: Michael Kelner et al. filed Feb. 27, 2023, which is a continuation of and claims priority to (ii) U.S. Utility application Ser. No. 17/103,780 entitled “AFFINITY ILLUDOFULVENE CONJUGATES”, inventor: Michael Kelner et al. filed Nov. 24, 2020, which issued as U.S. patent Ser. No. 11/591,295 and which claims priority to (iii) U.S. Provisional Application No. 62/940,096 entitled “AFFINITY ILLUDOFULVENE CONJUGATES”, inventor: Michael Kelner et al. filed Nov. 25, 2019, (iv) U.S. Provisional Application No. 63/116,772 entitled “AFFINITY ILLUDOFULVENE CONJUGATES”, inventor: Michael Kelner et al. filed Nov. 20, 2020, and (v) EP Patent Application No. 20209541.0 entitled “AFFINITY ILLUDOFULVENE CONJUGATES”, inventor: Michael Kelner et al. filed Nov. 24, 2020, which applications (i)-(v) are herein expressly incorporated by reference in their entireties and for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file MKEL-01049US4_ST26.XML, created Mar. 18, 2025, 441,306 bytes, machine format IBM-PC, MS-Windows operating system, is hereby incorporated by reference in its entirety and for all purposes.

### TECHNICAL FIELD

The present invention relates to compositions and methods for treating target molecules including cell populations with either a medicant or an affinity medicant conjugate including an antibody drug conjugate or a small molecule medicant conjugate.

### BACKGROUND ART

The present invention is directed to a medicant or an Affinity Medicant Conjugate (AMC) including illudofulvene based medicants, illudofulvene based Affinity Medicant Linker Conjugates (AMLC), illudofulvene based antibody-drug conjugates (ADC) and illudofulvene based medicant-linker (ML) compounds, as well as to compositions of the same, and to methods for their use in treating cancer, an autoimmune to methods of using illudofulvene based Ligand Linker Medicant (LLM) conjugates and illudofulvene based ML compounds in vitro, in situ, and in vivo for the detection, diagnosis or treatment of cells and associated pathological conditions.

### SUMMARY OF INVENTION

There exists a continuing need for chemotherapeutic agents for treating cancer. In particular there is a need for chemotherapeutic agents that have activity against cancers with resistant phenotypes and which can inhibit tumor growth and which have an adequate therapeutic index to be effective for in vivo treatment. In an embodiment of the invention, the chemotherapeutic agents can be delivered to tumors as a stand alone treatment. In an alternative embodiment of the invention, the chemotherapeutic agents can be delivered to a specific patient population having a specific tumors as a stand alone treatment. In another embodiment of the invention, the chemotherapeutic agents can be conjugated with an antibody to form an effective treatment. In another embodiment of the invention, the chemotherapeutic agents can be conjugated with an antibody, an antibody fragment, a receptor protein, a peptidic growth factor, an anti-angiogenic protein, a specific binding peptide, protease cleavable peptide, a glycopeptide, a peptide, a peptidic toxin, a protein toxin and an oligonucleotide and delivered to a specific patient population having a specific tumors to form an effective treatment. In various embodiments of the invention, the therapeutic treatment can be delivered in humans as well as in animals. For example, such therapeutic applications can include: cancer, adenocarcinoma, carcinoma, breast cancer, prostate cancer, ovarian cancer, endometrial cancer, neuroendocrine tumors, infertility, polycystic ovary syndrome, endometriosis, and precocious puberty. For example, veterinary and agricultural applications can include treatment of cancer, adenocarcinoma, carcinoma, ovarian cancer, endometrial cancer, neuroendocrine tumors, and endometriosis in farmyard and/or companion animals.

### BRIEF DESCRIPTION OF THE DRAWINGS

This invention is described with respect to specific embodiments thereof. Additional features can be appreciated from the FIG.s in which:

FIG. 1A shows analog 317; FIG. 1B shows analog 318; FIG. 1C shows analog 332; FIG. 1D shows analog 333; FIG. 1E shows analog 334; FIG. 1F shows analog 335; FIG. 1G shows analog 337; FIG. 1H shows analog 338; FIG. 1I shows analog 339; FIG. 1J shows analog 345; FIG. 1K shows analog 346; FIG. 1L shows analog 347; FIG. 1M shows analog 348; FIG. 1N shows analog 351; FIG. 1O shows analog 353; FIG. 1P shows analog 354; FIG. 1Q shows analog 356; FIG. 1R shows analog 357; FIG. 1S shows analog 359; FIG. 1T shows analog 361; FIG. 1U shows analog 362; FIG. 1V shows analog 363; FIG. 1W shows analog 364; FIG. 1X shows analog 366; FIG. 1Y shows analog 367; FIG. 1Z shows analog 368; FIG. 2A shows analog 369; FIG. 2B shows analog 370; FIG. 2C shows analog 371; FIG. 2D shows analog 372; FIG. 2E shows analog 373; FIG. 2F shows analog 374; FIG. 2G shows analog 377; FIG. 2H shows analog 378; FIG. 2I shows analog 379; FIG. 2J shows analog 380; FIG. 2K shows analog 381; FIG. 2L shows analog 382; FIG. 2M shows analog 383; FIG. 2N shows analog 384; FIG. 2O shows analog 389; FIG. 2P shows analog 392; FIG. 2Q shows analog 393; FIG. 2R shows analog 394; FIG. 2S shows analog 397; FIG. 2T shows analog 398; FIG. 2U shows analog 399; FIG. 2V shows analog 401; FIG. 2W shows analog 402; FIG. 2X shows analog 403; FIG. 2Y shows analog 404; FIG. 2Z shows analog 405; FIG. 3A shows analog 407; FIG. 3B shows analog 408; FIG. 3C shows analog 409 and FIG. 3D shows analog 410, according to various embodiments of the invention.

### DETAILED DESCRIPTION OF THE INVENTION

Definitions. As used herein, the term “receptor for a biologically active polypeptide” means a receptor which can bind a biologically active peptide conjugate.

As used herein, the term “cell population” is used to describe a set or subset of cells expressing a molecule such as a receptor.

The phrase ‘Other Drugs’ means docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, and rituximab.

‘Acylfulvene’ means an Illudofulvene subgroup with the following structural molecular formula:

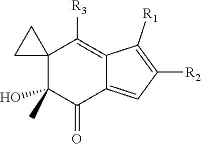

where R₁, R₂, R₃, R₈, R₉, R₁₀, R₁₁, are as set forth in Formula P1, as given herein.

‘Formula P1’ means R₁ represents —H, —CR₉R₈OH, —CHR₉CHR₈OH, —CHR₁₀CHR₉CHR₈OH, —CH₂CHR₁₀CHR₉CHR₈OH, —CR₉R₈OH, —CHR₉CHR₈OH, —CHR₁₀CHR₉CHR₈OH, —CH₂CHR₁₀CHR₉CHR₈OH, —C(═O)H, —CH₂(C═O)H, —CH₂CH₂(C═O)H, —CH₂CH₂CH₂(C═O)H, —CH₂CH₂CH₂CH₂(C═O)H, —CR₉R₈(C═O)H, —CHR₉CHR₈(C═O)H, —CH₂CHR₉CHR₈(C═O)H, —CR₉R₈(C═O)R₁₀, —CHR₉CHR₈(C═O)R₁₀, —CH₂CHR₉CHR₈(C═O)R₁₀, —CH₂CH₂CHR₉CHR₈(C═O)R₁₀, —CO₂H, —CHR₉CO₂H, —CHR₈CHR₉CO₂H, —CHR₁₀CHR₈CHR₉CO₂H, —CO₂R₁₀, —CHR₉CO₂R₁₀, —CHR₈CHR₉CO₂R₁₀, —CH₂CHR₈CHR₉CO₂R₁₀, —CHR₉CH₂CH₂CHR₈CO₂H, —CHR₉CH₂CH₂CHR₈CO₂R₁₀, —CR₈=CH₂, —CHR₈CH═CH₂, —CH₂CHR₈CH═CH₂, —CH₂CH₂CHR₈CH═CH₂, —CR₈=CHR₉, —CHR₈CR₉=CH₂, —CH₂CHR₈CR₉=CH₂, —CH₂CH₂CHR₈CR₉=CH₂, —CR₈=CR₉R₁₀, —CHR₈CH═CR₉R₁₀, —CH₂CHR₈CH═CHR₉R₁₀, —CH₂CH₂CHR₈CH═CHR₉R₁₀, —Cl, —Br, —I, —F, —NO₂, —NR₈R₉, —CH₂Cl, —CH₂Br, —CH₂I, —CH₂F, —CR₈Cl₂, —CR₈Br₂, —CR₈I₂, —CR₈F₂, —CCl₃, —CBr₃, —Cl₃, —CF₃, —CHR₈Cl, —CR₈R₉Cl, —CHR₈CHR₉Cl, —CHR₁₀CHR₈CHR₉Cl, —CH₂CHR₁₀CHR₈CHR₉Cl, —CHR₈Br, —CR₈R₉Br, —CHR₈CHR₉Br, —CHR₁₀CHR₈CHR₉Br, —CH₂CHR₁₀CHR₈CHR₉Br, —CHR₈I, —CR₈R₉I, —CHR₈CHR₉Br, —CHR₁₀CHR₈CHR₉I, —CH₂CHR₁₀CHR₈CHR₉I, —CR₈R₉NH₂, —CHR₈CHR₉NH₂, —CHR₁₀CHR₈CHR₉NH₂, —CH₂CH R₁₀CHR₈CHR₉NH₂, —CR₉R₁₀NHR₈, —CHR₉CHR₁₀NHR₈, —CH₂CHR₉CHR₁₀NHR₈, —CH₂CH₂CHR₉CHR₁₀NHR₈, —CHR₉NHR₈R₁₀, —CHR₉CH₂NHR₈R₁₀, —CH₂CH₂CHR₉NR₁₀R₈, —CH₂CH₂CH₂CH₂NHR₈, —CR₉R₈OR₁₀, —CH₂CR₈R₉OR₁₀, —CH₂CH₂CR₈R₉OR₁₀, —CH₂CH₂CH₂R₈R₉OR₁₀, —CH₂CH₂CH₂CH₂R₈R₉OR₁₀, —CHR₈OC(═O)CHR₉R₁₀, —CHR₈OC(═O)CHR₉R₁₀, —CH₂CH₂OC(═O)CHR₉R₁₀, —CH₂CH₂CHR₈OC(═O)CHR₉R₁₀, —CH₂CH₂CH₂CH₂OC(═O)CHR₉R₁₀, —CHR₈(═O)OCHR₉R₁₀, —CHR₈(═O)OCHR₉R₁₀, —CH₂CHR₈(═O)OCHR₉R₁₀, —CH₂CH₂CHR₈(═O)OCHR₉R₁₀, —CH₂CH₂CH₂CHR₈(═O)OCHR₉R₁₀, —R₁₂C═CR₉CH₂OH, —R₁₂C═CR₉C(═O)H, —R₁₂C═CR₉CH₂OR₁₀, —R₁₂C═CR₉C(═O) R₁₀, —CH₃, —CH₂CH₂, —CHR₈CH₂, —CHR₈CH₂CH₂, —CHR₈CHR₉CH₃, —OCH₃, —OCR₈R₉R₁₀, —OCH₂CR₈R₉R₁₀, —OCR₉R₈CHR₁₀, —OCHR₈CH₂CH₃, —OCHR₈CHR₉CH₃, —NR₈CH₃, —NR₈CH₂CH₃, —NR₉CHR₈CH₃, —NR₉CHR₈CH₂CH₃, —NR₁₀CHR₈CHR₉CH₃, —OCH₂OR₈, —OCHR₈OR₉, —OCHR₈CH₂OR₉, —OCHR₈CHR₉OR₁₀, —OC(═O)OR₈, —OCH₂C(═O)OR₈, —OCHR₉C(═O)OR₈, —CR₈(═N)H, —CH₂CR₈(═N)H, —CH₂CR₈(═N)H, —CH₂CH₂CR₈ (═N)H, —CH₂CH₂CH₂CR₈(═N)H, —CH₂CH₂CH₂CH₂CR₈(═N)H, —CR₈(═N)OH, —CH₂CR₈(═N)OH, —CH₂CR₈(═N)OH, —CH₂CH₂CR₈(═N)OH, —CH₂CH₂CH₂CR₈(═N)OH, —CH₂CH₂CH₂CH₂CR₈(═N)OH, —CR₈(═N)R₉, —CH₂CR₈(═N) R₉, —CH₂CR₈(═N) R₉, —CH₂CH₂CR₈ (═N) R₉, —CH₂CH₂CH₂CR₈(═N)R₉, —CH₂CH₂CH₂CH₂CR₈(═N)R₉, —CR₈(═N)OR₉, —CH₂CR₈(═N)OR₉, —CH₂CR₈(═N)O R₉, —CH₂CH₂CR₈ (═N)OR₉, —CH₂CH₂CH₂CR₈(═N)OR₉, —CH₂CH₂CH₂CH₂CR₈ (═N)OR₉, —CR₈(═N)NR₉, —CH₂CR₈(═N)NR₉, —CH₂CR₈(═N)NR₉, —CH₂CH₂CR₈ (═N)NR₉, —CH₂CH₂CH₂—CR₈(═N)NR₉, —CH₂CH₂CH₂CH₂CR₈ (═N)NR₉, —CR₈(═N)NR₉S(═O)₂R₁₀, —CH₂C(R₈)(═N)NR₉S(═O)₂R₁₀, —CH₂CH₂C(R₈)(═N)NR₉S(═O)₂R₁₀, —CH₂CH₂CH₂C(R₈)(═N)NR₉S(═O)₂R₁₀, —CH₂CH₂CH₂CH₂, —C(R₈)(═N)NR₉S(═O)₂R₁₀, —R₁₂N(R₈)C(═O)NR₉R₁₀, —R₁₂N(R₈)C(═S)NR₉R₁₀, —R₁₂N(OR₈)C(═O)NR₉R₁₀, —R₁₂N(OR₈)C(═S)NR₉R₁₀, —R₁₂OS(O₂)NH₂, —R₁₂NHS(O₂)NH₂, —R₁₂OS(O₂)NR₈R₉, —R₁₂NHS(O₂)NR₈R₉, —CH₂N(R₈)S(O₂)NR₉R₁₀, —CH₂CH₂N(R₈)S(O₂)NR₉R₁₀, —CH₂CH₂CH₂N(R₈)S(O₂)NR₉R₁₀, —CH₂N(R₈)S(O₂)CR₉R₁₀R₁₁, —CH₂CH₂N(R₈)S(O₂) CR₉R₁₀R₁₁, —CH₂CH₂CH₂N(R₈)S(O₂)CR₉R₁₀R₁₁, —N(R₈)C(═O)R₉, —CH₂N(R₈)C(═O)R₉, —CH₂CH₂N(R₈)C(═O)R₉, —CH₂CH₂CH₂N(R₈)C(═O)R₉, —CH₂N(R₈)(C═O)NR₉R₁₀, —CH₂CH₂N(R₈)(C═O)NR₉R₁₀, —CH₂CH₂N(R₈)(C═O)NR₉R₁₀, —CH₂N(R₈)(C═O)CR₉R₁₀R₁₁, —CH₂CH₂N(R₈)(C═O) CR₉R₁₀R₁₁, —CH₂CH₂N(R₈)(C═O) CR₉R₁₀R₁₁, —R₁₂N(OH)C(═O)NHOH, —R₁₂N(OH)C(═S)NHOH, —R₁₂N(OR₈)C(═O)NHOR₉, —R₁₂N(OR₈)C(═S)NHOR₉, —R₁₂OS(O₂)NHOH, —R₁₂NHS(O₂)NHOH, —R₁₂OS(O₂)NHOR₉, —R₁₂OS(O₂)N(R₈)OR₉, —R₁₂NR₈S(O₂)NHOR₉, —CR₉(═N)OR₈, —NH(OR₈), —C(C═O)NHR₈, —C(C═O)NR₉R₈, —NR₁₀(OR₈)C(═O)R9, —N(OR₈)C(═O)NR₉, —NR₈(R₉)SR₁₀, —N(R₈)S(═O)R₉, —NR(R₈)S(═O)₂R₉, —OC(═O)NR₈, —N(OR₈)C(═O)OR₉, —N(R₈)C(═S)R₉, —O(S(═O)₂R₈, —R₁₂O(S(═O)₂R₈, —O(S(═O)₂NR₈, —R₁₂O(S(═O)₂NR₈, —S(═O)R₈, —R₁₂S(═O)R₈, —S(═O)₂R₈, —R₁₂S(═O)₂R₈, —NR₁₀(R₉)S(═O)₂NHR₈, —NR₉(C═O)R₈, —NR₉(C═O)OR₈, —NR₉O(C═O)OR₈, —NR₉(C═O)NR₈R₁₀, —R₁₂N(R₉)S(═O)₂NHR₈, —R₁₂N(R₉)(C═O)R₈, —R₁₂N(R₉)(C═O)OR₈, —R₁₂N(R₉)(C═O)NR₈, —N(═NR₁₀)R₈, —R₉—N(═NR₁₀)R₈, —C(R₁₀)(═N—N═)CR₈R₉; —N₃, —CH₂N₃, —CH₂CH₂N₃, —CH₂CH₂CH₂N₃, —CH₂CH₂CH₂CH₂N₃, —CHR₈N₃, —CR₉R₈N₃, —CHR₉CHR₈N₃, —CH₂CHR₉CHR₈N₃, —CH₂CH₂CH₂CHR₈N₃, —C(R₈)═N—R₉, —CH₂C(R₈)═N—R₉, —CH₂CH₂C(R₈)═N—R₉, —CH₂CH₂CH₂C(R₈)═N—R₉, —N₃, —CH₂CH₂N₃, —CH₂CH₂CH₂N₃, —CH₂CH₂CH₂CH₂N₃; —CH₂NHC(═O)OC(CH₃)₃, —CH₂NOHC(═O)OC(CH₃)₃, —CH₂CH₂NHC(═O)OC(CH₃)₃, —CH₂CH₂NOHC(═O)OC(CH₃)₃, —CH₂CH₂CH₂NHC(═O)OC(CH₃)₃, —CH₂CH₂CH₂NOHC(═O)OC(CH₃)₃, —CH₂NHFmoc; —CH₂NOHFmoc; —CH₂CH₂NHFmoc; —CH₂CH₂NOHFmoc; —CH₂CH₂CH₂NH—Fmoc; —CH₂CH₂CH₂NOH—Fmoc, and R2 and R3 each independently represent —H, —OH, —CH₃, —OCH₃, —C(═O)CH₃, —OC(═O)CH₃, —C(═O)OCH₃, —C(═O)CH₂CH₃, —OC(═O)CH₂CH₃, —C(═O)OCH₂CH₃, —CH₂CH₃, —CH(CH₃)₂, —C(CH₃)₃, —CH₂CH₂CH₃, —CH₂CH(CH₃)₂, —CHC(CH₃)₃, —CH₂OH, —NH₂, —NHOH, —CH₂NH₂, —CH₂NHOH, —N₃, and (C1-C4)alkyl; where R₈, R₉, R₁₀, R₁₁ each independently represent —H, —OH, —CH₃, —CH₂CH₃, —CH₂═CH₂, —CH₂CH₂═CH₂, —CH₂CH₂CH₂═CH₂, —C(H)═O, —CH₂C(H)═O, —CH₂CH₂C(H)═O, —CH(CH₃)CH₃, —CH₂CH₂CH₃, —CH₂CH(CH₃)CH₃, —CHC(CH₃)₂CH₃, —C(═O)CH₃, —C(═O)CH₂CH₃, —C(═O)NH₂, —CH₂OH, —CH₂CH₂OH, —C(H)(OH)C(H₂)(OH), —OCH₃, —OC(CH₃)₃, —OCH₂CH₃, —OCH(CH₃)₂, —CO₂H, —C(═O)OCH₃, —C(═O)OCH₂CH₃, —OC(═O)CH₃, —OC(═O)CH₂CH₃, —OC(═O)OCH₃, —OC(═O)OCH₂CH₃, —C(CH₃)₃, —CH₂CH₂(CH₃)₂, —CHC(CH₃)₃, —CH₂CH₂OH, CH₂CH₂CH₂OH, —Cl, —Br, —I, —F, —CH₂Cl, —CH₂Br, —CH₂I, —CH₂F, —CHCl₂, —CHBr₂, —CHI₂, —CHF₂, —CCl₃, —CBr₃, —Cl₃, —CF₃, —NH₂, —CH₂NH₂, —NH(OH), —CH₂N(OH), —NH(OCH₃), —N(OCH₂CH₃), —CH₂NH(OCH₃), —CH₂N(OCH₂CH₃), —N₃, —CH₂N₃, —CH₂CH₂N₃, —CH═NH, —CH═NOH, —CH═NOCH₃, —SH, —SCH₃, —SCH₂CH₃, —NO₂, —CN, cyclopropane ring, saturated or unsaturated cyclobutane ring, saturated or unsaturated cyclopentane ring, saturated or unsaturated cyclohexane ring, benzene ring, phenolic ring, xylene ring, an amino acid(s), and (C₁-C₄)alkyl, and R12 represents —CH₂—, —CH₂CH₂—, —CH(CH₃)CH₂—, —C(CH₃)CH₂—, —CH═CH—, —O—, —S—, —O(C═O)—, —(C═O)O—, —NH—, —N(R₈)—, —N(OH)—, —CH₂—O—, —O—CH₂—, —CH₂CH₂—O—, —O—CH₂CH₂—, —S(═O)—, —(S═O)₂—, —NH(S═O)₂—, —N(OH)S(═O)₂—, —S(═O)₂NH—, —S(═O)₂N(OH)—, —NHS(═O)₂—, —N(OH)S(═O)₂—, —CH₂NH—, —CH₂N(R₈)—, —CH₂N(OH)—, —NHCH₂—, —N(R₈)CH₂—, —N(OH)CH₂—, —OC(C═O)O—, —OC(═O)NR₈—, —NR₈C(═O)O—.

‘Illudin’ means an Illudofulvene subgroup with the following structural molecular formula:

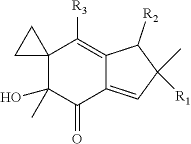

where R₁, R₂, R₃, are as set forth in Formula P2, as given herein.

‘Formula P2’ means where R₁, R₂, and R₃ each independently represent —H, —OH, —CH₃, —OCH₃, —C(═O)CH₃, —OC(═O)CH₃, —C(═O)OCH₃, —C(═O)CH₂CH₃, —OC(═O)CH₂CH₃, —C(═O)OCH₂CH₃, —CH₂CH₃, —CH(CH₃)₂, —C(CH₃)₃, —CH₂CH₂CH₃, —CH₂CH(CH₃)₂, —CHC(CH₃)₃, —CH₂OH, —NH₂, —CH₂NH₂, —N₃, —CH₂NHOH, —NHOH, —CH₂NHC(═O)OC(CH₃)₃, —CH₂NOHC(═O)OC(CH₃)₃, —C(═O)H, —C(═O)OH, —CH₂OSi(CH₃)₂C(CH₃)₃, —OS(═O)₂CH₃, —CH₂OS(═O)₂(C₄H₆)CH₃, —OC(═O)O(C₆H₄)NO₂, —OC(═O)N(CH₃)CH₂CH₂N(CH₃)C(═O)OC(CH₃)₃, —OC(C═O)N(CH₃)CH₂CH₂N(CH₃)H, —OC(═O)NHCH₂CH₂NH₂, —NH(FMOC), —NOH(FMOC), —CH₂NH(FMOC), —CH₂NOH(FMOC), —OSi(CH₂CH₂CH₂CH₃)₃, —OSi(CH₃)₂C(CH₃)₃, and (C₁-C₄)alkyl.

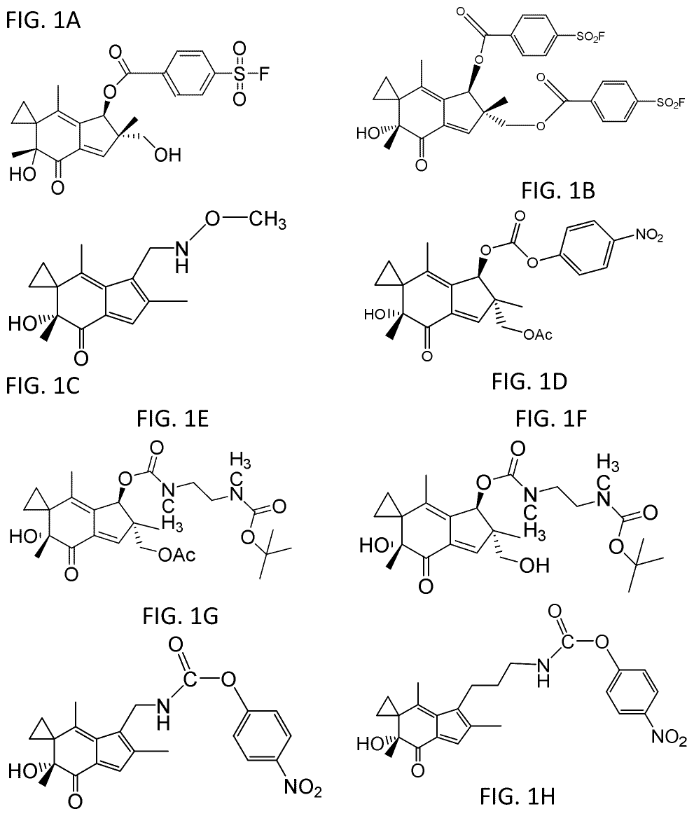

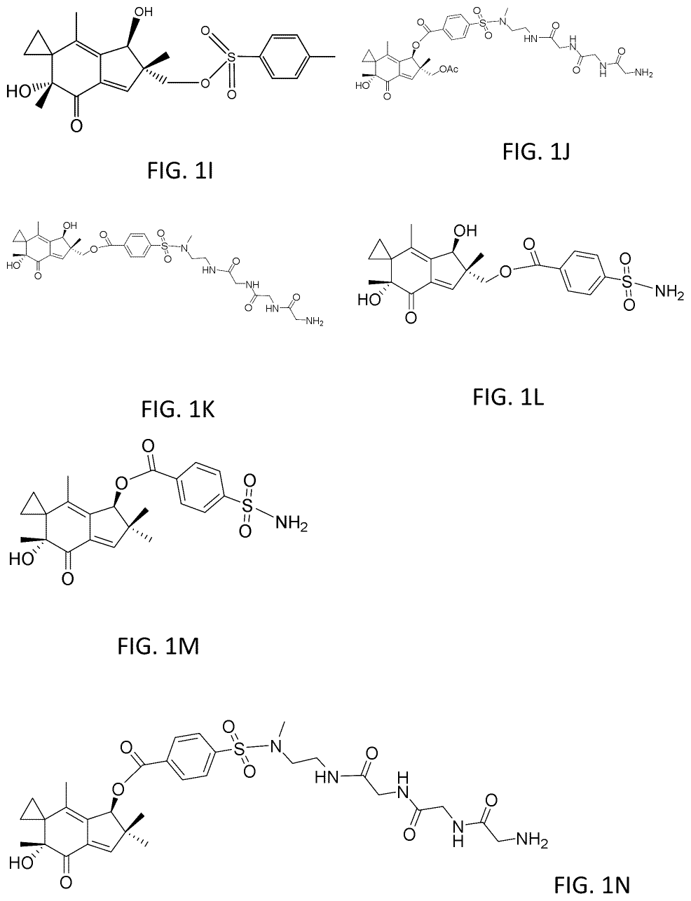

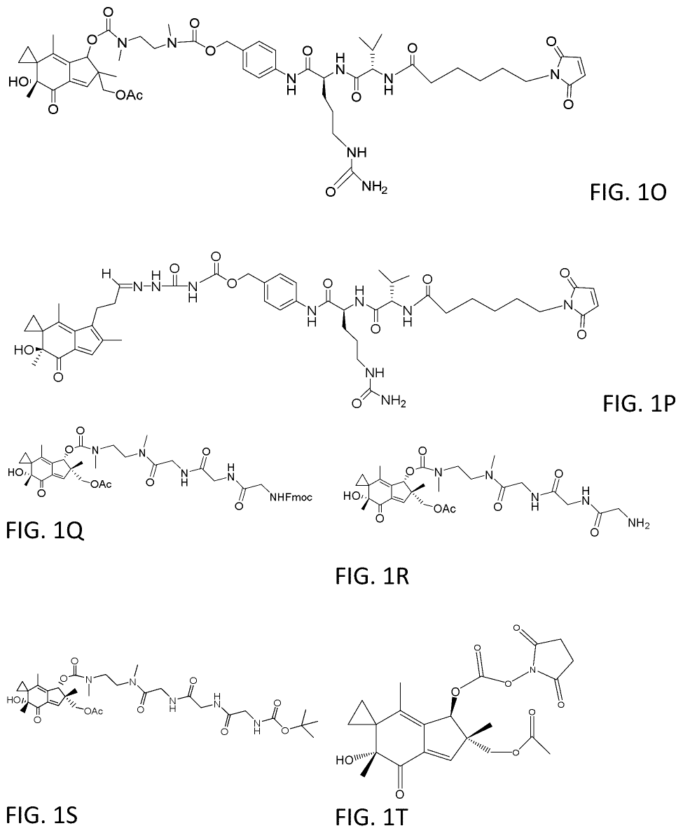

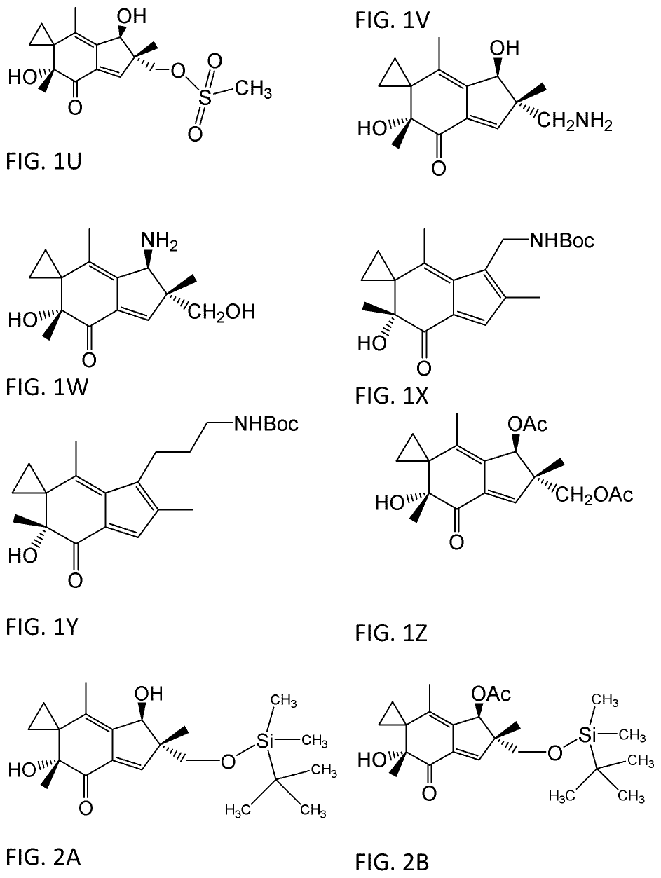

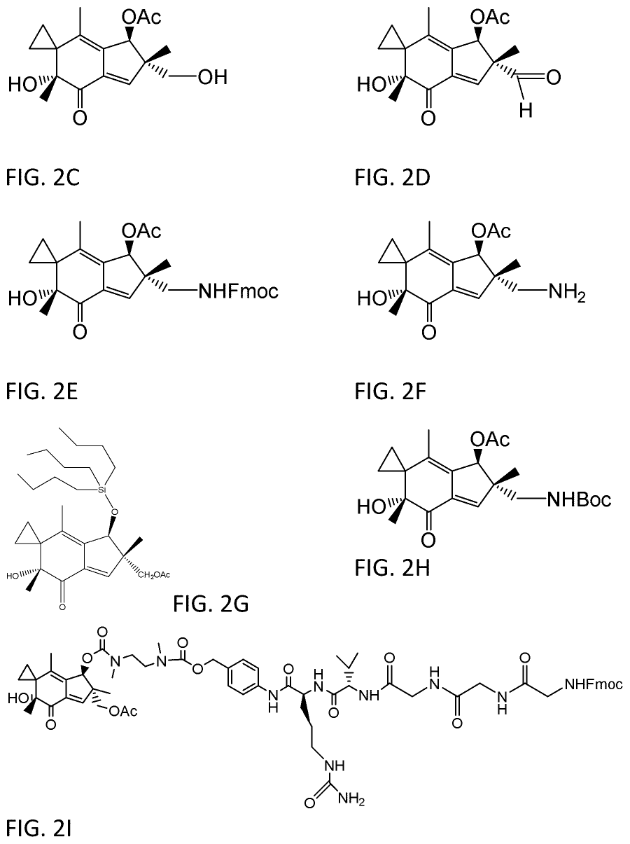

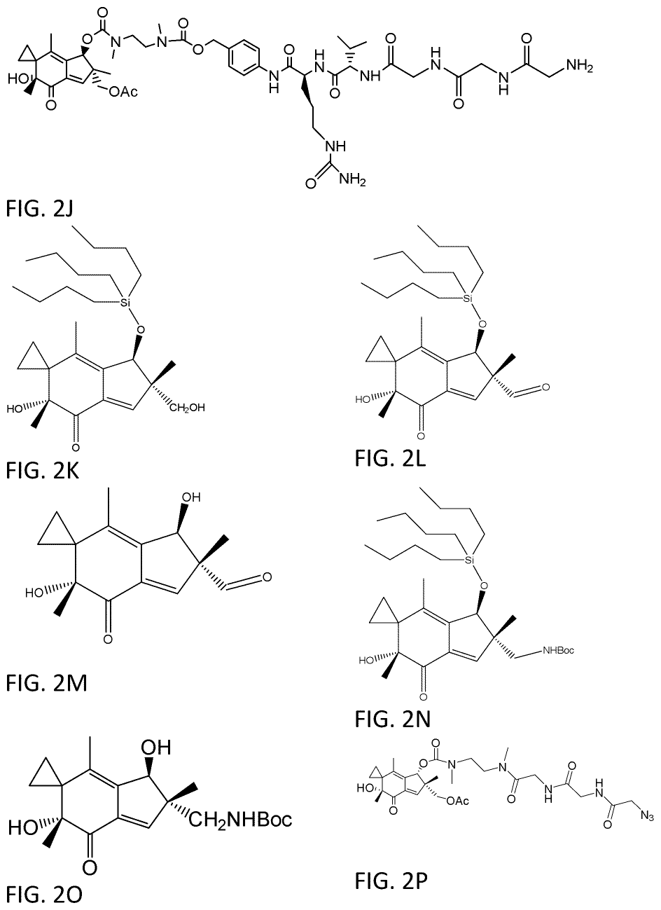

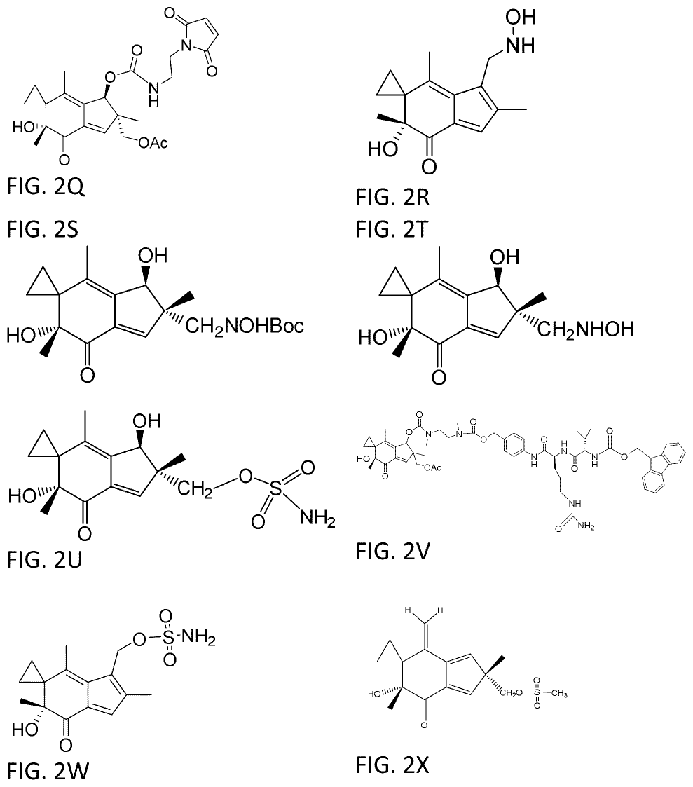

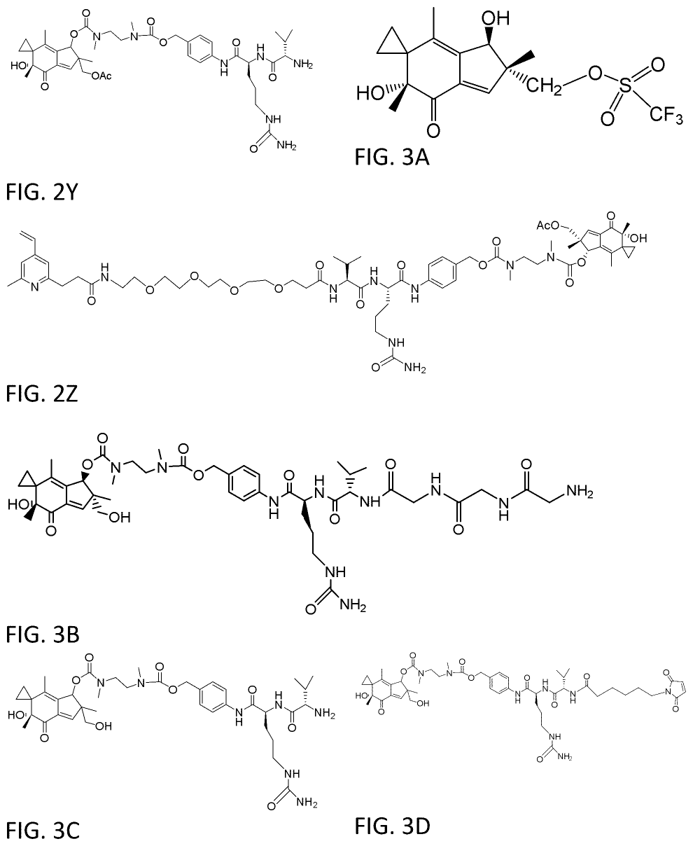

Table V is a listing of IUPAC names of the Illudofulvene Analog according to various embodiments of the present invention. FIGS. 1A-3D show the structure of analogs 317, 318, 332, 333, 334, 335, 337, 338, 339, 345, 346, 347, 348, 351, 353, 354, 356, 357, 359, 361, 362, 363, 364, 366, 367, 368, 369, 370, 371, 372, 373, 374, 377, 378, 379, 380, 381, 382, 383, 384, 389, 392, 393, 394, 397, 398, 399, 401, 402, 403, 404, 405, 407, 408, 409, and 410 respectively.

As used herein, the terms “analog”, “medicant” and “medicant moiety” are used interchangeably and comprise synthetic and naturally occurring drugs, toxins, nutraceuticals and other cytoactive, anti-inflammatory and bioactive molecules including Doxorubicin (Immunomedics), auristatins E (Seattle Genetics), auristatins F (Celdex), monomethyl auristatin E (MMAE) (Amgen), monomethyl auristatin F (MMAF) (Astelles), maytanasines (Immunogen), DM1 (Biotest), DM4 (Amgen), calicheamicin (CellTech), irinotecan, folate, SN38 (Immunomedics), Pyrrolobenzodiazepines (Seattle Genetics), MGBA a duocarmycin derivative (Medarex), thalidomides, taxanes, penicillins, Trastuzumab emtansine (Genentech for Breast cancer uses maytanasine derive DM-1). Some of the above analogs are stand alone drugs, but can be used as a medicant moiety in an affinity drug conjugate according to various embodiments of the invention.

As used herein, the phrase “peptide receptor” includes peptide hormone receptors, protein hormone receptors, chemotactic receptors and chemokine receptors.

As used herein, the term “receptor” includes growth factor receptors, peptide hormone receptors, peptide receptors, steroid hormone receptors, steroid receptors and lipid receptors.

As used herein, phrase “affinity medicant conjugate” is an Affinity Moiety covalently bound to a medicant moiety, and includes antibody medicant conjugates, where the antibody is directed to a specific receptor. As used herein the phrase ‘Affinity Moiety’ includes antibodies, antibody fragments, peptides, proteins, growth factors, steroids, and lipids, where the antibodies, antibody fragments, peptides, proteins, growth factors, steroids, folate or lipids have an affinity for a specific receptor, receptors, is processed by an enzyme to produce a ligand that has an affinity for a specific receptor or otherwise directs the Affinity Moiety to a specific subset of cells. A ‘medicant moiety’ includes a group bound to an Affinity Moiety, which when released acts as a medicant.

As used herein, the term “Affinity Moiety” (AM) is used to describe a chemical group or molecule that can bind a receptor or proteins. An AM is understood to have a minimum binding affinity greater than approximately 1×10⁻³ M affinity. As used herein, the term AM includes “ligands”, “ligand moieties”, “affinity unit” and an AM modified to include a linker. As used herein, the phrase “an affinity moiety directed to a peptide receptor” is used to describe a molecule or a portion of a molecule which has a binding affinity to the peptide receptor greater than approximately 1×10⁻¹⁰ M. In this range approximately means 1×10⁻⁹ M to 1×10⁻¹¹ M. In an embodiment of the invention, an AM directed to a peptide receptor has a binding affinity to the peptide receptor greater than approximately 1×10⁻¹² M. In this range approximately means 1×10⁻¹¹ M to 1×10⁻¹³ M.

As used herein, the term “cytoactive” (which is abbreviated as “CA”) is used to describe a small molecule that disrupts a cellular process, modulates a cellular process or otherwise affects the normal function of the cell. As used herein, the term “toxin” or “toxic” is used to describe a small molecule which interferes with RNA or DNA synthesis, causes RNA or DNA strand scission, blocks cell cycling, division, replication or is otherwise cytotoxic to the cell. As used herein, the term “toxin moiety” is used to describe a toxin modified to include a linker. As used herein, the phrase “a moiety possessing cell cytotoxicity” is used to describe a toxin moiety which when given in the concentration range of approximately 1×10⁻³ M to approximately 1×10⁻⁹ M results in inhibition of DNA synthesis or proliferation in an appropriate cultured cell line and/or when administered intravenously to an animal in the dosage approximately 1×10⁻⁴ g to approximately 1×10⁻⁹ g of the compound per kilogram of body weight of the animal results in in vivo cell death. As used herein, the term “ablated” is used to describe a reduction in the cell population of between approximately 50% and approximately 95%. In this range approximately means plus or minus five (5) percent. In an embodiment of the invention, a toxin moiety ablating a cell population reduces the cell population by approximately 100 percent. As used herein, the term “impaired” is used to describe a reduction in the cell population of between approximately 30% and approximately 50%. In this range approximately means plus or minus ten (10) percent.

As used herein, the term “linker” is used to describe one or more covalently bonded groups of atoms that are covalently bonded to a medicant moiety and an AM. For example a linker can be covalently bound to both an illudofulvene moiety and to an antibody or other ligand moiety with an affinity for a receptor.

As used herein, the term “non releasable linker” is used to describe a linker covalently bound to an AM and a medicant moiety in which the AM and the medicant moiety remain covalently bound to the linker after internalization and exposure to both reducing and acidic environments of vesicles within the cell. As used herein, the term “membrane permeability” is used to describe a compound comprising a linker covalently bound to an AM and an illudofulvene moiety, where the compound can diffuse across membranes within the cell.

As used herein, the term “transmembrane receptor” means a protein that spans the plasma membrane of a cell with the extracellular domain of the protein having the ability to bind an AM and the intracellular domain having an activity such as activation of G protein signaling which is induced upon the AM binding.

As used herein, the term “seven transmembrane receptor” is a transmembrane receptor including a transmembrane domain where the protein spans the cell membrane in seven (7) regions.

As used herein, the term “G-protein coupled receptor” means a seven transmembrane domain receptor which transduces a biological signal via G-protein coupling.

As used herein, the term “conjugated” or “conjugate” means a chemical compound that is formed by joining two or more compounds with one or more chemical bonds or linkers. In an embodiment of the invention, an antibody and a medicant form a conjugate.

As used herein, the term “antibody” herein is used in the broadest sense and specifically covers intact antibodies, monoclonal antibodies, polyclonal antibodies, mono-specific antibodies, multi-specific antibodies (e.g., bi-specific antibodies), and antibody fragments that exhibit the desired biological activity, including those antibodies directed against Alk, Alk fusion proteins, CD 2 (SEQ. ID. 001), CD3epsilon (SEQ. ID. 002), CD5 (SEQ. ID. 003), CD7 (SEQ. ID. 004), CD19 (SEQ. ID. 005), CD20 (SEQ. ID. 006), CD22 (SEQ. ID. 007), CD25 (SEQ. ID. 008), CD30 (SEQ. ID. 009), CD33 (SEQ. ID. 010), CD37 (SEQ. ID. 011), CD44 (SEQ. ID. 012), CD44v6 (SEQ. ID. 013), CD56 (SEQ. ID. 014), CD70 (SEQ. ID. 015), CD74 (SEQ. ID. 016), CD79 (SEQ. ID. 017), CD79b (SEQ. ID. 018), CD 80 (SEQ. ID. 019), CD 86 (SEQ. ID. 020), CD138 (syndecan 1) (SEQ. ID. 021), CAIX (SEQ. ID. 022), Integrin alphaVbeta 3 (SEQ. ID. 023), EphA2 (SEQ. ID. 024), Crypto1 (SEQ. ID. 025), CanAg (SEQ. ID. 026), ENPP3 (SEQ. ID. 027), Nectin-4 (SEQ. ID. 028), Mesothelin (SEQ. ID. 029), Lewis Y (SEQ. ID. 030), EGFRvIII (SEQ. ID. 031), SLC44A4 (SEQ. ID. 032), EBTR (endothelin) (SEQ. ID. 033), erbB2/neu/HER2 (SEQ. ID. 034), Transferrin receptor (SEQ. ID. 035), 55 kDa breast cancer antigen, 72 kDa TAA, GPNMB (osteoactivin) (SEQ. ID. 038), CA-IX (SEQ. ID. 039), CEA (CD66e) (SEQ. ID. 040), CEACAM5 (SEQ. ID. 041), PSMA (SEQ. ID. 042), CA125 (MUC16) (SEQ. ID. 043), Muc (CA6) (SEQ. ID. 044), Melanoma glycoprotein NMB (SEQ. ID. 045), IL-2R (SEQ. ID. 166 and 046), IL13R (SEQ. ID. 047), TACSTD2 (TROP2 or EGP1) (SEQ. ID. 048), Folate receptor 1 (SEQ. ID. 049), Mucin 16 (SEQ. ID. 050), Endothelin receptor ETB (SEQ. ID. 051), STEAP1 (SEQ. ID. 052), SLC44A4 (AGS-5) (SEQ. ID. 053), AGS-16 (SEQ. ID. 054), and Guanylyl cyclase C (SEQ. ID. 055). An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system. An antibody can be of any type or class (e.g., IgG, IgE, IgM, IgD, and IgA) or subclass (e.g., IgG1, IgG2, IgG3, IgG4, IgA1 and IgA2). The antibody can be derived from any suitable species. In some embodiments, the antibody is of human or murine origin. An antibody can be, for example, human, humanized or chimeric.

As used herein, the terms “specifically binds” and “specific binding” refer to antibody binding to a predetermined antigen. Typically, the antibody binds with an affinity of at least about 1×10⁷ M, and binds to the predetermined antigen with an affinity that is at least two-fold greater than its affinity for binding to a non-specific antigen (e.g., Bovine Serum Albumin, casein) other than the predetermined antigen or a closely-related antigen.

As used herein, the term “monoclonal antibody” as used herein refers to an antibody obtained from a population of substantially homogeneous antibodies, i.e., the individual antibodies comprising the population are identical except for possible naturally-occurring mutations that may be present in minor amounts and includes “chimeric” antibodies in which a portion of the heavy and/or light chain is identical to or homologous with the corresponding sequence of antibodies derived from a particular species or belonging to a particular antibody class or subclass, while the remainder of the chain(s) is identical to or homologous with the corresponding sequences of antibodies derived from another species or belonging to another antibody class or subclass, as well as fragments of such antibodies, so long as they exhibit the desired biological activity. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

As used herein, an “intact antibody” is one which comprises an antigen-binding variable region as well as a light chain constant domain (C_{L}) and heavy chain constant domains, C_{H1}, C_{H2}, C_{H3} and C_{H4}, as appropriate for the antibody class. The constant domains may be native sequence constant domains (e.g., human native sequence constant domains) or amino acid sequence variants thereof.

As used herein, the term an “intact antibody” may have one or more “effector functions”, which refers to those biological activities attributable to the Fc region (e.g., a native sequence Fc region or amino acid sequence variant Fc region) of an antibody. Examples of antibody effector functions include complement dependent cytotoxicity, antibody-dependent cell-mediated cytotoxicity (AMCC) and antibody-dependent cell-mediated phagocytosis.

As used herein, the term an “antibody fragment” comprises a portion of an intact antibody, preferably comprising the antigen-binding or variable region thereof. Examples of antibody fragments include Fab, Fab′, F(ab′)₂, and Fv fragments, di-abodies, tri-abodies, tetrabodies, linear antibodies, single-chain antibody molecules, scFv, scFv-Fc, multi-specific antibody fragments formed from antibody fragment(s), a fragment(s) produced by a Fab expression library, or an epitope-binding fragments of any of the above which immuno specifically bind to a target antigen (e.g., a cancer cell antigen, a viral antigen or a microbial antigen).

As used herein, the term “variable” in the context of an antibody refers to certain portions of the variable domains of the antibody that differ extensively in sequence and are used in the binding and specificity of each particular antibody for its particular antigen. This variability is concentrated in three segments called “hypervariable regions” in the light chain and the heavy chain variable domains. The more highly conserved portions of variable domains are called the Framework Regions (FRs). The variable domains of native heavy and light chains each comprise four FRs connected by three hypervariable regions.

As used herein, “isolated” means separated from other components of (a) a natural source, such as a plant or animal cell or cell culture, or (b) a synthetic organic chemical reaction mixture. As used herein, “purified” means that when isolated, the isolate contains at least 95%, and in another aspect at least 98%, of a compound (e.g., a conjugate) by weight of the isolate.

As used herein, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, an antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is a tumor cell, e.g., a breast, ovarian, stomach, endometrial, salivary gland, lung, kidney, colon, thyroid, pancreatic or bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells.

As used herein, the term “therapeutically effective amount” refers to an amount of a medicant effective to treat a disease or disorder in a mammal. In the case of cancer, the therapeutically effective amount of the medicant may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the medicant may inhibit the growth of and/or kill existing cancer cells, it may be cytostatic and/or cytotoxic. For cancer therapy, efficacy can, for example, be measured by assessing the time to disease progression (TTP) and/or determining the response rate (RR).

As used herein, the term “substantial amount” refers to a majority, i.e. greater than approximately fifty percent (50%) of a population, of a mixture or a sample. In this range approximately means plus or minus ten percent (10%).

As used herein, the term “intracellular metabolite” refers to a compound resulting from a metabolic process or reaction inside a cell on an Affinity Medicant Linker conjugate (e.g., an Antibody Drug Conjugate (AMC)). The metabolic process or reaction may be an enzymatic process such as proteolytic cleavage of a peptide linker of the AMC. Intracellular metabolites include, but are not limited to, antibodies and free medicant which have undergone intracellular cleavage after entry, diffusion, uptake or transport into a cell.

As used herein, the terms “intracellularly cleaved” and “intracellular cleavage” refer to a metabolic process or reaction inside a cell on an Affinity Medicant Linker conjugate (e.g., an Antibody Medicant conjugate (AMC) or the like), whereby the covalent attachment, e.g., the linker, between the Medicant moiety (M) and the Affinity unit (e.g., an antibody (Ab)) is broken, resulting in the free Medicant, or other metabolite of the conjugate dissociated from the antibody inside the cell. The cleaved moieties of the Affinity Medicant Linker conjugate are thus intracellular metabolites.

As used herein, the term “bioavailability” refers to the systemic availability (i.e., blood/plasma levels) of a given amount of a medicant administered to a patient. Bioavailability is an absolute term that indicates measurement of both the time (rate) and total amount (extent) of medicant that reaches the general circulation from an administered dosage form.

As used herein, the term “cytotoxic activity” refers to a cell-killing, a cytostatic or an anti-proliferative effect of an Affinity Medicant Linker conjugate or an intracellular metabolite of an Affinity Medicant Linker conjugate. Cytotoxic activity may be expressed as the IC₅₀ value, which is the concentration (molar or mass) per unit volume at which half the cells survive.

As used herein, the term “cytotoxic agent” as used herein refers to a substance that inhibits or inhibits the function of cells and/or causes destruction of cells. The term is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²p, ⁶⁰C, and radioactive isotopes of Lu), chemotherapeutic agents, and toxins such as small molecule toxins or enzymatically active toxins of bacterial, fungal, plant or animal origin, including synthetic analogs and derivatives thereof. In one aspect, the term does not include a radioactive isotope(s).

As used herein, a “disorder” is any condition that would benefit from treatment with an Affinity Medicant Linker Conjugate. This includes chronic and acute disorders or diseases including those pathological conditions which predispose a mammal to the disorder in question. Non-limiting examples of disorders to be treated herein include benign and malignant cancers; leukemia and lymphoid malignancies, neuronal, glial, astrocytal, hypothalamic and other glandular, macrophagal, epithelial, stromal and blastocoelic disorders; and inflammatory, angiogenic and immunologic disorders.

As used herein, the terms “cancer” and “cancerous” refer to or describe the physiological condition or disorder in mammals that is typically characterized by unregulated cell growth. A “tumor” comprises one or more cancerous cells.

As used herein, an “autoimmune disease” herein is a disease or disorder arising from and directed against an individual's own tissues or a co-segregate or manifestation thereof or resulting condition therefrom.

As used herein, an example of a “patient” includes, but is not limited to, a human, rat, mouse, guinea pig, monkey, pig, goat, cow, horse, dog, cat, bird and fowl. In an exemplary embodiment, the patient is a human.

As used herein, the terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder.

As used herein, in the context of cancer, the term “treating” includes any or all of inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

As used herein, in the context of an autoimmune disease, the term “treating” includes any or all of inhibiting replication of cells associated with an autoimmune disease state including, but not limited to, cells that produce an autoimmune antibody, lessening the autoimmune-antibody burden and ameliorating one or more symptoms of an autoimmune disease.

As used herein, in the context of an infectious disease, the term “treating” includes any or all of: inhibiting the growth, multiplication or replication of the pathogen that causes the infectious disease and ameliorating one or more symptoms of an infectious disease.

As used herein, the term “package insert” is used to refer to instructions customarily included in commercial packages of therapeutic products, that contain information about the indication(s), usage, dosage, administration, contraindications and/or warnings concerning the use of such therapeutic products.

As used herein, a “native sequence” polypeptide is one which has the same amino acid sequence as a polypeptide, e.g., a tumor-associated antigen receptor, derived from nature. Such native sequence polypeptides can be isolated from nature or can be produced by recombinant or synthetic means. Thus, a native sequence polypeptide can have the amino acid sequence of a naturally-occurring human polypeptide, a murine polypeptide, or a polypeptide from any other mammalian species.

As used herein, an “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the nucleic acid where, for example, the nucleic acid molecule is in a chromosomal location different from that of natural cells.

As used herein, the expression “control sequences” refers to nucleic acid sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, a nucleic acid is “operably linked” when it is placed into a functional relationship with another nucleic acid sequence. For example, DNA for a pre-sequence or secretory leader is operably linked to DNA encoding a polypeptide if it is expressed as a pre-protein that participates in the secretion of the polypeptide; a promoter or enhancer is operably linked to a coding sequence, for example, if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking can be accomplished by ligation at convenient restriction sites. If such sites do not exist, the synthetic oligonucleotide adaptors or linkers can be used in accordance with conventional practice.

As used herein, the terms “cell,” “cell line,” and “cell culture” are used interchangeably and all such designations include progeny. The words “transformants” and “transformed cells” include the primary subject cell and cultures or progeny derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein, “diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, and 3-bromopropyl.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfoxide” as used herein, means a moiety having the formula R—S(O)—R′, where R and R′ are alkyl groups as defined above. R and R′ may have a specified number of carbons (e.g., “C₁-C₄ alkylsulfoxide”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —OS(O)₂NR′R″, —NRS(O)₂NR′R″, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC (O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —OS(O)₂NR′R″, —NRS(O)₂NR′R″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, —OS(O)₂NR′—, —NRS(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C′″R′″)_{d}—, where s and d are independently integers of from 0 to 3, and X′ is —O—, —NR′—, —S—, —S(O)—, —S(O)₂—, —OS(O)₂NR′—, —NRS(O)₂NR′—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate, and salts of organic acids like glucuronic or galacturonic acids. Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers, and individual isomers are encompassed within the scope of the present invention. The compounds of the present invention do not include those that are known in the art to be too unstable to synthesize and/or isolate.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), carbon-13 (¹³C), or carbon-14 (¹⁴C). All isotopic variations of the compounds of the present invention, whether radioactive or not, are encompassed within the scope of the present invention.

Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

As used herein, an amino acid “derivative” includes an amino acid having substitutions or modifications by covalent attachment of a parent amino acid, such as, e.g., by alkylation, glycosylation, acetylation, phosphorylation, and the like. Further included within the definition of “derivative” is, for example, one or more analogs₁ of an amino acid with substituted linkages, as well as other modifications known in the art.

As used herein, a “natural amino acid” refers to arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine, unless otherwise indicated by context.

As used herein, a “protecting group” refers to a moiety that when attached to a reactive group in a molecule masks, reduces or prevents that reactivity. Representative hydroxy protecting groups include acyl groups, benzyl and trityl ethers, tetrahydropyranyl ethers, trialkylsilyl ethers and allyl ethers. Representative amino protecting groups include formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like. Examples of a “hydroxyl protecting group” include, but are not limited to, methoxymethyl ether, 2-methoxyethoxymethyl ether, tetrahydropyranyl ether, benzyl ether, p-methoxybenzyl ether, trimethylsilyl ether, triethylsilyl ether, triisopropyl silyl ether, t-butyldimethyl silyl ether, triphenylmethyl silyl ether, acetate ester, substituted acetate esters, pivaloate, benzoate, methanesulfonate and p-toluenesulfonate.

As used herein, a “leaving group” refers to a functional group that can be substituted by another functional group. Such leaving groups are well known in the art, and examples include, but are not limited to, a halide (e.g., chloride, bromide, and iodide), methanesulfonyl (mesyl), p-toluenesulfonyl (tosyl), trifluoromethylsulfonyl (triflate), and trifluoromethylsulfonate.

The phrase “pharmaceutically acceptable salt,” as used herein, refers to pharmaceutically acceptable organic or inorganic salts of a compound (e.g., a Medicant Linker compound, or an Affinity Medicant Linker conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and palmoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

As used herein, a “pharmaceutically acceptable solvate” or “solvate” refer to an association of one or more solvent molecules and a compound of the invention, e.g., an Affinity Medicant Linker conjugate or a Medicant Linker compound. Examples of solvents that form pharmaceutically acceptable solvates include, but are not limited to, water, isopropanol, ethanol, methanol, DMSO, ethyl acetate, acetic acid, and ethanolamine.

The following abbreviations are used herein and have the indicated definitions: Boc is N-(t-butoxycarbonyl), cit is citrulline, dap is dolaproine, DCM is dichloromethane, DIEA is N,N-diisopropylethylamine, dil is dolaisoleuine, DMF is N,N-dimethylformamide, DMSO is dimethylsulfoxide, doe is dolaphenine, dov is N,N-dimethylvaline, DTNB is 5,5′-dithiobis(2-nitrobenzoic acid), DTPA is diethylenetriaminepentaacetic acid, DTT is dithiothreitol, Fmoc is N-(9-fluorenylmethoxycarbonyl), gly is glycine, HATU is O-(7-azabenzotriazol-1-yl)-N,N,N′,N′-tetramethyluronium hexafluorophosphate, HBTU is 2-[1H-benzotriazole-1-yl]-1,1,3,3-tetramethylaminium hexafluorophosphate; HOBt is 1-hydroxybenzotriazole, HPLC is high pressure liquid chromatography, ile is isoleucine, lys is lysine, MeOH is methanol, MeVal is N-methyl-valine, PAB is p-aminobenzyl, PBS is phosphate-buffered saline (pH 7.4), Ph is phenyl, phe is L-phenylalanine, PyBrop is bromo tris-pyrrolidino phosphonium hexafluorophosphate, TFA is trifluoroacetic acid, UV is ultraviolet, and val is valine.

The following LU abbreviations are used herein and have the indicated definitions: Val Cit or vc is a valine-citrulline dipeptide site in protease cleavable linker; PABC is p-aminobenzylcarbamoyl; (Me)vc is N-methyl-valine citrulline, where the linker peptide bond has been modified to prevent its cleavage by cathepsin B; and MC(PEG)₆-OH is maleimidocaproyl-polyethylene glycol.

As used herein, a “pegylated compound” refers to a compound conjugated with two or more polyethylene glycol moieties or two or more polypropylene glycol moieties or a combination thereof.

As used herein, a “pro-peptide” includes pro-peptide, pre-peptide, pro-protein and pre-protein amino acid sequences including those amino acid sequences cleaved by enzymes disclosed in Table III.

Malignant neoplasia is the second most common cause of death in the United States behind cardiovascular disease. Chemotherapy has exerted a predominant role in increasing life spans for patients with a variety of tumors including Burkitt's lymphoma, acute lymphocytic leukemia and Hodgkin's disease. Further, new cancer chemotherapeutic agents and methods of care combined with early detection and treatment have resulted in decreases in the overall incidence of cancer and decreases in the death rates from all cancers combined. Responsive tumors represent only a small fraction of the various types of cancer. Further, agents such as cyclophosphamide, adriamycin, 5-fluorouracil and hexamethylmelamine, which are highly active against clinical solid tumors, are limited. Thus, patients with many types of malignancies remain at significant risk for relapse and mortality. After relapse, some patients can be re-induced into remission with their initial treatment regimen. However, higher doses of the initial chemotherapeutic agent or the use of additional agents are frequently required, indicating the development of at least partial medicant resistance. Evidence indicates medicant resistance can develop simultaneously to several agents, including medicant resistance to treatments to which the patient was not exposed. The development of multiple-medicant resistant tumors may be a function of tumor mass and constitutes a major cause of treatment failure. To overcome this medicant resistance, high-dose chemotherapy with or without radiation and allogenic or autologous bone marrow transplantation can be employed. The high-dose chemotherapy may employ the original medicant(s) or be altered to include additional agents. As a result, there remain many cancer patients for whom no or minimally effective therapy exists. Accordingly, there is a need for the development of novel chemotherapeutics with greater efficacy or safety, either as monotherapy or in combination with other chemotherapeutic agents, and such agents with the potential to overcome medicant resistance in cancer cells.

Illudins are toxic natural products produced by mushrooms of the genus Omphalotus. Syn-Illudins are semi-synthetic derivatives of Illudins. Acylfulvenes are also semi-synthetic derivatives of Illudins. Syn-Illudins and Acylfulvenes have each been chemically modified at select sites to allow their use as medicants. The modifications in the Syn-Illudins do not alter any of the cyclic rings (cyclopropane, cyclopentane, cyclohexane) of the basic Illudin chemical structure. The modifications of Acylfulvenes differ from Syn-Illudins in that an additional double bond (an unsaturated bond) has been created in the 5 membered (cyclopentane) ring.

Illudins function as alkylating agents that damage DNA and thereby block transcription. The blockage can be repaired through nucleotide excision. The toxicity of the illudins has prevented any applications in human tumor therapy. Acylfulvenes have been developed which exhibit promising antitumor activity with a better safety profile, as described in U.S. Pat. Nos. 5,439,936; 5,523,490 and 6,380,403 which are each herein expressly incorporated by reference in their entireties. (6′R)-6′-hydroxy-3′-(hydroxymethyl)-2′,4′,6′-trimethylspiro[cyclopropane-1,5′-inden]-7′(6′H)-one (analog 002, Table V) is an illudofulvene analog of illudin S which has demonstrated clinical activity with an acceptable safety profile in hormone-refractory prostate cancer. Most relevant to clinical applications, irofulven activity is independent of common resistance mechanisms such as the multi-medicant resistance phenotype, anti-apoptotic B-cell lymphoma 2 (Bcl-2) (SEQ. ID. 056) over expression, as well as tumor protein 53 (p53) (SEQ. ID. 057) and cyclin dependent kinase inhibitor 1 (p21/WAF1) (SEQ. ID. 058) mutations.

Growth factors, including peptides and proteins are critical mediators of a wide range of cell-cell communication. They are important endocrine, paracrine and autocrine messengers. Growth factors function as neurotransmitters and neuromodulators, regulate chemotaxis, immune function, development, cell growth, and can influence tumor cells. The receptors that recognize growth factors are highly selective and define specific cell populations. As a result, growth factor receptors are a large and important class of medicant (including drug) targets. In addition to physiologic noncancerous cell populations, these receptors can also be expressed in various cancer cell populations.

A polypeptide is a long, continuous, and unbranched chain of amino acids. A glycol-peptide is a peptide that contains one or more carbohydrate moieties covalently attached to the side chains of specific amino acids. A pro-peptide, is an inactive peptide that can be turned into an active form through a post translational modification that enzymatically cleaves the pro-peptide. Examples include pro-insulin (SEQ. ID. 059) and pro-opiomelanocortin (SEQ. ID. 060). Enzymatically cleaving the pro-peptide, allows for the peptide to be available on short notice and/or in large quantities. Some pro-peptides are secreted from the cell. Many of these are synthesized with an N-terminal signal peptide that targets the pro-peptide for secretion.

Cytokines are small proteins (approximately 5 to 20 kDa) that affect the behavior of other cells, and sometimes the releasing cell itself and are thereby important in cell signaling. Many specific cytokines can be released by a variety of different kinds of cells, e.g., macrophages, B lymphocytes, T lymphocytes, mast cells, endothelial cells, fibroblasts, and various stromal cells. Cytokines act through specific receptors, and are important in the humoral and cell-based immune responses. Cytokines also regulate the maturation, growth, and responsiveness of specific cell populations. Cytokines circulate in much higher concentrations than hormones and in contrast with hormones are made by a variety of different kinds of cells. Cytokines are important in host responses to infection, immune responses, inflammation, trauma, sepsis, cancer, and reproduction. As a result, cytokine receptors are upregulated in many forms of cancers.

A steroid is an organic compound that contains four cycloalkane rings joined to each other. Examples of steroids include the dietary lipid cholesterol and the sex hormones estradiol and testosterone. The core of a steroid molecule is composed of seventeen carbon atoms bonded together that take the form of four fused rings: three six-carbon atom rings and one five-carbon atom ring. A variety of functional groups can be attached to the four-ring core. Steroids can also vary depending on the oxidation state of the rings. A steroid hormone is a steroid that acts as a hormone. Steroid hormones can be grouped into five groups (glucocorticoids, mineralocorticoids, androgens, estrogens, and progesterones) based on the receptors to which they bind. Steroid hormones, particularly androgens, are essential not only for growth and development but also in the progression of many forms of cancer. As a result, steroid hormone receptors are upregulated in many forms of cancers.

The retinoic acid receptor (RAR) is a nuclear receptor which can also act as a transcription factor. The RAR can be activated by either all-trans retinoic acid or 9-cis retinoic acid. There are three RAR isoforms (alpha (SEQ. ID. 061), beta (SEQ. ID. 062), and gamma (SEQ. ID. 063)), each encoded by separate genes, where splice variants generate still further diversity in the expressed receptor. The retinoid X receptor (RXR) is a nuclear receptor activated by 9-cis retinoic acid. There are also three RXR isoforms (alpha (SEQ. ID. 064), beta (SEQ. ID. 065), and gamma (SEQ. ID. 066)), each encoded by separate genes. RXR hetero-dimerizes with subfamily 1 nuclear receptors including RAR. In the absence of ligand, the RAR/RXR dimer binds to retinoic acid response elements complexes with a co-repressor protein. Binding of agonist ligands to RAR results in dissociation of the co-repressor and recruitment of a co-activator protein that, in turn, promotes transcription of the downstream target gene into mRNA and thereby protein or other RNA signaling mechanisms.

Lipid metabolism is altered in many forms of cancer, including upregulation of de novo lipid synthesis. Cancer cells can also use alternative enzymes and pathways to facilitate the production of fatty acids. These newly synthesized lipids may support a number of cellular processes to promote cancer cell proliferation and survival. Elaidic acid or (E)-octadec-9-enoic acid is the trans isomer of oleic acid and is found in small quantities in caprine milk, bovine milk and some meats. It increases Cholesteryl Ester Transfer Protein (CETP) (SEQ. ID. 067) activity, which in turn raises levels of very low density lipoprotein and lowers levels of high density lipoprotein (HDL) cholesterol. CETP is found in plasma, where it is involved in the transfer of cholesteryl ester from HDL to other lipoproteins. Defects in the CETP gene are a cause of hyperalphalipoproteinemia 1.

An antibody is a protein made up of four peptide chains disulfide linked together to form a “Y”-shape. Antibodies are produced by plasma cells and are used by the immune system to identify and neutralize foreign antigens such as bacteria and viruses. The antibody recognizes a unique part of the antigen using each FAB portion of the protein (i.e., the tip of the “Y” portion of the antibody), allowing a specific high affinity binding interaction to occur. The binding interaction of different antibodies can target specific antigen epitopes. An antibody fragment containing one or both FAB portions can also target specific antigen epitopes.

The ability of the Illudofulvene analogs to inhibit tumor cell growth is shown in Table VII and Table VIII. Table VII shows the ability of Illudofulvene analogs to inhibit tumor cell growth. Table VIII shows screening data with the National Cancer Institute (NCI) Developmental Therapeutics Program (DTP) NCI 60 cell line screen assay comparing conventional anti cancer drugs Epothilone A, MMA,DM1 with the previously reported illudofulvene analogs (analog 002, analog 142, analog 159, analog 176) and with the illudofulvene analogs (analog 334 (FIG. 1AE), analog 362 (FIG. 1AU), analog 371 (FIG. 1BC), analog 383 (FIG. 1BM), and analog 394 (FIG. 1AE)). In Table VIII, ‘Cytotoxicity’ is defined as the ability to actually kill tumor cells was detected versus only inhibiting growth of tumor cells, while ‘MDR activity’ is the ability to equally kill a drug resistant daughter cell as compared to the non-drug resistant parent cell. The conventional anticancer drugs, Epothilone A, MMA and DM1 were able to inhibit the growth of cells in the NCI 60 cell line screen but were not able to actually kill tumor cells. That is they were not cytotoxic. Further, they were not MDR active. All of the illudofulvene analogs in Table VIII (analog 334, analog 362, analog 371, analog 383 and analog 394) were cytotoxic in the NCI 60 cell line assay. The illudofulvene analog 371 is the most potent compound at lysing the NCI 60 tumor cell line. The MV522 cell line is a lung-derived adenocarcinoma cell line. In various embodiments of the invention, the MV522 cell line represents a “target” cell line, That is an illudofulvene analog that exhibits toxicity against this solid tumor cell line shows a desirable result. The 839213 cell line represents a hematopoietic (non-solid) cell line. In various embodiments of the invention, the 8392B cell line is considered a “nontarget” cell line. The two hour toxicity data represents the concentration of a given analog for which a two hour exposure will inhibit 50% of the DNA synthesis activity in a given cell line. The 48 hour exposure data represents the concentration at which a given analog with a 48 hour exposure will inhibit the growth or viability in a given cell line as defined by the standard Trypan Blue Exclusion assay. As an example, analog 002 will inhibit the target MV522 cell line at 110 nM with only a 2 hour exposure but has no inhibitory effect on the nontarget 8392B cell line at 26,000 nM (26 pEM). Analog 002 with a prolonged exposure period (e.g. 48 hours) can eventually inhibit the nontarget cell line. In contrast, Analog 201 will inhibit the target MV522 cell line with only a 2 hour exposure (IC50=360 nM) but has minimal effect on the 8392B cell nontarget line with even a 48 hour exposure (IC50=26,000 nM) indicating superior anticancer activity as a monotherapeutic agent. In contrast to these two analogs, analog 224 displayed minimal toxicity as well as no differential toxicity between the target and nontarget cell line indicating it would have minimal properties as a monotherapeutic anticancer agent.

As used herein, a “growth factor” or an “anti-angiogenic protein” includes Adrenomedullin (SEQ. ID. 068), Angiopoietin (Ang) (SEQ. ID. 069, 106, 111, and 145), Autocrine motility factor (SEQ. ID. 070), Bone morphogenetic proteins (BMPs) (SEQ. ID. 071), Brain-derived neurotrophic factor (BDNF) (SEQ. ID. 072), Endostatin (SEQ. ID. 073), Endostar (SEQ. ID. 074), Epidermal growth factor (EGF) (SEQ. ID. 075), Erythropoietin (EPO) (SEQ. ID. 076), Fibroblast growth factor (FGF) (SEQ. ID. 077), Glial cell line-derived neurotrophic factor (GDNF) (SEQ. ID. 078), Granulocyte colony-stimulating factor (G-CSF) (SEQ. ID. 079), Granulocyte macrophage colony-stimulating factor (GM-CSF) (SEQ. ID. 080), Growth differentiation factor-9 (GDF9) (SEQ. ID. 081), Hepatocyte growth factor (HGF) (SEQ. ID. 082), Hepatoma-derived growth factor (HDGF) (SEQ. ID. 083), Insulin-like growth factor (IGF) (SEQ. ID. 084), Migration-stimulating factor (SEQ. ID. 085), Myostatin (GDF-8) (SEQ. ID. 086), Nerve growth factor (NGF) (SEQ. ID. 087) and other neurotrophins (SEQ. ID. 144), Platelet-derived growth factor (PDGF A) (SEQ. ID. 088), PDGF B (SEQ. ID 168), PDGF C (SEQ. ID. 036), PDGF D (SEQ. ID. 037), Thrombopoietin (TPO) (SEQ. ID. 089), Transforming growth factor alpha (TGF-α) (SEQ. ID. 090), Transforming growth factor beta (TGF-β) (SEQ. ID. 091), Tumor necrosis factor-alpha (TNF-α) (SEQ. ID. 092), Vascular endothelial growth factor (VEGF) (SEQ. ID. 093), and placental growth factor (PlGF) (SEQ. ID. 094).

As used herein, a “protein toxin” includes ricin A chain (SEQ. ID. 095), ricin B chain (SEQ. ID. 096), diphtheria toxin (SEQ. ID. 097), Pseudomonas aeurginosa exotoxin A (SEQ. ID. 098), r-gelonin (SEQ. ID. 099), saporin (SEQ. ID. 100), glycosylated protein toxins, deglcosylated protein toxins and protein toxin fragments which includes deglycosylated ricin A, deglycosylated ricin B, Pseudomonas aeurginosa exotoxin A PE40 fragment (SEQ. ID. 101) and Pseudomonas aeurginosa exotoxin A PE38 fragment (SEQ. ID. 102).

As used herein, a “steroid” includes cholesterol (5-cholesten-3beta-ol), pregnenolone (3beta-hydroxy-5-pregnen-20-one), 17-hydroxyprenenolone (3-beta,17-dihydroxy-5-pregnen-20-one), progesterone (4-pregnene-3,20-dione), 17-hydroxyprogesterone (17-hydroxy-4-pregnene-3,20-dione), androstenedione (4-androstene-3,17-dione), 4-hydroxyandrostenedione (4-hydroxy-4-androstene-3,17-dione), 11-beta-hydroxyandostenedione (11beta-4-androstene-3,17-dione), androstanediol (3-beta,17-beta-Androstanediol), androsterone (3-alpha-hydroxy-5alpha-androstan-17-one), epiandrosterone (3-beta-hydroxy-5alpha-androstan-17-one), adrenosterone (4-androstene-3,11,17-trione), dehydroepiandrosterone (3beta-hydroxy-5-androsten-17-one), dehydroepiandrosterone sulfate (3-beta-sulfooxy-5-androsten-17-one), testosterone (17beta-hydroxy-4-androsten-3-one), epitestosterone (17-alpha-hydroxy-4-androsten-3-one), 5-alpha-dihydrotesterone (17-beta-hydroxy-5alpha-androstan-3-one), 5-beta-dihydrotestosterone (17-beta-hydroxy-5beta-androstan-3-one), 11-beta-hydroxytesosterone (11-beta,17beta-dihydroxy-4-androsten-3-one), 11-ketotesosterone (17-beta-hydroxy-4-androsten-3,17-dione), estrogen (including: estrone (3-hydroxy-1,3,5(10)-estratrien-17-one), estradiol (1,3,5(10)-estratriene-3,17beta-diol), and estriol (1,3,5(10)-estratriene-3,16alpha,17beta-triol)), corticosterone (11-beta,21-dihydroxy-4-pregnene-3,20-dione), deoxycorticosterone (21-hydroxy-4-pregnene-3,20-dione), cortisol (11-beta,17,21-trihydroxy-4-pregnene-3,20-dione), 11-deoxycortisol (17,21-dihydroxy-4-pregnene-3,20-dione), cortisone (17,21-dihydroxy-4-pregnene-3,11,20-trione), 18-hydroxycorticosterone (11-beta,18,21-trihydroxy-4-pregnene-3,20-dione), 1-alpha-hydroxycorticosterone (1-alpha,11-beta,21-trihydroxy-4-pregnene-3,20-dione), and aldosterone (18,11-hemiacetal of 11beta,21-dihydroxy-3,20-dioxo-4-pregnen-18-al).

As used herein, a “Specific Binding Peptide” includes an “anti-angiogenic peptide” (SEQ. ID. 146) and an “integrin binding peptide” (SEQ. ID. 147). A “Specific Binding Peptide” includes integrin binding peptide RGD4C=CDCRGDFC (SEQ. ID. 147), integrin binding peptide RGD10 (SEQ. ID. 148), c(RGDyK) (SEQ. ID. 149), integrin binding peptide c(RGDfK) (SEQ. ID. 150), integrin binding peptide [c(RGDyK)]2 (SEQ. ID. 151), integrin binding peptide CAGKNFFWKTFTSC (SEQ. ID. 152), cilengitide (cyclic RGD pentapeptide) (SEQ. ID. 153), ATN-161 (peptide antagonist of integrin alpha5beta1) (SEQ. ID. 154), ATN-454 (Ac-PHSCN-NH₂) (peptide antagonist of integrin alpha5beta1) (SEQ. ID. 155), tumstatin T7 peptide TMPFLFCNVNDVCNFASRNDYSYWL (SEQ. ID. 156), tumstatin sequence 1 YSNS (SEQ. ID. 157), tumstatin sequence 2 YSNSG (SEQ. ID. 158), endostatin motif FLSSRLQDLYSIVRRADRAA (SEQ. ID. 159), endostatin motif IVRRADRAAVP (SEQ. ID. 160), laminin peptide A13 (RQVFQVAYIIIKA) (SEQ. ID. 161), laminin peptide C16 (KAFDITYVRLKF) (SEQ. ID. 162), laminin peptide C16S (DFKLFAVTIKYR) (SEQ. ID. 163), and VEGFR1 peptide (CPQPRPLC) (SEQ. ID. 164).

As used herein, a traditional linker includes linkers that can be formed from those reagents disclosed in Tables IA-ID, IIA-IID, IIIA-IIIC, IVA-IVC, VA-VB, and VIA-VID of U.S. Pat. No. 9,381,178.

As used herein, a “FSB linker” includes those linkers selected from the group consisting of 4-fluorosulfonyl benzoyl, 3-fluorosulfonyl benzoyl and 2-fluorosulfonyl benzoyl as depicted in FIG. 15 of U.S. Pat. No. 9,381,178.

As used herein, a “Mal1” linker includes a malonic linker and a maleimide linker covalently attached to an illudofulvene analog.

As used herein, a “protease” includes those enzymes disclosed in Table III.

As used herein, a “cytokine” includes chemokines, interferons, interleukins, lymphokines, tumor necrosis factor, neutrophil activating protein-2, monocyte chemotactic protein-1 and the like.

Despite recent advances in therapy, many patients with cancer invariably relapse and require additional treatments. Most of these patient's cancers become refractory to standard chemotherapy and/or radiation treatment regimens. The prognosis for these patients is poor and long term survival rates for metastatic solid tumor cancers remain very low. Thus, there is a need for the development of novel agents and treatment regimens that specifically target these recurring tumor cells and also produce less systemic toxicity. Target therapies, such as monoclonal antibodies, now provide a promising alternative to the conventional cytotoxic chemotherapy approach.

Monoclonal antibody based therapy has recently achieved considerable success in oncology and there are currently nine monoclonal antibodies (without a medicant attached) approved by the FDA as cancer therapeutics. As an example, HERCEPTIN® and RITUXAN® (both produced by Genentech, South San Francisco, California), are used to successfully treat breast cancer and non-Hodgkin's lymphoma, respectively. HERCEPTIN® is a recombinant DNA-derived humanized monoclonal antibody selectively binding to the extracellular domain of the Human Epidermal growth factor Receptor 2 (HER2) proto-oncogene whereas RITUXAN® is a genetically engineered chimeric murine/human monoclonal antibody directed against the CD20 antigen overexpressed on the surface of normal and malignant B lymphocytes.

Recent clinical evidence indicates that while the monoclonal antibody based therapies are effective at inducing remission, they do not always produce a complete cure, and relapses eventually occur in most patients. There is now a tremendous interest in the use of antibody medicant conjugates as a class of therapeutics that utilize the antigen-selectivity of monoclonal antibodies to deliver potent cytotoxic medicants to specific tumor cells. Antibody medicant conjugates are produced by attaching a cytotoxic agent to an antibody that binds specifically to a tumor-associated antigen.

In theory, antibody medicant conjugates can confer an increased therapeutic index to highly potent medicants by improving therapeutic efficacy and reducing systemic toxicity (by minimizing damage to normal tissues), although this goal has been elusive in achieving. The basis for the efficacy of antibody medicant conjugates is that they target tumor cells that preferentially express an antigen that is recognized by the associated antibody. In contrast, non-tumor cells either fail to express this antigen, or express the antigen at a very low level. In theory, only the tumor cells expressing the associated antibody are recognized and destroyed by the AMC, and other cells are left untouched and undamaged.

While different medicant classes have been tried for delivery via antibodies, only a few have proved efficacious for use as antibody medicant conjugates. The two main medicant classes used to date to produce antibody medicant conjugates are the auristatins (MMAE/N-methylvaline-valine-dolaisoleuine-dolaproine-norephedrine or MMAF/N-methylvaline-valine-dolaisoleuine-dolaproine-phenylalanine) and the maytansines (DM1 or DM4). Currently only two antibody medicant conjugates are approved by the U.S.F.D.A. and marketed; brentuximab vedotin (auristatin based) and ado-trastuzumab emtansine (maytansine based).

Illudofulvenes have several unique properties over agents traditional used to make medicants and/or antibody drug conjugates (ADCs). Firstly, these are the only agents known to function by inhibition of the DNA transcription-coupled repair pathway, see e.g., U.S. Pat. No. 10,285,955 entitled AFFINITY MEDICANT CONJUGATES, issued May 14, 2019, which is herein incorporated by reference inits entirety and for all purposes. No other toxin, drug or medicant inhibits this pathway. The result is that illudofulvenes are true cytotoxic agents whereas other agents traditionally used to produce ADCs (pyrrolobenzodiazepines, maytansines, fumagillols, dolstatins, auristatins, enadiynes, halichondrins, and tubulysins) are only cytostatic. In the NCI-DTP 60 cell line panel these other agents were capable of inhibiting tumor cell growth (IC₅₀ value), had some ability to block tumor cell growth (TGI value) but none were capable of actually causing tumor cell death or cytotoxicity (Table VI). The illudin derivatives, however, are capable of killing tumor cells at nanomolar concentrations (Table VI). This means that while ADCs developed using other toxins can stall tumor cell growth, they cannot actually kill the tumor cell. Once the effect of the drug has worn off the tumor cells will again grow and kill the patient. In contrast, the illudofulvenes actually kill the tumor cell with as little as a 2 hour exposure. Secondly, whereas tumor cells will undergo apoptosis or cell death with hours once the DNA transcription-coupled repair pathway is blocked, normal diploid non-tumor cells can survive for hours. This translates into a wide therapeutic window for ADCs developed with illudofulvenes. The two ADC agents currently FDA approved for administration deliver a dose of the associated toxin that is 300% higher than a lethal dose which is why these agents have severe systemic toxicity. In contrast, the comparable ADC developed with illudofulvenes will deliver a dose of the associated toxin that is 40% of a known non-toxic dose (estimated at 28% of a toxic dose and only 12% of a lethal dose). Thus, ADCs developed with illudofulvenes will have minimal systemic toxicity as compared to current agents. Thirdly, these agents are stable down to a pH of 2.0. An ADC is engulfed by a tumor cell, transported to the endosomes (pH<6.0) and then into the lysozomes (pH<4). Many agents used for ADCs will degrade in these low pH environment, whereas illudofulvenes are stable. 4). Cancer cells can become resistant to various toxins and drugs through the development of what is termed multi-drug resistance. This process is known to occur through several different mechanisms. Whereas other toxins and drugs are substrates for the most common MDR mechanisms (MDR1/gp170 and MRP/gp180), and cancer cells can become resistant to these agents, the illudofulvenes remain active against all MDR phenotypes regardless of the mechanism (see Table IV). Hence, if tumor cells have already developed multi-drug resistance prior to ADC with a conventional toxin, or during the administration of a course of the ADC, the ADC will have no efficacy. In contrast, ADCs developed with illudofulvenes will continue to kill cancer cells. Illudofulvenes as stand alone treatments or in ADCs can be coupled with a screen to select a patient population that will respond to the illudofulvenes, see e.g., U.S. patent application Ser. No. 16/708,005 entitled METHODS, COMPOSITIONS AND DEVICES FOR TREATING CANCER WITH ILLUDOFULVENES, filed Dec. 9, 2020, which is herein incorporated by reference inits entirety and for all purposes

The present invention is based on the discovery that illudofulvene are active as medicant delivery agents in vitro and in vivo and can be conjugated directly to a linker, via a variety of peptide or non-peptide bonds, and are active as medicant delivery agents in vitro and in vivo. Similar to other medicant classes used to produce antibody medicant conjugates, the illudofulvenes can be conjugated to a linker that allows subsequent coupling to a monoclonal antibody. Unlike previous medicant classes such as the auristatins (MMAE, MMAF, dolstatin-10), the maytansines (DM1 or DM4), the irinotecans and their metabolites (SN38), the calicheamicins (17-DMAG), the pyrrolobenzodiazepines (SJG-136), the duocarmycins (CC-1065), many of the illudofulvenes compounds do not require a linker and can be directly attached to a monoclonal antibody or fragment thereof by a variety of simple chemical reactions. In this sense, the lack of requirement for a linker or a spacer, the illudofulvenes compounds are unique. They will directly form covalent bonds with reactive groups on an AM such as a monoclonal antibody. In addition, because of their very small size and extreme cytotoxicity the illudofulvenes can be coupled directly to very small molecular weight entities (or affinity moieties) that allow tumor specific cytotoxicity without the concomitant requirement of use of a monoclonal antibody. Examples include the ability to link illudofulvenes directly to steroids which allow the medicant-affinity complex to kill cells overexpressing a specific steroid receptor (such as estrogen- or progesterone-positive breast cancer cells) or even to be chemically coupled to various lipids. The small size and extreme cytotoxicity illudofulvenes allows direct coupling to peptides which can preferentially bind to tumor cells (integrin binding peptides) or display anti-angiogenic properties to hinder tumor invasion. The illudofulvenes can also be coupled to specific peptides which actually renders the medicant-affinity complex non-toxic until the peptide is cleaved by a protease secreted by tumor cells. An example includes PSA (prostate specific antigen) secreted by prostate adenocarcinoma cells. Again, unlike previous medicant classes such as the auristatins (MMAE, MMAF, dolstatin-10), the maytansines (DM1 or DM4), the irinotecans and their metabolites (SN38), the calicheamicins (17-DMAG), the pyrrolobenzodiazepines (SJG-136), the duocarmycins (CC-1065), the illudofulvenes compounds do not require a linker and can be directly attached to a steroid or a peptide that will subsequently function as an AM and direct the associated complex to specific tumor cells. In an embodiment of the invention, an illudofulvenes is attached to either a Specific Binding Peptide or a peptide which when cleaved by a specific protease (see Table III) such as PSA generates an entity which is cytotoxic (see Table II).

Trastuzumab emtansine (Genentech for Breast cancer) uses maytanasine derive DM-1, a stable non-cleavable linker. Brentuximab vedotin (Seattle Genetics/Takeda for Hodgkin's Lymphoma) uses auristatin MMAE to anti-CD30, an enzyme sensitive cleavable linker.

The malonic linker, maleimide linker and SMCC [succinimidyl 4-(N-maleimidomethyl)-cyclohexane-1-carboxylate]linker can form active intermediates that react with sulfhydryl groups on an antibody. SMCC has been used to bind maytansine derivative DM1 to the monoclonal antibody Herceptin. The AMC was internalized where the Herceptin was degraded by proteases and DM1 was released into the cytosol. Further, Sulfo-SMCC [sulfosuccinimidyloxycarbonyl-α-methyl-α-(2-pyridyldithio)toluene]forms an active intermediate that reacts with sulfhydryl groups on an antibody. The resulting Sulfo-SMCC AMC is more water soluble than the SMCC AMC.

Compounds and Conjugates. The present invention is drawn to a series of compounds and conjugates containing a Medicant moiety (M) linked via its C terminus to a LU (LU). The LU can operate to provide a suitable release of M.

In one group of embodiments, the invention provides Medicant Linker compounds having Formula I: LU-M (I) or a pharmaceutically acceptable salt or solvate thereof where the medicant loading is represented by p, the average number of medicant molecules per affinity (e.g., an antibody) (e.g. of Formula II, IIa, IIa′). Medicant loading may range from 1 to 20 Medicant units (M) per Affinity unit (e.g., Ab or in Ab). Compositions of Formula IIa and Formula IIa′ include mixtures of antibodies conjugated with a range of medicants, from 1 to 20.

In some embodiments, p is from about 1 to about 8 Medicant units per Affinity unit. In some embodiments, p is 1. In some embodiments, p is from about 2 to about 8 Medicant units per Affinity unit. In some embodiments, p is from about 2 to about 6, 2 to about 5, or 2 to about 4 Medicant units per LU. In some embodiments, p is about 2, about 4, about 6 or about 8 Medicant units per Affinity unit.

The average number of Medicants units per Affinity unit in a preparation from a conjugation reaction may be characterized by conventional means such as mass spectroscopy, ELISA assay, and HPLC. The quantitative distribution of Affinity Medicant Linker conjugates in terms of p may also be determined. In some instances, separation, purification, and characterization of homogeneous Affinity Medicant Linker conjugates, where p is a certain value from Affinity Medicant Linker conjugates with other medicant loadings may be achieved by means such as reverse phase HPLC or electrophoresis.

Returning to Formula IIa′, the conjugates comprise an antibody covalently attached to one or more Medicant units (moieties) via a LU: A, a, W and w are as described above. The antibody medicant conjugate include pharmaceutically acceptable salts or solvates thereof.

The medicant loading is represented by p, the average number of Medicant units per antibody in a molecule of Formula II. Medicant loading may range from 1 to 20 medicants (M) per Ab or mAb. Compositions of the AMC of Formula IIa′ include mixtures of antibodies conjugated with a range of medicants, from 1 to 20. In some embodiments, p is from about 1 to about 8 Medicant units per antibody. In some embodiments, p is 1. In some embodiments, p is from about 2 to about 8 Medicant units per antibody. In some embodiments, p is from about 2 to about 6, 2 to about 5, or 2 to about 4 Medicant units per antibody. In some embodiments, p is about 2, about 4, about 6 or about 8 Medicant units per antibody.

The average number of medicants per antibody in preparations of AMCs from conjugation reactions may be characterized by conventional means such as UV/visible spectroscopy, mass spectrometry, ELISA assay, and HPLC. The quantitative distribution of AMCs in terms of p may also be determined. In some instances, separation, purification, and characterization of homogeneous AMCs where p is a certain value from AMC with other medicant loadings may be achieved by means such as reverse phase HPLC or electrophoresis.

For some antibody medicant conjugates, p may be limited by the number of attachment sites on the antibody. For example, where the attachment is a cysteine thiol, an antibody may have only one or several cysteine thiol groups, or may have only one or several sufficiently reactive thiol groups through which a LU may be attached. In some embodiments, the cysteine thiol is a thiol group of a cysteine residue that forms an interchain disulfide bond. In some embodiments, the cysteine thiol is a thiol group of a cysteine residue that does not form an interchain disulfide bond.

Typically, less than the theoretical maximums of medicant moieties are conjugated to an antibody during a conjugation reaction. An antibody may contain, for example, many lysine residues that do not react with the Medicant Linker compound intermediate or LU reagent. Only the most reactive lysine groups may react with an amine-reactive LU reagent. Generally, antibodies do not contain many, if any, free and reactive cysteine thiol groups which may be linked to a Medicant moiety via a LU. Most cysteine thiol residues in the antibodies exist as disulfide bridges and must be reduced with a reducing agent such as dithiothreitol (DTT). The antibody may be subjected to denaturing conditions to reveal reactive nucleophilic groups such as lysine or cysteine. The loading (medicant/antibody ratio) of an AMC may be controlled in several different manners, including: (i) limiting the molar excess of Medicant Linker compound intermediate or LU reagent relative to antibody, (ii) limiting the conjugation reaction time or temperature, and (iii) partial or limiting reductive conditions for cysteine thiol modification.

Where more than one nucleophilic group reacts with a Medicant Linker compound intermediate, or LU reagent followed by Medicant moiety reagent, then the resulting product is a mixture of Affinity Medicant Linker Conjugates (e.g., AMCs) with a distribution of one or more Medicant moieties per Affinity unit (e.g., an antibody). The average number of medicants per Affinity unit (e.g., antibody) may be calculated from the mixture by, for example, dual enzyme linked immune serum assay (ELISA) antibody assay, specific for antibody and specific for the medicant. Individual Affinity Medicant Linker Conjugate molecules may be identified in the mixture by mass spectroscopy, and separated by high performance liquid chromatography (HPLC), e.g., hydrophobic interaction chromatography. Thus, a homogeneous conjugate with a single loading value may be isolated from the conjugation mixture by electrophoresis or chromatography.

A “Linker Unit” (LU) is a bifunctional compound which can be used to link a Medicant unit and/or an Affinity unit to form an Affinity Medicant Linker conjugate. Such conjugates are useful, for example, in the formation of immuno conjugates directed against tumor associated antigens. Such conjugates allow the selective delivery of cytotoxic drugs to tumor cells. A LU includes a traditional linker, a 4-fluorosulfonyl benzoyl (4-FSB) linker, a 3-fluorosulfonyl benzoyl (3-FSB) linker a 2-fluorosulfonyl benzoyl (2-FSB) linker, a maleimide (Mal1) linker, an azlactone linker and a bridging amino acid.

A traditional linker is as defined in U.S. Pat. No. 9,381,178. A Stretcher Unit includes two or more Linker Units.

A bridging amino acid means —NH—C(R′)H—CO— or —N(R″)—C(R′)H—CO— including glycine, L-alanine, L-serine, L-threonine, L-cysteine, L-valine, L-leucine, L-isoleucine, L-methionine, L-proline, L-phenylalanine, L-tyrosine, L-tryptophan, L-aspartic acid, L-glutamic acid, L-asparagine, L-glutamine, L-histidine, L-lysine, L-arginine, L-homocysteine, L-selenocysteine, L-pyrrolysine, L-carnitine, L-hypusine, 2-aminoisobutyric acid, dehydroalanine, L-gamma-aminobutyric acid, L-ornithine, L-citrulline, L-α-Amino-n-butyric acid, L-Norvaline, L-Norleucine, L-Pipecolic acid, L-Alloisoleucine, L-α,β-diaminopropionic acid, L-α,γ-diaminobutyric acid, L-Allothreonine, L-α-Amino-n-heptanoic acid, L-Homoserine, β-Amino-n-butyric acid, β-Aminoisobutyric acid, γ-Aminobutyric acid, L-isovaline, L-Sarcosine, N-ethyl glycine, N-propyl glycine, N-isopropyl glycine, L-N-methyl alanine, L-N-ethyl alanine, N-methyl β-alanine, N-ethyl β-alanine, Isoserine, L-α-hydroxy-γ-aminobutyric acid, L-diaminopimelic acid, cystathione, L-aminoisobutyric acid, dehydroalanine, delta-aminolevulinic acid, 4-aminobenzoic acid, L-Hydroxyproline, Formylmethioinine, L-lanthionine, djenkolic acid, L-Pyroglutamic acid, Hypusine, L-carboxyglutamic acid, penicillamine, L-thialysine, quisqualic acid, L-canavine, L-azetidine-2-carboxylic acid, D-alanine, D-serine, D-threonine, D-cysteine, D-valine, D-leucine, D-isoleucine, D-methionine, D-proline, D-phenylalanine, D-tyrosine, D-tryptophan, D-aspartic acid, D-glutamic acid, D-asparagine, D-glutamine, D-histidine, D-lysine, D-arginine, D-homocysteine, D-selenocysteine, D-pyrrolysine, D-carnitine, D-hypusine, D-gamma-aminobutyric acid, D-ornithine, D-citrulline, D-α-Amino-n-butyric acid, D-Norvaline, D-Norleucine, D-Pipecolic acid, D-Alloisoleucine, D-α,β-diaminopropionic acid, D-α,γ-diaminobutyric acid, D-Allothreonine, D-α-Amino-n-heptanoic acid, D-Homoserine, D-isovaline, D-Sarcosine, D-N-methyl alanine, D-N-ethyl alanine, D-α-hydroxy-γ-aminobutyric acid, D-diaminopimelic acid, D-aminoisobutyric acid, D-Hydroxyproline, D-lanthionine, D-Pyroglutamic acid, D-carboxyglutamic acid, D-thialysine, quisqualic acid, D-canavine, D-azetidine-2-carboxylic acid. A ‘modified bridging amino acid’ means a bridging amino acid with R′ including a hydroxyl group that has been esterified, a bridging amino acid with R′ including a sulphur atom where the sulphur atom has been reacted with an alkyl or other organic group and/or a bridging amino acid with R′ including a primary amino group that has been converted into a secondary or tertiary amino group.

In one embodiment, the LU of the Medicant Linker compound and Affinity Medicant Linker conjugate has the formula: —W_{w}-Aₐ wherein -A- is a Stretcher Unit; a is 1 or 2; each —W— is independently an Amino Acid unit; w is independently an integer ranging from 1 to 20. In the Affinity Medicant Linker conjugate, the LU serves to attach the Medicant moiety and the AM.

The Affinity Moiety (AM) includes within its scope an Affinity Unit (AU) that specifically binds or reactively associates or complexes with a receptor, antigen or other receptive moiety associated with a given target-cell population. An AU is a molecule that binds to, complexes with, or reacts with a receptor, antigen or other receptive moiety of a cell population sought to be therapeutically or otherwise biologically modified. In one aspect, the AM acts to deliver the Medicant unit to the particular target cell population with which the AM interacts. Such AM's include, but are not limited to, proteins, polypeptides and peptides and include, antibodies, binding proteins, smaller molecular weight proteins, polypeptides, peptides, lectins, glycoproteins, non-peptides, vitamins, nutrient-transport molecules (such as, but not limited to, transferrin), or any other cell binding molecule or substance.

In an embodiment of the invention, an AM can form a bond to a Stretcher Unit. In an alternative embodiment of the invention, an AM can form a bond to the Stretcher Unit of the LU via a heteroatom of the AM. Heteroatoms that may be present on an AM include sulfur (in one embodiment, from a sulfhydryl group of an AM), oxygen (in one embodiment, from a carbonyl, carboxyl or hydroxyl group of an AM) and nitrogen (in one embodiment, from a primary or secondary amino group of an AM). These hetero atoms can be present on the AM in the AM's natural state, for example a naturally-occurring antibody, or can be introduced into the AM via chemical modification.

In one embodiment, an AM unit has a sulfhydryl group and the AM bonds to the LU via the sulfhydryl group's sulfur atom. In another embodiment, the AM has lysine residues that can react with activated esters (such esters include, but are not limited to, N-hydroxysuccinimide, pentafluorophenyl, and p-nitrophenyl esters) of the Stretcher Unit of the AM and thus form an amide bond consisting of the primary nitrogen atom of the AM and the carboxyl group of the AM. In yet another aspect, the AM has one or more lysine residues that can be chemically modified to introduce one or more sulfhydryl groups. The AM bonds to the LU via the sulfhydryl group's sulfur atom. The reagents that can be used to modify lysines include, but are not limited to, N-succinimidyl S-acetylthioacetate (SATA) and 2-Iminothiolane hydrochloride (Traut's Reagent).

In another embodiment, the AM can have one or more carbohydrate groups that can be chemically modified to have one or more sulfhydryl groups. The AM bonds to the LU (or a Stretcher Unit) via the sulfhydryl group's sulfur atom. In yet another embodiment, the AM can have one or more carbohydrate groups that can be oxidized to provide an aldehyde (—CHO) group. The corresponding aldehyde can form a bond with a reactive site on a Stretcher Unit. Reactive sites on a Stretcher Unit that can react with a carbonyl group on an AM include, but are not limited to, hydrazine and hydroxylamine.

Useful non-immunoreactive protein, polypeptide, or peptide affinity moieties include, but are not limited to, transferrin, epidermal growth factors (“EGF”), bombesin, gastrin, gastrin-releasing peptide, platelet-derived growth factor, IL-2, IL-6, transforming growth factors (“TOP”), such as TGF-.alpha. and TGF-.beta., vaccinia growth factor (“VGF”), insulin and insulin-like growth factors I and II, somatostatin, lectins and apoprotein from low density lipoprotein.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, antibody fragments, or chimeric monoclonal antibodies. Human monoclonal antibodies may be made by any of numerous techniques known in the art.

The antibody can also be a bispecific antibody. Methods for making bispecific antibodies are known in the art and are discussed infra.

The antibody can be a functionally active fragment, derivative or analog of an antibody that immunospecifically binds to target cells (e.g., cancer cell antigens, viral antigens, or microbial antigens) or other antibodies that bind to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary embodiment the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIA core assay).

Other useful antibodies include fragments of antibodies such as, but not limited to, F(ab′)₂ fragments, Fab fragments, Fvs, single chain antibodies, diabodies, triabodies, tetrabodies, scFv, scFv-FV, or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies are particularly desirable and can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. Other human antibodies can be obtained commercially from, for example, Abgenix, Inc. (now Amgen, Freemont, Calif.) and Medarex (Princeton, N.J.).

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. Human antibodies can also be produced using various techniques known in the art, including phage display libraries.

In other embodiments, the antibody is a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, preferably at least 10, 20 or 50 amino acid portion of the protein) that is not from an antibody. Preferably, the antibody or fragment thereof is covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor.

Antibodies immunospecific for a cancer cell antigen can be obtained commercially or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, literature publications, or by routine cloning and sequencing.

In a specific embodiment, known antibodies for the treatment of cancer can be used. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, RITUXAN® (rituximab; Genentech) which is a chimeric anti-CD20 monoclonal antibody for the treatment of patients with non-Hodgkin's lymphoma; OVAREX which is a murine antibody for the treatment of ovarian cancer; PANOREX (Glaxo Wellcome, N.C.) which is a murine IgG₂ₐ antibody for the treatment of colorectal cancer; Cetuximab ERBITUX (Imclone Systems Inc., NY) which is an anti-EGFR IgG chimeric antibody for the treatment of epidermal growth factor positive cancers, such as head and neck cancer; Vitaxin (MedImmune, Inc., MD) which is a humanized antibody for the treatment of sarcoma; CAMPATH I/H (Leukosite, MA) which is a humanized IgG₁ antibody for the treatment of chronic lymphocytic leukemia (CLL); SMART MI95 (Protein Design Labs, Inc., CA) and SGN-33 (Seattle Genetics, Inc., WA) which is a humanized anti-CD33 IgG antibody for the treatment of acute myeloid leukemia (AML); LYMPHOCIDE (Immunomedics, Inc., NJ) which is a humanized anti-CD22 IgG antibody for the treatment of non-Hodgkin's lymphoma; SMART ID10 (Protein Design Labs, Inc., CA) which is a humanized anti-HLA-DR antibody for the treatment of non-Hodgkin's lymphoma; ONCOLYM (Techniclone, Inc., CA) which is a radiolabeled murine anti-HLA-Dr10 antibody for the treatment of non-Hodgkin's lymphoma; ALLOMUNE (BioTransplant, CA) which is a humanized anti-CD2 mAb for the treatment of Hodgkin's Disease or non-Hodgkin's lymphoma; AVASTIN (Genentech, Inc., CA) which is an anti-VEGF humanized antibody for the treatment of lung and colorectal cancers; Epratuzamab (Immunomedics, Inc., NJ and Amgen, Calif.) which is an anti-CD22 antibody for the treatment of non-Hodgkin's lymphoma; and CEACIDE (Immunomedics, N.J.) which is a humanized anti-CEA antibody for the treatment of colorectal cancer.

Other antibodies useful in the treatment of cancer include, but are not limited to, antibodies against the following antigens (where exemplary cancers that can be treated with the antibody are in parentheses): Alk (adrenocarcinomas) (SEQ. ID. 103), CA125 (ovarian) (SEQ. ID. 104), CA15-3 (carcinomas) (SEQ. ID. 105), CA19-9 (carcinomas), L6 (carcinomas) (SEQ. ID. 107), Lewis Y (carcinomas) (SEQ. ID. 108), Lewis X (carcinomas) (SEQ. ID. 109), alpha fetoprotein (carcinomas) (SEQ. ID. 110), CA 242 (colorectal), placental alkaline phosphatase (carcinomas) (SEQ. ID. 112), prostate specific antigen (prostate) (SEQ. ID. 113), prostate specific membrane antigen (prostate) (SEQ. ID. 114), prostatic acid phosphatase (prostate) (SEQ. ID. 115), epidermal growth factor (carcinomas), MAGE-1 (carcinomas) (SEQ. ID. 117), MAGE-2 (carcinomas) (SEQ. ID. 118), MAGE-3 (carcinomas) (SEQ. ID. 119), MAGE-4 (carcinomas) (SEQ. ID. 120), anti-transferrin receptor (carcinomas) (SEQ. ID. 121), p97 (melanoma) (SEQ. ID. 122), MUC1 (breast cancer) (SEQ. ID. 123), CEA (colorectal) (SEQ. ID. 124), gp100 (melanoma) (SEQ. ID. 125), MART-1 (melanoma) (SEQ. ID. 126), IL-2 receptor (T-cell leukemia and lymphomas), CD2 (buccal mucosa) (SEQ. ID. 128), CD20 (non-Hodgkin's lymphoma) (SEQ. ID. 129), CD52 (leukemia) (SEQ. ID. 130), CD33 (leukemia), CD22 (lymphoma), beta human chorionic gonadotropin (carcinoma) (SEQ. ID. 133), CD38 (multiple myeloma) (SEQ. ID. 134), CD40 (lymphoma) (SEQ. ID. 135), CD80 (colorectal), CD86 (colorectal), mucin (carcinomas), P21 (carcinomas), MPG (melanoma) (SEQ. ID. 140), Neu oncogene product (carcinomas) and STEAP-1 (prostate).

Compositions and Methods of Administration. In other embodiments, described is a pharmaceutical composition including an effective amount of an Affinity Medicant Linker conjugate and/or a Medicant Linker compound and a pharmaceutically acceptable carrier or vehicle. The compositions are suitable for veterinary or human administration.

The present pharmaceutical compositions can be in any form that allows for the composition to be administered to a patient. For example, the composition can be in the form of a solid or liquid. Typical routes of administration include, without limitation, parenteral, ocular and intra-tumor. Parenteral administration includes subcutaneous injections, intravenous, intramuscular or intrasternal injection or infusion techniques. In one aspect, the compositions are administered parenterally. In a specific embodiment, the compositions are administered intravenously.

Pharmaceutical compositions can be formulated so as to allow an Affinity Medicant Linker conjugate and/or a Medicant Linker compound to be bioavailable upon administration of the composition to a patient. Compositions can take the form of one or more dosage units, where for example, a tablet can be a single dosage unit, and a container of an Affinity Medicant Linker conjugate and/or a Medicant Linker compound in liquid form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the Affinity Medicant Linker conjugate and/or a Medicant Linker compound, the manner of administration, and the composition employed.

The pharmaceutically acceptable carrier or vehicle can be solid or particulate, so that the compositions are, for example, in tablet or powder form. The carrier(s) can be liquid. In addition, the carrier(s) can be particulate.

The composition can be in the form of a liquid, e.g., a solution, emulsion or suspension. In a composition for administration by injection, one or more of a surfactant, preservative, wetting agent, dispersing agent, suspending agent, buffer, stabilizer and isotonic agent can also be included.

The liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, phosphates or amino acids and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

The amount of the Affinity Medicant Linker conjugate and/or a Medicant Linker compound that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of an Affinity Medicant Linker conjugate and/or a Medicant Linker compound such that a suitable dosage will be obtained. Typically, this amount is at least about 0.01% of an Affinity Medicant Linker conjugate and/or a Medicant Linker compound by weight of the composition. In an exemplary embodiment, pharmaceutical compositions are prepared so that a parenteral dosage unit contains from about 0.01% to about 2% by weight of the Affinity Medicant Linker conjugate and/or a Medicant Linker compound.

For intravenous administration, the composition can comprise from about 0.01 to about 100 mg of an Affinity Medicant Linker conjugate and/or a Medicant Linker compound per kg of the patient's body weight. In one aspect, the composition can include from about 1 to about 100 mg of an Affinity Medicant Linker conjugate and/or a Medicant Linker compound per kg of the patient's body weight. In another aspect, the amount administered will be in the range from about 0.1 to about 25 mg/kg of body weight of the Affinity Medicant Linker conjugate and/or a Medicant Linker compound.

Prior art ADC's such as Kadcyla or Adcetris deliver a dose of the associated toxin (auristatins MMAE or emtansine DM-1) that is three or more times the lethal dose (for that toxin) which results in severe systemic (or non-target) toxicity. In contrast, illudofulvenes ADC's (such as analog 189, analog 190, analog 217, analog 218, analog 219, analog 222, or analog 316 deliver less than one third (i.e., <⅓) of a lethal dose, minimizing the risk and severity of systemic toxicity. Illudofulvenes are true cytotoxic agents whereas other toxic agents used in prior art ADC's (e.g., pyrrolobenzodiazepines, maytansines, fumagillols, dolstatins, auristatins, enadiynes, halichondrins, and tubulysins) are only cytostatic. See Table VI (based on the NCI-DTP 60 cell line). Hence, other payloads, such as those used in Herceptin, Adcetris or Rituxin only stall tumor cell growth and do not actually kill the tumor cells. Other payloads (e.g., pyrrolobenzodiazepines, maytansines, fumagillols, dolstatins, auristatins, enadiynes, halichondrins, and tubulysins) are not active against multidrug phenotypes, notably the MDR1/GP170 and MRP/GP180 transport mechanisms (see Table IV). Illudofulvenes show the excellent effect of remaining active against all MDR phenotypes known regardless of the mechanism of resistance (see Table IV). Hence, if tumor cells have already developed multi-drug resistance to a prior art ADC with a prior art toxin, or develop multi-drug resistance during the administration of a course of the prior art ADC with a prior art toxin, then the ADC will have no efficacy. In contrast, ADCs developed with illudofulvenes have the advantageous effect that they will continue to kill cancer cells.

Generally, the dosage of an Affinity Medicant Linker conjugate and/or a Medicant Linker compound administered to a patient is typically about 0.01 mg/kg to about 20 mg/kg of the patient's body weight. In one aspect, the dosage administered to a patient is between about 0.01 mg/kg to about 10 mg/kg of the patient's body weight. In another aspect, the dosage administered to a patient is between about 0.1 mg/kg and about 10 mg/kg of the patient's body weight. In yet another aspect, the dosage administered to a patient is between about 0.1 mg/kg and about 5 mg/kg of the patient's body weight. In yet another aspect the dosage administered is between about 0.1 mg/kg to about 3 mg/kg of the patient's body weight. In yet another aspect, the dosage administered is between about 1 mg/kg to about 3 mg/kg of the patient's body weight.

The Affinity Medicant Linker conjugate and/or a Medicant Linker compound can be administered by any convenient route, for example by infusion or bolus injection. Administration can be systemic or local. Various delivery systems are known, e.g., encapsulation in liposomes, micro-particles, microcapsules, capsules, etc., and can be used to administer an Affinity Medicant Linker conjugate and/or a Medicant Linker compound. In certain embodiments, more than one Affinity Medicant Linker conjugate and/or a Medicant Linker compound is administered to a patient.

In specific embodiments, it can be desirable to administer one or more Affinity Medicant Linker conjugates and/or a Medicant Linker compound locally to the area in need of treatment. This can be achieved, for example, and not by way of limitation, by local infusion during surgery; topical application, e.g., in conjunction with a wound dressing after surgery; by injection; by means of a catheter; or by means of an implant, the implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue. In another embodiment, administration can be by direct injection at the site (or former site) of a manifestation of an autoimmune disease.

In yet another embodiment, the Affinity Medicant Linker conjugate and/or a Medicant Linker compound can be delivered in a controlled release system, such as but not limited to, a pump or various polymeric materials can be used. In yet another embodiment, a controlled-release system can be placed in proximity of the target of the Linker Affinity conjugate and/or a Medicant Linker compound, e.g., the liver, thus requiring only a fraction of the systemic dose.

The term “carrier” refers to a diluent, adjuvant or excipient, with which an Affinity Medicant Linker conjugate and/or a Medicant Linker compound is administered. Such pharmaceutical carriers can be liquids, such as water and oils, including those of petroleum, animal, vegetable or synthetic origin. The carriers can be saline, and the like. In addition, auxiliary, stabilizing and other agents can be used. In one embodiment, when administered to a patient, the Affinity Medicant Linker conjugate and/or the Medicant Linker compound and pharmaceutically acceptable carriers are sterile. Water is an exemplary carrier when the Affinity Medicant Linker conjugate and/or a Medicant Linker compound are administered intravenously. Saline solutions and aqueous dextrose and glycerol solutions can also be employed as liquid carriers, particularly for injectable solutions. The present compositions, if desired, can also contain minor amounts of wetting or emulsifying agents, or pH buffering agents.

The present compositions can take the form of solutions, pellets, powders, sustained-release formulations, or any other form suitable for use.

In an embodiment, the Affinity Medicant Linker conjugates and/or Medicant Linker compounds are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where an Affinity Medicant Linker conjugate and/or Medicant Linker compound is to be administered by infusion, it can be dispensed, for example, with an infusion bottle containing sterile pharmaceutical grade water or saline. Where the Affinity Medicant Linker conjugate and/or Medicant Linker compound is administered by injection, an ampoule of sterile water for injection or saline can be provided so that the ingredients can be mixed prior to administration.

The composition can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

Whether in solid or liquid form, the present compositions can include a pharmacological agent used in the treatment of cancer, an autoimmune disease or an infectious disease.

Treatment of Cancer. The Affinity Medicant Linker conjugates and Medicant Linker compounds are useful for inhibiting the multiplication of a tumor cell or cancer cell, causing apoptosis in a tumor or cancer cell, or for treating cancer in a patient. The Affinity Medicant Linker conjugates and/or Medicant Linker compounds can be used accordingly in a variety of settings for the treatment of animal cancers. The Affinity Medicant Linker Conjugates can be used to deliver a Medicant or Medicant unit to a tumor cell or cancer cell. Without being bound by theory, in one embodiment, the AM of an Affinity Medicant Linker conjugate binds to or associates with a cancer-cell or a tumor-cell-associated antigen, and the Affinity Medicant Linker conjugate can be taken up (internalized) inside a tumor cell or cancer cell through receptor-mediated endocytosis or other internalization mechanism. The antigen can be attached to a tumor cell or cancer cell or can be an extracellular matrix protein associated with the tumor cell or cancer cell. Once inside the cell, one or more specific peptide sequences within or at the Medicant unit's proximal end of the LU are hydrolytically cleaved by one or more tumor cell or cancer cell-associated proteases, resulting in release of the Medicant unit. The released Medicant unit is then free to migrate within the cell and induce cytotoxic or cytostatic activities. The Affinity Medicant Linker conjugate also can be cleaved by an intracellular protease to release the Medicant moiety. In an alternative embodiment, the Medicant or Medicant unit is cleaved from the Affinity Medicant Linker conjugate outside the tumor cell or cancer cell, and the Medicant or Medicant unit subsequently penetrates the cell.

The Affinity Medicant Linker conjugates provide conjugation-specific tumor or cancer medicant targeting, thus reducing general toxicity of the Medicant. The LUs stabilize the Affinity Medicant Conjugates in blood, yet are cleavable by tumor-specific proteases within the cell, liberating a Medicant unit.

In one embodiment, the AM binds to the tumor cell or cancer cell. In another embodiment, the AM binds to a tumor cell or cancer cell antigen which is on the surface of the tumor cell or cancer cell. In another embodiment, the AM binds to a tumor cell or cancer cell antigen which is an extracellular matrix protein associated with the tumor cell or cancer cell.

The specificity of the AM for a particular tumor cell or cancer cell can be important for determining those tumors or cancers that are most effectively treated. For example, an Affinity Medicant Linker conjugate and/or Medicant Linker compound having a BR96 AM can be useful for treating antigen positive carcinomas including those of the lung, breast, colon, ovaries, and pancreas. Affinity Medicant Linker conjugates having an anti-CD30 or an anti-CD70 binding affinity moiety can be useful for treating hematologic malignancies.

Other particular types of cancers that can be treated with an Affinity Medicant Linker conjugate and/or a Medicant Linker compound include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma blood-borne cancers, including but not limited to: acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma acute and chronic leukemias: lymphoblastic, myelogenous, lymphocytic, myelocytic leukemias Lymphomas: Hodgkin's disease, non-Hodgkin's Lymphoma, Multiple myeloma, Waldenstrom's macroglobulinemia, Heavy chain disease, Polycythemia vera.

Multi-Modality Therapy for Cancer. Cancers, including, but not limited to, a tumor, metastasis, or other disease or disorder characterized by uncontrolled cell growth, can be treated or inhibited by administration of an Affinity Medicant Linker conjugate or Medicant Linker compound.

In other embodiments, methods for treating cancer are provided, including administering to a patient in need thereof an effective amount of an Affinity Medicant Linker conjugate and a chemotherapeutic agent. In one embodiment the chemotherapeutic agent is that with which treatment of the cancer has not been found to be refractory. In another embodiment, the chemotherapeutic agent is that with which the treatment of cancer has been found to be refractory. The Affinity Medicant Linker conjugates can be administered to a patient that has also undergone surgery as treatment for the cancer.

In some embodiments, the patient also receives an additional treatment, such as radiation therapy. In a specific embodiment, the Affinity Medicant Linker conjugate is administered concurrently with the chemotherapeutic agent or with radiation therapy. In another specific embodiment, the chemotherapeutic agent or radiation therapy is administered prior or subsequent to administration of an Affinity Medicant Linker conjugate.

A chemotherapeutic agent can be administered over a series of sessions. Any one or a combination of the chemotherapeutic agents, such a standard of care chemotherapeutic agent(s), can be administered.

Additionally, methods of treatment of cancer with an Affinity Medicant Linker conjugate and/or a Medicant Linker compound are provided as an alternative to chemotherapy or radiation therapy where the chemotherapy or the radiation therapy has proven or can prove too toxic, e.g., results in unacceptable or unbearable side effects, for the subject being treated. The patient being treated can, optionally, be treated with another cancer treatment such as surgery, radiation therapy or chemotherapy, depending on which treatment is found to be acceptable or bearable.

Example 1. Synthesis of Medicant 113. The Wittig reaction was performed on analog 010. First 65 mg CH₃PPh₃Br (0.185 mmol) in anhydrous THF was cooled to −75° C. and stirred for 1 hour. Then 200 μL of n-butyl lithium (0.183 mmol) was added very slowly to the flask while maintaining temperature at −75° C., and a yellow precipitate formed. It was stirred for another 1.5 hours then analog 010 (50 mg, 0.183 mmol) was slowly added while maintaining temperature at −75° C., followed by stirring for 2.0 hours. The reaction was quenched with ammonium chloride, extracted with CH₂Cl₂, washed with water, NaHCO₃, and saline. Dried over Na₂SO₄and concentrated. The residue was eluted through a column (10% ethyl acetate in hexane) to give analog 113 as a solid.

Example 2. Synthesis of Medicant-Estrone 107. Analog 106 (see Example 13) (139 mg 0.384 mmol, 1 equiv.), DMAP (4 mg, 0.03 mmol, 0.08 equiv.) and estrone (104.4 mg, 0.384 mmol, 1 equiv.) were dissolved in CH₂Cl₂ (14 mL) at 0° C. To this solution was added CH₂Cl₂ solution of DCC (460 μL, 1 M, 0.46 mmol, 1.2 equiv.) through a syringe. After 0.5 hours the solution was raised to RT. After 2 hours the mixture was filtered and the filtrate was washed with dilute HCl (1.5%), saturated NaHCO₃ and brine in sequence. The organic phase was then dried and evaporated. The residue was eluted through a column (CH₂Cl₂/Methanol, 10:0.25) to give analog 107 (100 mg, 42%) as semisolid. Analog 107 can be subsequently linked to estrone.

Example 3. Preparation of Medicant-Estradiol 108. Analog 038 (58.5 mg, 0.2035 mmol), beta-estradiol (58.0 mg, 0.2150 mmol) and DMAP (5 mg. 0.048 mmol) were dissolved in CH₂Cl₂ (5.6 mL) at 0° C. To this solution was added CH₂Cl₂ solution of DCC (250 μL, 1 M, 0.244 mmol), stirred for 30 minutes, allowed to warm to RT then stirred for 1.5 hours. The filtrate was washed with dilute HCl (1.5%), saturated NaHCO₃ and brine in sequence. The organic phase was dried over Na₂SO₄, and evaporated. The residue was eluted through a column (100% CH₂Cl₂), fractions collected then eluted through a second column (CH₂C12plus 0.5% methanol), to give analog 108 (45 mg) as a solid.

Table I shows the cytotoxic data IC₅₀ values (micromolar, 2 hour exposure, N=3, mean±SD) for 108. MCF7 over express estrogen alpha-receptors. MCF7 cells are preferentially killed by 110 the acylfulvene-estrone analog and to a lesser extent 108 the acylfulvene-estradiol analog because estrone preferentially binds to alpha-receptor.

Example 4. Preparation of Medicant-Estradiol 109. Analog 106 (54.5 mg, 0.15 mmol, 1 equiv.), β-estradiol (40.5 mg, 0.15 mmol), and DMAP (1.8 mg, 0.015 mmol, 0.1 equiv.) were dissolved in CH₂Cl₂(5 mL) at 0° C. To this solution was added CH₂Cl₂ solution of DCC (165 μL, 1 M, 0.165 mmol, 1.1 equiv.). The mixture was raised to RT after 0.5 h. After another 2 h, the mixture was filtered. The filtrate was washed with dilute HCl (1.5%), saturated NaHCO₃ and brine in sequence. The organic phase was dried and evaporated. The residue was eluted through a column (CH₂Cl₂/Methanol 10:0.25) to give analog 109 (55 mg, 60%) as semisolid.

Example 5. Preparation of Medicant-Estrone 110. Analog 038 (68 mg, 0.2365 mmol), estrone (68.0 mg, 0.2160 mmol) and DMAP (5 mg. 0.048 mmol) were dissolved in CH₂Cl₂ (8.0 mL) at 0° C. To this solution was added CH₂Cl₂ solution of DCC (300 μL, 1 M, 0.283 mmol), stirred for 30 minutes, allowed to warm to RT then stirred for 0.5 hours. The filtrate was washed with dilute HCl (1.5%), saturated NaHCO₃ and brine in sequence. The organic phase was dried over Na₂SO₄, and evaporated. The residue was eluted through a column (100% CH₂Cl₂), fractions collected then eluted through a second column (CH₂C12plus 0.5% methanol), to give analog 110 (40 mg) as a solid.

Table I shows the cytotoxic data IC₅₀ values (micromolar, 2 hour exposure, N=3, mean±SD) for 110. MCF7 cells over express estrogen alpha-receptors. MCF7 cells are preferentially killed by the acylfulvene-estrone analog 110 and to a lesser extent by the acylfulvene-estradiol analog 108 because estrone preferentially binds to alpha-receptor. In contrast, illudin M killed both ER negative and ER positive cells to the same extent. The data in Table I demonstrates that analog 108 and analog 110 are preferentially cytotoxic to cells expressing large numbers of estrogen receptors on their surface.

Example 6. Preparation of Medicant-Testosterone 111. Analog 038 (52.5 mg, 0.182 mmol), testosterone (50.0 mg, 0.173 mmol) and DMAP (5 mg. 0.048 mmol) were dissolved in CH₂Cl₂ (8.0 mL) at 0° C. To this solution was added CH₂Cl₂ solution of DCC (250 μL, 1 M), stirred for 30 minutes, allowed to warm to RT then stirred for 2 hours. The filtrate was washed with dilute HCl (1.5%), saturated NaHCO₃ and brine in sequence. The organic phase was dried over Na₂SO₄, and evaporated. The residue was eluted through a column (100% CH₂Cl₂ plus 0.5% methanol), to give analog 111 (15 mg) as a solid.

Example 7. Preparation of Medicant-Androsterone 112. Analog 038 (29 mg), androsterone (25.0 mg) and DMAP (5 mg. 0.048 mmol) were dissolved in CH₂Cl₂(5.0 mL) at 0° C. To this solution was added CH₂Cl₂ solution of DCC (150 μL, 1 M), stirred for 30 minutes, allowed to warm to RT then stirred for 2 hours. The filtrate was washed with dilute HCl (1.5%), saturated NaHCO₃ and brine in sequence. The organic phase was dried over Na₂SO₄, and evaporated. The residue was eluted through a column (2:3 ethyl acetate: hexane) to give analog 112 (15 mg) as a solid.

In an embodiment of the present invention, an illudin2 moiety 1302 linked via a traditional linker 1240 to a steroid 1140 bind to receptors for the steroid and directs the illudin2 moiety 1302 to cell populations expressing the receptor. In an embodiment of the present invention, a steroid 1140 linked via a traditional linker 1240 to an illudin2 moiety 1302 acts as an AM for the steroid hormone receptor and directs the illudin2 moiety 1302 to tissues containing cells expressing the receptor. In an embodiment of the present invention, a steroid 1140 linked via a traditional linker 1240 to an illudin2 moiety 1302 acts as an AM for the steroid hormone receptor and directs the illudin2 moiety 1302 to tumors containing cells over-expressing the receptor compared to non tumor cells.

Example 4. Preparation of Medicant-Estradiol 109. Analog 106 (54.5 mg, 0.15 mmol, 1 equiv.), β-estradiol (40.5 mg, 0.15 mmol), and DMAP (1.8 mg, 0.015 mmol, 0.1 equiv.) were dissolved in CH₂Cl₂ (5 mL) at 0° C. To this solution was added CH₂Cl₂ solution of DCC (165 μL, 1 M, 0.165 mmol, 1.1 equiv.). The mixture was raised to RT after 0.5 h. After another 2 h, the mixture was filtered. The filtrate was washed with dilute HCl (1.5%), saturated NaHCO₃ and brine in sequence. The organic phase was dried and evaporated. The residue was eluted through a column (CH₂Cl₂/Methanol 10:0.25) to give analog 109 (55 mg, 60%) as semisolid.

Example 5. Preparation of Medicant-Estrone 110. Analog 038 (68 mg, 0.2365 mmol), estrone (68.0 mg, 0.2160 mmol) and DMAP (5 mg. 0.048 mmol) were dissolved in CH₂Cl₂ (8.0 mL) at 0° C. To this solution was added CH₂Cl₂ solution of DCC (300 μL, 1 M, 0.283 mmol), stirred for 30 minutes, allowed to warm to RT then stirred for 0.5 hours. The filtrate was washed with dilute HCl (1.5%), saturated NaHCO₃ and brine in sequence. The organic phase was dried over Na₂SO₄, and evaporated. The residue was eluted through a column (100% CH₂Cl₂), fractions collected then eluted through a second column (CH₂C12plus 0.5% methanol), to give analog 110 (40 mg) as a solid.

Table I shows the cytotoxic data IC₅₀ values (micromolar, 2 hour exposure, N=3, mean±SD) for 110. MCF7 cells over express estrogen alpha-receptors. MCF7 cells are preferentially killed by the acylfulvene-estrone analog 110 and to a lesser extent by the acylfulvene-estradiol analog 108 because estrone preferentially binds to alpha-receptor. In contrast, illudin M killed both ER negative and ER positive cells to the same extent. The data in Table I demonstrates that compounds 108 and 110 are preferentially cytotoxic to cells expressing large numbers of estrogen receptors on their surface.

Example 6. Preparation of Medicant-Testosterone 111. Analog 038 (52.5 mg, 0.182 mmol), testosterone (50.0 mg, 0.173 mmol) and DMAP (5 mg. 0.048 mmol) were dissolved in CH₂Cl₂ (8.0 mL) at 0° C. To this solution was added CH₂Cl₂ solution of DCC (250 μL, 1 M), stirred for 30 minutes, allowed to warm to RT then stirred for 2 hours. The filtrate was washed with dilute HCl (1.5%), saturated NaHCO₃ and brine in sequence. The organic phase was dried over Na₂SO₄, and evaporated. The residue was eluted through a column (100% CH₂Cl₂ plus 0.5% methanol), to give analog 111 (15 mg) as a solid.

Example 7. Preparation of Medicant-Androsterone 112. Analog 038 (29 mg), androsterone (25.0 mg) and DMAP (5 mg. 0.048 mmol) were dissolved in CH₂Cl₂ (5.0 mL) at 0° C. To this solution was added CH₂Cl₂ solution of DCC (150 μL, 1 M), stirred for 30 minutes, allowed to warm to RT then stirred for 2 hours. The filtrate was washed with dilute HCl (1.5%), saturated NaHCO₃ and brine in sequence. The organic phase was dried over Na₂SO₄, and evaporated. The residue was eluted through a column (2:3 ethyl acetate: hexane) to give analog 112 (15 mg) as a solid.

Example 13. Synthesis of Medicant 106. Illudin M (450 mg, 1.845 mmol, 1 equiv.), glutaric anhydride (2.10 g, 18.45 mmol, 10 equiv.) and DMAP (171 mg, 1.4 mmol, 0.76 equiv.) were dissolved in CH₂Cl₂ (5 mL) at RT. After 3.5 hours the mixture was taken up by CH₂Cl₂, which was washed with water, and brine in sequence. It was then dried and evaporated. The residue was eluted through a column (Hexane/EtOAc 4:1) to give analog 106 (365 mg, 55%) as a liquid. UV (CHCl₃) k nm (s): 309 (3387).

Analog 106 was generated from illudin M as outlined in Example 13. The carboxylic acid derivative was activated using DCC/DMAP to synthesize steroid AFC's 107 and 109. In addition, Irofulven carboxylic acid derivative, analog 038 was activated using DCC/DMAP to produce analogs 108, 110, 111, and 112. In general, carboxylate group containing compounds can be activated using a carbodiimide in the presence of an amino acid to form an azlactone. The azlactone formed will react spontaneously with primary amine groups on an amino acid, a peptide, an antibody, a protein, or another drug, and undergo ring opening with the formation of an amide bond. For proteins, antibodies and peptides the amino acids capable of reacting with the azlactone derivative includes arginine and lysine.

To form an Illudin derived azlactone active drug-linker moiety, either analog 106 or analog 038 can be activated by DCC/DMAP in the presence of a small amino acid such as glycine to form the azlactone. DCC cannot be added without the presence of an amine containing target (such as the glycine) or the activated carboxylate reacts with another carboxylate to form a symmetrical anhydride. The azlactone formed will react spontaneously with primary amine groups on a peptide, an antibody, a protein, or a medicant.

Example 14. Activation of analog 038 by DCC to form medicant-azlactone. Part A: Production of Azlactone from carboxylate Acylfulvene analog: Analog 038 (58.5 mg, 0.2035 mmol), and DMAP (5 mg. 0.048 mmol) were dissolved in CH₂Cl₂ (5.6 mL) at 0° C. The desired amino acid (such as glycine) was added in an equimolar amount. Note that amino acids having substitutions on the C4 carbon (such as alpha-methyl glycine or 2-dimethylglycine) are preferred over conventional amino acids as substitution cannot occur at the C4 position after ring-opening and all nucleophilic coupling reactions must occur at the C5 position, resulting only in the desired amide-bond formation with the amine-containing molecule. To this solution was added CH₂Cl₂ solution of DCC (250 μL, 1 M, 0.244 mmol), stirred for 30 minutes, allowed to warm to RT then stirred for 1.5 hours. The filtrate was washed with dilute HCl (1.5%), saturated NaHCO₃ and brine in sequence. The organic phase was dried over Na₂SO₄, and evaporated. The residue was eluted through a column (100% CH₂C12plus 0.5% methanol), to give the desired azlactone analog as a solid. Part B: Coupling of Azlactone to the protein component (reacting with primary amines on amino acids such as the one on lysine): The typical protein coupling reaction consists of the Azlactone suspended in buffer [25 mM sodium phosphate, 150 mM NaCl (pH 7.5)] and the desired amount of protein (20 μg to 5.0 mg) was added. The mixture was gently rocked for 60 minutes, then the reaction terminated by the addition of the blocking reagent, 1.0 ml of 1.0 M ethanolamine in 25 mM sodium pyrophosphate (titrated to pH 9.0 with HCl) Sample rocked gently for 5 minutes then the residual ethanolamine removed by dialysis or chromatography using pH 7.5 phosphate-NaCl buffer.

Example 15. Reaction of the medicant-azlactone product with an antibody. The azlactone derivative generated in Example 14 (note that other amino acids can be used in place of glycine) was then reacted with the desired peptide or protein or other compound containing a primary amino group at a 1:1 ratio in buffer (25 mM sodium phosphate, 150 mM sodium chloride, pH 7.5) with gentle rocking at RT for 60 minutes. The reaction was terminated by the addition of 1.0 mL of 25 mM ethanolamine (titrated to pH 9.00) with rocking for 5 minutes at RT). The drug-azlactone-ligand product can be purified by column chromatography or dialysis to remove the ethanolamine by-product.

Example 16. Synthesis of Medicant 114. (CH₃)₃S(O)I (110 mg, 0.4 mmol) and tBuOK(50 mg, 0.4 mmol) were dissolved in anhydrous DMSO (1 mL) and stirred at RT for 40 minutes at RT. Then analog 010 (50 mg, 0.2 mmol) in 1.0 mL of DMSO was added via syringe, and stirred for 3 hours. Reaction quenched with saturated NH₄Cl (1 mL), extracted with CH₂Cl₂, dried over Na₂SO₄, concentrated then chromatographed (2:3 hexane:ethyl acetate) to yield analog 114 (20 mg., 50% yield).

Example 17. Synthesis of Medicant 115. Analog 010 (40 mg) and NAHCO₃ (50 mg) are dissolved in 10 mL of 1:1 Ethanol and water mixture, then hydroxylamine hydrochloride (20 mg) was added, stirred for 30 minutes at RT. Water and ethyl acetate (1:1 mixture) was added, stirred, the organic layer was recovered, washed with saturated NaHCO₃ and then brine, dried over Na₂SO₄, concentrated then chromatographed (2:3 ethyl acetate: hexane) to yield analog 115.

Example 18. Synthesis of Medicant 116. SeO₂ (45 mg) and 500 mg SiO2 transferred into a dried RB flask, 5 mL of CH₂Cl₂ added, and stirred for 1 hour under nitrogen. Then 250 μL of tBuO₂H added and stirred for 15 minutes. Then 100 mg of Irofulven in 1 mL CH₂Cl₂ was added, and stirred for 3 hours at RT under a nitrogen atmosphere. Product was filtered, wash twice with water (25 mL), twice with brine (25 mL), dried over Na₂SO₄and concentrated then chromatographed (4:1 hexane: ethyl acetate) to yield analog 116.

Example 19. Synthesis of Medicant 116. Analog 117: Illudin S (100 mg, 0.378 mmol) and glutaric anhydride (215.46 mg, 1.89 mmol) are dissolved in 5 mL of CH₂Cl₂, and DMAP added (92.23 mg, 0.756 mmol), and stirred for 2 hours at RT. The CH₂Cl₂ was evaporated, 5 mL of water was added, and stirred for 1 hour. The solution was extracted with 10 mL of CH₂Cl₂, washed with water, dried over Na₂SO₄ and concentrated to yield analog 117 (120 mg).

Example 20. Synthesis of Analog 118: Analog 302 (75 mg), glutaric anhydride (20 mg) are dissolved in 5 mL of CH₂Cl₂, and DMAP added (42 mg), and stirred for 2 hours at RT. The CH₂Cl₂ was evaporated, 5 mL of water added, and stirred for 1 hour. Solution was extracted with 10 mL of CH₂Cl₂, washed with water, dried over Na₂SO₄ and concentrated to yield analog 118 (120 mg).

Example 21. Synthesis of Analog 119: Analog 114 (10 mg) was dissolved in 1.5 mL of acetone with 1.0 mL of 4N H₂SO₄, and contents stirred for 1.5 hours at RT. Then 10 mL of CH₂Cl₂ and 10 mL of water are added, extracted, and the organic layer recovered which was then washed with saturated NaHCO₃ and saline, dried over Na₂SO₄ and concentrated, and analog 119 recovered (analog 128 was a byproduct).

Example 22. Synthesis of Analog 120: Analog 010 (50 mg), NaHCO₃ are dissolved in 10 mL of 1:1 mixture of water and ethanol, then NH₂NH₂ (0.5 mL added with stirring at RT for one hour. The solution was extracted with CH₂Cl₂ twice, the organic layer recovered, washed with water, then NaHCO₃ solution, dried over Na₂SO₄, and evaporated to yield analog 120 (30 mg).

Example 23. Synthesis of Analog 121: Analog 010 (50 mg) and NaCO₂CH₃ (75 mg) are dissolved in 10 mL of 1:1 mixture of water and ethanol 1:1, then semicarbazide hydrochloride salt (H₂NNHCONH₂—HCl, 50 mg) added, and stirred for 2 hours at RT. The solution was extracted with CH₂Cl₂ twice, the organic layer recovered, washed with water, then NaHCO₃ solution, dried over Na₂SO₄, and evaporated then chromatographed (5% methanol in ethyl acetate) to yield analog 121.

Example 24. Synthesis of Analog 122: Analog 010 (50 mg) and NaCO₂CH₃ (75 mg) are dissolved in 5 mL of ethanol, then phenylhydrazide (50 mg) was added, stirred for 1 hour at RT. Then 5 mL of water was added, followed by extraction with ethyl acetate, washed with water, dried over Na₂SO₄ and concentrated and chromatographed (5% methanol in ethyl acetate) to yield analog 122.

Example 25. Synthesis of Analog 123: Analog 010 (50 mg) and NaCO₂CH₃ (75 mg) are dissolved in 10 mL of 1:1 water and ethanol, then H₂NNHTS (H₂NNHS(═O)₂(phenyl)methyl, 50 mg) was added, stirred for 2 hour at RT. Then 5 mL of water was added, followed by extraction with ethyl acetate, washed with water, dried over Na₂SO₄ and concentrated and chromatographed (5% methanol in ethyl acetate) to yield analog 123.

Example 26. Synthesis of Analog 124: Analog 115 (15 mg) and NaOAc(15 mg) are dissolved in acetic anhydride (1 mL) and stirred for 2 hours, then sodium acetate (300 mg) was added with stirring for 1 hour. Then the mixture was chromatographed (10% ethyl acetate in hexane) to give analog 124.

Example 27. Synthesis of Analog 125: Analog 010 (50 mg) and NaCO₂CH₃ (75 mg) are dissolved in 5 mL of ethanol, then the dinitrophenylhydrazide (50 mg) was added, stirred for 1 hour at RT. Then 5 mL of water was added, followed by extraction with ethyl acetate, washed with water, dried over Na₂SO₄ and concentrated and chromatographed (5% methanol in ethyl acetate) to yield analog 125.

Example 28. Synthesis of Analog 126: Analog 011 (40 mg), hydroxylamine (20 mg), NaHCO₃ (50 mg) are dissolved in 10 mL of ethanol and water (1:1) then stirred at RT for 90 minutes. Then the mixture was extracted with water (10 mL) and ethyl acetate (20 mL), the organic layer washed with saturated NaHCO₃ then brine, dried over Na₂SO₄ and concentrated, then chromatographed (2:3 ethyl acetate:hexane) to give analog 126.

Example 29. Synthesis of Analog 127: Analog 010 (100 mg) and NH₄Cl (1.5 equivalent) are dissolved in 1,4-dioxane (5 mL) and water (0.2 mL), then NaCN added (1.3 equivalents), stirred for 1 hour at RT. Then ethyl ether (20 mL) was added, the organic layer recovered, washed with water, washed with brine, then dried over Na₂SO₄, then chromatographed (2:3 ethyl acetate: hexane) to yield analog 127.

Example 30. Synthesis of Analog 128: Analog 114 (10 mg) was dissolved in 1.5 mL of acetone with 1.0 mL of 4N H₂SO₄, and contents stirred for 1.5 hours at RT. Then 10 mL of CH₂Cl₂ and 10 mL of water are added, extracted, and the organic layer recovered which was then washed with saturated NaHCO₃ and saline, dried over Na₂SO₄ and concentrated, and analog 128 recovered (analog 119 was a byproduct).

Example 31. Synthesis of Analog 129: Analog 001 (200 mg) was dissolved in anhydrous THF (10 mL) at RT then NaBH₄ (100 mg) was added slowly for 30 minutes. Reaction was quenched with 1 mL of water then extracted with ethyl acetate (10 mL), washed with saturated NaHCO₃, and dried over Na₂SO₄, then concentrated to yield analog 129. If need be the compound can be purified by chromatography (1:1 ethyl acetate: hexane).

Example 32. Analog 141: Analog 129 (200 mg) was dissolved in CH₂Cl₂ at RT, then 1,4-dimethyl but-2-ynedioate (1:1 equivalent) was added slowly and mixture allowed to react for one hour, then evaporated to yield analog 141 If need be the compound can be purified by chromatography (1:1 ethyl acetate: hexane).

Example 33. Synthesis of Analog 142: Analog 141 (100 ng) was dissolved in CH₂Cl₂ at RT then Dess-Martin Periodinane reagent (200 mg) added with stirring for 1 hour to yield analog 142. If need be the compound can be purified by chromatography (1:1 ethyl acetate: hexane).

Example 34. Synthesis of Analog 146: Analog 127 (35 mg, 0.117 mmol), DMAP (5 mg), and diimidazole (22 mg, 1.2 eq) were dissolved in anhydrous CH₂Cl₂ under an argon atmosphere, and stirred for 30 minutes. The solution was cooled to 20° C. then tributyl tin hydride (Bu₃SnH, 0.6 mL) and azobis isobutylnitrite (4 mg) were added with stirring for 30 minutes. The mixture was filtered then chromatographed (1:10 ethyl acetate: hexane) to remove impurities and starting materials, then chromatographed (2:3 ethyl acetate: hexane) to yield analog 146.

Example 35. Synthesis of Analog 147: Irofulven (10 mg) was dissolved in 3 mL of acetone and 1 M H₂SO₄ solution (1:1) with stirring at RT and 2-Mercaptobenzothiazole (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 147.

Example 36. Synthesis of Analog 148: Irofulven (10 mg) was dissolved in 3 mL of acetone and 1 M H₂SO₄ solution (1:1) with stirring at RT and 2-Mercaptobenzoxazole (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 Ethyl acetate: hexane) to give analog 148.

Example 37. Synthesis of Analog 149: Irofulven (10 mg) was dissolved in 4 mL of acetone and 1 M H₂SO₄ solution (1:1) with stirring at RT and thiol-imidazole (1 equivalent) was added, stirred for 24 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 149.

Example 38. Synthesis of Analog 150: Irofulven (10 mg) was dissolved in 4 mL of acetone and 1 M H₂SO₄ solution (1:1) with stirring at RT and 2-mercapto-5-methylbenzimidazole (1 equivalent) was added, stirred for 12 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 150.

Example 39. Synthesis of Analog 151: Irofulven (10 mg) was dissolved in 3 mL of acetone and 1 M H₂SO₄ solution (1:1) with stirring at RT and 1-phenyl-1,2,3,4-tetraazole-5-thiol (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 151.

Example 40. Synthesis of Analog 152: Irofulven (10 mg) was dissolved in 3 mL of acetone and 1 M H₂SO₄ solution (1:1) with stirring at RT and 2-mercapto-5-nitro benzimidazole (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 152.

Example 41. Synthesis of Analog 153: Irofulven (10 mg) was dissolved in 3 mL of acetone and 1 M H₂SO₄ solution (1:1) with stirring at RT and 1,2,4-Triazole-3-thiol (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 153.

Example 42. Synthesis of Analog 154: Irofulven (10 mg) was dissolved in 3 mL of acetone and 1 M H₂SO₄ solution (1:1) with stirring at RT and 2-sulfanylpteridin-4-ol (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 154.

Example 43. Synthesis of Analog 155: Irofulven (10 mg) was dissolved in 3 mL of acetone and 1 M H₂SO₄ solution (1:1) with stirring at RT and 4-(5-sulfanyl-1H-1,2,3,4-tetrazol-1-yl)phenol (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 155.

Example 44. Synthesis of Analog 156: Irofulven (10 mg) was dissolved in 3 mL of acetone and 1 M H₂SO₄ solution (1:1) with stirring at RT and 4-(5-sulfanyl-1-1,2,3,4-tetrazol-1-yl)benzoic acid (1 equivalent) was added, stirred for 2 hours, then partitioned between ethyl acetate and water. The organic extract was washed with saturated NaHCO₃ and saline until neutral, dried over MgSO₄, concentrated then chromatographed (1:1 ethyl acetate:hexane) to give analog 156.

Example 45. Synthesis of Analog 159: Illudin S (300 mg) was dissolved acetic anhydride (6 mL) and stirred for 15 minutes, then sodium acetate (300 mg) was added with stirring for 1 hour. Water (6 mL) was added, ethyl acetate extraction performed, washed with sodium bicarbonate solution, dried over Na₂SO₄, concentrated then chromatographed (2:3 ethyl acetate:hexane) to give analog 159.

Example 46. Synthesis of Analog 160: Analog 159 (60 mg) was dissolved in dry CH₂Cl₂ (6 mL) under nitrogen at RT and glutaric anhydride (100 mg) with DMAP (20 mg) was added with stirring for 30 minutes. The solvent was removed, water added, extracted with CH₂Cl₂, washed with water, dried over Na₂SO₄, concentrated then chromatographed (2:3 ethyl acetate: hexane) to give analog 160.

Example 47. Synthesis of Analog 161: Dehydroilludin S (300 mg) was dissolved acetic anhydride (6 mL) and stirred for 15 minutes, then sodium acetate (300 mg) was added with stirring for 1 hour. Water (6 mL) was added, ethyl acetate extraction performed, washed with sodium bicarbonate solution, dried over Na₂SO₄, concentrated then chromatographed (2:3 ethyl acetate:hexane) to give analog 161.

Example 48. Synthesis of Analog 162: Dehydroilludin S (60 mg) was dissolved in dry CH₂Cl₂ (6 mL) under nitrogen at RT and glutaric anhydride (150 mg) with DMAP (50 mg) was added with stirring for 30 minutes. The solvent was removed, water added, extracted with CH₂Cl₂, washed with water, dried over Na₂SO₄, concentrated then chromatographed (2:3 ethyl acetate:hexane) to give analog 162.

Example 49. Synthesis of Analog 163: Analog 159 (20.25 mg), DMAP (20 mg) are dissolved in dry CH₂Cl₂ (6 mL) at 0° C. under nitrogen atmosphere and stirred for 10 minutes. Then chloroacetyl chloride (0.2 mL) was added slowly and the mixture stirred for 30 minutes, warmed to RT with stirring over 15 minutes. Then water (6 mL) was added, mixed, and then extracted with CH₂Cl₂. The organic layer was washed with saturated NaHCO₃ followed by a saline wash, dried over Na₂SO₄ then chromatographed (2:3 ethyl acetate: hexane) to yield analog 163 (60% yield).

Example 50. Synthesis of Analog 164: Irofulven (50 mg), DMAP (40 mg) are dissolved in dry CH₂Cl₂ (6 mL) at 0° C. under nitrogen atmosphere and stirred for 10 minutes. Then chloroacetyl chloride (0.2 mL) was added slowly and the mixture stirred for 30 minutes, warmed to RT with stirring over 15 minutes. Then water (6 mL) was added, mixed, and then extracted with CH₂Cl₂. The organic layer was washed with saturated NaHCO₃ followed by a saline wash, dried over Na₂SO₄ then chromatographed (2:3 ethyl acetate: hexane) to yield analog 164 (60% yield).

Example 51. Synthesis of Analog 165: Analog 164 (40 mg) was dissolved in dry CH₂Cl₂ (6 mL) at RT under nitrogen atmosphere and stirred for 10 minutes. Then 1 mL of morpholine was added drop wise, with stirring for 30 minutes. The reaction was diluted with water (6 mL), extracted with CH₂Cl₂ (12 mL). The organic layer was washed with saturated NaHCO₃ then washed with saline, dried over Na₂SO₄ and chromatographed (2:3 ethyl acetate: hexane) to yield 165 (35% yield).

Example 52. Synthesis of Analog 166 and analog 167 (prepared together): Analog 160 (30 mg) was dissolved in methanol (4 mL) at 0° C., and 1N H₂SO₄ (1 mL) was added with stirring for 1 hour. Water (6 mL) was added, extracted with ethyl acetate, washed with NaHCO₃ then a brine solution, dried over MgSO₄, concentrated and then chromatographed (1:1 ethyl acetate:hexane) to yield analogs 166 and 167 in equal amounts.

Example 53. Synthesis of Analog 168: Analog 162 (20 mg) was dissolved in methanol (5 mL) at 0° C. and stirred for 10 minutes, then 1 mL of 1N H₂SO₄ in methanol was slowly added, followed by stirring for 30 minutes. Water was added, followed by an ethyl acetate extraction, washed with NaHCO₃ then a brine solution, dried over Na₂SO₄, concentrated then chromatographed (1:1 ethyl acetate:hexane) to yield analog 168.

Example 54. Synthesis of Analog 169: Dehydroilludin S (20 mg), DMAP (20 mg) are dissolved in dry CH₂Cl₂ (6 mL) at 0° C. under nitrogen atmosphere and stirred for 10 minutes. Then chloroacetyl chloride (0.2 mL) was added slowly and the mixture stirred for 30 minutes, warmed to RT with stirring over 15 minutes. Then water (6 mL) was added, mixed, then extracted with CH₂Cl₂. The organic layer was washed with saturated NaHCO₃ followed by a saline wash, dried over Na₂SO₄ then chromatographed (2:3 ethyl acetate: hexane) to yield analog 169 (60% yield).

Example 55. Synthesis of Analog 176: To a solution of analog 009 (266 umol), Boc protected leucine amino acid (300 umol) and DMAP (dimethylaminopyridine, 110 umol) in CH₂Cl₂ (2.5 mL) at 0° C. was added DCC (dicyclohexylcarbodiimide; 1.0M in CH₂Cl₂, 300 umol)/. The mixture was stirred for 35 minutes then 5 μL of water added to quench the reaction. The mixture was diluted with hexane and precipitate filtered off, solvent evaporated off and crude product chromatographed (2:1 hexanes-ethyl acetate) to give the desired Boc-protected derivative of 176 at 80% yield. The Boc group was removed by dissolving the Boc-protected derivative in a 1:1 mixture (2.0 mL) of 1,4-dioxane and 2M H₂SO₄, stirred for 18 hours, then partitioned between ethyl acetate and water. Aqueous layer was extracted with ethyl acetate and extracts discarded. Aqueous layer was neutralized with saturated NaHCO₃ and extracted again with ethyl acetate. Organic layer was washed with brine, dried with MgSO₃, solvent evaporated to yield the analog 009 amino acid derivative. As the amine derivative was unstable over prolonged periods of time it can be converted to the very stable trifluoroacetate salt by dissolving in CH₂Cl₂ adding the equal molar amount of trifluoroacetic acid and concentrating to dryness.

Example 56. Synthesis of Analog 178: Analog 009 (15 mg) was dissolved in CH₂Cl₂ (2.0 mL) under a nitrogen atmosphere at RT, succinic anhydride (1 equivalent) was added, followed by DMAP (10 mg) and stirring for 30 minutes. Solvent was removed and product recrystallized to give analog 178.

Example 57. Synthesis of Analog 179: To a solution of Analog 009 (266 μmol), Boc protected glycine amino acid (300 umol) and DMAP (dimethylaminopyridine, 110 umol) in CH₂Cl₂ (2.5 mL) at 0° C. was added DCC (dicyclohexylcarbodiimide; 1.0M in CH₂Cl₂, 300 umol)/. The mixture was stirred for 35 minutes then 5 μL of water added to quench the reaction. The mixture was diluted with hexane and precipitate filtered off, solvent evaporated off and crude product chromatographed (2:1 hexanes-ethyl acetate) to give the desired Boc-protected derivative of 179 at 80% yield. The Boc group was removed by dissolving the Boc-protected derivative in a 1:1 mixture (2.0 mL) of 1,4-dioxane and 2M H₂SO₄, stirred for 18 hours, then partitioned between ethyl acetate and water. Aqueous layer was extracted with ethyl acetate and extracts discarded. Aqueous layer was neutralized with saturated NaHCO₃ and extracted again with ethyl acetate. Organic layer was washed with brine, dried with MgSO₃, solvent evaporated to yield the analog 009 amino acid derivative. As the amine derivative was unstable over prolonged periods of time it can be converted to the very stable trifluoroacetate salt by dissolving in CH₂Cl₂ adding the equal molar amount of trifluoroacetic acid and concentrating to dryness.

Example 58. Synthesis of Analog 180: Illudin M (50 mg) was dissolved in dry benzene (10 mL) under a nitrogen atmosphere, and vanadyl acetylacetonate (VO(acac)₂, 1.2 mg) was added. Then t-butyl hydroperoxide (t-BuO₂H, 0.5 mL) in benzene was added drop wise with stirring for 30 minutes. A saturated solution of Na₂S₂O₃ was added (10 mL), then extraction with ethyl acetate, and the organic layer was dried over Na₂SO₄, concentrated then chromatographed) (1:1 ethyl acetate:hexane) to give analog 180.

Example 59. Synthesis of Analog 181: Analog 159 (40 mg) was dissolved in dry benzene (8 mL) under a nitrogen atmosphere, and vanadyl acetylacetonate (VO(acac)₂, 2 mg) was added. Then t-butyl hydroperoxide (t-BuO₂H, 0.5 mL) in benzene was added drop wise with stirring for 30 minutes. A saturated solution of Na₂S₂O₃ was added (10 mL), then extraction with ethyl acetate, followed by a brine wash, and the organic layer was then dried over Na₂SO₄, concentrated then chromatographed) (1:1 ethyl acetate:hexane) to give analog 181.

Example 60. Synthesis of Analog 189: To a solution of Irofulven (1.00 equivalent), maleimide (1.71 equivalent), triphenylphosphine (PPh₃, 1.71 equivalent) in 1.5 mL of THF at −40° C., was added DEAD (diethylazodicarboxylate; 1.68 equivalent). The mixture was stirred for 30 minutes then water (20 μL) added to quench the reaction. The mixture was concentrated on a rotary evaporator and crude product was chromatographed on a silica column (10:3 hexanes: ethyl acetate) to yield an orange compound (20% yield).

Example 61. Synthesis of Analog 190: To a solution of analog 009 (6-hydroxy-n-propylacylfulvene—structure below, 1.00 equivalent), maleimide (1.23 equivalent), triphenylphosphine (PPh₃, 1.13 equivalent) in 2.5 mL of THF at −40° C., was added DIAD (diisopropylcarbodiimide; 1.44 equivalent). The mixture was stirred for 1 hour then water (10 μL) added to quench the reaction. The mixture was concentrated on a rotary evaporator and crude product was chromatographed on a silica column (5:1→10:3 hexanes:ethyl acetate) to yield an orange compound (15% yield).

Example 62. Synthesis of Analog 196: To a solution of analog 009 (266 umol), Boc protected proline amino acid (300 umol) and DMAP (dimethylaminopyridine, 110 umol) in CH₂Cl₂ (2.5 mL) at 0° C. was added DCC (dicyclohexylcarbodiimide; 1.0M in CH₂Cl₂, 300 umol)/. The mixture was stirred for 35 minutes then 5 μL of water added to quench the reaction. The mixture was diluted with hexane and precipitate filtered off, solvent evaporated off and crude product chromatographed (2:1 hexanes-ethyl acetate) to give the desired Boc-protected derivative of 196 at 80% yield. The Boc group was removed by dissolving the Boc-protected derivative in a 1:1 mixture (2.0 mL) of 1,4-dioxane and 2M H₂SO₄, stirred for 18 hours, then partitioned between ethyl acetate and water. Aqueous layer was extracted with ethyl acetate and extracts discarded. Aqueous layer was neutralized with saturated NaHCO₃ and extracted again with ethyl acetate. Organic layer was washed with brine, dried with MgSO₃, solvent evaporated to yield the analog 009 amino acid derivative. As the amine derivative was unstable over prolonged periods of time it can be converted to the very stable trifluoroacetate salt by dissolving in CH₂Cl₂ adding the equal molar amount of trifluoroacetic acid and concentrating to dryness.

Example 63. Synthesis of Analog 198: Irofulven (26.3 mg, 107 umol), p-nitrophenol (16.2 mg, 116 umol) and PPh3 (30.8 mg, 117 umol) were dissolved in anhydrous THF (1.5 mL) at −40° C., the DEAD (25 μL, 160 umol) was added, followed by stirring for 30 minutes, then diluted with hexane. The precipitate was filtered off, solvent evaporated, and crude product chromatographed (6:1->2:1 hexane: ethyl acetate) to give analog 198 as a yellow product (18.5 mg, 47%).

Example 64. Analogs 199 and 200 (prepared together):: Irofulven (25.2 mg, 102 umol), phenol (11.5 mg, 122 umol) and PPh₃ (29.1 mg, 117 μmol) were dissolved in anhydrous THF (1.0 mL) at −40° C., the DEAD (25 μL, 192 μmol) was added, followed by stirring for 30 minutes, then diluted with hexane. The precipitate was filtered off, solvent evaporated, and crude product chromatographed (6:1->3:1 hexane: ethyl acetate) to give analog 199 (8.2 mg, 25%) and analog 200 (14.6 mg, 44%) as a yellow products.

Example 65. Synthesis of Analog 201 [6-(acetamidopropyl)acylfulvene]: To a solution of analog 195 (49.1 umol) and water (20 μL in THF (0.5 ml) was added a solution of O-acetyl-2-(diphenylphosphino)phenol (39.0 umol) in THF (0.5 mL). The mixture was stirred for 3 days at RT then concentrated. The crude product was chromatographed (100% ethyl acetate) to yield 8.2 mg of analog 201.

Example 66. Synthesis of Analog 202 (i.e., analog 211 linked to proline): Prepared via Staudinger ligation. To a solution of analog 195 (94 umol) in THF (1.2 mL), water (40 μL) was added, the was added N-Boc-proline, 2-(diphenylphosphino)phenyl ester (101 μmol) in THF (0.8 mL). The mixture was stirred for 3 days at RT then concentrated. The crude product was chromatographed (5:1->1:2 hexanes-ethyl acetate) to yield 31.4 mg (66.7 umol) of analog 202-Boc (71%). The analog 202-Boc was dissolved (66.7 umol) in dioxane (2.0 mL) and 2.0 mL of 2M H₂SO₄ was added, and the mixture was stirred overnight. Water and ethyl acetate was added, orange color appeared in the aqueous. The aqueous was extracted again with ethyl acetate and organic layer discarded. Sodium bicarbonate was added to aqueous until basic, re-extracted with ethyl acetate. The solution was dried with magnesium sulphate, concentrated to dryness, dissolved in CH₂Cl₂ and 8 mg of TFA added (1 drop). Analog 202 was obtained in an amount of 22.2 mg (69%).

Example 67. Synthesis of Analog 203: Analog 208 (9.2 mg, 16.5 umol) was dissolved in CH₂Cl₂ (1.5 mL), 1 drop of anisole added, then 0.5 mL of trifluoro acetic acid for 15 minutes. The mixture was concentrated, dissolved in water, then re-extracted with CH₂Cl₂, and the orange color remains in the aqueous phase, which was concentrated to give analog 203 as the orange colored TFA salt (10.0 mg).

Example 68. Synthesis of Analog 204: Although the Fmoc-Pro-OH would preferentially react with the primary hydroxyl group on Illudin S, the resulting ester linkage was not stable, as illudin S was recovered after storage in CDCl₃ for several days at RT. The secondary hydroxy group of illudin S was therefore used for coupling with peptides. The primary hydroxy group of illudin S first protected with a TBS group (TBSCl, Imidazole, and DMF, 92%) to produce analog 204.

Example 69. Synthesis of Analog 205: Analog 309 (20 mg, 0.050 mmol, 1 equiv.), triphenylphosphine (40 mg, 0.1525 mmol, 3 equiv.) was dissolved in THF (1 mL) at RT. After 20 hours a few drops of water was added and the mixture was heated up at 70° C. After 5 hours the solution was cooled down and evaporated. The residue was chromatographed (hexane/EtOAc/Et₃N 4:1:0.1) to give analog 205 (5.3 mg, 29%) as an oil.

Example 70. Synthesis of Analog 206: Analog 205 (14 mg, 0.037 mmol, 1 equiv.) was dissolved in CH₃CN (0.5 mL) and pyridine (0.1 mL) at 0° C. To this solution was added HF-Pyridine (7 μL, 0.245 mmol, 35 M, 6.6 equiv.). After 10 min K₂CO₃ (0.5 mL, 0.5 M) was added and this mixture was chromatographed (CH₂Cl₂/Methanol/Et₃N 5:0.5:0.1) to give analog 206 (10 mg, 68%) as an oil.

Example 71. Synthesis of Analog_207 (211-leucine): Prepared via Staudinger ligation. To a solution of analog 195 (101 umol) in THF (1.0 mL), water (40 μL) was added, then was added N-Boc-leucine,2-(diphenylphosphino)phenyl ester (95.9 μmol) in THF (1.2 mL). The mixture was stirred for 6 days at RT then concentrated. The crude product was chromatographed (1:1 hexanes-ethyl acetate) to yield 27.3 mg of analog 207-Boc. The analog 207-Boc was dissolved (16 μmol) in CH₂Cl₂ with 3 drops of anisole, TFA was added (0.3 mL), and the mixture was stirred for 15 minutes then concentrated. The crude material was dissolved in water then extracted with CH₂Cl₂. The aqueous layer was recovered and concentrated to yield 17.4 mg of the analog 207TFA salt (87%).

Example 72. Analog 208: The TFA salt of analog 196 (13.7 mg, 28.2 μmol) was dissolved in anhydrous DMF (2.5 mL), Boc-Serine-OH (9.6 mg, 47 umol) was added, ODHBT (13.0 mg, 79.4 umol), cooled to 0° C. under a nitrogen atmosphere. Next EDC (15.1 mg) was added followed by NMM (10 μL) to adjust pH, and the mixture stirred at 0° C. for 3 hours. The reaction was added to ethyl acetate/water mixture, and the orange product appeared in the organic layer. The aqueous layer was re-extracted with ethyl acetate, organic layers combined, washed with dilute NaHSO₄, water, saturated NaHCO₃, brine, then dried with MgSO₄. The organic layer was concentrated then chromatographed (1:3 hexane: ethyl acetate) to yield analog 208 as an orange residue (63% yield).

Example 73. Synthesis of Analog 209: The TFA salt of analog 196 (12.5 mg, 25.7 μmol) was dissolved in anhydrous DMF (2.5 mL), Boc-Serine-Ser OH (88.6 umol) was added, ODHBT (33.9 mg, 205 umol), cooled to 0° C. under a nitrogen atmosphere. Next EDC (142 umol) was added followed by NMM (10 μL) to adjust pH, and the mixture stirred at 0° C. but allowed to gradually warm as the ice melts. The mixture was stirred a total of 16 hour then 1 mL water added followed by stirring for 50 minutes. The reaction was added to ethyl acetate/water mixture, and the orange product appeared in the organic layer. The aqueous layer was re-extracted with ethyl acetate, organic layers combined, washed with dilute NaHSO₄, water, saturated NaHCO₃, brine, and then dried with MgSO₄. The organic layer was concentrated then chromatographed (10:1 ethyl acetate: methanol) to give analog 209 as an orange residue (5.9 mg, 36% yield).

Example 74. Synthesis of Analog 210 (Ac-Hyp-Ser-Ser-Chg-Gln-Ser-Ser-Pro-O—(CH₂)₃-acylfulvene): To a mixture of Analog 196 TFA salt (21.6 umol), the peptide Ac-Hyp-Ser-Ser-Chg-Gln-Ser-Ser-OH (30.3 umol), ODHBt (3,4,-dihydroxy-4-oxo-1,2,3-benzo-triazine-3-yl ester, 71.7 μmol) and NMM (N-methylmorpholine; 7.5 ul) in DMF (2.0 ml) at RT was added EDC (1-(3-dimethylaminopropyl)-3-ethylcarbodiimide hydrochloride, 68 μmol), the mixture stirred for 2 hours at RT, then diluted with 10 mL of water. Solution was directly chromatographed on a reverse phase C18 column (4:1->2:1, water/acetonitrile gradient) to yield 69% of analog 210.

Example 75. Synthesis of Analog 212 (Illudin M-proline) Illudin M (20 mg, 0.081 mmol, 1 equivalent), DMAP (1 mg, 0.008 mmol, 0.1 equiv.) and Fmoc-Pro-OH (33 mg, 0.097 mmol, 1.2 equiv.) were dissolved in CH₂Cl₂ (1 mL) at 0° C., to which was added a CH₂Cl₂ solution of DCC (100 μL, 0.1 mmol, 1 M, 1.2 equiv.). The temperature of the mixture gradually rose to 5° C. in 1.5 hours and then the mixture was filtered through a pad of Celite. The filtrate was concentrated and the residue was chromatographed (CH₂Cl₂/EtOAc 5:0.1-5:0.4) to give Illudin-M-proline-Fmoc protected analog (36 mg, 79%) as oil. The proton spectra of this oil showed that it was a mixture of two isomers (rotamers). And then this oil was dissolved in CH₂Cl₂ (4 mL) and treated with piperidine (1 mL) at 0° C. After 0.5 hours the solution was concentrated and the concentrate was chromatographed (CH₂Cl₂/Methanol 5:0.4) to give analog 212 (15 mg, 54%) as oil.

Example 76. Synthesis of Analog 213: Analog 204 was coupled with Fmoc-Pro-H (DMAP, CH₂Cl₂, DCC, 0° C., 85%), followed by deprotection of Fmoc group with 20% piperidine in CH₂Cl₂ to produce analog 213 in 78% yield.

Example 77. Synthesis of Analog 214 (Illudin S-Pro-Ser-Ser-HHOAc): The Fmoc protected peptide of H-Ser-Ser-OH was prepared by taking H-Ser-Ser-OH (50 mg, 0.26 mmol, 1 equiv.) and K₂CO₃ (89.7 mg, 0.65 mmol, 2.5 equiv.), dissolving in a mixture of water (4 mL) and dioxane (3 mL) at 0° C. To this solution Fmoc (67.3 mg, 0.26 mmol, 1 equiv.) was added in several portions. After 18 hours the mixture was acidified by KHSO₄ and the pH raised to 2.5. Then this mixture was taken up by ethyl acetate, which was washed with brine, dried, filtered and evaporated. The residue was chromatographed (CH₂Cl₂/Methanol/HOAc 5:1:0.1) to give 3.27 (75 mg, 70%) as a white solid. The analog 212 (Illudin S tosylate-Pro) (42.8 mg 0.09 mmol, 0.9 equiv.), and the Fmoc protected H-Ser-Ser-OH peptide (41.2 mg, 0.1 mmol, 1 equiv.) were dissolved in DMF (1.5 mL) at 0° C. To this solution was added NMM (22 μL, 0.2 mmol, 2 equiv.), ODHBt (29.4 mg, 0.18 mmol, 1.8 equiv.), and EDC (31.1 mg, 0.16 mmol, 1.6 equiv.). The solution temperature was then raised to RT and kept for 3 hours before it was taken up by ethyl acetate. The mixture was then washed with saturated sodium bicarbonate and brine. It was then dried, filtered and evaporated. The residue was chromatographed (CH₂Cl₂/Methanol 5:0.3) to give analog 214 (50.5 mg, 67%) as an oil.

Example 78. Synthesis of Analog 215: (Illudin S-Pro-Ser-Ser-Gln-Chg-Ser-Ser-Hyp-Ac) Analog 204 was coupled with Fmoc-Pro-H (DMAP, CH₂Cl₂, DCC, 0° C., 85%), followed by deprotection of Fmoc group with 20% piperidine in CH₂Cl₂ to produce analog 213 in 78% yield. Peptide conjugate, analog 215 was obtained from further coupling with hepta-peptide Ac-Hyp-Ser-Ser-Chg-Gln-Ser-Ser-OH (ODHBt, NMM, DMF, 0° C., 47%).

Example 79. Synthesis of Analog 216: (Illudin M-Pro-Ser-Ser-Gln-Chg-Ser-Ser-Hyp-Ac). Analog 212 was further coupled with the commercially available hepta-peptide Ac-Hyp-Ser-Ser-Chg-Gln-Ser-Ser-OH (ODHBt, NMM, DMF, EDC, 0° C.) to yield analog 216 at 33%. The low yield resulted from repeated chromatographic purification as the purity of the final raw product was estimated by HPLC to be only 70%.

Example 80. Synthesis of Analog 217: To a solution of Irofulven (1.00 equivalent), epsilon-maleimidocaproic acid (1.27 equivalent), DMAP (0.15 equivalent) in 1.0 mL of methylene chloride (CH₂Cl₂) at 0° C., was added DCC (dicyclohexylcarbodiimide; 1.27 equivalent) in methylene chloride (CH₂Cl₂). The mixture was stirred for 1.25 hours, diluted with hexane and precipitated was filtered. Residual solvent was evaporated off, and oil residue was chromatographed on a silica column (2:1 hexanes:ethyl acetate) to yield analog 217, an orange compound (77% yield).

Example 81. Synthesis of Analog 218: To a solution of Illudin M (1.00 equivalent), epsilon-maleimidocaproic acid (1.33 equivalent), DMAP (0.18 equivalent) in 1.0 mL of methylene chloride (CH₂Cl₂) at 0° C., was added DCC (dicyclohexylcarbodiimide; 1.33 equivalent) in methylene chloride (CH₂Cl₂). The mixture was stirred for 2.25 hours, diluted with hexane and precipitated was filtered. Residual solvent was evaporated off, and oil residue was chromatographed on a silica column (2:1 hexane:ethyl acetate) to yield analog 218, an orange compound (83% yield).

Example 82. Synthesis of Analog 219: Analog 204 (33.4 mg) was dissolved in 1.0 mL of anhydrous pyridine under a nitrogen atmosphere, then DMAP (5.1 mg) was added, followed by 4-fluorosulfonyl-benzoyl chloride (86.1 mg). The mixture was stirred for 90 minutes at RT The mixture was diluted with ethyl acetate, washed once with saturated copper sulfate solution, washed twice with water, then dried over MgSO₄, concentrated then chromatographed (20% ethyl acetate: hexane) to give analog 219.

Example 83. Synthesis of Analog 221: Prepared from Analog 207 by coupling with Mu-His-Ser-Ser-Lys(Fmoc)-Leu-Gln-OH in DIC/HOBt for 5 minutes, then 5% piperidine/DMF for 1 minute. Followed by TFA quenching to yield analog 221 at 21% yield.

Example 84. Synthesis of Analog 222:: Illudin M (63 mg) was dissolved in 1.0 mL of anhydrous pyridine under a nitrogen atmosphere, then DMAP (6.4 mg) was added, followed by 4-fluorosulfonyl-benzoyl chloride (86 mg). The mixture was stirred for 35 minutes at RT then chromatographed (20% ethyl acetate: hexane) to give analog 222 (70.9 mg).

Example 85. Synthesis of Analog 223: The disulfhydryl peptide CNGRC was first converted to a cyclic disulfide peptide by dissolving 355 mg in 3.0 mL DMSO, adding 9 mL of water, allowing to sit overnight at RT, followed by water removal on a rotoevaporator then DMSO removal under high vacuum. The TFA salt of analog 179 (14.5 mg) was dissolved in DMF (2.0 mL) and the CNGRC disulfide peptide added (19.0 mg), 60 μL of DIPEA was added, followed by gradual addition of a solution of Py-BOP (19.6 mg) and HOBt (8.9 mg) in DMF (2.0 mL) over 150 minutes at RT. The reaction was stopped by adding two drops of TFA and water. The mixture was applied to a reverse phase column and analog 223 was eluted with acetonitrile: water (1:4).

Example 86. Synthesis of Analog 224: Analog 001 (116 mg) was dissolved in ethanol (4.0 mL) with stirring, hydroxylamine hydrochloride (84.2 mg) added, Sodium acetate (233 mg) added, then refluxed for 70 minutes at 85° C. The ethanol was removed, then ethyl acetate (10 mL) added to dissolve crude product, then water (10 mL) added, the organic layer was washed with brine, dried over Na₂SO₄, concentrated then chromatographed (20% ethyl acetate: hexane) to give analog 224 (63.7 mg, 54% yield).

Example 87. Synthesis of Analog 225: Illudin S (439 mg) was dissolved in ethanol (15 mL) with stirring, hydroxylamine hydrochloride (233 mg) added, sodium acetate (933 mg) added, then refluxed for 130 minutes at 85° C. The solution was cooled to RT, filtered, ethanol was removed, then ethyl acetate (30 mL) added to dissolve crude product, then water (30 mL) added, the organic layer was washed with brine, dried over Na₂SO₄, concentrated then chromatographed (30%->50%, acetone: hexane) to give analog 225 (372 mg, 80% yield).

Example 88. Synthesis of Analog 226: Irofulven (37.6 mg) was dissolved with stirring in CH₂Cl₂, elaidic acid (180 mg. 1.3 equivalents) added, DMAP (15 mg) added, cooled to 0° C., then DCC (180 μL) in CH₂Cl₂ (640 μL) added. Reaction mixture stirred at 0° C. for 1 hour, then additional DCC (120 μL) added, and stirred for 2 more hours. Mixture chromatographed (20% ethyl acetate: hexane) to give analog 226 as a yellow oil (50.5 mg, 48% yield).

Example 89. Synthesis of Analog 227: Analog 009 (87 mg) was dissolved with stirring in CH₂Cl₂, elaidic acid (108 mg) added, DMAP (15.4 mg) added, cooled to 0° C., then DCC (0.5 mL) in CH₂Cl₂ (1.5 mL) added. Reaction mixture stirred at 0° C. for 3 hours, then the mixture directly chromatographed (20% ethyl acetate: hexane) to give analog 227 as a yellow oil (105 mg, 61% yield).

Example 90. Synthesis of Analog 228: Illudin S (86 mg) was dissolved with stirring in CH₂Cl₂, elaidic acid (202 mg) added, DMAP (15.4 mg) added, cooled to 0° C., then DCC (1.0 mL) in CH₂Cl₂ (3.0 mL) added. Reaction mixture stirred at 0° C. for 3 hours, then the mixture directly chromatographed (20% ethyl acetate: hexane) to give analog 228 as a yellow oil (198 mg, 77% yield).

Example 91. Synthesis of Analog 229: The elaidic ester of 0-diphenylphosphine phenol was first prepared by dissolving with stirring in 3.0 mL of CH₂Cl₂ the O-diphenylphosphine phenol (91.3 mg), elaidic acid (94.5 mg, 1 equivalent), DMAP (9.4 mg). The solution was cooled to 0° C. then DDC (0.44 mL, 1.0 M in CH₂Cl₂) was added with stirring for 3.5 hours. The precipitate was filtered off and discarded. The elaidic ester was chromatographed and concentrated to dryness then dissolved in THF (1.0 mL). Analog 195 (26.1 mg) was dissolved in THF (1.0 mL) and water (80 μL) added. The elaidic ester solution was slowly added to the analog 195 solution with stirring, and reacted for 22 hours at RT. The mixture was directly chromatographed (30% acetone: hexane) to give analog 229 (22.2 mg, 47% yield).

Example 92. Synthesis of Analog 230: Analog 308 (22 mg) was dissolved in anhydrous CH₂Cl₂ (1.5 mL), diisopropylethylamine (20 μL) added, and the mixture cooled to 0° C., then methylsulfonyl chloride added (15 μL), mixture stirred at 0° C. for 1 hour, and allowed to warm to RT while being stirred for an additional hour. The mixture was chromatographed (30% ethyl acetate in hexane) to yield analog 230 (35% yield).

Example 93. Synthesis of Analog 231: Analog 308 (16 mg) was dissolved in anhydrous CH₂Cl₂ (1.5 mL), diisopropylethylamine (20 μL) added, and the mixture cooled to 0° C., then tosyl chloride added (18.4 mg), mixture stirred at 0° C. for 1 hour, and allowed to warm to RT while being stirred for an additional 3 hours. The mixture was chromatographed (30% ethyl acetate in hexane) to yield analog 231 (8.6 mg).

Example 94. Synthesis of Analog 240: Analog 232 (25.1 mg) was dissolved in anhydrous CH₂Cl₂ (2.0 mL), 15 μL of acetic anhydride added, and the mixture cooled to RT, then DMAP added (5 mg), and stirred for 25 minutes. The mixture was partially concentrated then chromatographed (30% ethyl acetate in hexane) to yield analog 240 (26.6 mg, 93% yield).

Example 95. Synthesis of Analog 254: Analog 009 (51.4 mg), 4-carboxybenzene sulfonamide (59.4 mg), and DCC (39.6 mg) were dissolved in anhydrous DMF (1.0 mL) at RT, stirred, then DMAP (15 mg) added. The mixture was stirred for 2 hours at RT then solid material was filtered off. The mixture was then chromatographed (1:1 ethyl acetate: hexane) to give analog 254 (38.6 mg, 45% yield).

Example 96. Synthesis of Analog 255: Analog 009 (244.3 mg) and sulfamoyl chloride (157 mg) were dissolved in anhydrous DMAP (2.0 mL) at RT, and stirred for 3.5 hours. The mixture was concentrated under high vacuum then chromatographed (30% ethyl acetate in hexane) to give analog 255.

Example 97. Synthesis of Analog 259: Analog 255 (64.7 mg), (diacetoxyiodo)benzene (64.7 mg), dirhodiumtetraacetate or Rh₂(OAc)₄ and magnesium (16.8) dissolved in 5.0 mL of CH₂Cl₂ are heated to 70° C. and stirred for 7 hours. The mixture was filtered, concentrated, then chromatographed (1:1 ethyl acetate: hexane) to give analog 259.

Example 98. Synthesis of Analog 262 and 263 (prepared together): Analog 025 (44.7 mg) was dissolved in methanol (1.0 mL), Oxone® reagent (246 mg, 3 equivalents) was dissolved in water (1.0 mL). The oxone solution was slowly added to the methanol solution with stirring at RT for 3.5 hours, then an additional amount of Oxone reagent added followed by stirring for 1.5 hours. Then 2 mL of saturated sodium sulfite solution was added, followed by ethyl acetate extraction, dried over Na₂SO₄, concentrated then chromatographed (1:1 Ethyl acetate:hexane) to yield first analog 263 (21.4 mg) and then analog 262 (14.3 mg).

Example 99. Synthesis of Analog 284 and 289 (prepared together): Analog 034 (174 mg) and uracil (227 mg) are dissolved in CH₂Cl₂ with stirring and the mixture cooled to 0° C. Then SnCL₄ (148.8 μL) was slowly added. The mixture was stirred at 0° C. for 80 minutes, then concentrated, chromatographed (2->5% methanol: CH₂Cl₂) to give analog 284 (68.9 mg, 33% yield) and analog 289 (21.6 mg, 10% yield).

Example 100. Synthesis of Analog 285: Analog 034 (25 mg) was dissolved in ethanol, and O-(tert-Butyldimethylsilyl) hydroxylamine (25 mg) was added followed by stirring for 2 hours at RT. The secondary amine intermediate (9 mg) was recovered by chromatography (30% ethyl acetate:hexane), dissolved in CH₂Cl₂, and reacted with sulfamoyl chloride (ClSO₂NH₂, 5 mg) and DABCO (2 mg) with stirring for one hour, then additional sulfamoyl chloride (6 mg) was added with stirring for another 1.5 hours. The TPS blocked product was recovered by chromatography (30% ethyl acetate:hexane), and the TPS group was removed in THF by adding TBAF (Tetra-n-butylammonium fluoride). The TPS group can also be removed by dissolving the TPS product in pyridine and THE at 0° C., then adding HF-pyridine overnight. After TPS deblocking the mixture was chromatographed (50% ethyl acetate: hexane) to give analog 285.

Example 101. Synthesis of Analog 286 and analog 287 (prepared together): The ketone groups on 5-fluorouracil are first blocked with TMS groups by dissolving 5-fluorouracil (610 mg) and (NH₄)₂SO₄ in HMDS (10 mL) under a nitrogen atmosphere. The solution was refluxed at 142° C. for 2.5 hours, cooled to 60° C. and excess HMDS distilled off, then concentrated to dryness under high vacuum. Analog 034 (180 mg) and the di-TMS 5-fluorouracil are dissolved in CH₂Cl₂ (5.0 mL) with stirring and the mixture cooled to 0° C. Then SnCL₄ (120 μL) was slowly added drop wise. The mixture was stirred at 0° C. for 3.5 hours, then concentrated, chromatographed (80% ethyl acetate: hexane) to give analog 286 (18.9 mg, 9% yield) and analog 287 (84 mg, 38% yield).

Example 102. Synthesis of Analog 289: See the preparation of analog 284 for the preparation of analog 289 (284 and 289 prepared simultaneously then separated by chromatography).

Example 103. Analogs 299 and 300 (prepared together): Analogs 299 and 300 are prepared in equal amounts from Illudin S using the Mitsunobu reaction. Illudin S was directly reacted with HN₃ (PPh3, DEAD, benzene) at 0° C. under nitrogen for 45 minutes. Mitsunobu, O. Synthesis 1:1-28, 1981.

Example 104. Synthesis of Analog 301: Irofulven (31.6 mg, 0.128 mmol), 5-benzoylvaleric acid (35.8 mg, 0.174 mmol) and DMAP (4.7 mg) was dissolved in CH₂Cl₂ (2 mL) under a nitrogen atmosphere, cooled to 0° C., the DCC added (170 μL of 1.0M solution in CH₂Cl₂). The mixture was stirred for 60 minutes then diluted with hexane (10 mL) and filtered. The organic layer was further diluted with CH₂Cl₂, washed with water, then saturated NaHCO₃ then brine, dried with MgSO₄, concentrated, then dissolved in CH₂Cl₂, filtered and chromatographed (10:3 hexane:ethyl acetate), appropriate fractions collected, pooled, concentrated then chromatographed (3:1 hexane:ethyl acetate) to give analog 301 (23.2 mg, 42% yield).

Example 105. Analogs 302 and analog 303 (prepared together): Illudin S (100 mg, 0.378 mmol) was benzoylated by dissolving in pyridine (1.0 mL) then adding 3,5-dintirobenzoyl chloride (110 mg, 0.5 mmol) at RT and stirring for 24 hours. The mixture was poured onto crushed ice then extracted with CH₂Cl₂ (10 mL), which was washed twice with water (20 mL). The organic layer was dried over Na₂SO₄ and concentrated to yield analogs 302 and 303. The two analogs can be separated by column chromatography (1:1 hexane:ethyl acetate).

Example 106. Synthesis of Analog 304: Analog 009 (84.6 mg) was dissolved in anhydrous CH₂Cl₂ (3.0 mL), DCC added (81.2 mg), mixture cooled to 0° C., propiolic acid (35 μL) added, then the reaction started with DMAP (15 mg), stirred and allowed to warm to RT over 1 hour. The mixture was filtered to remove solids then chromatographed (30% ethyl acetate in hexane) to give analog 304 (60% yield).

Example 107. Synthesis of Analog 305: Analog 009 (99.1 mg) was dissolved in anhydrous CH₂Cl₂ (3.0 mL), pyridine (150 μL) added, then p-nitrophenylchloroformate and stirred for 3.5 hours at RT. The mixture was concentrated, hexane (20 mL) added, and precipitate filtered before chromatographing (50% ethyl acetate in hexane) to give analog 305 (50% yield).

Example 108. Synthesis of Analog 306: Analog 009 (244 mg) was dissolved in anhydrous CH₂Cl₂ (4.0 mL), tosyl chloride (181 mg) added, the mixture cooled to 0° C., to which an aliquot of pyridine (80 μL) was added. The mixture stirred at 0° C. for 1 hour, and allowed to warm to RT while being stirred for an additional 20 hours. The mixture was concentrated then chromatographed (50% ethyl acetate in hexane) to yield analog 306.

Example 109. Synthesis of Analog 307: A solution of 1.0 M N₃H in benzene was first prepared by mixing 654 mg N₃H, 0.65 mL water, in 10 mL of benzene. The mixture was cooled to 0° C., 0.5 mL of concentrated H₂SO₄ added, and allowed to warm slowly to RT and then stirred for 80 minutes. Next PPh₃ (590 mg) was dissolved in anhydrous THF (1.5 mL) and cooled to 0° C. Then 2.1 mL of N₃H 1.0 M solution was added, followed by DEAD (0.475 mL) then Illudin S (282 mg in 1.0 mL anhydrous THF). The mixture was stirred for 3 hours at 0° C., warmed, concentrated, followed by chromatography (30% ethyl acetate in hexane) to give analog 307.

Example 110. Synthesis of Analog 308: Analog 307 (100 mg) was dissolved in anhydrous THF (3.0 mL) at RT and PPH3 added (306 mg, 3 equivalents). The mixture was stirred for 5 hours at RT, then the reaction stooped by adding water (0.15 mL). The mixture was heated to 85° C. for 30 minutes, then concentrated and chromatographed (20% methanol in ethyl acetate) to give analog 308.

Example 111. Synthesis of Analog 309: Analog 204 was reacted with HN₃ (DEAD, THF) to yield the azide analog 309 at 68% yield.

Example 112. Synthesis of Analog 310: Irofulven (42.9 mg), 4-carboxybenzene sulfonamide (41.4 mg), and DCC (38.4 mg) were dissolved in anhydrous DMF (1.0 mL) at RT, stirred and then DMAP (10 mg) added. The mixture was stirred for 75 minutes at RT then solid material was filtered off. The mixture was then chromatographed (1:1 ethyl acetate: hexane) to give analog 310 (40% yield).

Example 113. Synthesis of Analog 311: Illudin M (32.4 mg), 4-carboxybenzene sulfonamide (39.7 mg), and DCC (24.4 mg) were dissolved in anhydrous DMF (1.0 mL) at RT, stirred, then DMAP (15 mg) added. The mixture was stirred for 75 minutes at RT, allowed to warm to RT, then stirred for 22 hours. The solid material was filtered off and the mixture was then chromatographed (1:1 ethyl acetate: hexane) to give analog 311 (35% yield).

Example 114. Synthesis of Analog 312: Irofulven (1.18 grams) was dissolved in anhydrous CH₂Cl₂ (4.0 mL), tosyl chloride (1.1 equivalent) added, the mixture cooled to 0° C., then pyridine (0.4 mL) added. The mixture stirred at 0° C. for 1 hour, and allowed to warm to RT while being stirred for an additional 3 hours. The mixture was concentrated then chromatographed (50% ethyl acetate in hexane) to yield analog 312.

Example 115. Synthesis of Analog 313: Analog 308 (31 mg) was dissolved in anhydrous CH₂Cl₂, cooled to 0° C., with stirring then diisopropylethylamine added (45 μL), then fluorophenylsulfonyl chloride added (36 μL) for 3 hours at 0° C. Mixture was directly chromatographed (20% ethyl acetate: hexane) to give analog 313 (23.3 mg).

Example 116. Synthesis of Analog 314: Analog 009 was dissolved in anhydrous CH₂Cl₂ (4.0 mL), tosyl chloride (1.1 equivalent) added, the mixture cooled to 0° C., then pyridine (0.4 mL) added. The mixture stirred at 0° C. for 1 hour, and allowed to warm to RT while being stirred for an additional 3 hours. The mixture was concentrated then chromatographed (50% ethyl acetate in hexane) to yield analog 314.

Example 117. Synthesis of Analog 315: Irofulven was dissolved in a solution of 2,5 dimethylpyrrole (4 fold excess molar solution) in 5 mL of dry CH₂Cl₂ at −78° C. Boron trifluoride (equivalent molar amount to the irofulven) was slowly added with stirring. The reaction was allowed to stir for 2 more hours at −78° C., then water slowly added. The mixture was extracted twice with 2 fold equivalent volumes of ethyl acetate, the organic extracts combined, washed with saturated NaHCO₃, water, brine, then dried over MgSO₄. The solution was concentrated under vacuum until a red residue remained, which was chromatographed on silica gel (50% ethyl acetate in hexane) to yield analog 315 (30% yield).

Example 118. Synthesis of Analog 316: Analog 316 was prepared by dissolving Illudin S (20 mg) in pyridine (0.5 mL) and then 4-fluorosulfonylbenzoyl chloride (equivalent molar amount) was added to the mixture in an ice bath. The solution was allowed to warm slowly and then react overnight. The liquid was then removed under reduced pressure until a crude residue remained. Rather than recrystallize from chloroform, the residue was instead chromatographed on a standard silica gel column using hexane-ethyl acetate (1:1). The mono-adduct (analog 316), a di-adduct and a small amount of unreacted Illudin S were recovered in separate eluates.

Example 119. N₃H 1.0 M Solution: A solution of 1.0 M N₃H in benzene was first prepared by mixing 654 mg N₃H, 0.65 mL water, in 10 mL of benzene. The mixture was cooled to 0° C., 0.5 mL of concentrated H₂SO₄ added, and allowed to warm slowly to RT and then stirred for 80 minutes.

Example 120. Synthesis of Analog 193: Irofulven (221 mg, 0.897 umol) was dissolved in anhydrous THF (1.5 mL), then PPh₃ (261 mg, 0.995 umol) was added, then 1.0 M N₃H solution (1.0 mL, 1.0 mmol) under nitrogen atmosphere. The solution was cooled to −40° C., and then DIAD (0.21 mL, 1.013 umol) added and stirred for 30 minutes at 0° C. then diluted with hexane, and filtered to remove precipitate. The mixture was concentrated then chromatographed (30% ethyl acetate: hexane) to give analog 193 (171 mg, 71%).

Example 121. Synthesis of Analog 195: Analog 009 (31.9 mg, 116 umol) was dissolved in anhydrous THF (3.0 mL), then PPh₃ (33 mg, 126 umol) was added, then 1.0 M N₃H solution (0.3061 mL) under nitrogen atmosphere. The solution was cooled to 0° C., DIAD (30 μL, 145 umol) added and stirred for 30 minutes at 0° C. then water (5 μL) was added to destroy the PPh₃. The mixture was concentrated then chromatographed (30% ethyl acetate: hexane) to give analog 195 (24.9 mg, 72%).

Example 122. Synthesis of Analog 317: Analog 219 (33 mg) was dissolved in anhydrous acetonitrile (0.5 mL) and pyridine (0.1 mL) at 0° C. under nitrogen. Then add HF-pyridine solution (7 uL, 0.245 mmol, 35M, 6.6 equiv). After 10 minutes K₂CO₃ was added (0.5 mL, 0.5M) and the mixture concentrated then chromatographed using CH₂Cl₂, methanol, triethylamine (5:0.5:0.1) to yield analog 317.

Example 123. Synthesis of Analog 318: Illudin S (30 mg) was dissolved in 1.0 mL of anhydrous pyridine under a nitrogen atmosphere, then DMAP (5.0 mg) was added, followed by excess 4-fluorosulfonyl-benzoyl chloride (190 mg). The mixture was stirred for 90 minutes at RT then diluted with 5.0 mL of ethyl acetate, washed once with saturated copper sulfate solution, washed twice with water, then dried over MgSO₄, concentrated then chromatographed (20% ethyl acetate: hexane) to give analog 318.

Example 124. Synthesis of Analog 332: Analog 034 (200 mg) was dissolved in ethanol (95%) at RT, then 300 mg of O-methylhydroxylamine hydrochloride (NH₂OMe HCl; Note O-methyl not the N-methyl compound) was added with sodium acetate (300 mg), stirred for 6 hours, concentrated, then chromatographed (15% ethyl acetate/hexanes) to yield the analog 332.

Example 125. Synthesis of Analog 333: Prepared by dissolving analog 159 and p-nitrophenylchloroformate in anhydrous CH₂Cl₂ under nitrogen for 5 hours at 0° C. with 1.5 equivalents of DIPEA and 1.0 equivalents of DMAP. The CH₂Cl₂ was removed and analog 333 was purified using a 7% ethyl acetate/hexane column. Yield was 70%.

Example 126. Synthesis of Analog 334: Prepared by dissolving analog 159 and p-nitrophenylchloroformate in anhydrous CH₂Cl₂ under nitrogen at 0° C. with 1.5 equivalents of DIPEA and 1.0 equivalents of DMAP. After 5 hours the Boc-N,N′-dimethylaminoethane was added and the mixture kept at 0° C. under nitrogen for 2 hours then allowed to warm up to RT and react overnight. The CH₂Cl₂ was removed and analog 334 purified using a 15% ethyl acetate/hexane column.

Example 127. Synthesis of Analog 335: Analog 334 (19 μmole) was dissolved in 1 mL of methanol and then sodium methoxide in methanol (0.5 M, 100 μmole) was added at RT with stirring for 30 minutes. The reaction was quenched by addition of excess acetic acid (40 μmole). The methanol was removed and analog 335 purified using a 25% ethyl acetate/hexane column.

Example 128. Synthesis of Analog 336: Analog 002 (200 mg) was placed in a dry round bottom, add 10 mL anhydrous CH₂Cl₂, stir at 0° C. under nitrogen, add aluminum trichloride (AlCl3). The mixture will turn dark orange, stir additional 10 min at 0° C., add 20 mL of ethylene oxide/CH₂Cl₂ solution at 0° C., stir for 30 minutes at 0° C. Wash twice with 10% HCl/ice cold water (2×50 mL), then twice with water (2×25 mL), dry over Na₂SO₄, concentrate under reduced pressure and chromatograph using ethyl acetate/hexane mixture.

Example 129. Synthesis of Analog 337: Analog 270 (244 mg) was dissolved in 5.0 mL of CH₂Cl₂ and DABCO added (153 mg), and solution cooled to 0° C. Then p-nitrophenylchloroformate (162 mg) was added with stirring for 3 hours at 0° C. Mixture was concentrated and chromatographed (50% ethyl acetate 50% hexanes) to yield 201 mg of analog 337 as a yellow liquid.

Example 130. Synthesis of Analog 338: Analog 211 (82 mg) was dissolved in 5.0 mL of CH₂Cl₂ and DABCO added (42 mg), and solution cooled to 0° C. Then p-nitrophenylchloroformate (51 mg) was added with stirring for 5 hours at 0° C. Mixture was concentrated and chromatographed (50% ethyl acetate 50% hexanes) to yield 63 mg of analog 338 as a yellow liquid.

Example 131. Synthesis of Analog 339: Illudin S is dissolved in anhydrous CH₂Cl₂ (4.0 mL), tosyl chloride (1.1 equivalent) added, the mixture cooled to OC, then pyridine (400 uL) added. The mixture stirred at OC for 1 hour, and allowed to warm to RT while being stirred for an additional 3 hours. The mixture is concentrated then chromatographed (50% ethyl acetate in hexane) to yield analog 339.

Example 132. Synthesis of Analog 340: Analog 270 (146 mg), DABCO (90 mg) was dissolved in DMF/CH₂Cl₂ (1:3; 5.0 mL), cooled to 0° C., then methane sulfonyl chloride (55 uL; Cl—SO₂—CH₃) added with stirring for 2 hours at 0° C., concentrated then chromatographed using ethyl acetate/hexane=(1:1) to yield analog 340 as a yellow liquid.

Example 133. Synthesis of Analog 347: Illudin S (35 mg) was dissolved in 1.0 mL of anhydrous pyridine under a nitrogen atmosphere, then DMAP (5.0 mg) was added, followed by 4-sulfamidobenzoyl chloride (90 mg). The mixture was stirred for 90 minutes at RT then diluted with ethyl acetate, washed once with saturated copper sulfate solution, washed twice with water, then dried over MgSO₄, concentrated, then chromatographed (20% ethyl acetate: hexane) to give analog 347.

Example 134. Synthesis of Analog 348: Illudin M (15 mg) was dissolved in 1.0 mL of anhydrous pyridine under a nitrogen atmosphere, then DMAP (3.0 mg) was added, followed by 4-sulfamidobenzoyl chloride (33 mg). The mixture was stirred for 90 minutes at RT then diluted with ethyl acetate, washed once with saturated copper sulfate solution, washed twice with water, then dried over MgSO₄, concentrated then chromatographed (20% ethyl acetate: hexane) to give analog 348.

Example 135. Synthesis of Analog 353: Analog 334 was dissolved in anhydrous CH₂Cl₂ at 0° C. and TFA added to a final concentration of 40% for 40 minutes to remove the Boc protecting group. The reaction was stopped by adding additional anhydrous CH₂Cl₂ to dilute the final TFA concentration to 10%, then solvent and TFA removed using a roto-evaporator without heat. Next the residue was dissolved in peptide synthesis grade DMF (so no solvent amines are present) and commercially available analog 388 was added at 0° C., followed by triethylamine (8.0 equivalents) with stirring overnight. After concentration the material was purified by preparative HPLC. Solvent A (aqueous) was 0.1% TFA in water. Solvent B (organic) was 60% acetonitrile, 39.9% water, and 0.1% TFA. HPLC gradient was initially 60% B increasing to 100% B over 45 minutes, with analog 353 eluting at approximately 40 minutes.

Example 136. Synthesis of Analog 356: Dissolve analog 334 in CH₂Cl₂ at 0° C., then add ice cold TFA to a final concentration of 25%, for 30 minutes to remove Boc group. Excess TFA removed by evaporating to dryness under vacuum, then add 0.5 ml CH₂Cl₂, evaporating to dryness under vacuum, repeat CH₂Cl₂ addition and evaporation a total of 3 times and keep on ice/cold the entire time. Add 1 ml dry DMF, add triethylamine (5 equiv), and keep on ice. Part B: Take Fmoc-triglycine-OH peptide (1.1 equiv) and dissolve in 3 mls dry DMF at RT, then add EDC (1.1 equiv) and NHS (N-hydroxysuccinimide, 1.1 equiv) to form the active ester, and cool on ice. Add solution B to A on ice, then allow to warm in chiller to RT overnight to produce 356 (yield ˜60%).

Example 137. Synthesis of Analog 357: Prepared by dissolving analog 359 in CH₂Cl₂ at 0° C., then adding ice cold TFA to a final concentration of 25%, for 30 minutes. Excess TFA removed by evaporating to dryness under vacuum, adding 0.5 ml CH₂Cl₂, evaporating to dryness under vacuum total of 3 times.

Example 138. Synthesis of Analog 359: Part A: Dissolve analog 334 in CH₂Cl₂ at 0° C., then adding ice cold TFA to a final concentration of 25%, for 30 minutes to remove Boc group. Excess TFA removed by evaporating to dryness under vacuum, adding 0.5 ml CH₂Cl₂, evaporating to dryness under vacuum, repeat CH₂Cl₂ addition and evaporation a total of 3 times, and keep on ice/cold the entire time. Add 1 ml dry DMF, add triethylamine (5 equiv), and keep on ice. Part B: Take Boc-triglycine-OH peptide (1.1 equiv) and dissolve in 3 ml dry DMF at RT, then add EDC (1.1 equiv) and NHS (N-hydroxysuccinimide, 1.1 equiv) to form the active ester, and cool on ice. Add solution from Part B to solution from Part A on ice, then allow to warm in chiller to RT overnight to produce analog 359.

Example 139. Synthesis of Analog 361: Add analog 159 and N,N′-Disuccinimidyl carbonate (1.1 equiv) in anhydrous dichloromethane, DIPEA (1.1 equiv) and DMAP (1.1 equiv), reflux for four hours.

Example 140. Synthesis of Analog 362: Illudin S and 1.2 equivalents of mesylate anhydride, and 1.2 equivalents of 2,4,6-collidine into dry CH₂Cl₂, use molecular sieves. Start the reaction at 0° C. (on ice) then let sit overnight, gradually warming up to RT. The reaction mixture remained colorless, and analog 362 was isolated as a white crystalline material using ethyl acetate/hexane chromatography.

Example 141. Synthesis of Analog 363: Parik-Doering reaction was used with analog 383 in which pyridine sulfur trioxide was used in combination with DIPEA and DMSO under strict anhydrous conditions. At 0° C. the reaction mix contained a minor amount of product compatible with analog 383 when analyzed by LC/MS. When the reaction was precooled to −20° C., allowed to react with illudin S, and then slowly warmed to 0° C., analog 383 was produced at −60% yield. Analog 383 was reacted with NH₂—Boc in the presence of TFA and triethylsilane under the same conditions that analog 002 was reacted with NH₂—Boc to produce analog 366. However, the yields were very low at <15%. Analog 383 and 1.1 equivalent of NH₂-Boc were dissolved in anhydrous DCM with molecular sieves, 0.2 equivalents of acetic acid added (to produce the —C═N—HR intermediate), and allowed to sit overnight. In the morning sodium triacetoxyborohydride was added (4 equivalents) and the reaction allowed to proceed for 6 hours to produce analog 389. Analog 389 was converted to the primary amine analog 363 using standard Boc deprotection method.

Example 142. Synthesis of Analog 365. Illudin S (150 mg) was reacted with Dess Martin Periodinane reagent (1.1 equiv) for 60 minutes at RT in 15 mls CH₂Cl₂ (no molecular sieves are used as traces of water accelerate the reaction). Solvent removed and analog 365 (110 mg) recovered using ethyl acetate/hexane chromatography.

Example 143. Synthesis of Analog 366. Add 5 mg of analog 002 into a small vial with 50 μL of acetone, added BocNH₂ 6.5 mg (2.5 equiv), started reaction with 20 ul of 1 N H₂SO₄, allowed to go overnight at RT. The yield was >80%.

Example 144. Synthesis of Analog 367: Analog 010 (80 mg) was dissolved in 3 ml of anhydrous acetonitrile with molecular sieves. Add 102 mg NH₂Boc (3 equiv), 140 ul Et₃SiH (3 equiv) stirred at RT for 30 minutes with sieves under nitrogen, added 63 μL of TFA (2.9 equiv). Within 30 minutes solution turning from yellow to red. Eventually turns dark brown (amine product). The solvent was removed, and compound isolated using ethyl acetate/hexane chromatography.

Example 145. Synthesis of Analog 368: Illudin S (185 mg, 0.522 mmol), DIPEA 329 mg/444 ul (5 equiv), was dissolved in 5 ml THF, add acetic anhydride 260 ul (5 equiv) and DMAP 62 mg (1 equiv) and monitor reaction at RT. To recover and purify use an acetone/hexane column (0% acetone, then 2, 4, 6% etc.).

Example 146. Synthesis of Analog 369. Illudin S (1.025 grams) and 385.1 mg of imidazole (1.46 equiv) into 8 mls dry DMF with molecular sieves and stirred for 25 minutes at RT under nitrogen, then 686 mg of TBDMSCl added (1.17 equiv) in two equal portions. The reaction was over in 20 minutes per TLC. Add saturated sodium bicarbonate to quench reaction, extracted ethyl acetate three times, organic phase recovered and washed with brine, and dried with anhydrous magnesium sulfate, then solvent removed under high vacuum and temperature of 45° C.

Example 147. Synthesis of Analog 370: Analog 369 was dissolved in DMF then added to 10 mls of CH₂Cl₂, then 316 mg of sodium acetate added (1 equiv), stirred for RT for 15 minutes, then acetic anhydride added (1.02 equiv), and allowed to react overnight. The reaction may not progress unless DMAP was added (0.2 equiv). The next day additional sodium acetate (150 mg, 0.3 equiv) and acetic anhydride (185 ul; 0.5 equiv) and DMAP (0.2 equiv) added. The next day solvent removed under high vacuum and temperature of 45° C.

Example 148. Synthesis of Analog 371: Analog 368 was dissolved in excess 7N NH₃/methanol and allow to react slowly for 48 to 72 hours at 40° C. then the primary acetate was selectively removed and analog 371 was primarily produced with a yield of ˜65% (illudin S was the other product at ˜35%).

Example 149. Synthesis of Analog 372: Dissolve 320 mg of #371 and 620 mg of Dess Martin (1.2 equiv) into 4 mls CH₂Cl₂ (no molecular sieves are used as traces of water accelerate the reaction), allowed to react for 45 minutes. Then added 3 mis of saturated bicarbonate, 3 mls of saturated Na₂S₂O₃ (to quench Dess Martin reagent), then 15 mls of diethyl ether. Wash the reaction mixture twice more with diethyl ether, dry with anhydrous sodium sulfate and then remove the ether. Store overnight then purified with column (elutes at 15% ethyl acetate/hexane).

Example 150. Synthesis of Analog 373: Place 4.5 mg of analog 372 in 200 ul anhydrous CH₂Cl₂ with several molecular sieves. Add triethylsilane (3.0 equiv), TFA (2.9 equiv) and FmocNH₂ (3.0 equiv) in dry THF at RT for 6 hours under nitrogen. Purify by ethyl acetate/hexane gradient column. Yield ˜40%.

Example 151. Synthesis of Analog 374: The Fmoc moiety of analog 373 was removed using 4-methylpyridine (20% final) in DMF at RT for 30 minutes.

Example 152. Synthesis of Analog 377: Dissolve analog 159 into dry CH₂Cl₂ with molecular sieves, add imidazole (1.1 equiv) and stir for 30 minutes at RT. Then 1.2 equiv of TBSCl (chlorotributyl silane) added. After 15 minutes add 0.3 equiv of additional TBSCL.

Example 153. Synthesis of Analog 378: 4.5 mg of analog 372 in 200 ul DMF with molecular sieves under nitrogen. Add triethylsilane (3.0 equiv), TFA (2.9 equiv) and BocNH₂ (3.0 equiv) at RT for 6 hours under nitrogen. Purify by ethyl acetate/hexane gradient column. Yield ˜40%.

Example 154. Synthesis of Analog 379: Analog 359: Part A: Dissolving analog 334 in CH₂Cl₂ at 0° C., then adding ice cold TFA to a final concentration of 25%, for 30 minutes to remove Boc group. Excess TFA removed by evaporating to dryness under vacuum, adding 0.5 ml CH₂Cl₂, evaporating to dryness under vacuum total of 3 times and keep on ice/cold the entire time. Add 1 ml dry DMF, triethylamine (5 equiv), and keep on ice. Part B: Take Fmoc-triglycine-Val-cit-PNP peptide (1.1 equiv) and dissolve in 3 mls of dry DMF at 0° C. Add solution B to A on ice, then allow to warm in chiller to RT overnight to produce 379.

Example 155. Synthesis of Analog 380: Prepare from analog 379 by removing Fmoc using 4-methylpyridine (20% final) in DMF at RT for 30 minutes.

Example 156. Synthesis of Analog 381: Prepare from analog 377 using lithium hydroxide (LiOH) (1.0 equiv) in 10% methanol/THF at 4° C. One must have methanol otherwise LiOH acts as a nucleophile and removes the TBS, not the acetate protective group. After 1 hour then add additional 0.2 equiv LiOH, then after 30 minutes remove THF/methanol. Purify immediately using ethyl acetate/hexane column to prevent degradation.

Example 157. Synthesis of Analog 382. Add 0.5 equiv of Dess Martin periodinane reagent to analog 381 at RT in CH₂Cl₂. Add another 0.5 equiv after 30 minutes, then another 0.2 equiv after 30 minutes. Added 3 ml saturated bicarbonate, 3 ml of saturated Na₂S₂O₃ (to quench Dess Martin reagent), then 15 mls of diethyl ether. Wash reaction mix 2 more times with diethyl ether, dry with anhydrous sodium sulfate and remove ether. The product can be stored overnight then purified with column.

Example 158. Synthesis of Analog 383: Prepared from Analog 382 by treating with 1.5% HCl/methanol at RT for 60 minutes. If one sees a smear by TLC, then leave at 4° C. overnight, and in the morning one will see a main product by TLC that was analog 383. Yield was poor as normally less than 25%.

Example 159. Synthesis of Analog 384: Analog 382 was dissolved in 1.5 ml anhydrous acetonitrile, then add BocNH₂ (3.0 equiv), then triethylsilane (3.0 equiv), followed by TFA (2.9 equiv) for 5 hours at RT. Best to add TFA in aliquots so as to not remove the protecting silyl group. Recover product using 10% acetone/hexane column (not ethyl acetate column), but still the yield was low at ˜15%.

Example 160. Synthesis of Analog 385: Illudin S (10 mg) was dissolved in anhydrous methanol with molecular sieves; add 0.5 equiv of Indium (II) chloride, 2.0 equiv of triethylsilane). Let sit at RT overnight, remove methanol, and purify using an ethyl acetate/hexane column.

Example 161. Synthesis of Analog 387: Dissolve the boc-triglycine (20 mg/ml) in DMF, then add EDC (0.98 equiv) and pentafluorophenol (1.15 equiv) at RT for 2 hours. Evaporate with high vacuum at 40° C. then add ethyl acetate. If solids present remove by filtration. Wash with saturated sodium bicarbonate solution to remove unreacted triglycine acid and unreacted pentafluorophenol, then wash with brine, dry and evaporate to a flaky white solid. (when an oil was obtained simply add diethyl ether to precipitate a flaky white solid).

Example 162. Synthesis of Analog 389: Analog 383 and 1.1 equivalent of BocNH₂ are dissolved in anhydrous CH₂Cl₂ with molecular sieves, 0.2 equivalents of acetic acid added (to produce the —C═N—HR intermediate), and allowed to sit overnight. In the morning sodium triacetoxyborohydride was added (4 equivalents) and the reaction allowed to proceed for 6 hours to produce analog 389. Product was purified using an ethyl acetate/hexane column. The yield was ˜25%.

Example 163: Synthesis of Analog 392: Part A: Dissolve analog 334 in CH₂Cl₂ at 0° C., then adding ice cold TFA to a final concentration of 25%, for 30 minutes to remove Boc group. Excess TFA removed by evaporating to dryness under vacuum, adding 0.5 ml CH₂Cl₂, evaporating to dryness under vacuum, repeat CH₂Cl₂ addition and evaporation a total of 3 times, and keep on ice/cold the entire time. Add 1 ml dry DMF, add triethylamine (5 equiv), and keep on ice. Part B: Take the Azide N₃-triglycine-OH peptide (1.1 equiv) and dissolve in 3 ml dry DMF at RT, then add EDC (1.1 equiv) and NHS (N-hydroxysuccinimide, 1.1 equiv) to form the active ester, and cool on ice. Add solution from Part B to solution from Part A on ice, then allow to warm in chiller to RT overnight to produce analog 392.

Example 164: Synthesis of Analog 394: Dissolve analog 235 into CH₂CL₂, chill on ice then add TFA to a final concentration of 10%, and allow to incubate on ice for 60 minutes. Solvent is removed using high vacuum, then the product is purified using an ethyl acetate/hexane column. The yield is ˜90%.

Example 165: Synthesis of Analog 397: Into a 50 ml round bottom flask is placed 400 mg of Illudin S and then 20 ml of anhydrous DCM added along with molecular sieves. The reaction is allowed to stir at RT for 30 minutes to extract any water contained in the Illudin S. Then 1.06 ml of DIPEA is added with stirring for 15 minutes. Next the mixture is cooled to −70° C., then 1.0 g of sulfur trioxide pyridine complex (Sigma Aldrich catalog #S7556) is dissolved into 1 ml of anhydrous DMSO containing molecular sieves at RT. The sulfur trioxide pyridine/DMSO oxidizing complex is slowly added to the Illudin S solution at −70° C. with stirring. The reaction is allowed to slowly warm to −20° C., and maintained at that temperature for 15 minutes. The reaction is quenched with 10 ml of water followed immediately by 10 ml of ethyl acetate. Next 300 μl of 20% aqueous ammonia hydroxide is added whereupon a bright red color appears in the aqueous portion. The organic portion is removed, and the aqueous portion is extracted 3 more times with the ammonia hydroxide/ethyl acetate procedure. The organic phases are combined, washed with saturated bicarbonate, and then washed with saturated brine. Finally the organic phase is dried then the solvent removed. The product is purified using by silica gel chromatography using an ethyl acetate/hexane gradient to yield the intermediate primary aldehyde. Yield is approximately 30%. This primary aldehyde of Illudin S is then dissolved in anhydrous THF, then 2 equivalents of anhydrous sodium sulfate added, and the mixture cooled to 4° C. under nitrogen. Then 3.0 equivalents of N-Boc Hydroxylamine are added. After 15 minutes, 3.5 pl of glacial acetic acid are added, followed by 6.0 equivalents of sodium triacetoxy borohydride. The reaction is allowed to proceed for 18 hours at 4° C. under nitrogen. The mixture is filtered and the organic solvent removed by vacuum. The product is purified using silica gel chromatography with an ethyl acetate/hexane gradient to yield the hydroxylamine Boc derivative analog 397.

Example 166: Synthesis of Analog 398: Analog 397 is dissolved in anhydrous THF, without molecular sieves, under nitrogen and cooled to 0° C. Then TFA is added to a final concentration of 35% for 25 minutes. The THF and TFA are removed by high vacuum without external heat to yield the hydroxylamine analog 398 (final yield approximately 20%).

Example 167: Synthesis of Analog 399: Illudin S (700 mg) is dissolved in 70 ml of anhydrous DCM and 5.0 ml of peptide synthesis grade DMF at RT. Then molecular sieves are added and the mixture is stirred at RT for 30 minutes under nitrogen, and 2,4,6-trimethylpyridine (5.7 equivalents) is slowly added until the Illudin S is dissolved. The mixture is then cooled to −4° C. and sulfamoyl chloride (1.2 equivalents) is slowly added over a 15 minute period. The reaction is allowed to proceed for 4 hours at −4° C., then allowed to slowly warm and react overnight at RT for 16 hours. The DCM/DMF reaction mixture is washed with saturated sodium bicarbonate to remove residual sulfonic and hydrochloric acids. The remaining organic phase is dried then removed with high vacuum. The product is purified using silica gel chromatography with an ethyl acetate/hexane gradient to yield analog 399. Yield is approximately 25%. The product should be stored at −20° C.

Example 168: Synthesis of Analog 401: Illudin S (500 mg) is dissolved in a mixture of 300 mg of sodium acetate and 6.0 ml of acetic anhydride. The reaction is allowed to proceed at RT with stirring and monitored. After 3 to 4 hours the reaction is quenched by adding 100 ml of saturated sodium bicarbonate solution. The aqueous solution is extracted four times with ethyl acetate and the organic fractions are combined, dried and removed by vacuum. The desired mono-acetate product is separated from the undesired residual Illudin S, di-acetate and secondary mono-acetate contaminating compounds by silica gel chromatography using an ethyl acetate/hexane gradient. The recovered mono-acetate product is dissolved in anhydrous CH₂Cl₂ under nitrogen at 0° C. with 1.15 equivalents of p-nitrophenylchloroformate. Next 1.5 equivalents of DIPEA and 1.0 equivalents of DMAP are slowly added with stirring. Then the Boc-N,N′-dimethylaminoethane reagent (3.0 equivalents) is slowly added and the mixture is kept at 0° C. under nitrogen for 2 hours then allowed to warm up to RT and react overnight. The CH₂Cl₂ is removed and the product is purified by silica gel chromatography using an ethyl acetate/hexane gradient to yield the desired product. If not used immediately this intermediate should be stored at −70° C. under an inert gas. The intermediate (41 mg) is dissolved in 1.5 ml of anhydrous CH₂Cl₂ under nitrogen at 0° C. into a 25 ml round bottom flask, and 50 μl of anisole is added. Next 1.2 ml of trifluoroacetic acid is slowly added in 6 equal aliquots. The reaction is left stirring on ice, under nitrogen, for 45 minutes. Then 10 ml of pre-cooled (−20° C.) CHCl₃ is quickly added. All liquid is quickly removed under high vacuum. The residue is placed back into the ice bath and 1.5 ml of pre-cooled (−20° C.) DMF added. After 15 minutes the Fmoc-Val-Cit-PAB-PNP linker (62 mg, Axis Pharma catalog #AP10017) is added with stirring. After 15 minutes triethylamine (217 μl) is added. The mixture is stirred under nitrogen at 0° C. for 1 hour, then allowed to slowly warm to RT. If desired, the CH₂Cl₂ is removed and the product is purified by silica gel chromatography using an ethyl acetate/hexane gradient to yield the desired product analog 401.

Example 169: Synthesis of Analog 404: Analog 401 can be redissolved in 1. 5 ml of anhydrous CH₂Cl₂ under nitrogen at 0° C., then 200 μl of triethylamine added, the vial sealed and allowed to react at 4° C. for 18 hours. The mixture is purified by silica gel chromatography using an ethanol/chloroform gradient to yield the desired product analog 404 (final yield approximately 20%).

Example 170: Synthesis of Analog 405: 50 mg of VP-PEG4-Acid (1-(6-methyl-4-vinylpyridin-2-yl)-3-oxo-7,110,13,16-tetraoxa-4-azanonadecan-19-oic acid) and N-hydroxysuccinimide (16 mg, 1.20 equivalents) are dissolved in 20 ml of anhydrous CH₂Cl₂ under nitrogen. The EDC (1-ethyl-3-(3-dimethylaminopropyl)carbodiimide, 21 mg, 1.20 equivalents) is added and allowed to react at RT for 24 hours to produce the compound VP-PEG4-NHS (2,5-dioxopyrrolidin-1-yl 1-(6-methyl-4-vinylpyridin-22-yl)-3-oxo-7,10,13,16-tetraoxa-4-azanondecane-19-oate). The CH₂Cl₂ mixture containing the VP-PEG4-NHS is washed with water, then quickly dried and the CH₂Cl₂ removed by vacuum. Next analog 404 (74 mg) is dissolved in 3.0 ml of DMF, and then added to the VP-PEG-NHS compound. The mixture is placed under nitrogen, cooled to 0° C., then 210 μl of triethylamine added, and the reaction allowed to continue overnight in the cold. The desired product, analog 405, is recovered using reverse phase HPLC (approximately 50% yield of initial starting material analog 404).

Example 171: Synthesis of Analog 402 and Analog 403: Illudin S (0.95 grams) is dissolved in 100 ml of water, and then 100 ml of hexane is added to form an upper organic layer. With rapid stirring, 2.06 ml of concentrated sulfuric acid is added slowly. After 1 hour the hexane is removed and another 100 ml of hexane is added. This process is repeated one more time. The hexane layers are combined washed with a saturated sodium bicarbonate solution to neutralize residual acid, dried with anhydrous magnesium, then solvent removed to yield a bright yellow oil (AF1). Next paraformaldehyde (0.66 g) is dissolved in 60 ml of water, and concentrated sulfuric acid (5.0 ml) is slowly added. The cloudy solution is slowly heated until the solution is clear. This paraformaldehyde solution is allowed to slowly cool to 30° C. Then the previously prepared bright yellow intermediate (AF1) is dissolved in acetone, and the mixture slowly added with continuous stirring for 24 hours. The reaction mixture is extracted three times with ethyl acetate (100 ml), the extracts are combined, washed with saturated sodium bicarbonate, dried over anhydrous magnesium sulfate, then the organic solvent removed by vacuum. The residue is purified by silica gel chromatography using an ethyl acetate/hexane gradient to yield a bright orange intermediate (IRO1). IRO1 is dissolved in anhydrous dichloromethane, under nitrogen, and 2,4,6-trimethylpyridine (3.0 equivalents) is added, then the mixture is cooled to 0° C. Sulfamoyl chloride (1.15 equivalents) is dissolved in anhydrous peptide-grade DMF (1 ml), and is slowly added to the dichloromethane solution, with mixing. The solution is mixed for one hour at 0° C., then slowly allowed to warm to RT, and continued to react for a total of 4 hours. The reaction mixture is concentrated by vacuum, then purified by silica gel chromatography using an ethyl acetate/hexane gradient to yield analog 402 and analog 403 (final yield of both products is approximately 15%).

Example 172: Synthesis of Analog 407: Illudin S (100 mg) is dissolved in 20 ml of anhydrous DCM and 5.0 ml of DMF at RT. Then molecular sieves are added and the mixture is stirred at RT for 30 minutes under nitrogen, and 2,4,6-trimethylpyridine (5.7 equivalents) is slowly added until the Illudin S is dissolved. The mixture is then cooled to −4° C. and the trifluormethanesulfanyl chloride (Aldrich Catalog #164798, 1.2 equivalents) is slowly added over a 15 minute period. The reaction is allowed to proceed for 4 hours at −4° C., and then allowed to slowly warm and react overnight at RT for 16 hours. The DCM/DMF reaction mixture is washed with saturated sodium bicarbonate to remove residual sulfonic and hydrochloric acids. The remaining organic phase is dried then removed with high vacuum. The product is purified using by silica gel chromatography using an ethyl acetate/hexane gradient to yield analog 407. Yield is approximately 15%. The product is unstable and should be stored at −70° C. under an inert gas.

Example 173: Synthesis of Analog 408: Illudin S (500 mg) is dissolved in a mixture of 300 mg of sodium acetate and 6.0 ml of acetic anhydride. The reaction is allowed to proceed at RT with stirring and monitored. After 3 to 4 hours the reaction is quenched by adding 100 ml of saturated sodium bicarbonate solution. The aqueous solution is extracted four times with ethyl acetate and the organic fractions are combined, dried and removed by vacuum. The desired mono-acetate product is separated from the undesired residual Illudin S, di-acetate and secondary mono-acetate contaminating compounds by silica gel chromatography using an ethyl acetate/hexane gradient. The recovered mono-acetate product is dissolved in anhydrous CH₂Cl₂ under nitrogen at 0° C. with 1.15 equivalents of p-nitrophenylchloroformate. Next 1.5 equivalents of DIPEA and 1.0 equivalents of DMAP are slowly added with stirring. After the Boc-N,N′-dimethylaminoethane reagent (3.0 equivalents) is added and the mixture is kept at 0° C. under nitrogen for an 2 hours then allowed to warm up to RT and react overnight. The CH₂Cl₂ is removed and the product is purified by silica gel chromatography using an ethyl acetate/hexane gradient to yield the desired intermediate product. If not used immediately this intermediate should be stored at −70° C. under an inert gas. The intermediate (41 mg) is dissolved in 1.5 ml of anhydrous CH₂Cl₂ under nitrogen at 0° C. into a 25 ml round bottom flask, and 50 μl of anisole is added. Next 1.2 ml of trifluoroacetic acid is slowly added in 6 equal aliquots. The reaction is left stirring on ice, under nitrogen, for 45 minutes. Then 10 ml of precooled (−20° C.) CHCl₃ added. All liquid is removed under high vacuum. The residue is placed back in to the ice bath and 1.5 ml of pre-cooled (−20° C.) DMF added. This solution is added to a solution of 3 ml of DMF containing the Fmoc-Gly3-Val-Cit-PNP peptide (Levena Biopharma Catalog #H1002, 1.1 equivalent) at 0° C. The solution is allowed to slowly warm to RT and react overnight. The desired intermediate is recovered by precipitation with excess diethyl ether. The precipitate is washed twice with ice cold ether, dried to remove the ether, then redissolved in 1.5 ml of DMF, 200 μl of triethylamine added, and the reaction allowed to proceed at 4° C. for 48 hours to produce analog 408 (final yield is approximately 20%).

Example 174: Synthesis of Analog 409: Illudin S (500 mg) is dissolved in a mixture of 300 mg of sodium acetate and 6.0 ml of acetic anhydride. The reaction is allowed to proceed at RT with stirring and monitored. The reaction is quenched after 3-4 hours by adding 100 ml of saturated sodium bicarbonate solution. The aqueous solution is extracted four times with ethyl acetate, the organic fractions are combined, dried and removed by vacuum. The desired mono-acetate product is separated from the undesired residual Illudin S, di-acetate and secondary mono-acetate contaminating compounds by silica gel chromatography using an ethyl acetate/hexane gradient. The recovered mono-acetate product is dissolved in anhydrous CH₂Cl₂ under nitrogen at 0° C. with 1.15 equivalents of p-nitrophenylchloroformate. Next is added with stirring 1.5 equivalents of DIPEA and 1.0 equivalents of DMAP. After the Boc-N,N′-dimethylaminoethane reagent (3.0 equivalents) is added and the mixture is kept at 0° C. under nitrogen for an additional 2 hours then allowed to warm up to RT and react overnight. The CH₂Cl₂ is removed and the product is purified by silica gel chromatography using an ethyl acetate/hexane gradient to yield the desired product. If not used immediately this intermediate should be stored at −70° C. under an inert gas. This intermediate (41 mg) is dissolved in 1.5 ml of anhydrous CH₂Cl₂ under nitrogen at 0° C. into a 25 ml round bottom flask, and 50 μl of anisole is added. Next 1.2 ml of trifluoroacetic acid is slowly added in 6 equal aliquots. The reaction is left stirring on ice, under nitrogen, for 45 minutes. Then 10 ml of precooled (−20° C.) CHCl₃ is added. All liquid is removed under high vacuum. The residue is placed back in to the ice bath and 1.5 ml of pre-cooled (−20° C.) peptide synthesis grade DMF added. After 15 minutes the Fmoc-Val-Cit-PAB-PNP linker (62 mg. Axis Pharma catalog #AP10017) is added with stirring. After 15 minutes then triethylamine (217 μl) is added. The mixture is stirred under nitrogen at 0° C. for 1 hour, then allowed to slowly warm to RT. If desired, the CH₂Cl₂ is removed and the product is purified by silica gel chromatography using an ethyl acetate/hexane gradient to yield the desired intermediate product. The intermediate can be redissolved in 1. 5 ml of anhydrous CH₂Cl₂ under nitrogen at 0° C., then 200 μl of triethylamine added, the vial sealed and allowed to react at 4 C for 18 hours. The desired intermediate is recovered by precipitation with excess ice-cold diethyl ether. The precipitate is washed twice with ice cold ether, dried to remove the ether, then redissolved in 1.5 ml of DMF and 200 μl of triethylamine is added, and the reaction allowed to proceed at 4° C. for 48 hours to produce analog 409 (final yield approximately 30%).

Example 175: Synthesis of Analog 410: Illudin S (500 mg) is dissolved in a mixture of 300 mg of sodium acetate and 6.0 mls of acetic anhydride. The reaction is allowed to proceed at RT with stirring and monitored. The reaction is quenched after 3-4 hours by adding 100 ml of saturated sodium bicarbonate solution. The aqueous solution is extracted four times with ethyl acetate, the organic fractions are combined, dried and removed by vacuum. The desired mono-acetate product is separated from the undesired residual Illudin S, di-acetate and secondary mono-acetate contaminating compounds by silica gel chromatography using an ethyl acetate/hexane gradient. The recovered mono-acetate product is dissolved in anhydrous CH₂Cl₂ under nitrogen at 0° C. with 1.15 equivalents of p-nitrophenylchloroformate. Next is added with stirring 1.5 equivalents of DIPEA and 1.0 equivalents of DMAP. After the Boc-N,N′-dimethylaminoethane reagent (3.0 equivalents) is added, the mixture is kept at 0° C. under nitrogen for an additional 2 hours then allowed to warm up to RT and react overnight. The CH₂Cl₂ is removed and the product is purified by silica gel chromatography using an ethyl acetate/hexane gradient to yield the desired product. If not used immediately this intermediate should be stored at −70° C. under an inert gas. This intermediate (41 mg) is dissolved in 1.5 mls of anhydrous CH₂Cl₂ under nitrogen at 0° C. into a 25 ml round bottom flask, and 50 μl of anisole is added. Next 1.2 ml of trifluoroacetic acid is slowly added in 6 equal aliquots. The reaction is left stirring on ice, under nitrogen, for 45 minutes. Then 10 ml of pre-cooled (−20° C.) CHCl₃ is added. All liquid is removed under high vacuum. The residue is placed back in to the ice bath and 1.5 ml of pre-cooled (−20° C.) DMF added. Next MC-Val-Cit-PAB-PNP (Levena Biopharma Catalog #VC1004, 1.2 equivalents) is directly added with stirring while maintaining the temperature at 0° C. Once all solid material is dissolved then triethylamine (8.0 equivalents) is added and the reaction continued at 4° C. for 48 hours. Analog 410 is recovered by preparative HPLC using an acetonitrile gradient (5% to 95%) with 0.05% trifluoroacetic acid (final yield approximately 15%).

Abbreviations used include: DMAP=4-dimethylaminopyridine; DCC=N,N′-dicycyclohexylcarbodiimide; ODHBt=3,4,-dihydroxy-4-oxo-1,2,3-benzo-triazine-3-yl ester; NMM=N-methylmorpholine; EDC=1-(3-dimethylaminopropyl)-3-ethylcarbodiimide hydrochloride; DIAD=diisopropyl azodicarboxylate; DEAD=diethyl azodicarboxylate; and DIPC=N,N′-diisopropylcarbodiimide. Boc₂O=anhydrous Boc; BocNH₂=t-butyl carbamate; DIPEA=N,N-diisopropylethylamine; ODHBt=3,4,-dihydroxy-4-oxo-1,2,3-benzo-triazine-3-yl ester (refers to ester made from HOOBt); HOOBt=(Hydroxy-3,4-dihydro-4-oxo-1,2,3-benzotriazine); NMM=N-methylmorpholine; RT—room temperature; TBDMSCl=t-butyldimethyl silyl chloride; TEA=triethanolamine.

The foregoing description of embodiments of the methods, systems, and components of the present invention has been provided for the purposes of illustration and description. It is not intended to be exhaustive or to limit the invention to the precise forms disclosed. Many modifications and variations will be apparent to one of ordinary skill in the relevant arts. For example, steps performed in the embodiments of the invention disclosed can be performed in alternate orders, certain steps can be omitted, and additional steps can be added. The embodiments were chosen and described in order to best explain the principles of the invention and its practical application, thereby enabling others skilled in the art to understand the invention for various embodiments and with various modifications that are suited to the particular used contemplated. Other embodiments are possible and are covered by the invention. Such embodiments will be apparent to persons skilled in the relevant art(s) based on the teachings contained herein. The breadth and scope of the present invention should not be limited by any of the above-described exemplary embodiments, but should be defined only in accordance with the following claims and their equivalents.

TABLE I
Cytotoxic data IC₅₀ values (micromolar, 2 hour exposure, N = 3,
mean ± SD) for Illudin M, analog 108 and analog 110
for cells expressing the estrogen receptor (ER)
(MCF7) and cells not expressing the ER (HT29).

| Analog | HT29 (ER Negative) | MCF7 (ER positive) |
|---|---|---|
| Illudin M | 0.52 ± 0.10 | 0.48 ± 0.13 |
| 108 | >55 | 14.1 ± 2.8 |
| 110 | >19 | 2.0 ± 0.1 |

TABLE II
Activity of PSA cleavable illudofulvene analogs (210, 215, 216, 221)
compared with Illudin S and illudofulvene precursor analogs (204, 207,
211, 212, 213, 214) against PSA negative and PSA positive cell line
(48 hour exposure, N = 3; mean ± SD; IC50 values in nM).

| | Prostate PC3 | Prostate DuPro | Prostate LnCAP |
|---|---|---|---|
| Analog | (negative PSA) | (trace PSA) | (positive PSA) |
| Illudin S | 16 ± 5 | 11 ± 3 | 15 ± 3 |
| 204 | n.t. | n.t. | 3,300 ± 1,000 |
| 207 | 880 ± 330 | 450 ± 40 | 560 ± 60 |
| 211 | 350 ± 80 | 280 ± 20 | 270 ± 50 |
| 212 | 120 ± 20 | 20 ± 2 | 120 ± 30 |
| 213 | 2,200 ± 100 | 360 ± 80 | 900 ± 200 |
| 214 | 300 ± 50 | 90 ± 10 | 190 ± 30 |
| 210 | 4,700 ± 500 | 3,500 ± 400 | 810 ± 130 |
| 215 | n.t. | n.t. | >20,000 |
| 216 | 190 ± 10 | 280 ± 60 | 190 ± 30 |
| 221 | >21,000 | 13,000 ± 1,000 | 800 ± 100 |

n.t. denotes not tested

TABLE III
Peptides cleaved by various proteases.

| Protease | Peptide | SEQ. ID’s |
|---|---|---|
| PSA | His-Ser-Ser-Lys-Leu-Gln-X | SEQ. ID. 104 |
| | Mu-His-Ser-Ser-Lys-Leu-Gln-X | SEQ. ID. 106 |
| | Mu-His-Ser-Ser-Lys-Leu-Gln-Lys-X | SEQ. ID. 108 |
| | Mu-His-Ser-Ser-Lys-Leu-EDA-Lys-X | |
| | Mc-His-Ser-Ser-Lys-Leu-Gln-X | |
| | Mc-His-Ser-Ser-Lys-Leu-Gln-X | |
| | Hyp-Ala-Ser-Chg-Gln-Ser-X | SEQ. ID. 111 |
| | Hyp-Ala-Ser-Chg-Gln-Ser-Leu-X | SEQ. ID. 116 |
| | Mu-Hyp-Ala-Ser-Chg-Gln-Ser-X | |
| | Mu-Hyp-Ala-Ser-Chg-Gln-Ser-Leu-X | |
| | Mc-Hyp-Ala-Ser-Chg-Gln-Ser-X | |
| | Mc-Hyp-Ala-Ser-Chg-Gln-Ser-Leu-X | |
| | Hyp-Ser-Ser-Chg-GIn-Ser-Ser-Pro-X | SEQ. ID. 127 |
| | Mu-Hyp-Ser-Ser-Chg-Gln-Ser-Ser-Pro-X | SEQ. ID. 131 |
| | Mc-Hyp-Ser-Ser-Chg-Gln-Ser-Ser-Pro-X | |
| | 4-O-Ac-Hyp-Ser-Ser-Chg-Gln-Ser-Ser-Pro-X | |
| | Arg-Arg-Ser-Ser-Tyr-Tyr-Ser-Gly-X | SEQ. ID. 132 |
| | Mu-Arg-Arg-Ser-Ser-Tyr-Tyr-Ser-Gly-X | SEQ. ID. 136 |
| | Mc-Arg-Arg-Ser-Ser-Tyr-Tyr-Ser-Gly-X | |
| | Mc-Ser-Ser-Lys-Tyr-Gln-Leu-X | |
| | Mu-Ser-Ser-Lys-Tyr-Gln-Leu-X | |
| | N-glutaryl-Hyp-Ala-Ser-chGly-Gln-Ser-Leu | SEQ. ID. 137 |
| | Mu-N-glutaryl-Hyp-Ala-Ser-chGly-Gln-Ser-Leu | |
| | Mc-N-glutaryl-Hyp-Ala-Ser-chGly-Gln-Ser-Leu | |
| Caspase-3 | Asp-Glu-Val-Asp-Pro-X | SEQ. ID. 138 |
| | Mu-Asp-Glu-Val-Asp-Pro-X | |
| | Mc-Asp-Glu-Val-Asp-Pro-X | |
| | Lys-Gly-Ser-Gly-Asp-Val-Glu-Gly-X | SEQ. ID. 139 |
| | Mu-Lys-Gly-Ser-Gly-Asp-Val-Glu-Gly-X | |
| | Mc-Lys-Gly-Ser-Gly-Asp-Val-Glu-Gly-X | |
| Cathepsin B | PLE-X | SEQ. ID. 141 |
| | Gly-Phe-Leu-Gly-X | |
| | Lys-Lys-Phe-D-Ala-X | SEQ. ID. 142 |
| | D-Ala-Phe-Lys-Lys-X | SEQ. ID. 144 |
| | Mc-Poly-L-glutamic acid-X | SEQ. ID. 145 |
| | Mc-Gly-Phe-Leu-Gly-X | |
| | Mc-Lys-Lys-Phe-D-Ala-X | |
| | Mc-D-Ala-Phe-Lys-Lys-X | |
| | Mu-Poly-L-glutamic acid-X | |
| | Mu-Gly-Phe-Leu-Gly-X | |
| | Mu-Lys-Lys-Phe-D-Ala-X | |
| | Mu-D-Ala-Phe-Lys-Lys-X | |
| | Val-Cit-X | |
| FAP | Lys-Gln-Glu-Gln-Asn-Pro-Gly-Ser-Thr-X | SEQ. ID. 146 |
| | Mu-Lys-Gln-Glu-Gln-Asn-Pro-Gly-Ser-Thr-X | |
| | Mc-Lys-Gln-Glu-Gln-Asn-Pro-Gly-Ser-Thr-X | |
| Kallikrein 2 | Gly-Lys-Ala-Phe-Arg-Arg-X | SEQ. ID. 171 |
| | Mu-Gly-Lys-Ala-Phe-Arg-Arg-X | |
| | Mc-Gly-Lys-Ala-Phe-Arg-Arg-X | |
| MMP-2/-9/ | Glu-Pro-Cit-Gly-Hof-Tyr-Leu-X | SEQ. ID. 172 |
| | Mu-Glu-Pro-Cit-Gly-Hof-Tyr-Leu-X | |
| | Mc-Glu-Pro-Cit-Gly-Hof-Tyr-Leu-X | |
| | Gly-Ile-Leu-Gly-Val-Pro-X | SEQ. ID. 173 |
| | Mu-Gly-Ile-Leu-Gly-Val-Pro-X | |
| | Mc-Gly-Ile-Leu-Gly-Val-Pro-X | |
| | Gly-Pro-Leu-Gly-Ile-Ala-Gly-Gln-X | SEQ. ID. 174 |
| | Mu-Gly-Pro-Leu-Gly-lle-Ala-Gly-Gln-X | |
| | Mc-Gly-Pro-Leu-Gly-Ile-Ala-Gly-Gln-X | |
| MMP-7 | Lys-Arg-Ala-Leu-Gly-Leu-Pro-Gly | SEQ. ID. 175 |
| | Mu-Lys-Arg-Ala-Leu-Gly-Leu-Pro-Gly | |
| | Mc-Lys-Arg-Ala-Leu-Gly-Leu-Pro-Gly | |
| | Arg-Pro-Leu-Ala-Leu-Trp-Arg-Ser | SEQ. ID. 176 |
| | Mu-Arg-Pro-Leu-Ala-Leu-Trp-Arg-Ser | |
| | Mc-Arg-Pro-Leu-Ala-Leu-Trp-Arg-Ser | |
| TOP | Ala-L-L-Ala-L-Ile | |
| | Mu-Ala-L-L-Ala-L-lle | |
| | Mc-Ala-L-L-Ala-L-Ile | |
| uPA | D-Ala-Phe-Lys or | SEQ. ID. 177 |
| | D-Ala-Phe-Lys-PABC | |
| Cathepsin K | Gly-Gly-Pro-Nle-X | SEQ. ID. 178 |
| | Mu-Gly-Gly-Pro-Nle-X | |
| | Mc-Gly-Gly-Pro-Nle-X | |
| Plasmin | D-Ala-Phe-Lys-Lys-X | SEQ. ID. 179 |
| | Mu-D-Ala-Phe-Lys-Lys-X | |
| | Mc-D-Ala-Phe-Lys-Lys-X | |
| | D-Ala-Phe-Lys-X | |
| | Mu-D-Ala-Phe-Lys-X | |
| | Mc-D-Ala-Phe-Lys-X | |
| Thrombin | Poly-L-Lys-Gly-D-Phe-Pip-Arg-Ser-Gly-Gly- | SEQ. ID. 180 |
| | Gly-Gly-Gly-X | |
| Trypsin | Poly-L-Lysine-Gly-Ala-Ser-D-Arg-Phe-Thr- | SEQ. ID. 181 |
| | Gly-X | |

In Table III, the letter ‘X’ denotes the end attached to the medicant, Chg denotes cyclohexyl glycine, Cit denotes citrulline, EDA denotes ethanyl-D-Alanine, Hof denotes homophenylalanine, Hyp denotes 4-hydroxyproline, Mc denotes morpholinocarbonyl (carboxy-terminal protecting group), Mu denotes 4-morpholine-carbonyl (amino-terminal protecting group), Nle denotes norleucine, PABC denotes para-aminobenzoylcarboxyl, PLE denotes Poly-L-glutamic acid, and Pip denotes pipecolic acid.

TABLE IV
Mechanisms of Drug Resistance.

| Mechanism of Multi- | Resistance to |
|---|---|
| drug Resistance | illudofulvenes |
| Gp170/MDR1 | No |
| Gp180/MRP | No |
| Topoisomerase I | No |
| Topoisomerase II | No |
| MVP/LRP (vault) | No |
| Thiol content/GST pi | No |
| DNA repair | No |
| Myc expression | No |
| Bcl-2 expression | No |
| BRCA status | No |
| P53 status | No |
| P21 status | No |
| MGMT expression | No |
| Microtubulin alteration | No |

TABLE V
IUPAC names of the Illudofulvene analogs.

| | Illudo- | |
|---|---|---|
| Entry | fulvene | |
| # | Analog | IUPAC Name of Illudofulvene Analog |
| 1 | 001 | (R)-6′-hydroxy-2′,4′,6′-trimethylspiro[cyclopropane- |
| | | 1,5′-inden]-7′(6′H)-one |
| 2 | 002 | (6′R)-6′-hydroxy-3′-(hydroxymethyl)-2′,4′,6′- |
| | | trimethylspiro[cyclopropane-1,5′-inden]-7′(6′H)-one |
| 3 | 003 | (6′R,6′′′R)-3′,3″′-methylenebis(6′-hydroxy-2′,4′,6′- |
| | | trimethylspiro[cyclopropane-1,5′-inden]-7′(6′H)-one) |
| 4 | 004 | (R)-3′-bromo-6′-hydroxy-2′,4′,6′- |
| | | trimethylspiro[cyclopropane-1,5′-inden]-7′(6′H)-one |
| 5 | 005 | (R)-6′-hydroxy-3′-iodo-2′,4′,6′- |
| | | trimethylspiro[cyclopropane-1,5′-inden]-7′(6′H)-one |
| 6 | 006 | (R)-6′-hydroxy-3′-(4-hydroxybenzyl)-2′,4′,6′- |
| | | trimethylspiro[cyclopropane-1,5′-inden]-7′(6′H)-one |
| 7 | 007 | (R)-6′-hydroxy-3′-(4-methoxybenzyl)-2′,4′,6′- |
| | | trimethylspiro[cyclopropane-1,5′-inden]-7′(6′H)-one |
| 8 | 008 | (R)-((6′-hydroxy-2′,4′,6′-trimethyl-7′-oxo-6′,7′- |
| | | dihydrospiro[cyclopropane-1,5′-inden]- |
| | | 3′-yl)methoxy)methyl acetate |
| 9 | 009 | (R)-6′-hydroxy-3′-(3-hydroxypropyl)-2′,4′,6′- |
| | | trimethylspiro[cyclopropane-1,5′-inden]-7′(6′H)-one |
| 10 | 010 | (R)-3-(6′-hydroxy-2′,4′,6′-trimethyl-7′-oxo-6′,7′- |
| | | dihydrospiro[cyclopropane-1,5′-inden]-3′-yl)propanal |
| 11 | 011 | (R)-6′-hydroxy-2′,4′,6′-trimethyl-7′-oxo-6′,7′- |
| | | dihydrospiro[cyclopropane-1,5′-indene]-3′-carbaldehyde |
| 12 | 012 | (R)-6′-hydroxy-2′,4′,6′-trimethyl-3′- |
| | | nitrospiro[cyclopropane-1,5′-inden]-7′(6′H)-one |
| 13 | 013 | 4-hydroxy-5-(((R)-6′-hydroxy-2′,4′,6′-trimethyl-7′- |
| | | oxo-6′,7′-dihydrospiro[cyclopropane-1,5′-inden]-3′- |
| | | yl)methyl)cyclohexane-1,3-dicarbaldehyde |
| 14 | 014 | (4a′S,7′R,9b′S)-7′-hydroxy-4a′,7′,9′-trimethyl-4a′, |
| | | 9b′-dihydro-4′H-spiro[cyclopropane-1,8′- |
| | | indeno[1,2-d][1,3]dioxin]-6′(7′H)-one |
| 15 | 015 | (R)-3′-(hydroxymethyl)-2′,4′,6′-trimethyl-7′-oxo-6′,7′- |
| | | dihydrospiro[cyclopropane-1,5′-inden]-6′-yl acetate |
| 16 | 016 | (R)-3′-(ethoxymethyl)-6′-hydroxy-2′,4′,6′- |
| | | trimethylspiro[cyclopropane-1,5′-inden]-7′(6′H)-one |
| 17 | 017 | (6′R,6″′R)-3′,3″′-(oxybis(methylene))bis(6′-hydroxy- |
| | | 2′,4′,6′-trimethylspiro[cyclopropane- |
| | | 1,5′-inden]-7′(6′H)-one) |
| 18 | 018 | (R)-6′-hydroxy-2′,4′,6′-trimethyl-3′- |
| | | ((((2R,3S,4R,5R,6S)-3,4,5-trihydroxy-6- |
| | | (hydroxymethyl)tetrahydro-2H-pyran-2-yl)oxy)- |
| | | methyl)spiro[cyclopropane-1,5′-inden]-7′(6′H)-one |
| 19 | 019 | (6′R)-3′-((2,3-dihydroxypropoxy)methyl)-6′- |
| | | hydroxy-2′,4′,6′-trimethylspiro[cyclopropane- |
| | | 1,5′-inden]-7′(6′H)-one |
| 20 | 020 | (R)-3′-((2-bromoethoxy)methyl)-6′-hydroxy-2′,4,6′- |
| | | trimethylspiro[cyclopropane-1,5′-inden]-7′(6′H)-one |
| 21 | 021 | (R)-6′-hydroxy-3′-(((2-methoxypropan-2-yl) |
| | | oxy)methyl)-2′,4′,6′-trimethylspiro[cyclopropane- |
| | | 1,5′-inden]-7′(6′H)-one |
| 22 | 022 | (R)-6′-hydroxy-3′-((2-hydroxyethoxy)methyl)-2′,4′,6′- |
| | | trimethylspiro[cyclopropane-1,5′-inden]-7′(6′H)-one |
| 23 | 023 | (R)-6′-hydroxy-3′-(((4-hydroxyphenyl)thio)methyl)- |
| | | 2′,4′,6′-trimethylspiro[cyclopropane- |
| | | 1,5′-inden]-7′(6′H)-one |
| 24 | 024 | (R)-3′-((benzylthio)methyl)-6′-hydroxy-2′,4′,6′- |
| | | trimethylspiro[cyclopropane-1,5′-inden]-7′(6′H)-one |
| 25 | 025 | methyl (R)-2-(((6′-hydroxy-2′,4′,6′-trimethyl-7′-oxo- |
| | | 6′,7′-dihydrospiro[cyclopropane-1,5′-inden]-3′- |
| | | yl)methyl)thio)acetate |
| 26 | 026 | (R)-6′-hydroxy-2′,4′,6′-trimethyl-3′-((p-tolylthio)- |
| | | methyl)spiro[cyclopropane-1,5′-inden]-7′(6′H)-one |
| 27 | 027 | (R)-(6′-hydroxy-2′,4′,6′-trimethyl-7′-oxo-6′,7′- |
| | | dihydrospiro[cyclopropane-1,5′-inden]-3′- |
| | | yl)methyl phenyl carbonate |
| 28 | 028 | (R)-(6′-hydroxy-2′,4′,6′-trimethyl-7′-oxo-6′,7′- |
| | | dihydrospiro[cyclopropane-1,5′-inden]-3′- |
| | | yl)methyl benzoate |
| 29 | 029 | (R)-2-(((6′-hydroxy-2′,4′,6′-trimethyl-7′-oxo-6′,7′- |
| | | dihydrospiro[cyclopropane-1,5′-inden]-3′- |
| | | yl)methyl)thio)acetic acid |
| 30 | 030 | methyl (R)-2-(((6′-hydroxy-1′-((2-methoxy-2- |
| | | oxoethyl)thio)-2′,4′,6′-trimethyl-7′-oxo-6′,7′- |
| | | dihydrospiro[cyclopropane-1,5′-inden]-3′- |
| | | yl)methyl)thio)acetate |
| 31 | 031 | methyl 2-((((6′R)-6′,7a′-dihydroxy-1′-((2-methoxy-2- |
| | | oxoethyl)thio)-2′,4′,6′-trimethyl-7′-oxo-1′,6′,7′,7a′- |
| | | tetrahydrospiro[cyclopropane-1,5′-inden]- |
| | | 3′-yl)methyl)thio)acetate |
| 32 | 032 | (6′R)-3′-(((2,3-dihydroxypropyl)thio)methyl)-6′- |
| | | hydroxy-2′,4′,6′-trimethylspiro[cyclopropane- |
| | | 1,5′-inden]-7′(6′H)-one |
| 33 | 033 | 7′-methyl-4′H-dispiro[cyclopropane-1,6′- |
| | | indene-5′,2″-[1,3]dioxolan]-4′-one |
| 34 | 034 | (R)-(6′-hydroxy-2′,4′,6′-trimethyl-7′-oxo-6′,7′- |
| | | dihydrospiro[cyclopropane-1,5′-inden]- |
| | | 3′-yl)methyl acetate |
| 35 | 035 | 6′-hydroxy-4′-methylspiro[cyclopropane- |
| | | 1,5′-inden]-7′(6′H)-one |
| 36 | 036 | (R)-3′-((lH-imidazol-1-yl)methyl)-6′-hydroxy-2′,4′,6′- |
| | | trimethylspiro[cyclopropane-1,5′-inden]-7′(6′H)-one |
| 37 | 037 | 1-carboxy-2-((((R)-6′-hydroxy-2′,4′,6′-trimethyl-7′- |
| | | oxo-6′,7′-dihydrospiro[cyclopropane-1,5′-inden]- |
| | | 3′-yl)methyl)thio)ethan-1-aminium |
| 38 | 038 | (R)-3-(6′-hydroxy-2′,4′,6′-trimethyl-7′-oxo-6′,7′- |
| | | dihydrospiro[cyclopropane-1,5′-inden]-3′- |
| | | yl)propanoic acid |
| 39 | 039 | (R)-3′-(3,3-dimethoxypropyl)-6′-hydroxy-2′,4′,6′- |
| | | trimethylspiro[cyclopropane-1,5′-inden]-7′(6′H)-one |
| 40 | 040 | (R)-3′-(3,3-diethoxypropyl)-6′-hydroxy-2′,4′,6′- |
| | | trimethylspiro[cyclopropane-1,5′-inden]-7′(6′H)-one |
| 41 | 041 | (R,Z)-3-(6′-hydroxy-2′,4′,6′-trimethyl-7′-oxo-6′,7′- |
| | | dihydrospiro[cyclopropane-1,5′-inden]-3′- |
| | | yl)acrylaldehyde |
| 42 | 042 | (R)-3′-(hydroxymethyl)-4′,6′-dimethyl-6′- |
| | | ((triethylsilyl)oxy)spiro[cyclopropane-1,5′- |
| | | inden]-7′(6′H)-one |
| 43 | 043 | (R)-6′-hydroxy-2′,4′,6′-trimethyl-3′- |
| | | (((triethylsilyl)oxy)methyl)spiro[cyclopropane- |
| | | 1,5′-inden]-7′(6′H)-one |
| 44 | 044 | (R)-2′,4,6′-trimethyl-6′-((triethylsilyl)oxy)-3′- |
| | | (((triethylsilyl)oxy)methyl)spiro[cyclopropane- |
| | | 1,5′-inden]-7′(6′H)-one |
| 45 | 045 | methyl 2-((7-hydroxy-5-(2-hydroxyethyl)-3- |
| | | (hydroxymethyl)-2,4,6-trimethyl-1H-inden- |
| | | 1-yl)thio)acetate |
| 46 | 046 | (R)-3-(6′-hydroxy-2′,4′,6′-trimethyl-7′-oxo-6′,7′- |
| | | dihydrospiro[cyclopropane-1,5′-inden]-3′- |
| | | yl)propyl acetate |
| 47 | 047 | (6′R)-3′-(2-(1,7-dihydroxy-2,4,6-trimethyl-1H- |
| | | inden-5-yl)ethyl)-6′-hydroxy-2′,4′,6′- |
| | | trimethylspiro[cyclopropane-1,5′-inden]- |
| | | 7′(6′H)-one |
| 48 | 048 | (R)-6′-hydroxy-2′,4′,6′-trimethyl-1′-(p-tolylthio)- |
| | | 3′-((p-tolylthio)methyl)spiro[cyclopropane- |
| | | 1,5′-inden]-7′(6′H)-one |
| 49 | 049 | (R)-6′-hydroxy-2′,4′,6′-trimethyl-3′-(p-tolylthio)- |
| | | spiro[cyclopropane-1,5′-inden]-7′(6′H)-one |
| 50 | 050 | (R)-6′-hydroxy-2′,4′,6′-trimethyl-1,3′-bis(p- |
| | | tolylthio)spiro[cyclopropane-1,5′-inden]-7′(6′H)-one |
| 51 | 051 | (R)-2-(2-(((6′-hydroxy-2′,4′,6′-trimethyl-7′-oxo-6′,7′- |
| | | dihydrospiro[cyclopropane-1,5′-inden]-3′- |
| | | yl)methyl)thio)acetoxy)ethyl 2-mercaptoacetate |
| 52 | 052 | ethane-1,2-diyl bis(2-((((R)-6′-hydroxy-2′,4′,6′- |
| | | trimethyl-7′-oxo-6′,7′-dihydrospiro[cyclopropane- |
| | | 1,5′-inden]-3′-yl)methyl)thio)acetate) |
| 53 | 053 | (R)-3′-((2-(2-bromoethoxy)ethoxy)methyl)-6′- |
| | | hydroxy-2′,4′,6′-trimethylspiro[cyclopropane- |
| | | 1,5′-inden]-7′(6′H)-one |
| 54 | 054 | (R)-6′-hydroxy-1′-(((R)-6′-hydroxy-2′,4′,6′- |
| | | trimethyl-7′-oxo-6′,7′-dihydrospiro- |
| | | [cyclopropane-1,5′-inden]-3′-yl)methyl)-3′- |
| | | (hydroxymethyl)-2′,4′,6′-trimethylspiro- |
| | | [cyclopropane-1,5′-inden]-7′(6′H)-one |
| 55 | 055 | 5-(2-hydroxyethyl)-1-((4-hydroxyphenyl)thio)-3- |
| | | (((4-hydroxyphenyl)thio)methyl)-2,4,6-trimethyl- |
| | | 1H-inden-7-ol |
| 56 | 056 | (R)-6′-hydroxy-3′-((4-hydroxyphenyl)thio)-2′,4′,6′- |
| | | trimethylspiro[cyclopropane-1,5′-inden]-7′(6′H)-one |
| 57 | 057 | (R)-6′-hydroxy-1′-((4-hydroxyphenyl)thio)-2′,4′,6′- |
| | | trimethylspiro[cyclopropane-1,5′-inden]-7′(6′H)-one |
| 58 | 058 | (R)-6′-hydroxy-1′,3′-bis((4-hydroxyphenyl)thio)-2′,4′,6′- |
| | | trimethylspiro[cyclopropane-1,5′-inden]-7′(6′H)-one |
| 59 | 059 | (6′S,7′R)-4′-methyl-6′-((triethylsilyl)oxy)-6′,7′- |
| | | dihydrospiro[cyclopropane-1,5′-inden]-7′-ol |
| 60 | 060 | (R)-7′-methyl-4′H-dispiro[cyclopropane-1,6′- |
| | | indene-5′,2″-[1,3]dioxolan]-4′-ol |
| 61 | 061 | (S)-4′-methyl-6′-((triethylsilyl)oxy)spiro- |
| | | [cyclopropane-1,5′-inden]-7′(6′H)-one |
| 62 | 062 | (R)-6′-hydroxy-2′-(hydroxymethyl)-4′,6′- |
| | | dimethylspiro[cyclopropane-1,5′-inden]-7′(6′H)-one |
| 63 | 063 | (R)-6′-hydroxy-2′,3′-bis(hydroxymethyl)-4′,6′- |
| | | dimethylspiro[cyclopropane-1,5′-inden]-7′(6′H)-one |
| 64 | 064 | N-acetyl-S-(((R)-6′-hydroxy-2′,4′,6′-trimethyl- |
| | | 7′-oxo-6′,7′-dihydrospiro[cyclopropane- |
| | | 1,5′-inden]-3′-yl)methyl)-L-cysteine |
| 65 | 065 | (R)-2-acetamido-3-((((R)-6′-hydroxy-4′,6′-dimethyl- |
| | | 7′-oxo-6′,7′-dihydrospiro[cyclopropane-1,5′-inden]-3′- |
| | | yl)methyl)thio)-N-((S)-1-phenylethyl)propanamide |
| 66 | 066 | (S)-2-acetamido-3-((((R)-6′-hydroxy-2′,4′,6′- |
| | | trimethyl-7′-oxo-6′,7′-dihydrospiro[cyclopropane- |
| | | 1,5′-inden]-3′-yl)methyl)thio)-N-((S)-1- |
| | | phenylethyl)propanamide |
| 67 | 067 | 4-methyl-2,3-dihydro-5H-indeno[5,6-b]furan-5-one |
| 68 | 068 | 5-hydroxy-6-(2-hydroxyethyl)-7-methyl-1H- |
| | | inden-1-one |
| 69 | 069 | 5-(2-hydroxyethoxy)-6-(2-hydroxyethyl)-7- |
| | | methyl-1H-inden-1-one |
| 70 | 070 | (3a′R,4′R)-4′-hydroxy-7′-methyl-3a′,4′-dihydro- |
| | | 1′H-dispiro[cyclopropane-1,6′-indene- |
| | | 5′,2″-[1,3]dioxolan]-1′-one |
| 71 | 071 | (3a′R,4′R)-7′-methyl-4′-((triethylsilyl)oxy)- |
| | | 3a′,4′-dihydro-1′H-dispiro[cyclopropane- |
| | | 1,6′-indene-5′,2″-[1,3]dioxolan]-1′-one |
| 72 | 072 | (7′R,7a′R)-7′-hydroxy-4′-methyl-7′,7a′-dihydrospiro- |
| | | [cyclopropane-1,5′-indene]-3,6′-dione |
| 73 | 073 | (7′R,7a′R)-4′-methyl-7′-((triethylsilyl)oxy)-7′,7a′- |
| | | dihydrospiro[cyclopropane-1,5′-indene]-3′,6′-dione |
| 74 | 074 | (6′R)-3′-((((2,2-dimethyl-1,3-dioxolan-4-yl)methyl)- |
| | | thio)methyl)-6′-hydroxy-2′,4′,6′-trimethylspiro- |
| | | [cyclopropane-1,5′-inden]-7′(6′H)-one |
| 75 | 075 | (R)-(6′-hydroxy-4′,6′-dimethyl-7′-oxo-6′,7′- |
| | | dihydrospiro[cyclopropane-1,5′-inden]-2′- |
| | | yl)methyl acetate |
| 76 | 076 | (R)-(6′-hydroxy-4′,6′-dimethyl-7′-oxo-6′,7′- |
| | | dihydrospiro[cyclopropane-1,5′-indene]- |
| | | 2′,3′-diyl)bis(methylene) diacetate |
| 77 | 077 | (R)-(6′-hydroxy-3′-(hydroxymethyl)-4′,6′- |
| | | dimethyl-7′-oxo-6′,7′-dihydrospiro- |
| | | [cyclopropane-1,5′-inden]-2′- |
| | | yl)methyl acetate |
| 78 | 078 | (R)-(6′-hydroxy-2′-(hydroxymethyl)-4′,6′- |
| | | dimethyl-7′-oxo-6′,7′-dihydrospiro- |
| | | [cyclopropane-1,5′-inden]-3′-yl)methyl acetate |
| 79 | 079 | (R)-6′-hydroxy-2′-(methoxymethyl)-4′,6′- |
| | | dimethylspiro[cyclopropane-1,5′-inden]-7′(6′H)-one |
| 80 | 080 | (R)-6′-hydroxy-3′-(methoxymethyl)-2′,4′,6′- |
| | | trimethylspiro[cyclopropane-1,5′-inden]-7′(6′H)-one |
| 81 | 081 | (R)-6′-hydroxy-2′-(hydroxymethyl)-3′- |
| | | (methoxymethyl)-4′,6′-dimethylspiro- |
| | | [cyclopropane-1,5′-inden]-7′(6′H)-one |
| 82 | 082 | (R)-6′-hydroxy-2′,3′-bis(methoxymethyl)-4′,6′- |
| | | dimethylspiro[cyclopropane-1,5′-inden]-7′(6′H)-one |
| 83 | 083 | (R)-2-acetamido-3-((((R)-6′-hydroxy-2′,4′,6′- |
| | | trimethyl-7′-oxo-6′,7′-dihydrospiro- |
| | | [cyclopropane-1,5′-inden]-3′-yl)methyl)thio)- |
| | | N-((S)-1-((2-(((S)-4-methyl-1- |
| | | oxopentan-2-yl)amino)-2-oxoethyl)amino)-1- |
| | | oxo-3-phenylpropan-2-yl)propanamide |
| 84 | 084 | (S)-2-((R)-2-acetamido-3-((((R)-6′-hydroxy- |
| | | 2′,4′,6′-trimethyl-7′-oxo-6′,7′-dihydrospiro- |
| | | [cyclopropane-1,5′-inden]-3′-yl)methyl)thio)- |
| | | propanamido)-4-methyl-N-(2-oxo-2-(((R)-1-oxo-3- |
| | | phenylpropan-2-yl)amino)ethyl)pentanamide |
| 85 | 085 | (S)-2-((R)-2-acetamido-3-((((R)-6′-hydroxy- |
| | | 2′,4′,6′-trimethyl-7′-oxo-6′,7′-dihydrospiro- |
| | | [cyclopropane-1,5′-inden]-3′-yl)methyl)- |
| | | thio)propanamido)-4-methyl-N-((S)- |
| | | 4-methyl-1-oxo-1-(((R)-1-oxo-3-phenylpropan- |
| | | 2-yl)amino)pentan-2-yl)pentanamide |
| 86 | 086 | (R)-2-acetamido-3-((((R)-6′-hydroxy-2′,4′, |
| | | 6′-trimethyl-7′-oxo-6′,7′-dihydrospiro- |
| | | [cyclopropane-1,5′-inden]-3′-yl)methyl)thio)- |
| | | N-(2-oxo-2-(((R)-1-oxo-3- |
| | | phenylpropan-2-yl)amino)ethyl)propanamide |
| 87 | 087 | (S)-2-((R)-2-acetamido-3-((((R)-6′-hydroxy- |
| | | 2′,4′,6′-trimethyl-7′-oxo-6′,7′-dihydrospiro- |
| | | [cyclopropane-1,5′-inden]-3′-yl)methyl)thio)- |
| | | propanamido)-4-methyl-N-((R)-4-methyl-1- |
| | | (((S)-4-methyl-1-oxopentan-2-yl)amino)- |
| | | 1-oxopentan-2-yl)pentanamide |
| 88 | 088 | (R)-(6′-acetoxy-2′,4′,6′-trimethyl-7′-oxo-6′,7- |
| | | dihydrospiro[cyclopropane-1,5′-inden]-3′- |
| | | yl)methyl acetate |
| 89 | 089 | N5-((R)-1-((carboxymethyl)amino)-3-((((R)-6′- |
| | | hydroxy-2′,4′,6′-trimethyl-7′-oxo-6′,7′- |
| | | dihydrospiro[cyclopropane-1,5′-inden]-3′- |
| | | yl)methyl)thio)-1-oxopropan-2-yl)-D-glutamine |
| 90 | 090 | (R)-2′-(hydroxymethyl)-4′,6′-dimethyl-7′-oxo-6′,7′- |
| | | dihydrospiro[cyclopropane-1,5′-inden]-6′-yl acetate |
| 91 | 091 | (R)-3-(6′-hydroxy-2′,4′,6′-trimethyl-7′-oxo-6′,7′- |
| | | dihydrospiro[cyclopropane-1,5′-inden]-3′- |
| | | yl)propanoic acid |
| 92 | 092 | (R)-6′-hydroxy-3′-(3-methoxypropyl)-2′,4′,6′- |
| | | trimethylspiro[cyclopropane-1,5′-inden]-7′(6′H)-one |
| 93 | 093 | (R)-3′-(3,3-diethoxypropyl)-6′-hydroxy-2′,4′,6- |
| | | trimethylspiro[cyclopropane-1,5′-inden]-7′(6′H)-one |
| 94 | 094 | (R)-3-(6′-hydroxy-2′,4′,6′-trimethyl-7′-oxo-6′,7′- |
| | | dihydrospiro[cyclopropane-1,5′-inden]-3′- |
| | | yl)propyl acetate |
| 95 | 095 | (R)-6′-hydroxy-3′-(3-methoxypropyl)-2′,4′,6′- |
| | | trimethylspiro[cyclopropane-1,5′-inden]-7′(6′H)-one |
| 96 | 096 | (R)-6′-hydroxy-2′,4′,6′-trimethyl-3′-(((1-methyl- |
| | | 1H-imidazol-2-yl)thio)methyl)spiro- |
| | | [cyclopropane-1,5′-inden]-7′(6′H)-one |
| 97 | 097 | S-(((R)-6′-hydroxy-2′,4′,6′-trimethyl-7′-oxo-6′,7′- |
| | | dihydrospiro[cyclopropane-1,5′-inden]-3′- |
| | | yl)methyl)homocysteine |
| 98 | 098 | ((S)-3-((((R)-6′-hydroxy-2′,4′,6′-trimethyl-7′-oxo-6′, |
| | | 7′-dihydrospiro[cyclopropane-1,5′-inden]-3′- |
| | | yl)methyl)thio)-2-methylpropanoyl)proline |
| 99 | 099 | (2′S,6′R)-6′-hydroxy-2′-(hydroxymethyl)-2′,4′,6′- |
| | | trimethylspiro[cyclopropane-1,5′- |
| | | indene]-3′,7′(2′H,6′H)-dione |
| 100 | 100 | S-(((R)-6′-hydroxy-2′,4′,6′-trimethyl-7′-oxo- |
| | | 6′,7′-dihydrospiro[cyclopropane-1,5′- |
| | | inden]-3′-yl)methyl)-N-S-(((R)-6′-hydroxy- |
| | | 2′,4′,6′-trimethyl-7′-oxo-6′,7′-dihydrospiro- |
| | | [cyclopropane-1,5′-inden]-3′-yl)methyl)cysteinyl- |
| | | L-asparaginylglycyl-L-arginylcysteine |
| 101 | 101 | S-(((R)-6′-hydroxy-2′,4′,6′-trimethyl-7′-oxo-6′,7′- |
| | | dihydrospiro[cyclopropane-1,5′-inden]-3′-yl)methyl)- |
| | | N-S-(((R)-6′-hydroxy-2′,4′,6′-trimethyl- |
| | | 7′-oxo-6′,7′-dihydrospiro[cyclopropane-1,5′- |
| | | inden]-3′-yl)methyl)cysteinyl-L-arginylglycyl- |
| | | L-asparaginylcysteine |
| 102 | 102 | S-(((R)-6′-hydroxy-2′,4′,6′-trimethyl-7′-oxo-6′,7′- |
| | | dihydrospiro[cyclopropane-1,5′-inden]-3′- |
| | | yl)methyl)cysteinyl-L- |
| | | asparaginylglycyl-L-arginylcysteine |
| 103 | 103 | (R)-(6′-acetoxy-2′-(hydroxymethyl)-4′,6′-dimethyl- |
| | | 7′-oxo-6′,7′-dihydrospiro[cyclopropane- |
| | | 1,5′-inden]-3′-yl)methyl acetate |
| 104 | 104 | (R)-8′-hydroxy-6′,8′-dimethyl-1′,5′-dihydrospiro- |
| | | [cyclopropane-1,7′-indeno[1,2-e][1,3]dioxepin]- |
| | | 9′(8′H)-one |
| 105 | 105 | (E)-2-((2R,4S)-4-hydroxy-2-((1R,2S)-2- |
| | | hydroxy-4,4-dimethylcyclopentyl)-2- |
| | | methylcyclobutylidene)propanal |
| 106 | 106 | 5-(((3′S,6′R)-6′-hydroxy-2′,2′,4′,6′-tetramethyl-7′- |
| | | oxo-2′,3′,6′,7′-tetrahydrospiro[cyclopropane-1,5′- |
| | | inden]-3′-yl)oxy)-5-oxopentanoic acid |
| 107 | 107 | (3′S,6′R)-6′-hydroxy-2′,2′,4′,6′-tetramethyl-7′-oxo- |
| | | 2′,3′,6′,7′-tetrahydrospiro[cyclopropane-1,5′-inden]- |
| | | 3′-yl ((13S)-13-methyl-17-oxo- |
| | | 7,8,9,11,12,13,14,15,16,17-decahydro-6H- |
| | | cyclopenta[a]phenanthren-3-yl) glutarate |
| 108 | 108 | (13S)-17-hydroxy-13-methyl-7,8,9,11,12,13, |
| | | 14,15,16,17-decahydro-6H-cyclopenta- |
| | | [a]phenanthren-3-yl 3-((R)-6′-hydroxy- |
| | | 2′,4′,6′-trimethyl-7′-oxo-6′,7′-dihydrospiro- |
| | | [cyclopropane-1,5′-inden]-3′-yl)propanoate |
| 109 | 109 | (13S)-17-hydroxy-13-methyl-7,8,9,11,12,13, |
| | | 14,15,16,17-decahydro-6H-cyclopenta- |
| | | [a]phenanthren-3-yl ((3′S,6′R)-6′-hydroxy- |
| | | 2′,2′,4′,6′-tetramethyl-7′-oxo-2′,3′,6′,7′- |
| | | tetrahydrospiro[cyclopropane-1,5′-inden]- |
| | | 3′-yl) glutarate |
| 110 | 110 | (13S)-13-methyl-17-oxo-7,8,9,11,12,13,14, |
| | | 15,16,17-decahydro-6H-cyclopenta[a]- |
| | | phenanthren-3-yl 3-((R)-6′-hydroxy-2′,4′,6′- |
| | | trimethyl-7′-oxo-6′,7′-dihydrospiro[cyclopropane- |
| | | 1,5′-inden]-3′-yl)propanoate |
| 111 | 111 | (10R,13S)-10,13-dimethyl-3-oxo-2,3,6,7,8,9, |
| | | 10,11,12,13,14,15,16,17-tetradecahydro-1H- |
| | | cyclopenta[a]phenanthren-17-yl 3-(6′-hydroxy- |
| | | 2′,4′-dimethyl-7′-oxo-6′,7′-dihydrospiro- |
| | | [cyclopropane-1,5′-inden]-3′-yl)propanoate |
| 112 | 112 | (13S)-10,13-dimethyl-17-oxohexadecahydro-1H- |
| | | cyclopenta[a]phenanthren-3-yl 3-((R)-6′-hydroxy- |
| | | 2′,4′,6′-trimethyl-7′-oxo-6′,7′-dihydrospiro- |
| | | [cyclopropane-1,5′-inden]-3′-yl)propanoate |
| 113 | 113 | (R)-3′-(but-3-en-1-yl)-6′-hydroxy-2′,4′,6′- |
| | | trimethylspiro[cyclopropane-1,5′-inden]-7′(6′H)-one |
| 114 | 114 | (6′R)-6′-hydroxy-2′,4′,6′-trimethyl-3′-(2-(oxiran-2- |
| | | yl)ethyl)spiro[cyclopropane-1,5′-inden]-7′(6′H)-one |
| 115 | 115 | (R)-3-(6′-hydroxy-2′,4′,6′-trimethyl-7′-oxo-6′,7′- |
| | | dihydrospiro[cyclopropane-1,5′-inden]-3′- |
| | | yl)propanal oxime |
| 116 | 116 | (R)-3′-(tert-butoxymethyl)-6′-hydroxy-2′,4′,6′- |
| | | trimethylspiro[cyclopropane-1,5′-inden]-7′(6′H)-one |
| 117 | 117 | 5-(((2′S,6′R)-3′-((4-carboxybutanoyl)oxy)- |
| | | 6′-hydroxy-2′,4′,6′-trimethyl-7′-oxo- |
| | | 2′,3′,6′,7′-tetrahydrospiro[cyclopropane-1,5′- |
| | | inden]-2′-yl)methoxy)-5-oxopentanoic acid |
| 118 | 118 | 5-(((2′S,6′R)-2′-(((3,5-dinitrobenzoyl)oxy)- |
| | | methyl)-6′-hydroxy-2′,4′,6′-trimethyl-7′-oxo-2′, |
| | | 3′,6′,7′-tetrahydrospiro[cyclopropane- |
| | | 1,5′-inden]-3′-yl)oxy)-5-oxopentanoic acid |
| 119 | 119 | (6′R)-3′-(3,4-dihydroxybutyl)-6′-hydroxy-2′,4′,6′- |
| | | trimethylspiro[cyclopropane-1,5′-inden]-7′(6′H)-one |
| 120 | 120 | (R)-6′-hydroxy-3′-(3-((3-((S)-6′-hydroxy-2′,4′,6′- |
| | | trimethyl-7′-oxo-6′,7′-dihydrospiro- |
| | | [cyclopropane-1,5′-inden]-3′-yl)propylidene)- |
| | | hydrazineylidene)propyl)-2′,4′,6′-trimethylspiro- |
| | | [cyclopropane-1,5′-inden]-7′(6′H)-one |
| 121 | 121 | (R)-2-(3-(6′-hydroxy-2′,4′,6′-trimethyl-7′-oxo-6′,7′- |
| | | dihydrospiro[cyclopropane-1,5′-inden]-3′- |
| | | yl)propylidene)hydrazine-1-carboxamide |
| 122 | 122 | (R)-6′-hydroxy-2′,4′,6′-trimethyl-3′-(3-(2- |
| | | phenylhydrazineylidene)propyl)spiro- |
| | | [cyclopropane-1,5′-inden]-7′(6′H)-one |
| 123 | 123 | (R)-N′-(3-(6′-hydroxy-2′,4′,6′-trimethyl-7′-oxo-6′,7′- |
| | | dihydrospiro[cyclopropane-1,5′-inden]-3′-yl)- |
| | | propylidene)-4-methylbenzenesulfonohydrazide |
| 124 | 124 | (R)-3-(6′-hydroxy-2′,4′,6′-trimethyl-7′-oxo-6′,7′- |
| | | dihydrospiro[cyclopropane-1,5′-inden]-3′- |
| | | yl)propanal O-acetyl oxime |
| 125 | 125 | (R)-3′-(3-(2-(2,4-dinitrophenyl)hydrazineylidene)- |
| | | propyl)-6′-hydroxy-2′,4′,6′-trimethylspiro- |
| | | [cyclopropane-1,5′-inden]-7′(6′H)-one |
| 126 | 126 | (R)-6′-hydroxy-2′,4′,6′-trimethyl-7′-oxo-6′,7′- |
| | | dihydrospiro[cyclopropane-1,5′-indene]-3′- |
| | | carbaldehyde oxime |
| 127 | 127 | 2-hydroxy-4-((R)-6′-hydroxy-2′,4′,6′-trimethyl- |
| | | 7′-oxo-6′,7′-dihydrospiro[cyclopropane- |
| | | 1,5′-inden]-3′-yl)butanenitrile |
| 128 | 128 | (6′R)-6′-hydroxy-3′-(3-hydroxybutyl)-2′,4′,6′- |
| | | trimethylspiro[cyclopropane-1,5′-inden]- |
| | | 7′(6′H)-one |
| 129 | 129 | (6′R)-2′,4′,6′-trimethyl-6′,7′-dihydrospiro- |
| | | [cyclopropane-1,5′-indene]-6′,7′-diol |
| 130 | 130 | (R)-6′-hydroxy-3′-(3-(hydroxyamino)propyl)- |
| | | 2′,4′,6′-trimethylspiro[cyclopropane-1,5′- |
| | | inden]-7′(6′H)-one |
| 131 | 131 | (R)-N-benzyl-3-(6′-hydroxy-2′,4′,6′-trimethyl-7′- |
| | | oxo-6′,7′-dihydrospiro[cyclopropane-1,5′- |
| | | inden]-3′-yl)propanamide |
| 132 | 133 | (E)-7-(chloromethylene)-5-hydroxy-5,9- |
| | | dimethylspiro[3.5]non-8-en-6-one |
| 133 | 134 | (E)-6-(chloromethylene)-4-hydroxy-4,8- |
| | | dimethylspiro[2.5]oct-7-en-5-one |
| 134 | 135 | ((2′S,6′R)-3′,6′-dihydroxy-2′,4′,6′-trimethyl-7′- |
| | | oxo-2′,3′,6′,7′-tetrahydrospiro[cyclopropane- |
| | | 1,5′-inden]-2′-yl)methyl 4-nitrobenzoate |
| 135 | 136 | ((2′S,6′R)-3′,6′-dihydroxy-2′,4′,6′-trimethyl-7′- |
| | | oxo-2′,3′,6′,7′-tetrahydrospiro[cyclopropane- |
| | | 1,5′-inden]-2′-yl)methyl 4-(N- |
| | | acetoxyacetamido)benzoate |
| 136 | 137 | ((2′S,6′R)-6′-hydroxy-2′,4′,6′-trimethyl-3′,7′- |
| | | dioxo-2′,3′,6′,7′-tetrahydrospiro[cyclopropane- |
| | | 1,5′-inden]-2′-yl)methyl 4-nitrobenzoate |
| 137 | 138 | ((2′S,6′R)-6′-hydroxy-2′,4′,6′-trimethyl-3′,7′- |
| | | dioxo-2′,3′,6′,7′-tetrahydrospiro[cyclopropane- |
| | | 1,5′-inden]-2′-yl)methyl 4-(N- |
| | | acetoxyacetamido)benzoate |
| 138 | 139 | (2′S,6′R)-6′-hydroxy-2′,4′,6′-trimethyl-2′-(((4- |
| | | nitrobenzoyl)oxy)methyl)-7′-oxo-2′,3′,6′,7′- |
| | | tetrahydrospiro[cyclopropane-1,5′-inden]-3′- |
| | | yl 4-nitrobenzoate |
| 139 | 140 | ((2′S,6′R)-3′-((4-(N-acetoxyacetamido)- |
| | | benzoyl)oxy)-6′-hydroxy-2′,4′,6′-trimethyl-7′- |
| | | oxo-2′,3′,6′,7′-tetrahydrospiro[cyclopropane-1,5′- |
| | | inden]-2′-yl)methyl 4-(N-acetoxyacetamido)benzoate |
| 140 | 141 | dimethyl (5′R)-4′,5′-dihydroxy-5′,7′,9′-trimethyl-4′, |
| | | 5′-dihydro-1′H-spiro[cyclopropane-1,6′-[1,3a]- |
| | | ethenoindene]-2′,3′-dicarboxylate |
| 141 | 142 | dimethyl (5′R)-5′-hydroxy-5′,7′,9′-trimethyl-4′-oxo- |
| | | 4′,5′-dihydro-1′H-spiro[cyclopropane-1,6′- |
| | | [1,3a]ethenoindene]-2′,3′-dicarboxylate |
| 142 | 143 | (R)-6′-hydroxy-1′,2′,4′,6′-tetramethylspiro- |
| | | [cyclopropane-1,5′-inden]-7′(6′H)-one |
| 143 | 144 | (R)-2-((2′-ethyl-6′-hydroxy-4′,6′-dimethyl-7′- |
| | | oxo-6′,7′-dihydrospiro[cyclopropane-1,5′- |
| | | inden]-3′-yl)methoxy)ethyl acetate |
| 144 | 145 | (R)-5-((6′-hydroxy-2′,4′,6′-trimethyl-7′-oxo- |
| | | 6′,7′-dihydrospiro[cyclopropane-1,5′-inden]- |
| | | 3′-yl)methoxy)-5-oxopentanoic acid |
| 145 | 146 | (R)-4-(6′-hydroxy-2′,4′,6′-trimethyl-7′-oxo- |
| | | 6′,7′-dihydrospiro[cyclopropane-1,5′- |
| | | inden]-3′-yl)butanenitrile |
| 146 | 147 | (R)-3′-((benzo[d]thiazol-2-ylthio)methyl)-6′- |
| | | hydroxy-2′,4′,6′-trimethylspiro- |
| | | [cyclopropane-1,5′-inden]-7′(6′H)-one |
| 147 | 148 | (R)-3′-((benzo[d]oxazol-2-ylthio)methyl)-6′- |
| | | hydroxy-2′,4′,6′-trimethylspiro- |
| | | [cyclopropane-1,5′-inden]-7′(6′H)-one |
| 148 | 149 | (R)-6′-hydroxy-2′,4′,6′-trimethyl-3′-(((1-methyl- |
| | | 1H-tetrazol-5-yl)thio)methyl)spiro- |
| | | [cyclopropane-1,5′-inden]-7′(6′H)-one |
| 149 | 150 | (R)-6′-hydroxy-2′,4′,6′-trimethyl-3′-(((5-methyl- |
| | | 1H-benzo[d]imidazol-2-yl)thio)methyl)spiro- |
| | | [cyclopropane-1,5′-inden]-7′(6′H)-one |
| 150 | 151 | (R)-6′-hydroxy-2′,4′,6′-trimethyl-3′-(((1-phenyl- |
| | | 1H-tetrazol-5-yl)thio)methyl)spiro- |
| | | [cyclopropane-1,5′-inden]-7′(6′H)-one |
| 151 | 152 | (R)-6′-hydroxy-2′,4′,6′-trimethyl-3′-(((5-nitro- |
| | | 1H-benzo[d]imidazol-2-yl)thio)methyl)spiro- |
| | | [cyclopropane-1,5′-inden]-7′(6′H)-one |
| 152 | 153 | (R)-3′-(((1H-1,2,4-triazol-3-yl)thio)methyl)-6′- |
| | | hydroxy-2′,4′,6′-trimethylspiro- |
| | | [cyclopropane-1,5′-inden]-7′(6′H)-one |
| 153 | 154 | (R)-6′-hydroxy-3′-(((4-hydroxypteridin-2-yl) |
| | | thio)methyl)-2′,4′,6′-trimethylspiro- |
| | | [cyclopropane-1,5′-inden]-7′(6′H)-one |
| 154 | 155 | (R)-6′-hydroxy-3′-(((1-(4-hydroxyphenyl)-1H- |
| | | tetrazol-5-yl)thio)methyl)-2′,4′,6′-trimethylspiro- |
| | | [cyclopropane-1,5′-inden]-7′(6′H)-one |
| 155 | 156 | (R)-4-(5-(((6′-hydroxy-2′,4′,6′-trimethyl-7′-oxo- |
| | | 6′,7′-dihydrospiro [cyclopropane-1,5′-inden]- |
| | | 3′-yl)methyl)thio)-1H-tetrazol-1- |
| | | yl)phenyl acetate |
| 156 | 157 | 7′-methyl-4′H-dispiro[cyclobutane-1,6′- |
| | | indene-5′,2″-[1,3]dioxolan]-4′-one |
| 157 | 158 | 5-hydroxy-2,2,6,8a-tetramethyl-2,3,3a,8,8a,8b- |
| | | hexahydro-1H-cyclobuta[d]- |
| | | cyclopenta[b]oxepin-7(5H)-one |
| 158 | 159 | ((6′R)-3′,6′-dihydroxy-2′,4′,6′-trimethyl-7′- |
| | | oxo-2′,3′,6′,7′-tetrahydrospiro- |
| | | [cyclopropane-1,5′-inden]-2′-yl)methyl acetate |
| 159 | 160 | 5-(((6′R)-2′-(acetoxymethyl)-6′-hydroxy-2′,4′,6′- |
| | | trimethyl-7′-oxo-2′,3′,6′,7′-tetrahydrospiro- |
| | | [cyclopropane-1,5′-inden]-3′-yl)oxy)-5- |
| | | oxopentanoic acid |
| 160 | 161 | ((6′R)-6′-hydroxy-2′,4′,6′-trimethyl-3′,7′-dioxo- |
| | | 2′,3′,6′,7′-tetrahydrospiro[cyclopropane- |
| | | 1,5′-inden]-2′-yl)methyl acetate |
| 161 | 162 | 5-(((6′R)-6′-hydroxy-2′,4′,6′-trimethyl-3′,7′- |
| | | dioxo-2′,3′,6′,7′-tetrahydrospiro[cyclopropane- |
| | | 1,5′-inden]-2′-yl)methoxy)-5- |
| | | oxopentanoic acid |
| 162 | 163 | (6′R)-2′-(acetoxymethyl)-6′-hydroxy-2′,4′,6′- |
| | | trimethyl-7′-oxo-2′,3′,6′,7′-tetrahydrospiro- |
| | | [cyclopropane-1,5′-inden]-3′-yl 2-chloroacetate |
| 163 | 164 | (R)-(6′-hydroxy-2′,4′,6′-trimethyl-7′-oxo-6′,7′- |
| | | dihydrospiro[cyclopropane-1,5′-inden]-3′-yl)methyl |
| | | 2-chloroacetate |
| 164 | 165 | (R)-(6′-hydroxy-2′,4′,6′-trimethyl-7′-oxo-6′,7′- |
| | | dihydrospiro[cyclopropane-1,5′-inden]-3′- |
| | | yl)methyl 2-morpholinoacetate |
| 165 | 166 | (6′R)-2′-(acetoxymethyl)-6′-hydroxy-2′,4′,6′- |
| | | trimethyl-7′-oxo-2′,3′,6′,7′-tetrahydrospiro- |
| | | [cyclopropane-1,5′-inden]-3′-yl methyl glutarate |
| 166 | 167 | (6′R)-6′-hydroxy-2′-(hydroxymethyl)-2′,4′,6′- |
| | | trimethyl-7′-oxo-2′,3′,6′,7′-tetrahydrospiro- |
| | | [cyclopropane-1,5′-inden]-3′-yl methyl glutarate |
| 167 | 168 | ((6′R)-6′-hydroxy-2′,4′,6′-trimethyl-3′,7′-dioxo- |
| | | 2′,3′,6′,7′-tetrahydrospiro[cyclopropane-1,5′- |
| | | inden]-2′-yl)methyl methyl glutarate |
| 168 | 169 | ((6′R)-6′-hydroxy-2′,4′,6′-trimethyl-3′,7′-dioxo- |
| | | 2′,3′,6′,7′-tetrahydrospiro[cyclopropane- |
| | | 1,5′-inden]-2′-yl)methyl 2-chloroacetate |
| 169 | 170 | 5-(2-hydroxyethyl)-3-(hydroxymethyl)- |
| | | 2,4,6-trimethyl-1H-inden-7-ol |
| 170 | 171 | 6-(2-hydroxyethyl)-2,5,7-trimethyl-1- |
| | | methylene-1H-inden-4-ol |
| 171 | 172 | 6-ethyl-2,5,7-trimethyl-1-methylene-1H- |
| | | inden-4-ol |
| 172 | 173 | 2-(4-hydroxy-2,5,7-trimethyl-1-methylene-1H- |
| | | inden-6-yl)ethyl acetate |
| 173 | 174 | 5-(2-hydroxyethyl)-3-(hydroxymethyl)-2,4,6- |
| | | trimethyl-1H-indene-1,7-diol |
| 174 | 175 | (2S,3S,4R,5S,6R)-2-(acetoxymethyl)-6-(((R)-6′- |
| | | hydroxy-2′,4′,6′-trimethyl-7′-oxo-6′,7′- |
| | | dihydrospiro[cyclopropane-1,5′-inden]-3′-yl)- |
| | | methoxy)tetrahydro-2H-pyran-3,4,5-triyl triacetate |
| 175 | 176 | 3-((R)-6′-hydroxy-2′,4′,6′-trimethyl-7′-oxo-6′,7′- |
| | | dihydrospiro[cyclopropane-1,5′-inden]-3′-yl)- |
| | | propyl leucinate |
| 176 | 177 | (R)-5-(3-(6′-hydroxy-2′,4′,6′-trimethyl-7′-oxo- |
| | | 6′,7′-dihydrospiro[cyclopropane-1,5′-inden]- |
| | | 3′-yl) propoxy)-5-oxopentanoic acid |
| 177 | 178 | (R)-4-(3-(6′-hydroxy-2′,4′,6′-trimethyl-7′-oxo- |
| | | 6′,7′-dihydrospiro[cyclopropane-1,5′-inden]- |
| | | 3′-yl)propoxy)-4-oxobutanoic acid |
| 178 | 179 | (R)-3-(6′-hydroxy-2′,4′,6′-trimethyl-7′-oxo-6′,7′- |
| | | dihydrospiro[cyclopropane-1,5′-inden]-3′- |
| | | yl)propyl glycinate |
| 179 | 180 | (1a′R,3′R,7′S,7a′R)-3′,7′-dihydroxy-1a′,3′,6′,6′- |
| | | tetramethyl-6′,7′-dihydro-1a′H-spiro[cyclopropane- |
| | | 1,2′-indeno[3a,4-b]oxiren]-4′(3′H)-one |
| 180 | 181 | ((1a′R,3′R,6′S,7′S,7a′R)-3′,7′-dihydroxy-1a′,3′,6′- |
| | | trimethyl-4′-oxo-3′,4′,6′,7′-tetrahydro-1a′H- |
| | | spiro[cyclopropane-1,2′-indeno[3a,4-b]- |
| | | oxiren]-6′-yl)methyl acetate |
| 181 | 182 | (2′R,7′S,7a′S)-2′-chloro-7′-hydroxy-2′,4′- |
| | | dimethyl-1′,2′,7′,7a′-tetrahydrospiro- |
| | | [cyclopropane-1,5′-indene]-3′,6′-dione |
| 182 | 183 | (2′S,7′S,7a′S)-7′-hydroxy-2′-isopropoxy-2′,4′- |
| | | dimethyl-1′,2′,7′,7a′-tetrahydrospiro- |
| | | [cyclopropane-1,5′-indene]-3′,6′-dione |
| 183 | 184 | (R)-1-hydroxy-1-((6′-hydroxy-2′,4′,6′-trimethyl- |
| | | 7′-oxo-6′,7′-dihydrospiro- |
| | | [cyclopropane-1,5′-inden]-3′-yl)methyl)urea |
| 184 | 185 | (S)-6′-hydroxy-2′,4′,6′-trimethylspiro- |
| | | [cyclopropane-1,5′-inden]-7′(6′H)-one |
| 185 | 186 | (6′S,7′R)-6′,7′-dihydroxy-2′,4′,6′-trimethyl-7′,7a′- |
| | | dihydrospiro[cyclopropane-1,5′-inden]-3′(6′H)-one |
| 186 | 187 | (S)-6′-hydroxy-3′-(hydroxymethyl)-2′,4′,6′- |
| | | trimethylspiro[cyclopropane-1,5′-inden]-7′(6′H)-one |
| 187 | 188 | (6′S,6″′S)-3′,3″′-methylenebis(6′-hydroxy-2′,4′,6′- |
| | | trimethylspiro[cyclopropane-1,5′- |
| | | inden]-7′(6′H)-one) |
| 188 | 189 | (R)-1-((6′-hydroxy-2′,4′,6′-trimethyl-7′-oxo- |
| | | 6′,7′-dihydrospiro[cyclopropane-1,5′-inden]- |
| | | 3′-yl)methyl)-1H-pyrrole-2,5-dione |
| 189 | 190 | (R)-1-(3-(6′-hydroxy-2′,4′,6′-trimethyl-7′-oxo- |
| | | 6′,7′-dihydrospiro[cyclopropane-1,5′-inden]- |
| | | 3′-yl)propyl)-1H-pyrrole-2,5-dione |
| 190 | 191 | 6′-hydroxy-4′,6′-dimethylspiro[cyclobutane- |
| | | 1,5′-inden]-7′(6′H)-one |
| 191 | 192 | (R)-2-((6′-hydroxy-2′,4′,6′-trimethyl-7′-oxo- |
| | | 6′,7′-dihydrospiro[cyclopropane-1,5′-inden]- |
| | | 3′-yl)methyl)isoindoline-1,3-dione |
| 192 | 193 | (R)-3′-(azidomethyl)-6′-hydroxy-2′,4′,6′- |
| | | trimethylspiro[cyclopropane-1,5′-inden]- |
| | | 7′(6′H)-one |
| 193 | 194 | (R)-3′-(((R)-3′-(azidomethyl)-6′-hydroxy- |
| | | 2′,4′,6′-trimethyl-7′-oxo-6′,7′-dihydrospiro- |
| | | [cyclopropane-1,5′-inden]-1′-yl)methyl)-6′- |
| | | hydroxy-2′,4′,6′-trimethylspiro- |
| | | [cyclopropane-1,5′-inden]-7′(6′H)-one |
| 194 | 195 | (R)-3′-(3-azidopropyl)-6′-hydroxy-4′,6′- |
| | | dimethylspiro[cyclopropane- |
| | | 1,5′-inden]-7′(6′H)-one |
| 195 | 196 | 3-((R)-6′-hydroxy-2′,4′,6′-trimethyl-7′-oxo- |
| | | 6′,7′-dihydrospiro[cyclopropane-1,5′-inden]- |
| | | 3′-yl)propyl L-prolinate |
| 196 | 197 | (R)-2-(3-(6′-hydroxy-2′,4′,6′-trimethyl-7′-oxo- |
| | | 6′,7′-dihydrospiro[cyclopropane-1,5′-inden]- |
| | | 3′-yl)propyl)isoindoline-1,3-dione |
| 197 | 198 | (R)-6′-hydroxy-2′,4′,6′-trimethyl-3′-((4- |
| | | nitrophenoxy)methyl)spiro[cyclopropane- |
| | | 1,5′-inden]-7′(6′H)-one |
| 198 | 199 | (R)-6′-hydroxy-2′,4′,6′-trimethyl-3′- |
| | | (phenoxymethyl)spiro[cyclopropane- |
| | | 1,5′-inden]-7′(6′H)-one |
| 199 | 200 | (R)-6′-hydroxy-3′-(2-hydroxybenzyl)-2′,4′, |
| | | 6′-trimethylspiro[cyclopropane- |
| | | 1,5′-inden]-7′(6′H)-one |
| 200 | 201 | (R)-N-(3-(6′-hydroxy-2′,4′,6′-trimethyl-7′-oxo- |
| | | 6′,7′-dihydrospiro[cyclopropane-1,5′-inden]- |
| | | 3′-yl)propyl)acetamide |
| 201 | 202 | (S)-N-(3-((R)-6′-hydroxy-2′,4′,6′-trimethyl-7′-oxo- |
| | | 6′,7′-dihydrospiro[cyclopropane-1,5′-inden]- |
| | | 3′-yl)propyl)pyrrolidine-2-carboxamide |
| 202 | 203 | 3-((R)-6′-hydroxy-2′,4′,6′-trimethyl-7′-oxo-6′,7′- |
| | | dihydrospiro[cyclopropane-1,5′-inden]- |
| | | 3′-yl)propyl L-seryl-L-prolinate |
| 203 | 204 | 2′-(((tert-butyldimethylsilyl)oxy)methyl)-3′,6′- |
| | | dihydroxy-2′,4′,6′-trimethyl-2′,3′- |
| | | dihydrospiro[cyclopropane-1,5′-inden]-7′(6′H)-one |
| 204 | 205 | (2′R,3′S,6′R)-3′-amino-2′-(((tert-butyldimethyl- |
| | | silyl) oxy)methyl)-6′-hydroxy-2′,4′,6′- |
| | | trimethyl-2′,3′-dihydrospiro[cyclopropane-1,5′- |
| | | inden]-7′(6′H)-one |
| 205 | 206 | (2′R,3′S,6′R)-3′-amino-6′-hydroxy-2′- |
| | | (hydroxymethyl)-2′,4′,6-trimethyl-2′,3′- |
| | | dihydrospiro[cyclopropane-1,5′-inden]- |
| | | 7′(6′H)-one |
| 206 | 207 | (S)-2-amino-N-(3-((R)-6′-hydroxy-2′,4′,6′- |
| | | trimethyl-7′-oxo-6′,7′-dihydrospiro- |
| | | [cyclopropane-1,5′-inden]-3′-yl)- |
| | | propyl)-4-methylpentanamide |
| 207 | 208 | 3-((R)-6′-hydroxy-2′,4′,6′-trimethyl-7′-oxo-6′,7′- |
| | | dihydrospiro[cyclopropane-1,5′-inden]-3′-yl)- |
| | | propyl (tert-butoxycarbonyl)-L-seryl-L-prolinate |
| 208 | 209 | 3-((R)-6′-hydroxy-2′,4′,6′-trimethyl-7′-oxo-6′,7′- |
| | | dihydrospiro[cyclopropane-1,5′-inden]- |
| | | 3′-yl)propyl L-seryl-L-seryl-L-prolinate |
| 209 | 210 | 3-((R)-6′-hydroxy-2′,4′,6′-trimethyl-7′-oxo-6′,7′- |
| | | dihydrospiro[cyclopropane-1,5′-inden]-3′-yl)propyl |
| | | ((S)-2-((S)-2-((S)-2-((2S,4R)-1-acetyl-4- |
| | | hydroxypyrrolidine-2-carboxamido)-3- |
| | | hydroxypropanamido)-3-hydroxypropanamido)- |
| | | 2-cyclohexylacetyl)-L-glutaminyl- |
| | | L-seryl-L-seryl-L-prolinate |
| 210 | 211 | (R)-3′-(3-aminopropyl)-6′-hydroxy-2′,4′,6′- |
| | | trimethylspiro[cyclopropane-1,5′-inden]-7′(6′H)-one |
| 211 | 212 | (3′S,6′R)-6′-hydroxy-2′,2′,4′,6′-tetramethyl- |
| | | 7′-oxo-2′,3′,6′,7′-tetrahydrospiro- |
| | | [cyclopropane-1,5′-inden]-3′-yl L-prolinate |
| 212 | 213 | (2′S,3′R,6′R)-2′-(((tert-butyldimethylsilyl)oxy)- |
| | | methyl)-6′-hydroxy-2′,4′,6′-trimethyl-7′-oxo- |
| | | 2′,3′,6′,7′-tetrahydrospiro[cyclopropane- |
| | | 1,5′-inden]-3′-yl L-prolinate |
| 213 | 214 | (2′S,3′R,6′R)-2′-(((tert-butyldimethylsilyl)oxy)- |
| | | methyl)-6′-hydroxy-2′,4′,6′-trimethyl-7′- |
| | | oxo-2′,3′,6′,7′-tetrahydrospiro[cyclopropane- |
| | | 1,5′-inden]-3′-yl L-seryl-L-seryl-L-prolinate |
| 214 | 215 | (2′S,3′R,6′R)-2′-((tert-butyldimethylsilyl)oxy)- |
| | | methyl)-6′-hydroxy-2′,4′,6′-trimethyl-7′-oxo- |
| | | 2′,3′,6′,7′-tetrahydrospiro[cyclopropane-1,5′- |
| | | inden]-3′-yl ((S)-2-((S)-2-((S)-2-((2S,4R)-1- |
| | | acetyl-4-hydroxypyrrolidine-2-carboxamido)- |
| | | 3-hydroxypropanamido)-3- |
| | | hydroxypropanamido)-2-cyclohexylacetyl)- |
| | | L-glutaminyl-L-seryl-L-seryl-L-prolinate |
| 215 | 216 | (3′S,6′R)-6′-hydroxy-2′,2′,4′,6′-tetramethyl- |
| | | 7′-oxo-2′,3′,6′,7′-tetrahydrospiro- |
| | | [cyclopropane-1,5′-inden]-3′-yl ((S)-2-((S)- |
| | | 2-((S)-2-((2S,4R)-1-acetyl-4- |
| | | hydroxypyrrolidine-2-carboxamido)-3- |
| | | hydroxypropanamido)-3-hydroxy- |
| | | propanamido)-2-cyclohexylacetyl)- |
| | | L-glutaminyl-L-seryl-L-seryl-L-prolinate |
| 216 | 217 | (R)-(6′-hydroxy-2′,4′,6′-trimethyl-7′-oxo-6′,7′- |
| | | dihydrospiro[cyclopropane-1,5′-inden]-3′-yl)- |
| | | methyl 6-(2,5-dioxo-2,5-dihydro- |
| | | 1H-pyrrol-1-yl)hexanoate |
| 217 | 218 | (3′S,6′R)-6′-hydroxy-2′,2′,4′,6′-tetramethyl- |
| | | 7′-oxo-2′,3′,6′,7′-tetrahydrospiro- |
| | | [cyclopropane-1,5′-inden]-3′-yl 6-(2,5-dioxo- |
| | | 2,5-dihydro-1H-pyrrol-1-yl)hexanoate |
| 218 | 219 | (2′S,3′R,6′R)-2′-((tert-butyldimethylsilyl)oxy)- |
| | | methyl)-6′-hydroxy-2′,4′,6′-trimethyl-7′- |
| | | oxo-2′,3′,6′,7′-tetrahydrospiro[cyclopropane- |
| | | 1,5′-inden]-3′-yl 4-(fluorosulfonyl)benzoate |
| 219 | 220 | (R)-1-acetoxy-1-((6′-hydroxy-2′,4′,6′-trimethyl- |
| | | 7′-oxo-6′,7′-dihydrospiro[cyclopropane- |
| | | 1,5′-inden]-3′-yl)methyl)urea |
| 220 | 221 | (S)-2-((3S,6S,9S,12S,15S)-3-((1H-imidazol-4- |
| | | yl)methyl)-12-(4-aminobutyl)-6,9-bis- |
| | | (hydroxymethyl)-15-isobutyl-1-morpholino- |
| | | 1,4,7,10,13-pentaoxo-2,5,8,11,14- |
| | | pentaazahexadecan-16-amido)-N1-((S)-1- |
| | | ((3-((R)-6′-hydroxy-2′,4′,6′-trimethyl-7′-oxo-6′,7′- |
| | | dihydrospiro[cyclopropane-1,5′-inden]-3′-yl)- |
| | | propyl)amino)-4-methyl-1-oxopentan-2- |
| | | yl)pentanediamide |
| 221 | 222 | (3′S,6′R)-6′-hydroxy-2′,2′,4′,6′-tetramethyl-7′- |
| | | oxo-2′,3′,6′,7′-tetrahydrospiro[cyclopropane- |
| | | 1,5′-inden]-3′-yl 4-(fluorosulfonyl)benzoate |
| 222 | 223 | 3-((R)-6′-hydroxy-2′,4′,6′-trimethyl-7′-oxo-6′,7′- |
| | | dihydrospiro[cyclopropane-1,5′-inden]-3′- |
| | | yl)propyl ((4R,7S,13S)-13-(2- |
| | | amino-2-oxoethyl)-7-(3-guanidinopropyl)- |
| | | 6,9,12,15-tetraoxo-1,2-dithia-5,8,14- |
| | | triazacycloheptadecane-4-carbonyl)glycinate |
| 223 | 224 | (R,E)-6′-hydroxy-2′,4′,6′-trimethylspiro- |
| | | [cyclopropane-1,5′-inden]-7′(6′H)-one oxime |
| 224 | 225 | (2′R,3′R,6′R,E)-2′,3′,6′-trihydroxy-2′,4′,6′-trimethyl- |
| | | 2′,3′-dihydrospiro[cyclopropane-1,5′-inden]- |
| | | 7′(6′H)-one oxime |
| 225 | 226 | (R)-(6′-hydroxy-2′,4′,6′-trimethyl-7′-oxo-6′,7′- |
| | | dihydrospiro[cyclopropane-1,5′-inden]- |
| | | 3′-yl)methyl (E)-octadec-9-enoate |
| 226 | 227 | (R)-3-(6′-hydroxy-2′,4′,6′-trimethyl-7′-oxo-6′,7′- |
| | | dihydrospiro[cyclopropane-1,5′-inden]- |
| | | 3′-yl)propyl (E)-octadec-9-enoate |
| 227 | 228 | (2′S,6′R)-6′-hydroxy-2′,4′,6′-trimethyl-2′-((((E)- |
| | | octadec-9-enoyl)oxy)methyl)-7′-oxo-2′,3′,6′,7′- |
| | | tetrahydrospiro[cyclopropane- |
| | | 1,5′-inden]-3′-yl (E)-octadec-9-enoate |
| 228 | 229 | (R,E)-N-(3-(6′-hydroxy-2′,4′,6′-trimethyl-7′-oxo- |
| | | 6′,7′-dihydrospiro[cyclopropane-1,5′-inden]- |
| | | 3′-yl)propyl)octadec-9-enamide |
| 229 | 230 | N-((3′R,6′R)-6′-hydroxy-2′,2′,4′,6′-tetramethyl-7′- |
| | | oxo-2′,3′,6′,7′-tetrahydrospiro[cyclopropane- |
| | | 1,5′-inden]-3′-yl)methanesulfonamide |
| 230 | 231 | N-((3′R,6′R)-6′-hydroxy-2′,2′,4′,6′-tetramethyl-7′- |
| | | oxo-2′,3′,6′,7′-tetrahydrospiro[cyclopropane-1,5′- |
| | | inden]-3′-yl)-4-methylbenzenesulfonamide |
| 231 | 232 | (R)-3-(6′-hydroxy-2′,4′,6′-trimethyl-7′-oxo-6′,7′- |
| | | dihydrospiro[cyclopropane-1,5′-inden]-3′- |
| | | yl)propyl hydroxycarbamate |
| 232 | 233 | ethyl (R)-hydroxy((6′-hydroxy-2′,4′,6′-trimethyl- |
| | | 7′-oxo-6′,7′-dihydrospiro[cyclopropane- |
| | | 1,5′-inden]-3′-yl)methyl)carbamate |
| 233 | 234 | benzyl (R)-hydroxy((6′-hydroxy-2′,4′,6′-trimethyl- |
| | | 7′-oxo-6′,7′-dihydrospiro[cyclopropane- |
| | | 1,5′-inden]-3′-yl)methyl)carbamate |
| 234 | 235 | tert-butyl (R)-hydroxy((6′-hydroxy-2′,4′,6′- |
| | | trimethyl-7′-oxo-6′,7′-dihydrospiro- |
| | | [cyclopropane-1,5′-inden]-3′-yl)methyl)carbamate |
| 235 | 236 | (R)-6′-hydroxy-3′-(hydroxymethyl)-1′,2′,4′,6′- |
| | | tetramethylspiro[cyclopropane-1,5′-inden]-7′(6′H)-one |
| 236 | 237 | (R)-3-(6′-hydroxy-2′,4′,6′-trimethyl-7′-oxo-6′,7′- |
| | | dihydrospiro[cyclopropane-1,5′-inden]-3′-yl)propyl |
| | | (2-bromoethyl)carbamate |
| 237 | 238 | (R)-3-(6′-hydroxy-2′,4′,6′-trimethyl-7′-oxo-6′,7′- |
| | | dihydrospiro[cyclopropane-1,5′-inden]-3′-yl)propyl |
| | | (2-chloroethyl)carbamate |
| 238 | 239 | (R)-3-(6′-hydroxy-2′,4′,6′-trimethyl-7′-oxo-6′,7′- |
| | | dihydrospiro[cyclopropane-1,5′-inden]-3′-yl)propyl |
| | | (2-hydroxyethyl)carbamate |
| 239 | 240 | (R)-3-(6′-hydroxy-2′,4′,6′-trimethyl-7′-oxo-6′,7′- |
| | | dihydrospiro[cyclopropane-1,5′-inden]-3′-yl)propyl |
| | | acetoxy(acetyl)carbamate |
| 240 | 241 | (R)-N-(3-(6′-hydroxy-2′,4′,6′-trimethyl-7′-oxo-6′,7′- |
| | | dihydrospiro[cyclopropane-1,5′-inden]-3′- |
| | | yl)propyl)methanesulfonamide |
| 241 | 242 | (R)-N-(3-(6′-hydroxy-2′,4′,6′-trimethyl-7′-oxo-6′,7′- |
| | | dihydrospiro[cyclopropane-1,5′-inden]-3′-yl)propyl)- |
| | | 4-methylbenzenesulfonamide |
| 242 | 243 | (R)-3-(6′-hydroxy-2′,4′,6′-trimethyl-7′-oxo-6′,7′- |
| | | dihydrospiro[cyclopropane-1,5′-inden]-3′-yl)propyl |
| | | (2-fluoroethyl)carbamate |
| 243 | 244 | (R)-1-hydroxy-1-(3-(6′-hydroxy-2′,4′,6′-trimethyl- |
| | | 7′-oxo-6′,7′-dihydrospiro- |
| | | [cyclopropane-1,5′-inden]-3′-yl)propyl)urea |
| 244 | 245 | (R)-1-hydroxy-1-(3-(6′-hydroxy-2′,4′,6′-trimethyl- |
| | | 7′-oxo-6′,7′-dihydrospiro- |
| | | [cyclopropane-1,5′-inden]-3′-yl)propyl)thiourea |
| 245 | 246 | (R)-3-(6′-hydroxy-2′,4′,6′-trimethyl-7′-oxo-6′,7′- |
| | | dihydrospiro[cyclopropane-1,5′-inden]-3′-yl)- |
| | | propyl morpholine-4-carboxylate |
| 246 | 247 | (R)-3-(6′-hydroxy-2′,4′,6′-trimethyl-7′-oxo-6′,7′- |
| | | dihydrospiro[cyclopropane-1,5′-inden]-3′-yl)- |
| | | propyl morpholine-4-carboxylate |
| 247 | 248 | (R)-3-(6′-hydroxy-2′,4′,6′-trimethyl-7′-oxo-6′,7′- |
| | | dihydrospiro[cyclopropane-1,5′-inden]-3′-yl)- |
| | | propyl [1,4′-bipiperidine]-1′-carboxylate |
| 248 | 249 | (R)-6′-hydroxy-2′-(hydroxymethyl)-3′,4′,6′- |
| | | trimethylspiro[cyclopropane-1,5′-inden]- |
| | | 7′(6′H)-one |
| 249 | 250 | ((1a′R,2′S,3′R,6′R,7a′S)-3′-acetoxy-6′-hydroxy- |
| | | 2′,4′,6′-trimethyl-7′-oxo-1′,1a′,2′,3′,6′,7′- |
| | | hexahydrospiro[cyclopropane-1,5′- |
| | | cyclopropa[c]inden]-2′-yl)methyl acetate |
| 250 | 251 | ((1a′S,2′S,3′R,6′R,7a′R)-3′-acetoxy-6′-hydroxy- |
| | | 2′,4′,6′-trimethyl-7′-oxo-1′,1a′,2′,3′,6′,7′- |
| | | hexahydrospiro[cyclopropane-1,5′- |
| | | cyclopropa[c]inden]-2′-yl)methyl acetate |
| 251 | 252 | (1a′R,3′S,6′R,7a′S)-3′,6′-dihydroxy-2′,2′,4′,6′- |
| | | tetramethyl-1′,1a′,2′,3′-tetrahydrospiro- |
| | | [cyclopropane-1,5′-cyclopropa[c]inden]- |
| | | 7′(6′H)-one |
| 252 | 253 | (1a′S,3′S,6′R,7a′R)-3′,6′-dihydroxy-2′,2′,4′,6′- |
| | | tetramethyl-1′,1a′,2′,3′-tetrahydrospiro- |
| | | [cyclopropane-1,5′-cyclopropa[c]inden]-7′(6′H)-one |
| 253 | 254 | (R)-3-(6′-hydroxy-2′,4′,6′-trimethyl-7′-oxo-6′,7′- |
| | | dihydrospiro[cyclopropane-1,5′-inden]-3′-yl)- |
| | | propyl 4-sulfamoylbenzoate |
| 254 | 255 | (R)-3-(6′-hydroxy-2′,4′,6′-trimethyl-7′-oxo-6′,7′- |
| | | dihydrospiro[cyclopropane-1,5′-inden]-3′- |
| | | yl)propyl sulfamate |
| 255 | 256 | 3-((R)-6′-hydroxy-2′,4′,6′-trimethyl-7′-oxo-6′,7′- |
| | | dihydrospiro[cyclopropane-1,5′-inden]-3′-yl)propyl |
| | | 1-(3-((R)-6′-hydroxy-2′,4′,6′-trimethyl- |
| | | 7′-oxo-6′,7′-dihydrospiro[cyclopropane-1,5′- |
| | | inden]-3′-yl)propyl)-1H-1,2,3-triazole-4-carboxylate |
| 256 | 257 | 3-((R)-6′-hydroxy-2′,4′,6′-trimethyl-7′-oxo-6′,7′- |
| | | dihydrospiro[cyclopropane-1,5′-inden]-3′-yl)propyl |
| | | 1-(((R)-6′-hydroxy-2′,4′,6′-trimethyl-7′-oxo- |
| | | 6′,7′-dihydrospiro[cyclopropane-1,5′-inden]- |
| | | 3′-yl)methyl)-1H-1,2,3-triazole-4-carboxylate |
| 257 | 258 | (4-carboxy-4-(4-carboxy-4-((3-((R)-6′-hydroxy- |
| | | 2′,4′,6′-trimethyl-7′-oxo-6′,7′-dihydrospiro- |
| | | [cyclopropane-1,5′-inden]-3′-yl)propyl)- |
| | | amino)butanamido)butanoyl)glutamic acid |
| 258 | 259 | (R)-3′-((S)-2,2-dioxido-1,2,3-oxathiazinan-4-yl)- |
| | | 6′-hydroxy-2′,4′,6′-trimethylspiro- |
| | | [cyclopropane-1,5′-inden]-7′(6′H)-one |
| 259 | 260 | (R)-N-(3-(6′-hydroxy-2′,4′,6′-trimethyl-7′-oxo-6′, |
| | | 7′-dihydrospiro[cyclopropane-1,5′-inden]-3′-yl)- |
| | | propyl)-[1,4′-bipiperidine]-1′-carboxamide |
| 260 | 261 | (R)-3-(6′-hydroxy-2′,4′,6′-trimethyl-7′-oxo-6′,7′- |
| | | dihydrospiro[cyclopropane-1,5′-inden]-3′- |
| | | yl)propyl 1H-imidazole-1-carboxylate |
| 261 | 262 | methyl (R)-2-(((6′-hydroxy-2′,4′,6′-trimethyl- |
| | | 7′-oxo-6′,7′-dihydrospiro[cyclopropane-1,5′- |
| | | inden]-3′-yl)methyl)sulfonyl)acetate |
| 262 | 263 | methyl 2-((((R)-6′-hydroxy-2′,4′,6′-trimethyl-7′-oxo- |
| | | 6′,7′-dihydrospiro[cyclopropane-1,5′-inden]- |
| | | 3′-yl)methyl)sulfinyl)acetate |
| 263 | 264 | N-[3-(6′-hydroxy-2′,6′-dimethyl-7′-oxo-6′,7′- |
| | | dihydrospiro[cyclopropane-1,5′- |
| | | inden]-3′-yl)propyl]sulfuric diamide |
| 264 | 265 | N-hydroxy-N′-[3-(6′-hydroxy-2′,6′-dimethyl-7′- |
| | | oxo-6,7-dihydrospiro[cyclopropane-1,5′- |
| | | inden]-3′-yl)propyl]sulfuric diamide |
| 265 | 266 | N-[3-(6′-hydroxy-2′,6′-dimethyl-7′-oxo-6′,7′- |
| | | dihydrospiro[cyclopropane-1,5′-inden]-3′- |
| | | yl)propyl]-N-methoxysulfuric diamide |
| 266 | 267 | (R)-2-amino-N-((6′-hydroxy-2′,4′,6′-trimethyl-7′- |
| | | oxo-6′,7′-dihydrospiro[cyclopropane-1,5′-inden]- |
| | | 3′-yl)methyl)-N-methoxyacetamide |
| 267 | 268 | (R)-2,2,2-trifluoro-N-(3-(6′-hydroxy-2′,4′,6′- |
| | | trimethyl-7′-oxo-6′,7′-dihydrospiro- |
| | | [cyclopropane-1,5′-inden]-3′-yl)propyl)acetamide |
| 268 | 269 | (R)-(6′-hydroxy-2′,4′,6′-trimethyl-7′-oxo-6′,7′- |
| | | dihydrospiro[cyclopropane-1,5′-inden]-3′-yl)methyl |
| | | (4-methoxyphenyl)sulfamate |
| 269 | 270 | (R)-3′-(aminomethyl)-6′-hydroxy-2′,4′,6′- |
| | | trimethylspiro[cyclopropane-1,5′-inden]-7′(6′H)-one |
| 270 | 271 | (R)-N-((6′-hydroxy-2′,4′,6′-trimethyl-7′-oxo-6′,7′- |
| | | dihydrospiro[cyclopropane-1,5′-inden]-3′- |
| | | yl)methyl)methanesulfonamide |
| 271 | 272 | (5S,6S,7S)-3-(((3-((R)-6′-hydroxy-2′,4′,6′- |
| | | trimethyl-7′-oxo-6′,7′-dihydrospiro- |
| | | [cyclopropane-1,5′-inden]-3′-yl)propanoyl)- |
| | | oxy)methyl)-8-oxo-7-(2-(thiophen-2-yl)acetamido)- |
| | | 5-thia-1-azabicyclo[4.2.0]oct-2-ene-2- |
| | | carboxylic acid 5-oxide |
| 272 | 273 | (5S,6S,7S)-3-((((3-((R)-6′-hydroxy-2′,4′,6′- |
| | | trimethyl-7′-oxo-6′,7′-dihydrospiro- |
| | | [cyclopropane-1,5′-inden]-3′-yl)propyl)- |
| | | carbamoyl)oxy)methyl)-8-oxo-7-(2-(thiophen-2- |
| | | yl)acetamido)-5-thia-1-azabicyclo[4.2.0]oct-2- |
| | | ene-2-carboxylic acid 5-oxide |
| 273 | 274 | (6S,7S)-3-((((3-((R)-6′-hydroxy-2′,4′,6′- |
| | | trimethyl-7′-oxo-6′,7′-dihydrospiro- |
| | | [cyclopropane-1,5′-inden]-3′-yl)propyl)- |
| | | carbamoyl)oxy)methyl)-8-oxo-7-(2-(thiophen-2- |
| | | yl)acetamido)-5-thia-1-azabicyclo[4.2.0]oct- |
| | | 2-ene-2-carboxylic acid 5,5-dioxide |
| 274 | 275 | N-(((R)-6′-hydroxy-2′,4′,6′-trimethyl-7′-oxo- |
| | | 6′,7′-dihydrospiro[cyclopropane-1,5′-inden]-3′- |
| | | yl)methyl)pyrrolidine-2-carboxamide |
| 275 | 276 | 2-amino-N-(((R)-6′-hydroxy-2′,4′,6′-trimethyl-7′- |
| | | oxo-6′,7′-dihydrospiro[cyclopropane-1,5′-inden]- |
| | | 3′-yl)methyl)-4-methylpentanamide |
| 276 | 277 | (R)-1-hydroxy-3-((6′-hydroxy-2′,4′,6′-trimethyl- |
| | | 7′-oxo-6′,7′-dihydrospiro[cyclopropane- |
| | | 1,5′-inden]-3′-yl)methyl)urea |
| 277 | 278 | (R)-1-((6′-hydroxy-2′,4′,6′-trimethyl-7′-oxo- |
| | | 6′,7′-dihydrospiro[cyclopropane-1,5′-inden]- |
| | | 3′-yl)methyl)-3-methoxyurea |
| 278 | 279 | (R)-1-((6′-hydroxy-2′,4′,6′-trimethyl-7′-oxo- |
| | | 6′,7′-dihydrospiro[cyclopropane-1,5′-inden]- |
| | | 3′-yl)methyl)-3-(2-hydroxyethyl)urea |
| 279 | 280 | (R)-1-(3-(6′-hydroxy-2′,4′,6′-trimethyl-7′-oxo- |
| | | 6′,7′-dihydrospiro[cyclopropane-1,5′-inden]- |
| | | 3′-yl)propyl)-3-(2-hydroxyethyl)urea |
| 280 | 281 | (R)-1-(2-chloroethyl)-3-(3-(6′-hydroxy-2′,4′,6′- |
| | | trimethyl-7′-oxo-6′,7′-dihydrospiro- |
| | | [cyclopropane-1,5′-inden]-3′-yl)propyl)urea |
| 281 | 282 | (R)-1-(2-chloroethyl)-3-((6′-hydroxy-2′,4′,6′- |
| | | trimethyl-7′-oxo-6′,7′-dihydrospiro- |
| | | [cyclopropane-1,5′-inden]-3′-yl)methyl)urea |
| 282 | 283 | N-[(6′-hydroxy-2′,6′-dimethyl-7′-oxo-6′,7′- |
| | | dihydrospiro[cyclopropane-1,5′-inden]- |
| | | 3′-yl)methyl]sulfuric diamide |
| 283 | 284 | (R)-3-((6′-hydroxy-2′,4′,6′-trimethyl-7′-oxo- |
| | | 6′,7′-dihydrospiro[cyclopropane-1,5′-inden]-3′- |
| | | yl)methyl)pyrimidine-2,4(1H,3H)-dione |
| 284 | 285 | N-hydroxy-N-[(6′-hydroxy-2′,6′-dimethyl-7′-oxo- |
| | | 6′,7′-dihydrospiro[cyclopropane-1,5′-inden]- |
| | | 3′-yl)methyl]sulfuric diamide |
| 285 | 286 | (R)-5-fluoro-1-((6′-hydroxy-2′,4′,6′-trimethyl-7′- |
| | | oxo-6′,7′-dihydrospiro[cyclopropane-1,5′-inden]- |
| | | 3′-yl)methyl)pyrimidine-2,4(1H,3H)-dione |
| 286 | 287 | (R)-5-fluoro-3-((6′-hydroxy-2′,4′,6′-trimethyl-7′-oxo- |
| | | 6′,7′-dihydrospiro[cyclopropane-1,5′-inden]- |
| | | 3′-yl)methyl)pyrimidine-2,4(lH,3H)-dione |
| 287 | 288 | (R)-1-hydroxy-3-(3-(6′-hydroxy-2′,4′,6′-trimethyl- |
| | | 7′-oxo-6′,7′-dihydrospiro[cyclopropane- |
| | | 1,5′-inden]-3′-yl)propyl)urea |
| 288 | 289 | (R)-1-((6′-hydroxy-2′,4′,6′-trimethyl-7′-oxo-6′,7′- |
| | | dihydrospiro[cyclopropane-1,5′-inden]- |
| | | 3′-yl)methyl)pyrimidine-2,4(1H,3H)-dione |
| 289 | 290 | ((S)-6′-hydroxy-2′,4′,6′-trimethyl-7′-oxo-6′,7′- |
| | | dihydrospiro[cyclopropane-1,5′-inden]-3′- |
| | | yl)methyl 5-oxo-5-(((S)-1,2,3,10-tetramethoxy- |
| | | 9-oxo-5,6,7,9-tetrahydrobenzo[a]heptalen-7- |
| | | yl)amino)pentanoate |
| 290 | 291 | 3-((S)-6′-hydroxy-2′,4′,6′-trimethyl-7′-oxo-6′,7′- |
| | | dihydrospiro[cyclopropane-1,5′-inden]-3′-yl)- |
| | | propyl 5-oxo-5-(((S)-1,2,3,10-tetramethoxy- |
| | | 9-oxo-5,6,7,9-tetrahydrobenzo[a]heptalen-7- |
| | | yl)amino)pentanoate |
| 291 | 292 | N1-(((S)-6′-hydroxy-2′,4′,6′-trimethyl-7′-oxo-6′,7′- |
| | | dihydrospiro[cyclopropane-1,5′-inden]-3′-yl)methyl)- |
| | | N5-((S)-1,2,3,10-tetramethoxy-9-oxo-5,6,7,9- |
| | | tetrahydrobenzo[a]heptalen-7-yl)glutaramide |
| 292 | 293 | 3-((S)-6′-hydroxy-2′,4′,6′-trimethyl-7′-oxo-6′,7′- |
| | | dihydrospiro[cyclopropane-1,5′-inden]-3′-yl)-N-((S)- |
| | | 1,2,3,10-tetramethoxy-9-oxo-5,6,7,9- |
| | | tetrahydrobenzo [a]heptalen-7-yl)propanamide |
| 293 | 294 | 2-amino-N-(((R)-6′-hydroxy-2′,4′,6′-trimethyl- |
| | | 7′-oxo-6′,7′-dihydrospiro[cyclopropane-1,5′- |
| | | inden]-3′-yl)methyl)-N-methoxypropanamide |
| 294 | 295 | 2-amino-N-(((R)-6′-hydroxy-2′,4′,6′-trimethyl-7′- |
| | | oxo-6′,7′-dihydrospiro[cyclopropane-1,5′-inden]- |
| | | 3′-yl)methyl)-N-methoxy-4-methylpentanamide |
| 295 | 296 | 2-amino-N-(((R)-6′-hydroxy-2′,4′,6′-trimethyl-7′- |
| | | oxo-6′,7′-dihydrospiro[cyclopropane-1,5′-inden]-3′- |
| | | yl)methyl)-N-methoxy-4-(methylthio)butanamide |
| 296 | 297 | 2-amino-N-(((R)-6′-hydroxy-2′,4′,6′-trimethyl-7′- |
| | | oxo-6′,7′-dihydrospiro[cyclopropane-1,5′-inden]- |
| | | 3′-yl)methyl)-3-(1H-indol-3-yl)- |
| | | N-methoxypropanamide |
| 297 | 298 | (R)-3-(6′-hydroxy-2′,4′,6′-trimethyl-7′-oxo-6′,7′- |
| | | dihydrospiro[cyclopropane-1,5′-inden]-3′- |
| | | yl)propyl (tert-butoxycarbonyl)glycinate |
| 298 | 299 | (2′S,3′R,6′R)-2′-(azidomethyl)-3′,6′-dihydroxy- |
| | | 2′,4′,6′-trimethyl-2′,3′-dihydrospiro- |
| | | [cyclopropane-1,5′-inden]-7′(6′H)-one |
| 299 | 300 | (2′R,3′R,6′R)-3′-azido-6′-hydroxy-2′- |
| | | (hydroxymethyl)-2′,4′,6′-trimethyl-2′,3′- |
| | | dihydrospiro[cyclopropane-1,5′-inden]- |
| | | 7′(6′H)-one |
| 300 | 301 | (R)-6′-hydroxy-2′,4′,6′-trimethyl-7′-oxo-6′,7′- |
| | | dihydrospiro[cyclopropane-1,5′-inden]-3′-yl |
| | | 6-oxo-6-phenylhexanoate |
| 301 | 302 | ((2′S,3′R,6′R)-3′,6′-dihydroxy-2′,4′,6′-trimethyl-7′- |
| | | oxo-2′,3′,6′,7′-tetrahydrospiro[cyclopropane-1, |
| | | 5′-inden]-2′-yl)methyl 3,5-dinitrobenzoate |
| 302 | 303 | (2′S,3′R,6′R)-2′-(((3,5-dinitrocyclohexa-2,4-diene-1- |
| | | carbonyl)oxy)methyl)-6′-hydroxy-2′,4′,6′-trimethyl- |
| | | 7′-oxo-2′,3′,6′,7′-tetrahydrospiro[cyclopropane- |
| | | 1,5′-inden]-3′-yl 3,5-dinitrobenzoate |
| 303 | 304 | (R)-3-(6′-hydroxy-2′,4′,6′-trimethyl-7′-oxo-6′,7′- |
| | | dihydrospiro[cyclopropane-1,5′-inden]-3′- |
| | | yl)propyl propiolate |
| 304 | 305 | (R)-3-(6′-hydroxy-2′,4′,6′-trimethyl-7′-oxo-6′,7′- |
| | | dihydrospiro[cyclopropane-1,5′-inden]-3′- |
| | | yl)propyl (4-nitrophenyl) carbonate |
| 305 | 306 | (R)-3-(6′-hydroxy-2′,4′,6′-trimethyl-7′-oxo-6′,7′- |
| | | dihydrospiro[cyclopropane-1,5′-inden]-3′- |
| | | yl)propyl 4-methylbenzenesulfonate |
| 306 | 307 | (3′R,6′R)-3′-azido-6′-hydroxy-2′,2′,4′,6′-tetramethyl- |
| | | 2′,3′-dihydrospiro[cyclopropane-1,5′- |
| | | inden]-7′(6′H)-one |
| 307 | 308 | (3′R,6′R)-3′-amino-6′-hydroxy-2′,2′,4′,6′- |
| | | tetramethyl-2′,3′-dihydrospiro- |
| | | [cyclopropane-1,5′-inden]-7′(6′H)-one |
| 308 | 309 | (2′R,3′S,6R)-3′-azido-2′-(((tert-butyldimethylsilyl)- |
| | | oxy)methyl)-6′-hydroxy-2′,4′,6′-trimethyl-2′,3′- |
| | | dihydrospiro[cyclopropane-1,5′-inden]- |
| | | 7′(6′H)-one |
| 309 | 310 | (R)-(6′-hydroxy-2′,4′,6′-trimethyl-7′-oxo-6′,7′- |
| | | dihydrospiro[cyclopropane-1,5′-inden]-3′-yl)- |
| | | methyl 4-sulfamoylbenzoate |
| 310 | 311 | (3′S,6′R)-6′-hydroxy-2′,2′,4′,6′-tetramethyl-7′-oxo-2′, |
| | | 3′,6′,7′-tetrahydrospiro[cyclopropane-1,5′- |
| | | inden]-3′-yl 4-sulfamoylbenzoate |
| 311 | 312 | (R)-(6′-hydroxy-2′,4′,6′-trimethyl-7′-oxo-6′,7′- |
| | | dihydrospiro[cyclopropane-1,5′-inden]-3′-yl)- |
| | | methyl 4-methylbenzenesulfonate |
| 312 | 313 | 2,3,4,5,6-pentafluoro-N-((3′R,6′R)-6′-hydroxy- |
| | | 2′,2′,4′,6′-tetramethyl-7′-oxo-2′,3′,6′,7′- |
| | | tetrahydrospiro[cyclopropane-1,5′- |
| | | inden]-3′-yl)benzenesulfonamide |
| 313 | 314 | (R)-3-(6′-hydroxy-2′,4′,6′-trimethyl-7′-oxo-6′,7′- |
| | | dihydrospiro[cyclopropane-1,5′-inden]-3′- |
| | | yl)propyl 4-methylbenzenesulfonate |
| 314 | 315 | (R)-3′-((2,5-dimethyl-1H-pyrrol-3-yl)methyl)- |
| | | 6′-hydroxy-2′,4′,6′-trimethylspiro- |
| | | [cyclopropane-1,5′-inden]-7′(6′H)-one |
| 315 | 316 | ((2′S,3′R,6′R)-3′,6′-dihydroxy-2′,4′,6′-trimethyl-7′- |
| | | oxo-2′,3′,6′,7′-tetrahydrospiro- |
| | | [cyclopropane-1,5′-inden]-2′-yl)methyl 4- |
| | | (fluorosulfonyl)benzoate |
| 316 | 317 | (2′S,3′R,6′R)-6′-hydroxy-2′-(hydroxymethyl)- |
| | | 2′,4′,6′-trimethyl-7′-oxo-2′,3′,6′,7′- |
| | | tetrahydrospiro[cyclopropane-1,5′-inden]- |
| | | 3′-yl 4-(fluorosulfonyl)benzoate |
| 317 | 318 | ((2′S,3′R,6′R)-3′-((4-(fluorosulfonyl)benzoyl)- |
| | | oxy)-6′-hydroxy-2′,4′,6′-trimethyl-7′-oxo- |
| | | 2′,3′,6′,7′-tetrahydrospiro[cyclopropane- |
| | | 1,5′-inden]-2′-yl)methyl 4-(fluorosulfonyl)benzoate |
| 318 | 332 | (R)-6′-hydroxy-3′-((methoxyamino)methyl)-2′,4′,6′- |
| | | trimethylspiro[cyclopropane-1,5′-inden]-7′(6′H)-one |
| 319 | 333 | ((2′S,3′R,6′R)-6′-hydroxy-2′,4′,6′-trimethyl-3′-(((4- |
| | | nitrophenoxy)carbonyl)oxy)-7′-oxo-2′,3′,6′,7′- |
| | | tetrahydrospiro[cyclopropane-1,5′-inden]-2′- |
| | | yl)methyl acetate |
| 320 | 334 | ((2′S,3′R,6′R)-3′-(((2-((tert-butoxycarbonyl)- |
| | | (methyl)amino)ethyl)(methyl)carbamoyl)oxy)- |
| | | 6′-hydroxy-2′,4′,6′-trimethyl-7′-oxo-2′,3′,6′,7′- |
| | | tetrahydrospiro[cyclopropane-1,5′-inden]-2′- |
| | | yl)methyl acetate |
| 321 | 335 | tert-butyl ((2′S,3′R,6′R)-6′-hydroxy-2′- |
| | | (hydroxymethyl)-2′,4′,6′-trimethyl-7′-oxo- |
| | | 2′,3′,6′,7′-tetrahydrospiro[cyclopropane-1,5′- |
| | | inden]-3′-yl) ethane-1,2-diylbis(methylcarbamate) |
| 322 | 337 | 4-nitrophenyl (R)-((6′-hydroxy-2′,4′,6′-trimethyl- |
| | | 7′-oxo-6′,7′-dihydrospiro[cyclopropane- |
| | | 1,5′-inden]-3′-yl)methyl)carbamate |
| 323 | 338 | 4-nitrophenyl (R)-(3-(6′-hydroxy-2′,4′,6′- |
| | | trimethyl-7′-oxo-6′,7′-dihydrospiro- |
| | | [cyclopropane-1,5′-inden]-3′-yl)propyl)carbamate |
| 324 | 339 | ((2′S,3′R,6′R)-3′,6′-dihydroxy-2′,4′,6′-trimethyl-7′- |
| | | oxo-2′,3′,6′,7′-tetrahydrospiro[cyclopropane-1,5′- |
| | | inden]-2′-yl)methyl 4-methylbenzenesulfonate |
| 325 | 340 | (R)-N-((6′-hydroxy-2′,4′,6′-trimethyl-7′-oxo-6′,7′- |
| | | dihydrospiro[cyclopropane-1,5′-inden]-3′- |
| | | yl)methyl)methanesulfonamide |
| 326 | 345 | (2′S,3′R,6′R)-2′-(acetoxymethyl)-6′-hydroxy- |
| | | 2′,4′,6′-trimethyl-7′-oxo-2′,3′,6′,7′- |
| | | tetrahydrospiro[cyclopropane-1,5′-inden]-3′-yl |
| | | 4-(N-(2-(2-(2-(2-aminoacetamido)acetamido)- |
| | | acetamido)ethyl)-N-methylsulfamoyl)benzoate |
| 327 | 346 | ((2′S,3′R,6′R)-3′,6′-dihydroxy-2′,4′,6′-trimethyl- |
| | | 7′-oxo-2′,3′,6′,7′-tetrahydrospiro[cyclopropane- |
| | | 1,5′-inden]-2′-yl)methyl 4-(N-(2-(2-(2-(2- |
| | | aminoacetamido)acetamido)acetamido)ethyl)- |
| | | N-methylsulfamoyl)benzoate |
| 328 | 347 | ((2′S,3′R,6′R)-3′,6′-dihydroxy-2′,4′,6′-trimethyl-7′- |
| | | oxo-2′,3′,6′,7′-tetrahydrospiro[cyclopropane-1,5′- |
| | | inden]-2′-yl)methyl 4-sulfamoylbenzoate |
| 330 | 348 | (3′S,6′R)-6′-hydroxy-2′,2′,4′,6′-tetramethyl-7′- |
| | | oxo-2′,3′,6′,7′-tetrahydrospiro[cyclopropane- |
| | | 1,5′-inden]-3′-yl 4-sulfamoylbenzoate |
| 331 | 351 | (3′S,6′R)-6′-hydroxy-2′,2′,4′,6′-tetramethyl-7′- |
| | | oxo-2′,3′,6′,7′-tetrahydrospiro[cyclopropane- |
| | | 1,5′-inden]-3′-yl 4-(N-(2-(2-(2-(2- |
| | | aminoacetamido)acetamido)acetamido)ethyl)- |
| | | N-methylsulfamoyl)benzoate |
| 332 | 353 | ((6′R)-3′-(((2-((((4-((S)-2-((S)-2-(6-(2,5- |
| | | dioxo-2,5-dihydro-1H-pyrrol-1-yl)- |
| | | hexanamido)-3-methylbutanamido)-5- |
| | | ureidopentanamido)benzyl)oxy)carbonyl)(methyl)- |
| | | amino)ethyl)(methyl)carbamoyl)oxy)-6′- |
| | | hydroxy-2′,4′,6′-trimethyl-7′-oxo-2′,3′,6′,7′- |
| | | tetrahydrospiro[cyclopropane-1,5′-inden]- |
| | | 2′-yl)methyl acetate |
| 333 | 354 | 4-((S)-2-((S)-2-(6-(2,5-dioxo-2,5-dihydro-1H- |
| | | pyrrol-1-yl)hexanamido)-3-methylbutanamido)- |
| | | 5-ureidopentanamido)benzyl(2-(3- |
| | | ((S)-6′-hydroxy-2′,4′,6′-trimethyl-7′-oxo-6′,7′- |
| | | dihydrospiro[cyclopropane-1,5′-inden]-3′- |
| | | yl)propylidene)hydrazine-1-carbonyl)carbamate |
| 334 | 356 | ((2′S,3′S,6′R)-3′-(((1-(9H-fluoren-9-yl)-13- |
| | | methyl-3,6,9,12-tetraoxo-2-oxa- |
| | | 4,7,10,13-tetraazapentadecan-15-yl)(methyl)- |
| | | carbamoyl)oxy)-6′-hydroxy-2′,4′,6′-trimethyl- |
| | | 7′-oxo-2′,3′,6′,7′-tetrahydrospiro[cyclopropane- |
| | | 1,5′-inden]-2′-yl)methyl acetate |
| 335 | 357 | ((2′S,3′S,6′R)-3′-(((2-(2-(2-(2-aminoacetamido)- |
| | | acetamido)-N-methylacetamido)ethyl)- |
| | | (methyl)carbamoyl)oxy)-6′-hydroxy- |
| | | 2′,4′,6′-trimethyl-7′-oxo-2′,3′,6′,7′-tetrahydrospiro- |
| | | [cyclopropane-1,5′-inden]-2′-yl)methyl acetate |
| 336 | 359 | ((2′S,3′S,6′R)-6′-hydroxy-2′,4′,6′-trimethyl-3′- |
| | | ((methyl(2,2,14-trimethyl-4,7,10,13- |
| | | tetraoxo-3-oxa-5,8,11,14-tetraazahexadecan- |
| | | 16-yl)carbamoyl)oxy)-7′-oxo-2′,3′,6′,7′- |
| | | tetrahydrospiro[cyclopropane-1,5′-inden]-2′- |
| | | yl)methyl acetate |
| 337 | 361 | ((2′S,3′R,6′R)-3′-((((2,5-dioxopyrrolidin-1-yl)- |
| | | oxy)carbonyl)oxy)-6′-hydroxy-2′,4′,6′- |
| | | trimethyl-7′-oxo-2′,3′,6′,7′-tetrahydrospiro- |
| | | [cyclopropane-1,5′-inden]-2′-yl)methyl acetate |
| 338 | 362 | ((2′S,3′R,6′R)-3′,6′-dihydroxy-2′,4′,6′-trimethyl-7′- |
| | | oxo-2′,3′,6′,7′-tetrahydrospiro[cyclopropane-1,5′- |
| | | inden]-2′-yl)methyl methanesulfonate |
| 339 | 363 | (2′S,3′R,6′R)-2′-(aminomethyl)-3′,6′-dihydroxy- |
| | | 2′,4′,6′-trimethyl-2′,3′-dihydrospiro- |
| | | [cyclopropane-1,5′-inden]-7′(6′H)-one |
| 340 | 364 | (2′R,3′R,6′R)-3′-amino-6′-hydroxy-2′- |
| | | (hydroxymethyl)-2′,4′,6′-trimethyl-2′,3′- |
| | | dihydrospiro[cyclopropane-1,5′-inden]- |
| | | 7′(6′H)-one |
| 341 | 366 | tert-butyl (R)-((6′-hydroxy-2′,4′,6′-trimethyl- |
| | | 7′-oxo-6′,7′-dihydrospiro[cyclopropane- |
| | | 1,5′-inden]-3′-yl)methyl)carbamate |
| 342 | 367 | tert-butyl (R)-(3-(6′-hydroxy-2′,4′,6′-trimethyl- |
| | | 7′-oxo-6′,7′-dihydrospiro[cyclopropane- |
| | | 1,5′-inden]-3′-yl)propyl)carbamate |
| 343 | 368 | ((2′S,3′R,6′R)-3′-acetoxy-6′-hydroxy-2′,4′,6′- |
| | | trimethyl-7′-oxo-2′,3′,6′,7′-tetrahydrospiro- |
| | | [cyclopropane-1,5′-inden]-2′-yl)methyl acetate |
| 344 | 369 | (2′S,3′R,6′R)-2′-(((tert-butyldimethylsilyl)- |
| | | oxy)methyl)-3′,6′-dihydroxy-2′,4′,6′-trimethyl- |
| | | 2′,3′-dihydrospiro[cyclopropane-1,5′- |
| | | inden]-7′(6′H)-one |
| 345 | 370 | (2′S,3′R,6′R)-2′-(((tert-butyldimethylsilyl)- |
| | | oxy)methyl)-6′-hydroxy-2′,4′,6′-trimethyl-7′-oxo- |
| | | 2′,3′,6′,7′-tetrahydrospiro[cyclopropane- |
| | | 1,5′-inden]-3′-yl acetate |
| 346 | 371 | (2′S,3′R,6′R)-6′-hydroxy-2′-(hydroxymethyl)- |
| | | 2′,4′,6′-trimethyl-7′-oxo-2′,3′,6′,7′-tetrahydrospiro- |
| | | [cyclopropane-1,5′-inden]-3′-yl acetate |
| 347 | 372 | (2′R,3′R,6′R)-2′-formyl-6′-hydroxy-2′,4′,6′- |
| | | trimethyl-7′-oxo-2′,3′,6′,7′-tetrahydrospiro- |
| | | [cyclopropane-1,5′-inden]-3′-yl acetate |
| 348 | 373 | (2′S,3′R,6′R)-2′-(((((9H-fluoren-9- |
| | | yl)methoxy)carbonyl)amino)methyl)-6′-hydroxy- |
| | | 2′,4′,6′-trimethyl-7′-oxo-2′,3′,6′,7′-tetrahydrospiro- |
| | | [cyclopropane-1,5′-inden]-3′-yl acetate |
| 349 | 374 | (2′S,3′R,6′R)-2′-(aminomethyl)-6′-hydroxy- |
| | | 2′,4′,6′-trimethyl-7′-oxo-2′,3′,6′,7′-tetrahydrospiro- |
| | | [cyclopropane-1,5′-inden]-3′-yl acetate |
| 350 | 377 | ((2′S,3′R,6′R)-6′-hydroxy-2′,4′,6′-trimethyl-7′-oxo- |
| | | 3′-((tributylsilyl)oxy)-2′,3′,6′,7′-tetrahydrospiro- |
| | | [cyclopropane-1,5′-inden]-2′-yl)methyl acetate |
| 351 | 378 | (2′S,3′R,6′R)-2′-((tert-butoxycarbonyl)amino) |
| | | methyl)-6′-hydroxy-2′,4′,6′-trimethyl-7′-oxo-2′,3′, |
| | | 6′,7′-tetrahydrospiro[cyclopropane-1,5′-inden]- |
| | | 3′-yl acetate |
| 352 | 379 | ((2′S,3′R,6′R)-3′-(((2-((((4-((14S,17S)-1-(9H- |
| | | fluoren-9-yl)-14-isopropyl-3,6,9,12,15- |
| | | pentaoxo-17-(3-ureidopropyl)-2-oxa- |
| | | 4,7,10,13,16-pentaazaoctadecan-18-amido)- |
| | | benzyl)oxy)carbonyl)(methyl)amino)ethyl)- |
| | | (methyl)carbamoyl)oxy)-6′-hydroxy-2′,4′,6′- |
| | | trimethyl-7′-oxo-2′,3′,6′,7′-tetrahydrospiro- |
| | | [cyclopropane-1,5′-inden]-2′-yl)methyl acetate |
| 353 | 380 | ((2′S,3′R,6′R)-3′-(((2-((((4-((2S,5S)-14-amino- |
| | | 5-isopropyl-4,7,10,13-tetraoxo-2-(3- |
| | | ureidopropyl)-3,6,9,12-tetraazatetradecanamido)- |
| | | benzyl)oxy)carbonyl)(methyl)amino)ethyl)- |
| | | (methyl)carbamoyl)oxy)-6′-hydroxy-2′,4′,6′- |
| | | trimethyl-7′-oxo-2′,3′,6′,7′-tetrahydrospiro- |
| | | [cyclopropane-1,5′-inden]-2′-yl)methyl acetate |
| 354 | 381 | (2′S,3′R,6′R)-6′-hydroxy-2′-(hydroxymethyl)-2′,4′,6′- |
| | | trimethyl-3′-((tributylsilyl)oxy)-2′,3′-dihydrospiro- |
| | | [cyclopropane-1,5′-inden]-7′(6′H)-one |
| 355 | 382 | (2′R,3′R,6′R)-6′-hydroxy-2′,4′,6′-trimethyl-7′-oxo- |
| | | 3′-((tributylsilyl)oxy)-2′,3′,6′,7′-tetrahydrospiro- |
| | | [cyclopropane-1,5′-indene]-2′-carbaldehyde |
| 356 | 383 | (2′R,3′R,6′R)-3′,6′-dihydroxy-2′,4′,6′-trimethyl- |
| | | 7′-oxo-2′,3′,6′,7′-tetrahydrospiro- |
| | | [cyclopropane-1,5′-indene]-2′-carbaldehyde |
| 357 | 384 | tert-butyl (((2′S,3′R,6′R)-6′-hydroxy-2′,4′,6′- |
| | | trimethyl-7′-oxo-3′-((tributylsilyl)oxy)- |
| | | 2′,3′,6′,7′-tetrahydrospiro[cyclopropane- |
| | | 1,5′-inden]-2′-yl)methyl)carbamate |
| 358 | 389 | tert-butyl (((2′S,3′R,6′R)-3′,6′-dihydroxy-2′,4′,6′- |
| | | trimethyl-7′-oxo-2′,3′,6′,7′-tetrahydrospiro- |
| | | [cyclopropane-1,5′-inden]-2′-yl)methyl)- |
| | | carbamate |
| 359 | 392 | ((2′S,3′S,6′R)-3′-(((2-(2-(2-(2-azidoacetamido)- |
| | | acetamido)-N-methylacetamido)ethyl)(methyl)- |
| | | carbamoyl)oxy)-6′-hydroxy-2′,4′,6′-trimethyl- |
| | | 7′-oxo-2′,3′,6′,7′-tetrahydrospiro[cyclopropane- |
| | | 1,5′-inden]-2′-yl)methyl acetate |
| 360 | 393 | ((2′S,3′R,6′R)-3′-(((2-(2,5-dioxo-2,5-dihydro-1H- |
| | | pyrrol-1-yl)ethyl)carbamoyl)oxy)-6′-hydroxy-2′, |
| | | 4′,6′-trimethyl-7′-oxo-2′,3′,6′,7′-tetrahydrospiro- |
| | | [cyclopropane-1,5′-inden]-2′-yl)methyl acetate |
| 361 | 394 | (R)-6′-hydroxy-3′-((hydroxyamino)methyl)-2′,4′,6′- |
| | | trimethylspiro[cyclopropane-1,5′-inden]-7′(6′H)-one |
| 362 | 397 | tert-butyl (((2′S,3′R,6′R)-3′,6′-dihydroxy-2′,4′,6′- |
| | | trimethyl-7′-oxo-2′,3′,6′,7′-tetrahydrospiro- |
| | | [cyclopropane-1,5′-inden]-2′- |
| | | yl)methyl)(hydroxy)carbamate |
| 363 | 398 | (2′S,3′R,6′R)-3′,6′-dihydroxy-2′-((hydroxyamino)- |
| | | methyl)-2′,4′,6′-trimethyl-2′,3′-dihydrospiro- |
| | | [cyclopropane-1,5′-inden]-7′(6′H)-one |
| 364 | 399 | ((2′S,3′R,6′R)-3′,6′-dihydroxy-2′,4′,6′-trimethyl-7′- |
| | | oxo-2′,3′,6′,7′-tetrahydrospiro[cyclopropane- |
| | | 1,5′-inden]-2′-yl)methyl sulfamate |
| 365 | 401 | ((6′R)-3′-(((2-((((4-((S)-2-((S)-2-((((9H-fluoren-9- |
| | | yl)methoxy)carbonyl)amino)-3-methylbutanamido)- |
| | | 5-ureidopentanamido)benzyl)oxy)carbonyl)- |
| | | (methyl)amino)ethyl)(methyl)carbamoyl)oxy)-6′- |
| | | hydroxy-2′,4′,6′-trimethyl-7′-oxo-2′,3′,6′,7′- |
| | | tetrahydrospiro[cyclopropane-1,5′-inden]-2′-yl)- |
| | | methyl acetate |
| 366 | 402 | (R)-(6′-hydroxy-2′,4′,6′-trimethyl-7′-oxo-6′,7′- |
| | | dihydrospiro[cyclopropane-1,5′-inden]-3′-yl)- |
| | | methyl sulfamate |
| 367 | 403 | ((2′S,6′R)-6′-hydroxy-2′,6′-dimethyl-4′-methylene- |
| | | 7′-oxo-2′,4′,6′,7′-tetrahydrospiro[cyclopropane- |
| | | 1,5′-inden]-2′-yl)methyl methanesulfonate |
| 368 | 404 | ((6′R)-3′-(((2-((((4-((S)-2-((S)-2-amino-3- |
| | | methylbutanamido)-5-ureidopentanamido)- |
| | | benzyl)oxy)carbonyl)(methyl)amino)ethyl)- |
| | | (methyl)carbamoyl)oxy)-6′-hydroxy-2′,4′,6′-trimethyl- |
| | | 7′-oxo-2′,3′,6′,7′-tetrahydrospiro- |
| | | [cyclopropane-1,5′-inden]-2′-yl)methyl acetate |
| 369 | 405 | ((2′S,3′S,6′R)-6′-hydroxy-3′-(((2-((((4-((2S,5S)- |
| | | 5-isopropyl-25-(6-methyl-4-vinylpyridin- |
| | | 2-yl)-4,7,23-trioxo-2-(3-ureidopropyl)- |
| | | 10,13,16,19-tetraoxa-3,6,22- |
| | | triazapentacosanamido)benzyl)oxy)carbonyl)- |
| | | (methyl)amino)ethyl)(methyl)carbamoyl)oxy)- |
| | | 2′,4′,6′-trimethyl-7′-oxo-2′,3′,6′,7′-tetrahydrospiro- |
| | | [cyclopropane-1,5′-inden]-2′-yl)methyl acetate |
| 370 | 407 | ((2′S,3′R,6′R)-3′,6′-dihydroxy-2′,4′,6′-trimethyl- |
| | | 7′-oxo-2′,3′,6′,7′-tetrahydrospiro[cyclopropane- |
| | | 1,5′-inden]-2′-yl)methyl |
| | | trifluoromethanesulfonate |
| 371 | 408 | 4-((2S,5S)-14-amino-5-isopropyl-4,7,10,13- |
| | | tetraoxo-2-(3-ureidopropyl)-3,6,9,12- |
| | | tetraazatetradecanamido)benzyl |
| | | ((2′S,3′R,6′R)-6′-hydroxy-2′-(hydroxymethyl)- |
| | | 2′,4′,6′-trimethyl-7′-oxo-2′,3′,6′,7′- |
| | | tetrahydrospiro[cyclopropane-1,5′-inden]-3′-yl) |
| | | ethane-1,2-diylbis(methylcarbamate) |
| 372 | 409 | 4-((S)-2-((S)-2-amino-3-methylbutanamido)-5- |
| | | ureidopentanamido)benzyl ((6′R)-6′- |
| | | hydroxy-2′-(hydroxymethyl)-2′,4′,6′- |
| | | trimethyl-7′-oxo-2′,3′,6′,7′-tetrahydrospiro- |
| | | [cyclopropane-1,5′-inden]-3′-yl) ethane- |
| | | 1,2-diylbis(methylcarbamate) |
| 373 | 410 | 4-((S)-2-((S)-2-(6-(2,5-dioxo-2,5-dihydro- |
| | | 1H-pyrrol-1-yl)hexanamido)-3- |
| | | methylbutanamido)-5-ureidopentanamido)benzyl |
| | | ((6′R)-6′-hydroxy-2′-(hydroxymethyl)-2′,4′,6′- |
| | | trimethyl-7′-oxo-2′,3′,6′,7′-tetrahydrospiro- |
| | | [cyclopropane-1,5′-inden]-3′-yl) ethane- |
| | | 1,2-diylbis(methylcarbamate) |

TABLE VI
Summary NCI DTP 60 Cell Line Data.

| | Mean GI50 | | Mean TGI | | Mean LD50 | |
|---|---|---|---|---|---|---|
| NAME/NSC | inhibition | | cytostatic | | cytotoxic | |
| Pyrrolo- | 7 | nM | 302 | nM | >23,000 | nM* |
| benzodiazepines | | | | | | |
| 694501 | | | | | | |
| Maytansine** | 19 | nM | 318 | nM | 49,200 | nM |
| 153858 | | | | | | |
| Fumagillol | 6,130 | nM | 9,850 | nM | >50,000 | nM |
| 642492 | | | | | | |
| Dolstatin-10 | 17 | nM | 2,680 | nM | >50,000 | nM |
| 376128 | | | | | | |
| Auristatins | 1.4 | nM | 902** | nM | >5,000 | nM** |
| 654663 | | | | | | |
| Enadiyne 157365 | 2,900 | nM | >100,000 | nM | >100,000 | nM |
| Halichondrin B | 1.2 | nM | 199 | nM | >1,000 | nM |
| 609395 | | | | | | |
| Tubulysin A | 12 | nM | 1,318 | nM | >10,000 | nM |
| Illudin S | 10 | nM | 64 | nM | 511 | nM |
| Illudin M | 3 | nM | 20 | nM | 291 | nM |

TABLE VII
Ability of Illudofulvene analogs to inhibit tumor cell growth.

| | Mean IC50 value (nM) ± SD, N = 3 unless otherwise indicated | | | |
|---|---|---|---|---|
| Analog | MV522 Target Cell Line | | 8392B Nontarget Cell Line | |
| Number | 2 hr exposure | 48 hr exposure | 2 hr exposure | 48 hr exposure |
| 001 | 2200 ± 100 | 350 ± 20 | | 830 ± 100 |
| 002 | 110 ± 40 | 70 ± 10 | 26000 ± 4500 | 800 ± 100 |
| 004 | 4200 | 600 | | |
| 008 | 870 ± 90 | 630 ± 80 | 12200 ± 700 | 15100 ± 2200 |
| 009 | 500 ± 30 | 850 ± 180 | 47100 ± 11000 | 43200 ± 2300 |
| 010 | 8900 ± 1500 | 170 ± 60 | 29400 ± 1600 | 14500 ± 1700 |
| 011 | 4900 ± 900 | 1200 (N = 2) | >100000 | 40400 ± 6700 |
| 012 | 5150 ± 1350 | 320 ± 90 | 42200 ± 5000 | 18800 ± 2800 |
| 013 | 5100 ± 700 | 270 ± 130 | 11900 ± 1300 | 4200 ± 400 |
| 014 | 115 ± 30 | 460 ± 120 | 9650 ± 200 | 1100 ± 300 |
| 015 | 1800 ± 200 | 480 ± 110 | 810 ± 260 | 1300 ± 150 |
| 016 | 490 ± 130 | 440 ± 90 | >100000 | 870 ± 60 |
| 017 | 2400 ± 360 | 320 ± 60 | 14700 ± 900 | |
| 018 | 8800 ± 2900 | | 4200 ± 1300 | |
| 019 | 470 ± 60 | 660 ± 80 | >75000 | |
| 020 | 530 ± 140 | 230 ± 10 | 25000 ± 3100 | |
| 021 | 2400 ± 1000 | 930 ± 250 | 34400 ± 9400 | |
| 022 | 700 ± 200 | 680 ± 180 | 31700 ± 1400 | |
| 023 | 2900 ± 1140 | 2750 ± 500 | >138000 | |
| 024 | 1800 ± 200 | 1200 ± 300 | 12800 ± 2100 | |
| 025 | 1300 ± 310 | 1200 ± 100 | >25000 | |
| 030 | | >3000 | | |
| 031 | | >3000 | | |
| 032 | 600 ± 190 | 210 ± 30 | >30000 | |
| 033 | 10000 ± 1100 | 4600 ± 200 | 29900 ± 3300 | |
| 034 | 1400 ± 170 | 490 ± 40 | >100000 | 4400 ± 200 |
| 035 | 5600 ± 600 | | >150000 | |
| 037 | 26000 ± 5000 | 29200 ± 2300 | >85000 | |
| 038 = | 750 ± 60 | | 24900 ± 8000 | |
| 091 | | | | |
| 039 = | 1500 ± 240 | 600 ± 40 | 24600 ± 2400 | 820 ± 250 |
| 092 | | | | |
| 040 = | 3400 ± 360 | 700 ± 90 | 24000 ± 3300 | 5200 ± 470 |
| 093 | | | | |
| 060 | 19400 ± 1800 | | 27600 ± 3000 | |
| 062 | 2600 ± 300 | 660 ± 200 | 37100 ± 2300 | |
| 063 | 43000 ± 5700 | 580 ± 250 | | |
| 064 | 28000 ± 4600 | 1200 ± 300 | | |
| 065 | 6200 ± 1100 | 2500 ± 1200 | | |
| 075 | 19600 ± 9700 | | 62000 ± 3600 | |
| 076 | 24000 ± 6100 | | 39500 ± 7200 | |
| 077 | 9200 ± 1200 | | | |
| 078 | 20400 ± 6300 | | >100000 | |
| 079 | 7700 ± 3500 | | >100000 | |
| 080 | 8800 ± 2400 | | >100000 | |
| 081 | >80000 | | >80000 | |
| 082 | 50600 ± 7100 | | >100000 | |
| 083 | | 37200 ± 2900 | | >42000 |
| 084 | | 28200 ± 1400 | | >42000 |
| 085 | >40000 | >40000 | | |
| 087 | >40000 | 24700 ± 3900 | >40000 | |
| 089 | 19300 ± 5700 | 15500 ± 2800 | >60000 | |
| 090 | 2500 ± 400 | 2900 ± 400 | 1600 ± 200 | 3800 ± 300 |
| 094 | 800 ± 100 | 210 ± 20 | 9000 ± 1700 | 110 ± 10 |
| 096 | 2700 ± 400 | 6200 ± 600 | >88000 | >3000 |
| 097 | 2900 ± 100 | | >82000 | |
| 098 | 18800 ± 2500 | 4600 ± 250 | >65000 | 11700 ± 1800 |
| 099 | 8400 ± 1100 | 1800 ± 200 | 4000 ± 400 | 300 ± 20 |
| 100 | >10000 | 1700 ± 500 | | |
| 101 | >8000 | >7500 | | |
| 102 | >13000 | 1300 ± 100 | | |
| 103 | 31800 ± 4900 | 5900 ± 400 | 12100 ± 2000 | 2300 ± 200 |
| 104 | 6300 ± 400 | 6000 ± 500 | 36400 ± 6500 | 2700 ± 600 |
| 105 | 7300 ± 1200 | 2100 ± 400 | >100000 | |
| 106 | 5200 ± 1000 | | >83000 | |
| 107 | >50000 | 1600 ± 100 | >50000 | |
| 108 | 12300 ± 2300 | 520 ± 50 | >55000 | 6000 ± 1600 |
| 109 | >50000 | | >50000 | |
| 110 | >55000 | 1400 ± 100 | >55000 | 25300 ± 2100 |
| 111 | 16700 ± 2100 | 11900 ± 2800 | 34600 ± 2100 | 10200 ± 1000 |
| 112 | 10000 ± 2000 | 6700 ± 1200 | 14900 ± 100 | 5200 ± 300 |
| 113 | 85000 ± 700 | 14100 ± 3000 | >93000 | 7800 ± 1000 |
| 114 | 1500 ± 100 | 260 ± 70 | 25100 ± 1000 | 700 ± 100 |
| 115 | 1500 ± 100 | 70 ± 5 | 1600 ± 700 | 630 ± 60 |
| 116 | 400 ± 100 | 1000 ± 50 | 7000 ± 400 | 170 ± 30 |
| 117 | 1100 ± 100 | 100 ± 30 | 7900 ± 1600 | 10 ± 2 |
| 118 | 14000 ± 2000 | 740 ± 120 | 24500 ± 4500 | 2000 ± 400 |
| 119 | 1100 ± 70 | 270 ± 40 | >33000 | >10000 |
| 120 | 2800 ± 900 | 600 ± 100 | 19100 ± 4600 | 510 ± 110 |
| 121 | 300 ± 10 | 90 ± 10 | 15200 ± 6000 | 1300 ± 500 |
| 122 | 6400 ± 300 | 2400 ± 300 | 14500 ± 1200 | 1100 ± 300 |
| 123 | 1900 ± 400 | 600 ± 60 | 450 ± 30 | 2400 ± 500 |
| 124 | 2800 ± 700 | 870 ± 350 | >30000 | 2400 ± 550 |
| 125 | 3700 ± 600 | 1200 ± 200 | 15500 ± 1400 | 600 ± 100 |
| 126 | 2100 ± 500 | 900 ± 100 | >30000 | 330 ± 80 |
| 127 | 870 ± 30 | 340 ± 90 | >30000 | 100 ± 40 |
| 128 | 840 ± 230 | 370 ± 50 | >35000 | 800 ± 70 |
| 129 | >136000 | 19700 ± 1900 | >136000 | 39400 ± 9200 |
| 130 | 700 ± 100 | 130 ± 40 | 27,000 ± 7000 | 4400 ± 500 |
| 133 | 58800 ± 6600 | 15800 ± 2600 | 12200 ± 2300 | 2700 ± 400 |
| 134 | 50000 ± 6000 | 28000 ± 4000 | 43900 ± 5100 | 8500 ± 2000 |
| 135 | 1600 ± 300 | 22 ± 4 | 70 ± 20 | 22 ± 2 |
| 136 | 430 ± 10 | 130 ± 10 | >6200 | 25 ± 2 |
| 137 | 850 ± 110 | 1200 ± 100 | 8500 ± 1200 | 710 ± 60 |
| 138 | 2100 ± 200 | 1000 ± 200 | 5400 ± 200 | 820 ± 230 |
| 139 | 6400 ± 900 | 3400 ± 500 | 11600 ± 900 | 2600 ± 1000 |
| 140 | 17100 ± 5100 | >14000 | 12700 ± 300 | >14000 |
| 141 | 11400 ± 1000 | 3700 ± 800 | 13700 ± 1900 | 1100 ± 140 |
| 142 | 90 ± 10 | 24 ± 7 | 6400 ± 1100 | 80 ± 6 |
| 143 | 43500 ± 11300 | 11400 ± 1800 | 56500 ± 20000 | 3600 ± 700 |
| 146 | 2500 ± 400 | 740 ± 280 | 13,000 ± 1200 | |
| 147 | >76000 | 26100 ± 12900 | >76000 | 43800 ± 3000 |
| 148 | 17100 ± 1100 | 6800 ± 1100 | 61000 ± 11600 | 6700 ± 1600 |
| 149 | 2900 ± 1000 | 1500 500 | 44600 ± 1400 | 4100 ± 900 |
| 150 | 9500 ± 1600 | 1400 ± 400 | 59000 ± 5500 | 10600 ± 800 |
| 151 | 7900 ± 400 | 4200 ± 1600 | 25500 ± 1200 | 6600 ± 2300 |
| 152 | | 6400 ± 1200 | 49000 ± 7700 | 9100 ± 100 |
| 153 | 8700 ± 2700 | 10900 ± 3400 | >90000 | 15800 ± 9600 |
| 154 | >70000 | 61300 ± 10000 | >70000 | 46,700 ± 13100 |
| 155 | 8200 ± 1200 | 3600 ± 400 | 17,000 ± 4000 | 9100 ± 1100 |
| 156 | 7200 ± 500 | 3100 ± 100 | 32,300 ± 9,400 | 5500 ± 1200 |
| 157 | >400,000 | >123,000 | >350,000 | 13100 ± 1600 |
| 158 | >175,000 | >175,000 | >200,000 | 61,000 ± 9,000 |
| 159 | 2700 ± 400 | 120 ± 10 | 13,700 ± 4,200 | <10 nM |
| 160 | 1900 ± 200 | 500 ± 200 | 52,400 ± 17,800 | 3200 ± 1100 |
| 161 | 2800 ± 500 | 3300 ± 700 | 13,800 ± 3,400 | >10,000 |
| 163 | 3500 ± 800 | 820 ± 40 | 18600 ± 800 | 910 ± 100 |
| 164 | | 70 ± 10 | 3500 ± 1600 | 130 ± 40 |
| 165 | 7700 ± 1100 | 290 ± 40 | 11000 ± 3300 | 11000 ± 1000 |
| 166 | 6500 ± 600 | 7200 ± 1900 | 6500 ± 2100 | 6000 ± 1500 |
| 167 | 14800 ± 2200 | | 18500 ± 2300 | |
| 169 | 7100 ± 600 | | 2300 ± 600 | |
| 177 | 7500 ± 800 | 1900 ± 800 | 73000 ± 5000 | 4100 ± 1300 |
| 178 | 21000 ± 4000 | 1000 ± 100 | 32000 ± 9000 | >8000 |
| 180 | 19900 ± 300 | >4000 | 5200 ± 1800 | 660 ± 50 |
| 182 | 99000 ± 12000 | 38000 ± 8200 | 39000 ± 7000 | 18700 ± 2700 |
| 183 | >120,000 | >275,000 | >120,000 | >235,000 |
| 184 | 800 ± 300 | 210 ± 20 | >100,000 | >10000 |
| 185 | 1700 ± 600 | 1900 ± 100 | | |
| 186 | 144000 ± 32000 | 70000 ± 16000 | 79000 ± 24000 | 48000 ± 2000 |
| 187 | 1300 ± 400 | 900 ± 200 | 3200 ± 800 | 3200 ± 700 |
| 189 | 8900 ± 2500 | 6100 ± 2600 | 41,000 ± 3700 | |
| 190 | 19,000 ± 4000 | >9,000 | 56,000 ± 2000 | >9,000 |
| 191 | >140,000 | 49,000 ± 13000 | >140,000 | 15000 ± 4000 |
| 192 | 1,600 ± 200 | 700 ± 100 | 8700 ± 1700 | 200 ± 30 |
| 193 | 1400 ± 400 | 2500 ± 600 | 48,000 ± 7000 | >11,000 |
| 195 | 1400 ± 200 | 390 ± 120 | 21,000 ± 6000 | 4300 ± 1200 |
| 196 | 840 ± 100 | 450 ± 120 | 80,000 ± 5000 | >9,200 |
| 197 | 950 ± 70 | 500 ± 100 | 9500 ± 400 | 11,300 ± 100 |
| 198 | 700 ± 100 | 2800 ± 600 | >8,200 | >82,000 |
| 199 | 4700 ± 600 | 2500 ± 1100 | >93,000 | >9,300 |
| 201 | 360 ± 110 | 260 ± 70 | 13,000 ± 1700 | 26,000 ± 7000 |
| 202 | 1200 ± 100 | 650 ± 100 | >62,000 | >6200 |
| 203 | 760 ± 170 | 940 ± 330 | 48,000 ± 6000 | >5500 |
| 204 | 220 ± 40 | 1600 ± 300 | 4100 ± 800 | 8600 ± 800 |
| 205 | 8400 ± 2200 | 1200 ± 4400 | >185,000 | >2,600 |
| 206 | 610 ± 40 | 230 ± 20 | 20,000 ± 1000 | 8200 ± 200 |
| 207 | 570 ± 60 | 410 ± 60 | | |
| 208 | 1200 ± 100 | 930 ± 160 | 25,000 ± 3000 | |
| 209 | 3900 ± 1100 | 610 ± 100 | >90,000 | |
| 210 | 40,000 ± 4000 | 5500 ± 600 | | |
| 211 | 470 ± 120 | 430 ± 100 | 59,000 ± 9000 | |
| 212 | 80 ± 10 | 55 ± 5 | | |
| 213 | 2300 ± 700 | 1700 ± 700 | | |
| 214 | 2900 ± 800 | 360 ± 30 | | |
| 215 | 26,000 ± 3000 | 490 ± 120 | | |
| 216 | 460 ± 60 | 150 ± 40 | | |
| 217 | 2,200 ± 100 | 2,200 ± 100 | 43,000 ± 4,000 | >7,000 |
| 218 | 10,000 ± 3,000 | 600 ± 200 | 15,000 ± 6,000 | 600 ± 100 |
| 219 | >52,000 | >52,00 | >52,000 | >52,000 |
| 220 | 90 ± 10 | 130 ± 10 | 101,000 ± 18,000 | 40,000 ± 3,000 |
| 221 | >21,000 | 2,500 ± 200 | >21,000 | >21,000 |
| 222 | 5,000 ± 100 | 1,100 ± 100 | 9,300 ± 200 | 330 ± 60 |
| 223 | 20,000 ± 3,700 | 2,700 ± 300 | >185,000 | >55,000 |
| 224 | >200,000 | >130,000 | >200,000 | >130,000 |
| 225 | 47,000 ± 4,000 | 55,000 ± 11,000 | >350,000 | 33,000 ± 13,000 |
| 226 | >59,000 | >59,000 | >59,000 | >59,000 |
| 227 | >57,000 | 4,400 ± 700 | >57,000 | 16,000 ± 4,000 |
| 228 | >38,000 | >38,000 | 24,000 ± 3,000 | >38,000 |
| 229 | >56,000 | >2,000 | >56,000 | >2,000 |
| 230 | 620 ± 80 | 100 ± 10 | 38,000 ± 5,000 | 1,000 ± 200 |
| 231 | 1,500 ± 100 | 280 ± 10 | 14,000 ± 4,000 | |
| 232 | 700 ± 100 | 460 ± 60 | 42,000 ± 6,000 | 3,300 ± 600 |
| 233 | 3,200 ± 300 | 350 ± 80 | >150,000 | 2,400 ± 700 |
| 234 | 3,000 ± 300 | 1,100 ± 400 | 24,000 ± 6,000 | 9,000 ± 1,000 |
| 235 | 3,500 ± 400 | 2,200 ± 400 | 49,000 ± 6,000 | 6,500 ± 1,600 |
| 236 | 49,000 ± 11,000 | 29,000 ± 5,000 | 48,000 ± 10,000 | |
| 237 | 1,200 ± 300 | 730 ± 140 | 22,000 ± 1,000 | 6,600 ± 900 |
| 238 | 780 ± 190 | 57 ± 8 | 23,000 ± 2,000 | 4,700 ± 1,200 |
| 239 | 420 ± 60 | 70 ± 20 | 39,000 ± 3,000 | 28,000 ± 4,000 |
| 240 | 2,900 ± 100 | 1,300 ± 200 | >24,000 | 1,300 ± 100 |
| 241 | 560 ± 90 | 110 ± 20 | >28,000 | 18,000 ± 4,000 |
| 242 | 2,400 ± 400 | 580 ± 150 | 18,000 ± 2,000 | 2,900 ± 600 |
| 243 | 2,200 ± 500 | 670 ± 240 | 64,000 ± 10,000 | 26,000 ± 6,000 |
| 244 | 1,600 ± 400 | 150 ± 10 | 87,000 ± 11,000 | 35,000 ± 7,000 |
| 245 | 3,400 ± 1000 | 440 ± 90 | 79,000 ± 7,000 | 14,000 ± 1,700 |
| 246 | 2,800 ± 260 | 1,900 ± 450 | 14,000 ± 2,000 | 6,200 ± 1,300 |
| 247 | 6,100 ± 2,000 | 1,200 ± 250 | 10,000 ± 1,400 | 7,100 ± 1,700 |
| 248 | 830 ± 100 | 200 ± 25 | 23,000 ± 1,000 | 610 ± 120 |
| 249 | 4,100 ± 820 | 420 ± 100 | 18,000 ± 3,500 | 19,000 ± 3,800 |
| 250 | 99,000 ± 21,000 | 137,000 ± 14,000 | >275,000 | 137,000 ± 10,000 |
| 251 | 128,000 ± 4,000 | 51,000 ± 1,000 | >275,000 | 82,000 ± 8,000 |
| 252 | >380,000 | 33,000 ± 3,000 | >380,000 | >380,000 |
| 253 | >380,000 | >38,000 | >380,000 | >380,000 |
| 254 | 2,700 ± 800 | 1,100 ± 100 | 43,000 ± 6,000 | >65,000 |
| 255 | 2,900 ± 500 | 55 ± 2 | 119,000 ± 15,000 | 99,000 ± 4,000 |
| 256 | 1,500 ± 200 | 880 ± 200 | 7,500 ± 800 | 7,100 ± 300 |
| 257 | 2,800 ± 600 | 320 ± 30 | 25,000 ± 2,000 | 26,000 ± 3,000 |
| 258 | >45,000 | >45,000 | >45,000 | >45,000 |
| 259 | 16000 ± 3000 | 2400 ± 200 | >85,000 | 4700 ± 400 |
| 260 | 1600 ± 500 | 150 ± 20 | >64,000 | 19000 ± 4500 |
| 261 | 6300 ± 1100 | 1000 ± 150 | 64000 ± 2000 | 38000 ± 2100 |
| 262 | 8700 ± 1300 | 3900 ± 570 | 287000 ± 14000 | 73000 ± 17000 |
| 263 | 2000 ± 300 | 1400 ± 200 | 124000 ± 18000 | 39000 ± 7000 |
| 264 | 1400 ± 100 | 76 ± 17 | >85,000 | 54000 ± 20000 |
| 265 | 810 ± 20 | 8 ± 1 | 1100 ± 200 | 250 ± 80 |
| 266 | 140 ± 20 | 70 ± 18 | 56000 ± 15000 | 32000 ± 7000 |
| 267 | 900 ± 160 | 160 ± 20 | >90,000 | 28000 ± 8000 |
| 268 | 2100 ± 200 | 330 ± 90 | 54,000 ± 16000 | >8,000 |
| 269 | 11000 ± 3000 | 850 ± 320 | 52000 ± 4000 | >7,000 |
| 270 | 8000 ± 1500 | 1300 ± 100 | >84,000 | 7100 ± 700 |
| 271 | 1700 ± 200 | 200 ± 90 | >93,000 | >9,300 |
| 272 | >46,000 | >4,700 | >47,000 | >4,700 |
| 273 | 30000 ± 5000 | >1,500 | >45,000 | >4,500 |
| 274 | 39000 ± 3000 | 1200 ± 300 | >46,000 | >4,500 |
| 275 | 1500 ± 300 | 370 ± 40 | >62,000 | >6,200 |
| 276 | 1500 ± 200 | 760 ± 100 | >61,000 | >6,100 |
| 277 | 760 ± 70 | 190 ± 20 | 31,000 ± 6000 | 9,800 ± 1000 |
| 278 | 1000 ± 100 | 270 ± 10 | >94000 | >9,400 |
| 279 | 1700 ± 400 | 190 ± 20 | >90000 | >9,000 |
| 280 | 2400 ± 800 | <80 | >83000 | >2,800 |
| 281 | 1800 ± 700 | 170 ± 10 | 27000 ± 2000 | 5000 ± 700 |
| 282 | 680 ± 60 | 110 ± 10 | >85000 | >8,500 |
| 283 | 2900 ± 1200 | 300 ± 20 | 40000 ± 4000 | >9,300 |
| 284 | 13,600(N = 2) | 340 ± 20 | | >8,800 |
| 285 | 3800 ± 1100 | 310 ± 20 | 84000 ± 9000 | 2000 ± 100 |
| 286 | 48000 ± 10000 | 6300 ± 200 | 51000 ± 1700 | >8,800 |
| 287 | 455000 ± 22000 | 1100 ± 100 | 567000 ± 17000 | 4700 ± 400 |
| 288 | 1800 ± 600 | 150 ± 20 | 11000 ± 3200 | ~9,000 |
| 289 | 51 ± 4 | 530 ± 150 | >290000 | >8,800 |
| 294 | 960 ± 170 | | | |
| 295 | 200 ± 44 | | | |
| 296 | 250 (N = 2) | | | |
| 297 | 2200 (N = 1) | | | |
| 298 | >7000 | | | |

TABLE VIII
Screening of Analogs from the National Cancer
Institute (NCI) Developmental Therapeutics Program
(DTP) NCI 60 cell line screen assay.

| Analog | Activity Value* | Cytotoxicity | MDR Activity |
|---|---|---|---|
| Epothilone A | +31 | No | No |
| MMAE | +21 | No | No |
| DM1 | +8 | No | No |
| 002 | −15 | Yes | Yes |
| 142 | −5 | Yes | Yes |
| 159 | −25 | Yes | Yes |
| 176 | −11 | Yes | Yes |
| 334 | −11 | Yes | Yes |
| 362 | −9 | Yes | Yes |
| 371 | −18 | Yes | Yes |
| 383 | −1 | Yes | Yes |
| 394 | −2 | Yes | Yes |

*Activity Value = the lower or more negative the activity value is an indication of the more active the compound was against tumor cells in the 60 cell line panel. A positive value indicates tumor cells could grow but at a slower rate. A value of Zero (0) indicates that while no cell growth was detected there was no evidence of cell death occurring (i.e., when the drug is removed the cells could grow again). A negative value indicates that the compounds actually lyse tumor cells.

What is claimed is:

1. A method of ablating one or more cancer cells in a mammal in need thereof comprising:
(a) determining from a test sample that the mammal has a cancer;
(b) determining an affinity moiety that targets a marker expressed on the cancer;
(c) generating an affinity drug conjugate comprising:
the affinity moiety;
a composition comprising a compound selected from the group consisting of:
(6′-hydroxy-2′,4′,6′-trimethyl-3′-(((4-nitrophenoxy)carbonyl)oxy)-7′-oxo-2′,3′,6′,7′-tetrahydrospiro[cyclopropane-1,5′-inden]-2′-yl)methyl acetate; (3′-(((2-((tert-butoxycarbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; tert-butyl (6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate); (3′,6′-dihydroxy-2′,4′,6′-trimethyl-7′-oxo-2′,3′,6′,7′-tetrahydrospiro[cyclopropane-1,5′-inden]-2′-yl)methyl 4-methylbenzenesulfonate; 2′-(acetoxymethyl)-6′-hydroxy-2′,4′,6′-trimethyl-7′-oxo-2′,3′,6′,7′-tetrahydrospiro[cyclopropane-1,5′-inden]-3′-yl 4-(N-(2-(2-(2-(2-aminoacetamido)acetamido)acetamido)ethyl)-N-methylsulfamoyl)benzoate; (3′,6′-dihydroxy-2′,4′,6′-trimethyl-7′-oxo-2′,3′,6′,7′-tetrahydrospiro[cyclopropane-1,5′-inden]-2′-yl)methyl 4-(N-(2-(2-(2-(2-aminoacetamido)acetamido)acetamido)ethyl)-N-methylsulfamoyl)benzoate; 6′-hydroxy-2′,2′,4′,6′-tetramethyl-7′-oxo-2′,3′,6′,7′-tetrahydrospiro[cyclopropane-1,5′-inden]-3′-yl 4-(N-(2-(2-(2-(2-aminoacetamido)acetamido)acetamido)ethyl)-N-methylsulfamoyl)benzoate; (3′-(((2-((((4-((S)-2-((S)-2-(6-(2,5-dioxo-2,5-dihydro-1H-pyrrol-1-yl)hexanamido)-3-methylbutanamido)-5-ureidopentanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (3′-(((1-(9H-fluoren-9-yl)-13-methyl-3,6,9,12-tetraoxo-2-oxa-4,7,10,13-tetraazapentadecan-15-yl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (3′-(((2-(2-(2-(2-aminoacetamido)acetamido)-N-methylacetamido)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (6′-hydroxy-2′,4′,6′-trimethyl-3′-((methyl(2,2,14-trimethyl-4,7,10,13-tetraoxo-3-oxa-5,8,11,14-tetraazahexadecan-16-yl)carbamoyl)oxy)-7′-oxo-2′,3′,6′,7′-tetrahydrospiro[cyclopropane-1,5′-inden]-2′-yl)methyl acetate; (3′-((((2,5-dioxopyrrolidin-1-yl)oxy)carbonyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (3′,6′-dihydroxy-2′,4′,6′-trimethyl-7′-oxo-2′,3′,6′,7′-tetrahydrospiro[cyclopropane-1,5′-inden]-2′-yl)methyl methanesulfonate; 2′-(aminomethyl)-3′,6′-dihydroxy-2′,4′,6′-trimethyl-2′,3′-dihydrospiro [cyclopropane-1,5′-inden]-7′(6′H)-one; 2′-(((tert-butyldimethylsilyl)oxy)methyl)-6′-hydroxy-2′,4′,6′-trimethyl-7′-oxo-2′,3′,6′,7′-tetrahydrospiro[cyclopropane-1,5′-inden]-3′-yl acetate; 6′-hydroxy-2′-(hydroxymethyl)-2′,4′,6′-trimethyl-7′-oxo-2′,3′,6′,7′-tetrahydrospiro[cyclopropane-1,5′-inden]-3′-yl acetate; 2′-formyl-6′-hydroxy-2′,4′,6′-trimethyl-7′-oxo-2′,3′,6′,7′-tetrahydrospiro[cyclopropane-1,5′-inden]-3′-yl acetate; 2′-(((((9H-fluoren-9-yl)methoxy)carbonyl)amino)methyl)-6′-hydroxy-2′,4′,6′-trimethyl-7′-oxo-2′,3′,6′,7′-tetrahydrospiro[cyclopropane-1,5′-inden]-3′-yl acetate; 2′-(aminomethyl)-6′-hydroxy-2′,4′,6′-trimethyl-7′-oxo-2′,3′,6′,7′-tetrahydrospiro[cyclopropane-1,5′-inden]-3′-yl acetate; (6′-hydroxy-2′,4′,6′-trimethyl-7′-oxo-3′-((tributylsilyl)oxy)-2′,3′,6′,7′-tetrahydrospiro[cyclopropane-1,5′-inden]-2′-yl)methyl acetate; 2′-(((tert-butoxycarbonyl)amino)methyl)-6′-hydroxy-2′,4′,6′-trimethyl-7′-oxo-2′,3′,6′,7′-tetrahydrospiro[cyclopropane-1,5′-inden]-3′-yl acetate; (3′-(((2-((((4-((14S,17S)-1-(9H-fluoren-9-yl)-14-isopropyl-3,6,9,12,15-pentaoxo-17-(3-ureidopropyl)-2-oxa-4,7,10,13,16-pentaazaoctadecan-18-amido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (3′-(((2-((((4-((2S,5S)-14-amino-5-isopropyl-4,7,10,13-tetraoxo-2-(3-ureidopropyl)-3,6,9,12-tetraazatetradecanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (6′-hydroxy-2′-(hydroxymethyl)-2′,4′,6′-trimethyl-3′-((tributylsilyl)oxy)-2′,3′-dihydrospiro[cyclopropane-1,5′-inden]-7′(6′H)-one; 6′-hydroxy-2′,4′,6′-trimethyl-7′-oxo-3′-((tributylsilyl)oxy)-2′,3′,6′,7′-tetrahydrospiro[cyclopropane-1,5′-indene]-2′-carbaldehyde; tert-butyl ((6′-hydroxy-2′,4′,6′-trimethyl-7′-oxo-3′-((tributylsilyl)oxy)-2′,3′,6′,7′-tetrahydrospiro[cyclopropane-1,5′-inden]-2′-yl)methyl)carbamate; tert-butyl ((3′,6′-dihydroxy-2′,4′,6′-trimethyl-7′-oxo-2′,3′,6′,7′-tetrahydrospiro[cyclopropane-1,5′-inden]-2′-yl)methyl)carbamate; (3′-(((2-(2-(2-(2-azidoacetamido)acetamido)-N-methylacetamido)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (3′-(((2-(2,5-dioxo-2,5-dihydro-1H-pyrrol-1-yl)ethyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; tert-butyl ((3′,6′-dihydroxy-2′,4′,6′-trimethyl-7′-oxo-2′,3′,6′,7′-tetrahydrospiro[cyclopropane-1,5′-inden]-2′-yl)methyl)(hydroxy)carbamate; 3′,6′-dihydroxy-2′-((hydroxyamino)methyl)-2′,4′,6′-trimethyl-2′,3′-dihydrospiro[cyclopropane-1,5′-inden]-7′(6′H)-one; (3′,6′-dihydroxy-2′,4′,6′-trimethyl-7′-oxo-2′,3′,6′,7′-tetrahydrospiro[cyclopropane-1,5′-inden]-2′-yl)methyl sulfamate; (3′-(((2-((((4-((S)-2-((S)-2-((((9H-fluoren-9-yl)methoxy)carbonyl)amino)-3-methylbutanamido)-5-ureidopentanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (6′-hydroxy-2′,6′-dimethyl-4′-methylene-7′-oxo-2′,4′,6′,7′-tetrahydrospiro[cyclopropane-1,5′-inden]-2′-yl)methyl methanesulfonate; (3′-(((2-((((4-((S)-2-((S)-2-amino-3-methylbutanamido)-5-ureidopentanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (6′-hydroxy-3′-(((2-((((4-((2S,5S)-5-isopropyl-25-(6-methyl-4-vinylpyridin-2-yl)-4,7,23-trioxo-2-(3-ureidopropyl)-10,13,16,19-tetraoxa-3,6,22-triazapentacosanamido)benzyl)oxy)carbonyl)(methyl)amino)ethyl)(methyl)carbamoyl)oxy)-2′,4′,6′-trimethyl-7′-oxo-2′,3′,6′,7′-tetrahydrospiro[cyclopropane-1,5′-inden]-2′-yl)methyl acetate; (3′,6′-dihydroxy-2′,4′,6′-trimethyl-7′-oxo-2′,3′,6′,7′-tetrahydrospiro[cyclopropane-1,5′-inden]-2′-yl)methyl trifluoromethanesulfonate; 4-(14-amino-5-isopropyl-4,7,10,13-tetraoxo-2-(3-ureidopropyl)-3,6,9,12-tetraazatetradecanamido)benzyl ((2'S,3′R,6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate); 4-(2-((S)-2-amino-3-methylbutanamido)-5-ureidopentanamido)benzyl ((6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate); 4-(2-((S)-2-(6-(2,5-dioxo-2,5-dihydro-1H-pyrrol-1-yl)hexanamido)-3-methylbutanamido)-5-ureidopentanamido)benzyl ((6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate) or a pharmaceutically acceptable salt thereof; and
(d) treating the mammal with the affinity drug conjugate.

2. The method of claim 1, where the composition comprises at least an R-enantiomer of the compound.

3. The method of claim 1, where the composition comprises at least an S-enantiomer of the compound.

4. The method of claim 1, where the composition comprises an enantiomeric mixture.

5. The method of claim 1, where the affinity drug conjugate comprises a physiologically compatible carrier.

6. The method of claim 1, where the affinity drug conjugate is a liposomal particle.

7. The method of claim 1, where the affinity drug conjugate is a nanoparticle.

8. The method of claim 1, where the affinity drug conjugate is a PEGylated compound.

9. The method of claim 1, where the affinity drug conjugate one or both slows and stops infiltration of the one or more cancer cells into peripheral organs.

10. The method of claim 1, where the mammal is a human.

11. A method of treating cancer in a mammal in need thereof comprising:
(a) determining from a test sample that the mammal has a cancer;
(b) determining an affinity moiety that targets a marker expressed on the cancer;
(c) generating an affinity drug conjugate comprising:
the affinity moiety;
a composition comprising a compound selected from the group consisting of:
((2'S,3′R,6′R)-6′-hydroxy-2′,4′,6′-trimethyl-3′-(((4-nitrophenoxy)carbonyl)oxy)-7′-oxo-2′,3′,6′,7′-tetrahydrospiro[cyclopropane-1,5′-inden]-2′-yl)methyl acetate; ((2'S,3′R,6′R)-3′-(((2-((tert-butoxycarbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; tert-butyl ((2'S,3′R,6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate); ((2'S,3′R,6′R)-3′,6′-dihydroxy-2′,4′,6′-trimethyl-7′-oxo-2′,3′,6′,7′-tetrahydrospiro[cyclopropane-1,5′-inden]-2′-yl)methyl 4-methylbenzenesulfonate; (2'S,3′R,6′R)-2′-(acetoxymethyl)-6′-hydroxy-2′,4′,6′-trimethyl-7′-oxo-2′,3′,6′,7′-tetrahydrospiro[cyclopropane-1,5′-inden]-3′-yl 4-(N-(2-(2-(2-(2-aminoacetamido)acetamido)acetamido)ethyl)-N-methylsulfamoyl)benzoate; ((2'S,3′R,6′R)-3′,6′-dihydroxy-2′,4′,6′-trimethyl-7′-oxo-2′,3′,6′,7′-tetrahydrospiro[cyclopropane-1,5′-inden]-2′-yl)methyl 4-(N-(2-(2-(2-(2-aminoacetamido)acetamido)acetamido)ethyl)-N-methylsulfamoyl)benzoate; (3'S,6′R)-6′-hydroxy-2′,2′,4′,6′-tetramethyl-7′-oxo-2′,3′,6′,7′-tetrahydrospiro[cyclopropane-1,5′-inden]-3′-yl 4-(N-(2-(2-(2-(2-aminoacetamido)acetamido)acetamido)ethyl)-N-methylsulfamoyl)benzoate; ((6′R)-3′-(((2-((((4-((S)-2-((S)-2-(6-(2,5-dioxo-2,5-dihydro-1H-pyrrol-1-yl)hexanamido)-3-methylbutanamido)-5-ureidopentanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,3'S,6′R)-3′-(((1-(9H-fluoren-9-yl)-13-methyl-3,6,9,12-tetraoxo-2-oxa-4,7,10,13-tetraazapentadecan-15-yl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,3'S,6′R)-3′-(((2-(2-(2-(2-aminoacetamido)acetamido)-N-methylacetamido)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,3'S,6′R)-6′-hydroxy-2′,4′,6′-trimethyl-3′-((methyl(2,2,14-trimethyl-4,7,10,13-tetraoxo-3-oxa-5,8,11,14-tetraazahexadecan-16-yl)carbamoyl)oxy)-7′-oxo-2′,3′,6′,7′-tetrahydrospiro[cyclopropane-1,5′-inden]-2′-yl)methyl acetate; ((2'S,3′R,6′R)-3′-((((2,5-dioxopyrrolidin-1-yl)oxy)carbonyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,3′R,6′R)-3′,6′-dihydroxy-2′,4′,6′-trimethyl-7′-oxo-2′,3′,6′,7′-tetrahydrospiro[cyclopropane-1,5′-inden]-2′-yl)methyl methanesulfonate; (2'S,3′R,6′R)-2′-(aminomethyl)-3′,6′-dihydroxy-2′,4′,6′-trimethyl-2′,3′-dihydrospiro[cyclopropane-1,5′-inden]-7′(6′H)-one; (2'S,3′R,6′R)-2′-(((tert-butyldimethylsilyl)oxy)methyl)-6′-hydroxy-2′,4′,6′-trimethyl-7′-oxo-2′,3′,6′,7′-tetrahydrospiro[cyclopropane-1,5′-inden]-3′-yl acetate; (2'S,3′R,6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl acetate; (2′R,3′R,6′R)-2′-formyl-6′-hydroxy-2′,4′,6′-trimethyl-7′-oxo-2′,3′,6′,7′-tetrahydrospiro[cyclopropane-1,5′-inden]-3′-yl acetate; (2'S,3′R,6′R)-2′-(((((9H-fluoren-9-yl)methoxy)carbonyl)amino)methyl)-6′-hydroxy-2′,4′,6′-trimethyl-7′-oxo-2′,3′,6′,7′-tetrahydrospiro[cyclopropane-1,5′-inden]-3′-yl acetate; (2'S,3′R,6′R)-2′-(aminomethyl)-6′-hydroxy-2′,4′,6′-trimethyl-7′-oxo-2′,3′,6′,7′-tetrahydrospiro[cyclopropane-1,5′-inden]-3′-yl acetate; ((2'S,3′R,6′R)-6′-hydroxy-2′,4′,6′-trimethyl-7′-oxo-3′-((tributylsilyl)oxy)-2′,3′,6′,7′-tetrahydrospiro[cyclopropane-1,5′-inden]-2′-yl)methyl acetate; (2'S,3′R,6′R)-2′-(((tert-butoxycarbonyl)amino)methyl)-6′-hydroxy-2′,4′,6′-trimethyl-7′-oxo-2′,3′,6′,7′-tetrahydrospiro[cyclopropane-1,5′-inden]-3′-yl acetate; ((2'S,3′R,6′R)-3′-(((2-((((4-((14S,17S)-1-(9H-fluoren-9-yl)-14-isopropyl-3,6,9,12,15-pentaoxo-17-(3-ureidopropyl)-2-oxa-4,7,10,13,16-pentaazaoctadecan-18-amido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,3′R,6′R)-3′-(((2-((((4-((2S,5S)-14-amino-5-isopropyl-4,7,10,13-tetraoxo-2-(3-ureidopropyl)-3,6,9,12-tetraazatetradecanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; (2'S,3′R,6′R)-6′-hydroxy-2′-(hydroxymethyl)-2′,4′,6′-trimethyl-3′-((tributylsilyl)oxy)-2′,3′-dihydrospiro[cyclopropane-1,5′-inden]-7′(6′H)-one; (2′R,3′R,6′R)-6′-hydroxy-2′,4′,6′-trimethyl-7′-oxo-3′-((tributylsilyl)oxy)-2′,3′,6′,7′-tetrahydrospiro[cyclopropane-1,5′-indene]-2′-carbaldehyde; tert-butyl (((2'S,3′R,6′R)-6′-hydroxy-2′,4′,6′-trimethyl-7′-oxo-3′-((tributylsilyl)oxy)-2′,3′,6′,7′-tetrahydrospiro[cyclopropane-1,5′-inden]-2′-yl)methyl)carbamate; tert-butyl (((2'S,3′R,6′R)-3′,6′-dihydroxy-2′,4′,6′-trimethyl-7′-oxo-2′,3′,6′,7′-tetrahydrospiro[cyclopropane-1,5′-inden]-2′-yl)methyl)carbamate; ((2'S,3'S,6′R)-3′-(((2-(2-(2-(2-azidoacetamido)acetamido)-N-methylacetamido)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,3′R,6′R)-3′-(((2-(2,5-dioxo-2,5-dihydro-1H-pyrrol-1-yl)ethyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; tert-butyl (((2'S,3′R,6′R)-3′,6′-dihydroxy-2′,4′,6′-trimethyl-7′-oxo-2′,3′,6′,7′-tetrahydrospiro[cyclopropane-1,5′-inden]-2′-yl)methyl)(hydroxy)carbamate; (2'S,3′R,6′R)-3′,6′-dihydroxy-2′-((hydroxyamino)methyl)-2′,4′,6′-trimethyl-2′,3′-dihydrospiro[cyclopropane-1,5′-inden]-7′(6′H)-one; ((2'S,3′R,6′R)-3′,6′-dihydroxy-2′,4′,6′-trimethyl-7′-oxo-2′,3′,6′,7′-tetrahydrospiro[cyclopropane-1,5′-inden]-2′-yl)methyl sulfamate; ((6′R)-3′-(((2-((((4-((S)-2-((S)-2-((((9H-fluoren-9-yl)methoxy)carbonyl)amino)-3-methylbutanamido)-5-ureidopentanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,6′R)-6′-hydroxy-2′,6′-dimethyl-4′-methylene-7′-oxo-2′,4′,6′,7′-tetrahydrospiro[cyclopropane-1,5′-inden]-2′-yl)methyl methanesulfonate; ((6′R)-3′-(((2-((((4-((S)-2-((S)-2-amino-3-methylbutanamido)-5-ureidopentanamido)benzyl)oxy)carbonyl)(methyl)amino)ethyl)(methyl)carbamoyl)oxy)-6′-hydroxy-2′,4′,6′-trimethyl-7′-oxo-2′,3′,6′,7′-tetrahydrospiro[cyclopropane-1,5′-inden]-2′-yl)methyl acetate; ((2'S,3'S,6′R)-6′-hydroxy-3′-(((2-((((4-((2S,5S)-5-isopropyl-25-(6-methyl-4-vinylpyridin-2-yl)-4,7,23-trioxo-2-(3-ureidopropyl)-10,13,16,19-tetraoxa-3,6,22-triazapentacosanamido)benzyl)oxy)carbonyl)(methyl)amino)ethyl)(methyl)carbamoyl)oxy)-2′,4′,6′-trimethyl-7′-oxo-2′,3′,6′,7′-tetrahydrospiro[cyclopropane-1,5′-inden]-2′-yl)methyl acetate; ((2'S,3′R,6′R)-3′,6′-dihydroxy-2′,4′,6′-trimethyl-7′-oxo-2′,3′,6′,7′-tetrahydrospiro[cyclopropane-1,5′-inden]-2′-yl)methyl trifluoromethanesulfonate; 4-((2S,5S)-14-amino-5-isopropyl-4,7,10,13-tetraoxo-2-(3-ureidopropyl)-3,6,9,12-tetraazatetradecanamido)benzyl ((2'S,3′R,6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate); 4-((S)-2-((S)-2-amino-3-methylbutanamido)-5-ureidopentanamido)benzyl ((6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate); and 4-((S)-2-((S)-2-(6-(2,5-dioxo-2,5-dihydro-1H-pyrrol-1-yl)hexanamido)-3-methylbutanamido)-5-ureidopentanamido)benzyl ((6′R)-6′-hydroxy-2′-(hydroxymethyl)-2′,4′,6′-trimethyl-7′-oxo-2′,3′,6′,7′-tetrahydrospiro[cyclopropane-1,5′-inden]-3′-yl) ethane-1,2-diylbis(methylcarbamate) or a pharmaceutically acceptable salt thereof; and
(d) treating the mammal with the affinity drug conjugate.

12. The method of claim 11, where the affinity drug conjugate comprises a physiologically compatible carrier.

13. The method of claim 11, where the affinity drug conjugate is a liposomal particle.

14. The method of claim 11, where the affinity drug conjugate is a nanoparticle.

15. The method of claim 11, where the affinity drug conjugate is a PEGylated compound.

16. The method of claim 11, further comprising an enantiomeric mixture of at least one enantiomer of the composition.

17. The method of claim 16, where the affinity drug conjugate is a liposomal particle.

18. The method of claim 16, where the affinity drug conjugate is a nanoparticle.

19. The method of claim 16, where the affinity drug conjugate is a PEGylated compound.

20. The method of claim 11, where the mammal is a human.


SEQUENCE LISTING (181 sequences)

SEQ ID NO: 1 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 2 - protein, 209 aa, Homo sapiens
CDMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 3 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 4 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 5 - protein, 556 aa, Homo sapiens [sequence omitted]

SEQ ID NO: 6 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 7 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 8 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 9 - protein, 594 aa, Homo sapiens
RVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 10 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 11 - protein, 281 aa, Homo sapiens
MSAQESCLSLIKYFLFVFNLFFFVLGSLIFCFGIWILIDKTSFVSFVGLAFVPLQIWSKVLAISGIFTMGIALLGCVGALKELRCLLGLYFGMLLLLFATQITLGILISTQRAQLERSLRDVVEKTIQKYGTNPEETAAEESWDYVQFQLRCCGWHYPQDWFQVLILRGNGSEAHRVPCSCYNLSATNDSTILDKVILPQLSRLGHLARSRHSADICAVPAESHIYREGCAQGLQKWLHNNLISIVGICLGVGLLELGFMTLSIFLCRNLDHVYNRLARYR

SEQ ID NO: 12 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 13 - protein, 429 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 14 - protein, 848 aa, Homo sapiens [sequence omitted]

SEQ ID NO: 15 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 16 - protein, 296 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 17 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 18 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 19 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 20 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 21 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAVGLVGLIFAVCLVGFMLYRMKKKDEGSYSLEEPKQANGGAYQKPTKQEEFYA

SEQ ID NO: 22 - protein, 459 aa, Homo sapiens
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 23 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHIFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 24 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 25 - protein, 188 aa, Homo sapiens
MDCRKMARFSYSVIWIMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 26 - protein, 264 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 27 - protein, 875 aa, Homo sapiens [sequence omitted]

SEQ ID NO: 28 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 29 - protein, 622 aa, Homo sapiens [sequence omitted]

SEQ ID NO: 30 - protein, 360 aa, Homo sapiens
DPLGAAKPQWPWRRCLAALLFQLLVAVCFFSYLRVSRDDATGSPRAPSGSSRQDTTPTRPTLLILLWTWPFHIPVALSRCSEMVPGTADCHITADRKVYPQADTVIVHHWDIMSNPKSRLPPSPRPQGQRWIWFNLEPPPNCQHLEALDRYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALDFCKACWKLQQESRYQTVRSIAAWFT

SEQ ID NO: 31 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 32 - protein, 710 aa, Homo sapiens
MGGKQRDEDDEAYGKPVKYDPSFRGPIKNRSCTDVICCVLFLLFILGYIVVGIVAWLYGDPRQVLYPRNSTGAYCGMGENKDKPYLLYFNIFSCILSSNIISVAENGLQCPTPQVCVSSCPEDPWTVGKNEFSQTVGEVFYTKNRNFCLPGVPWNMTVITSLQQELCPSFLLPSAPALGRCFPWTNVTPPALPGITNDTTIQQGISGLIDSLNARDISVKIFEDFAQSWYWILVALGVALVLSLLFILLLRLVAGPLVLVLILGVLGVLAYGIYYCWEEYRVLRDKGASISQLGFTTNLSAYQSVQETWLAALIVLAVLEAILLLMLIFLRQRIRIAIALLKEASKAVGQMMSTMFYPLVTFVLLLICIAYWAMTALYLATSGQPQYVLWASNISSPGCEKVPINTSCNPTAHLVNSSCPGLMCVFQGYSSKGLIQRSVFNLQIYGVLGLFWTLNWVLALGQCVLAGAFASFYWAFHKPQDIPTFPLISAFIRTLRYHTGSLAFGALILTLVQIARVILEYIDHKLRGVQNPVARCIMCCFKCCLWCLEKFIKFLNRNAYIMIAIYGKNFCVSAKNAFMLLMRNIVRVVVLDKVTDLLLFFGKLLVVGGVGVLSFFFFSGRIPGLGKDFKSPHLNYYWLPIMTSILGAYVIASGFFSVFGMCVDTLFLCFLEDLERNNGSLDRPYYMSKSLLKILGKKNEAPPDNKKRKK

SEQ ID NO: 33 - protein, 442 aa, Homo sapiens
MQPPPSLCGRALVALVLACGLSRIWGEERGFPPDRATPLLQTAEIMTPPTKTLWPKGSNASLARSLAPAEVPKGDRTAGSPPRTISPPPCQGPIEIKETFKYINTVVSCLVFVLGIIGNSTLLRIIYKNKCMRNGPNILIASLALGDLLHIVIDIPINVYKLLAEDWPFGAEMCKLVPFIQKASVGITVLSLCALSIDRYRAVASWSRIKGIGVPKWTAVEIVLIWVVSVVLAVPEAIGFDIITMDYKGSYLRICLLHPVQKTAFMQFYKTAKDWWLFSFYFCLPLAITAFFYTLMTCEMLRKKSGMQIALNDHLKQRREVAKTVFCLVLVFALCWLPLHLSRILKLTLYNQNDPNRCELLSFLLVLDYIGINMASLNSCINPIALYLVSKRFKNCFKSCLCCWCQSFEEKQSLEEKQSCLKFKANDHGYDNFRSSNKYSSS

SEQ ID NO: 34 - protein, 1255 aa, Homo sapiens [sequence omitted]

SEQ ID NO: 35 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 36 - protein, 334 aa, Homo sapiens
MPLLPVLLVGTISKHLDWHRKEEKEHLKGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 37 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 38 - protein, 572 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 39 - protein, 701 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVAL

SEQ ID NO: 40 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 41 - protein, 701 aa, Homo sapiens; identical to SEQ ID NO: 39

SEQ ID NO: 42 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 43 - protein, 22152 aa, Homo sapiens
  REGION 13877..13878: note = misc_feature - Xaa can be any naturally occurring amino acid
  SITE 13880: note = misc_feature - Xaa can be any naturally occurring amino acid
  SITE 13887: note = misc_feature - Xaa can be any naturally occurring amino acid
  REGION 13890..13891: note = misc_feature - Xaa can be any naturally occurring amino acid
  SITE 13893: note = misc_feature - Xaa can be any naturally occurring amino acid
  SITE 13903: note = misc_feature - Xaa can be any naturally occurring amino acid
  REGION 13913..13914: note = misc_feature - Xaa can be any naturally occurring amino acid
  SITE 13916: note = misc_feature - Xaa can be any naturally occurring amino acid
  REGION 13928..13929: note = misc_feature - Xaa can be any naturally occurring amino acid
  SITE 13938: note = misc_feature - Xaa can be any naturally occurring amino acid
  REGION 13940..13941: note = misc_feature - Xaa can be any naturally occurring amino acid
  REGION 14569..14571: note = misc_feature - Xaa can be any naturally occurring amino acid
  SITE 14575: note = misc_feature - Xaa can be any naturally occurring amino acid
  SITE 14579: note = misc_feature - Xaa can be any naturally occurring amino acid
  SITE 14581: note = misc_feature - Xaa can be any naturally occurring amino acid
  REGION 14587..14591: note = misc_feature - Xaa can be any naturally occurring amino acid
  REGION 14593..14594: note = misc_feature - Xaa can be any naturally occurring amino acid
  REGION 14725..14727: note = misc_feature - Xaa can be any naturally occurring amino acid
  SITE 14731: note = misc_feature - Xaa can be any naturally occurring amino acid
  SITE 14735: note = misc_feature - Xaa can be any naturally occurring amino acid
  SITE 14737: note = misc_feature - Xaa can be any naturally occurring amino acid
  REGION 14743..14747: note = misc_feature - Xaa can be any naturally occurring amino acid
  REGION 14749..14750: note = misc_feature - Xaa can be any naturally occurring amino acid
  REGION 15661..15663: note = misc_feature - Xaa can be any naturally occurring amino acid
  SITE 15667: note = misc_feature - Xaa can be any naturally occurring amino acid
  SITE 15671: note = misc_feature - Xaa can be any naturally occurring amino acid
  SITE 15673: note = misc_feature - Xaa can be any naturally occurring amino acid
  REGION 15679..15683: note = misc_feature - Xaa can be any naturally occurring amino acid
  REGION 15685..15686: note = misc_feature - Xaa can be any naturally occurring amino acid
  REGION 15972..15974: note = misc_feature - Xaa can be any naturally occurring amino acid
  SITE 15978: note = misc_feature - Xaa can be any naturally occurring amino acid
  SITE 15982: note = misc_feature - Xaa can be any naturally occurring amino acid
  SITE 15984: note = misc_feature - Xaa can be any naturally occurring amino acid
  REGION 15990..15994: note = misc_feature - Xaa can be any naturally occurring amino acid
  REGION 15996..15997: note = misc_feature - Xaa can be any naturally occurring amino acid
  SITE 16008: note = misc_feature - Xaa can be any naturally occurring amino acid
  SITE 16015: note = misc_feature - Xaa can be any naturally occurring amino acid
  SITE 16017: note = misc_feature - Xaa can be any naturally occurring amino acid
  SITE 16021: note = misc_feature - Xaa can be any naturally occurring amino acid
  SITE 16025: note = misc_feature - Xaa can be any naturally occurring amino acid
  SITE 16034: note = misc_feature - Xaa can be any naturally occurring amino acid
  SITE 16037: note = misc_feature - Xaa can be any naturally occurring amino acid
  SITE 16040: note = misc_feature - Xaa can be any naturally occurring amino acid
  SITE 16046: note = misc_feature - Xaa can be any naturally occurring amino acid
  SITE 16051: note = misc_feature - Xaa can be any naturally occurring amino acid
  REGION 16053..16055: note = misc_feature - Xaa can be any naturally occurring amino acid
  SITE 16058: note = misc_feature - Xaa can be any naturally occurring amino acid
  REGION 16062..16063: note = misc_feature - Xaa can be any naturally occurring amino acid
  SITE 16065: note = misc_feature - Xaa can be any naturally occurring amino acid
  SITE 16072: note = misc_feature - Xaa can be any naturally occurring amino acid
  REGION 16075..16076: note = misc_feature - Xaa can be any naturally occurring amino acid
  SITE 16078: note = misc_feature - Xaa can be any naturally occurring amino acid
  SITE 16088: note = misc_feature - Xaa can be any naturally occurring amino acid
  REGION 16268..16269: note = misc_feature - Xaa can be any naturally occurring amino acid
  SITE 16278: note = misc_feature - Xaa can be any naturally occurring amino acid
  REGION 16280..16281: note = misc_feature - Xaa can be any naturally occurring amino acid
  REGION 16373..16374: note = misc_feature - Xaa can be any naturally occurring amino acid
  SITE 16376: note = misc_feature - Xaa can be any naturally occurring amino acid
  SITE 16383: note = misc_feature - Xaa can be any naturally occurring amino acid
  REGION 16386..16387: note = misc_feature - Xaa can be any naturally occurring amino acid
  SITE 16389: note = misc_feature - Xaa can be any naturally occurring amino acid
  SITE 16399: note = misc_feature - Xaa can be any naturally occurring amino acid
  REGION 16409..16410: note = misc_feature - Xaa can be any naturally occurring amino acid
  SITE 16412: note = misc_feature - Xaa can be any naturally occurring amino acid
  REGION 16424..16425: note = misc_feature - Xaa can be any naturally occurring amino acid
  SITE 16434: note = misc_feature - Xaa can be any naturally occurring amino acid
  REGION 16436..16437: note = misc_feature - Xaa can be any naturally occurring amino acid
  REGION 16439..16441: note = misc_feature - Xaa can be any naturally occurring amino acid
  SITE 16445: note = misc_feature - Xaa can be any naturally occurring amino acid
  SITE 16449: note = misc_feature - Xaa can be any naturally occurring amino acid
  SITE 16451: note = misc_feature - Xaa can be any naturally occurring amino acid
  REGION 16457..16461: note = misc_feature - Xaa can be any naturally occurring amino acid
  REGION 16463..16464: note = misc_feature - Xaa can be any naturally occurring amino acid
  REGION 16841..16842: note = misc_feature - Xaa can be any naturally occurring amino acid
  SITE 16844: note = misc_feature - Xaa can be any naturally occurring amino acid
  SITE 16851: note = misc_feature - Xaa can be any naturally occurring amino acid
  REGION 16854..16855: note = misc_feature - Xaa can be any naturally occurring amino acid
  SITE 16857: note = misc_feature - Xaa can be any naturally occurring amino acid
  SITE 16867: note = misc_feature - Xaa can be any naturally occurring amino acid
  REGION 16877..16878: note = misc_feature - Xaa can be any naturally occurring amino acid
  SITE 16880: note = misc_feature - Xaa can be any naturally occurring amino acid
  REGION 16892..16893: note = misc_feature - Xaa can be any naturally occurring amino acid
  SITE 16902: note = misc_feature - Xaa can be any naturally occurring amino acid
  REGION 16904..16905: note = misc_feature - Xaa can be any naturally occurring amino acid
  REGION 16907..16909: note = misc_feature - Xaa can be any naturally occurring amino acid
  SITE 16913: note = misc_feature - Xaa can be any naturally occurring amino acid
  SITE 16917: note = misc_feature - Xaa can be any naturally occurring amino acid
  SITE 16919: note = misc_feature - Xaa can be any naturally occurring amino acid
  REGION 16925..16929: note = misc_feature - Xaa can be any naturally occurring amino acid
  REGION 16931..16932: note = misc_feature - Xaa can be any naturally occurring amino acid
  REGION 17465..17466: note = misc_feature - Xaa can be any naturally occurring amino acid
  SITE 17468: note = misc_feature - Xaa can be any naturally occurring amino acid
  SITE 17475: note = misc_feature - Xaa can be any naturally occurring amino acid
  REGION 17478..17479: note = misc_feature - Xaa can be any naturally occurring amino acid
  SITE 17481: note = misc_feature - Xaa can be any naturally occurring amino acid
  SITE 17491: note = misc_feature - Xaa can be any naturally occurring amino acid
  REGION 17501..17502: note = misc_feature - Xaa can be any naturally occurring amino acid
  SITE 17504: note = misc_feature - Xaa can be any naturally occurring amino acid
  REGION 17516..17517: note = misc_feature - Xaa can be any naturally occurring amino acid
  SITE 17526: note = misc_feature - Xaa can be any naturally occurring amino acid
  REGION 17528..17529: note = misc_feature - Xaa can be any naturally occurring amino acid
  REGION 17531..17533: note = misc_feature - Xaa can be any naturally occurring amino acid
  SITE 17537: note = misc_feature - Xaa can be any naturally occurring amino acid
  SITE 17541: note = misc_feature - Xaa can be any naturally occurring amino acid
  SITE 17543: note = misc_feature - Xaa can be any naturally occurring amino acid
  REGION 17549..17553: note = misc_feature - Xaa can be any naturally occurring amino acid
  REGION 17555..17556: note = misc_feature - Xaa can be any naturally occurring amino acid
  SITE 17567: note = misc_feature - Xaa can be any naturally occurring amino acid
  SITE 17574: note = misc_feature - Xaa can be any naturally occurring amino acid
  SITE 17576: note = misc_feature - Xaa can be any naturally occurring amino acid
  SITE 17580: note = misc_feature - Xaa can be any naturally occurring amino acid
  SITE 17584: note = misc_feature - Xaa can be any naturally occurring amino acid
  SITE 17593: note = misc_feature - Xaa can be any naturally occurring amino acid
  SITE 17596: note = misc_feature - Xaa can be any naturally occurring amino acid
  SITE 17599: note = misc_feature - Xaa can be any naturally occurring amino acid
  SITE 17605: note = misc_feature - Xaa can be any naturally occurring amino acid
  SITE 17610: note = misc_feature - Xaa can be any naturally occurring amino acid
  REGION 17612..17614: note = misc_feature - Xaa can be any naturally occurring amino acid
  SITE 17617: note = misc_feature - Xaa can be any naturally occurring amino acid
  REGION 17621..17622: note = misc_feature - Xaa can be any naturally occurring amino acid
  SITE 17624: note = misc_feature - Xaa can be any naturally occurring amino acid
  SITE 17631: note = misc_feature - Xaa can be any naturally occurring amino acid
  REGION 17777..17778: note = misc_feature - Xaa can be any naturally occurring amino acid
  SITE 17780: note = misc_feature - Xaa can be any naturally occurring amino acid
  SITE 17787: note = misc_feature - Xaa can be any naturally occurring amino acid
  REGION 17790..17791: note = misc_feature - Xaa can be any naturally occurring amino acid
  SITE 17793: note = misc_feature - Xaa can be any naturally occurring amino acid
  SITE 17803: note = misc_feature - Xaa can be any naturally occurring amino acid
  REGION 17813..17814: note = misc_feature - Xaa can be any naturally occurring amino acid
  SITE 17816: note = misc_feature - Xaa can be any naturally occurring amino acid
  REGION 17828..17829: note = misc_feature - Xaa can be any naturally occurring amino acid
  SITE 17838: note = misc_feature - Xaa can be any naturally occurring amino acid
  REGION 17840..17841: note = misc_feature - Xaa can be any naturally occurring amino acid
  REGION 17843..17845: note = misc_feature - Xaa can be any naturally occurring amino acid
  SITE 17849: note = misc_feature - Xaa can be any naturally occurring amino acid
  SITE 17853: note = misc_feature - Xaa can be any naturally occurring amino acid
  SITE 17855: note = misc_feature - Xaa can be any naturally occurring amino acid
  REGION 17861..17865: note = misc_feature - Xaa can be any naturally occurring amino acid
  REGION 17867..17868: note = misc_feature - Xaa can be any naturally occurring amino acid
  SITE 17879: note = misc_feature - Xaa can be any naturally occurring amino acid
  SITE 17886: note = misc_feature - Xaa can be any naturally occurring amino acid
  SITE 17888: note = misc_feature - Xaa can be any naturally occurring amino acid
  SITE 17892: note = misc_feature - Xaa can be any naturally occurring amino acid
  SITE 17896: note = misc_feature - Xaa can be any naturally occurring amino acid
  SITE 17905: note = misc_feature - Xaa can be any naturally occurring amino acid
  SITE 17908: note = misc_feature - Xaa can be any naturally occurring amino acid
  SITE 17911: note = misc_feature - Xaa can be any naturally occurring amino acid
  SITE 17917: note = misc_feature - Xaa can be any naturally occurring amino acid
  SITE 17922: note = misc_feature - Xaa can be any naturally occurring amino acid
  REGION 17924..17926: note = misc_feature - Xaa can be any naturally occurring amino acid
  SITE 17929: note = misc_feature - Xaa can be any naturally occurring amino acid
  REGION 17933..17934: note = misc_feature - Xaa can be any naturally occurring amino acid
  SITE 17936: note = misc_feature - Xaa can be any naturally occurring amino acid
  SITE 17943: note = misc_feature - Xaa can be any naturally occurring amino acid
  REGION 17946..17947: note = misc_feature - Xaa can be any naturally occurring amino acid
  SITE 17949: note = misc_feature - Xaa can be any naturally occurring amino acid
  SITE 17959: note = misc_feature - Xaa can be any naturally occurring amino acid
  REGION 18089..18090: note = misc_feature - Xaa can be any naturally occurring amino acid
  SITE 18092: note = misc_feature - Xaa can be any naturally occurring amino acid
  SITE 18099: note = misc_feature - Xaa can be any naturally occurring amino acid
  REGION 18102..18103: note = misc_feature - Xaa can be any naturally occurring amino acid
  SITE 18105: note = misc_feature - Xaa can be any naturally occurring amino acid
  SITE 18115: note = misc_feature - Xaa can be any naturally occurring amino acid
  REGION 18125..18126: note = misc_feature - Xaa can be any naturally occurring amino acid
  SITE 18128: note = misc_feature - Xaa can be any naturally occurring amino acid
  REGION 18140..18141: note = misc_feature - Xaa can be any naturally occurring amino acid
  SITE 18150: note = misc_feature - Xaa can be any naturally occurring amino acid
  REGION 18152..18153: note = misc_feature - Xaa can be any naturally occurring amino acid
  REGION 18155..18157: note = misc_feature - Xaa can be any naturally occurring amino acid
  SITE 18161: note = misc_feature - Xaa can be any naturally occurring amino acid
  SITE 18165: note = misc_feature - Xaa can be any naturally occurring amino acid
  SITE 18167: note = misc_feature - Xaa can be any naturally occurring amino acid
  REGION 18173..18177: note = misc_feature - Xaa can be any naturally occurring amino acid
  REGION 18179..18180: note = misc_feature - Xaa can be any naturally occurring amino acid
  SITE 18191: note = misc_feature - Xaa can be any naturally occurring amino acid
  SITE 18198: note = misc_feature - Xaa can be any naturally occurring amino acid
  SITE 18200: note = misc_feature - Xaa can be any naturally occurring amino acid
  SITE 18204: note = misc_feature - Xaa can be any naturally occurring amino acid
  SITE 18208: note = misc_feature - Xaa can be any naturally occurring amino acid
  SITE 18217: note = misc_feature - Xaa can be any naturally occurring amino acid
  SITE 18220: note = misc_feature - Xaa can be any naturally occurring amino acid
  SITE 18223: note = misc_feature - Xaa can be any naturally occurring amino acid
  SITE 18229: note = misc_feature - Xaa can be any naturally occurring amino acid
  SITE 18234: note = misc_feature - Xaa can be any naturally occurring amino acid
  REGION 18236..18238: note = misc_feature - Xaa can be any naturally occurring amino acid
  SITE 18241: note = misc_feature - Xaa can be any naturally occurring amino acid
  REGION 18245..18246: note = misc_feature - Xaa can be any naturally occurring amino acid
  SITE 18248: note = misc_feature - Xaa can be any naturally occurring amino acid
  SITE 18255: note = misc_feature - Xaa can be any naturally occurring amino acid
  REGION 18258..18259: note = misc_feature - Xaa can be any naturally occurring amino acid
  SITE 18261: note = misc_feature - Xaa can be any naturally occurring amino acid
  SITE 18271: note = misc_feature - Xaa can be any naturally occurring amino acid
  REGION 18401..18402: note = misc_feature - Xaa can be any naturally occurring amino acid
  SITE 18404: note = misc_feature - Xaa can be any naturally occurring amino acid
  SITE 18411: note = misc_feature - Xaa can be any naturally occurring amino acid
  REGION 18414..18415: note = misc_feature - Xaa can be any naturally occurring amino acid
  SITE 18417: note = misc_feature - Xaa can be any naturally occurring amino acid
  SITE 18427: note = misc_feature - Xaa can be any naturally occurring amino acid
  REGION 18437..18438: note = misc_feature - Xaa can be any naturally occurring amino acid
  SITE 18440: note = misc_feature - Xaa can be any naturally occurring amino acid
  REGION 18452..18453: note = misc_feature - Xaa can be any naturally occurring amino acid
  SITE 18462: note = misc_feature - Xaa can be any naturally occurring amino acid
  REGION 18464..18465: note = misc_feature - Xaa can be any naturally occurring amino acid
  REGION 18467..18469: note = misc_feature - Xaa can be any naturally occurring amino acid
  SITE 18473: note = misc_feature - Xaa can be any naturally occurring amino acid
  SITE 18477: note = misc_feature - Xaa can be any naturally occurring amino acid
  SITE 18479: note = misc_feature - Xaa can be any naturally occurring amino acid
  REGION 18485..18489: note = misc_feature - Xaa can be any naturally occurring amino acid
  REGION 18491..18492: note = misc_feature - Xaa can be any naturally occurring amino acid
  SITE 18503: note = misc_feature - Xaa can be any naturally occurring amino acid
  SITE 18510: note = misc_feature - Xaa can be any naturally occurring amino acid
  SITE 18512: note = misc_feature - Xaa can be any naturally occurring amino acid
  SITE 18516: note = misc_feature - Xaa can be any naturally occurring amino acid
  SITE 18520: note = misc_feature - Xaa can be any naturally occurring amino acid
  SITE 18529: note = misc_feature - Xaa can be any naturally occurring amino acid
  SITE 18532: note = misc_feature - Xaa can be any naturally occurring amino acid
  SITE 18535: note = misc_feature - Xaa can be any naturally occurring amino acid
  SITE 18541: note = misc_feature - Xaa can be any naturally occurring amino acid
  SITE 18546: note = misc_feature - Xaa can be any naturally occurring amino acid
  REGION 18548..18550: note = misc_feature - Xaa can be any naturally occurring amino acid
  SITE 18553: note = misc_feature - Xaa can be any naturally occurring amino acid
  REGION 18557..18558: note = misc_feature - Xaa can be any naturally occurring amino acid
  SITE 18560: note = misc_feature - Xaa can be any naturally occurring amino acid
  SITE 18567: note = misc_feature - Xaa can be any naturally occurring amino acid
  REGION 18570..18571: note = misc_feature - Xaa can be any naturally occurring amino acid
  SITE 18573: note = misc_feature - Xaa can be any naturally occurring amino acid
  SITE 18583: note = misc_feature - Xaa can be any naturally occurring amino acid
  REGION 18713..18714: note = misc_feature - Xaa can be any naturally occurring amino acid
  SITE 18716: note = misc_feature - Xaa can be any naturally occurring amino acid
  SITE 18723: note = misc_feature - Xaa can be any naturally occurring amino acid
  REGION 18726..18727: note = misc_feature - Xaa can be any naturally occurring amino acid
  SITE 18729: note = misc_feature - Xaa can be any naturally occurring amino acid
  SITE 18739: note = misc_feature - Xaa can be any naturally occurring amino acid
  REGION 18749..18750: note = misc_feature - Xaa can be any naturally occurring amino acid
  SITE 18752: note = misc_feature - Xaa can be any naturally occurring amino acid
  REGION 18764..18765: note = misc_feature - Xaa can be any naturally occurring amino acid
  SITE 18774: note = misc_feature - Xaa can be any naturally occurring amino acid
  REGION 18776..18777: note = misc_feature - Xaa can be any naturally occurring amino acid
  REGION 18779..18781: note = misc_feature - Xaa can be any naturally occurring amino acid
  SITE 18785: note = misc_feature - Xaa can be any naturally occurring amino acid
  SITE 18789: note = misc_feature - Xaa can be any naturally occurring amino acid
  SITE 18791: note = misc_feature - Xaa can be any naturally occurring amino acid
  REGION 18797..18801: note = misc_feature - Xaa can be any naturally occurring amino acid
  REGION 18803..18804: note = misc_feature - Xaa can be any naturally occurring amino acid
  SITE 18815: note = misc_feature - Xaa can be any naturally occurring amino acid
  SITE 18822: note = misc_feature - Xaa can be any naturally occurring amino acid
  SITE 18824: note = misc_feature - Xaa can be any naturally occurring amino acid
  SITE 18828: note = misc_feature - Xaa can be any naturally occurring amino acid
  SITE 18832: note = misc_feature - Xaa can be any naturally occurring amino acid
  SITE 18841: note = misc_feature - Xaa can be any naturally occurring amino acid
  SITE 18844: note = misc_feature - Xaa can be any naturally occurring amino acid
  SITE 18847: note = misc_feature - Xaa can be any naturally occurring amino acid
  SITE 18853: note = misc_feature - Xaa can be any naturally occurring amino acid
  SITE 18858: note = misc_feature - Xaa can be any naturally occurring amino acid
  REGION 18860..18862: note = misc_feature - Xaa can be any naturally occurring amino acid
  SITE 18865: note = misc_feature - Xaa can be any naturally occurring amino acid
  REGION 18869..18870: note = misc_feature - Xaa can be any naturally occurring amino acid
  SITE 18872: note = misc_feature - Xaa can be any naturally occurring amino acid
  SITE 18879: note = misc_feature - Xaa can be any naturally occurring amino acid
  REGION 18882..18883: note = misc_feature - Xaa can be any naturally occurring amino acid
  SITE 18885: note = misc_feature - Xaa can be any naturally occurring amino acid
  SITE 18895: note = misc_feature - Xaa can be any naturally occurring amino acid
  REGION 19091..19093: note = misc_feature - Xaa can be any naturally occurring amino acid
  SITE 19097: note = misc_feature - Xaa can be any naturally occurring amino acid
  SITE 19101: note = misc_feature - Xaa can be any naturally occurring amino acid
  SITE 19103: note = misc_feature - Xaa can be any naturally occurring amino acid
  REGION 19109..19113: note = misc_feature - Xaa can be any naturally occurring amino acid
  REGION 19115..19116: note = misc_feature - Xaa can be any naturally occurring amino acid
  SITE 19127: note = misc_feature - Xaa can be any naturally occurring amino acid
  SITE 19134: note = misc_feature - Xaa can be any naturally occurring amino acid
  SITE 19136: note = misc_feature - Xaa can be any naturally occurring amino acid
  SITE 19140: note = misc_feature - Xaa can be any naturally occurring amino acid
  SITE 19144: note = misc_feature - Xaa can be any naturally occurring amino acid
  SITE 19153: note = misc_feature - Xaa can be any naturally occurring amino acid
  SITE 19156: note = misc_feature - Xaa can be any naturally occurring amino acid
  SITE 19159: note = misc_feature - Xaa can be any naturally occurring amino acid
  SITE 19165: note = misc_feature - Xaa can be any naturally occurring amino acid
  SITE 19170: note = misc_feature - Xaa can be any naturally occurring amino acid
  REGION 19172..19174: note = misc_feature - Xaa can be any naturally occurring amino acid
  SITE 19177: note = misc_feature - Xaa can be any naturally occurring amino acid
  REGION 19181..19182: note = misc_feature - Xaa can be any naturally occurring amino acid
  SITE 19184: note = misc_feature - Xaa can be any naturally occurring amino acid
  SITE 19191: note = misc_feature - Xaa can be any naturally occurring amino acid
  REGION 19194..19195: note = misc_feature - Xaa can be any naturally occurring amino acid
  SITE 19197: note = misc_feature - Xaa can be any naturally occurring amino acid
  SITE 19207: note = misc_feature - Xaa can be any naturally occurring amino acid
  REGION 19337..19338: note = misc_feature - Xaa can be any naturally occurring amino acid
  SITE 19340: note = misc_feature - Xaa can be any naturally occurring amino acid
  SITE 19347: note = misc_feature - Xaa can be any naturally occurring amino acid
  REGION 19350..19351: note = misc_feature - Xaa can be any naturally occurring amino acid
  SITE 19353: note = misc_feature - Xaa can be any naturally occurring amino acid
  SITE 19363: note = misc_feature - Xaa can be any naturally occurring amino acid
  REGION 19373..19374: note = misc_feature - Xaa can be any naturally occurring amino acid
  SITE 19376: note = misc_feature - Xaa can be any naturally occurring amino acid
  REGION 19388..19389: note = misc_feature - Xaa can be any naturally occurring amino acid
  SITE 19398: note = misc_feature - Xaa can be any naturally occurring amino acid
  REGION 19400..19401: note = misc_feature - Xaa can be any naturally occurring amino acid
  REGION 19403..19405: note = misc_feature - Xaa can be any naturally occurring amino acid
  SITE 19409: note = misc_feature - Xaa can be any naturally occurring amino acid
  SITE 19413: note = misc_feature - Xaa can be any naturally occurring amino acid
  SITE 19415: note = misc_feature - Xaa can be any naturally occurring amino acid
  REGION 19421..19425: note = misc_feature - Xaa can be any naturally occurring amino acid
  REGION 19427..19428: note = misc_feature - Xaa can be any naturally occurring amino acid
  SITE 19439: note = misc_feature - Xaa can be any naturally occurring amino acid
  SITE 19446: note = misc_feature - Xaa can be any naturally occurring amino acid
  SITE 19448: note = misc_feature - Xaa can be any naturally occurring amino acid
  SITE 19452: note = misc_feature - Xaa can be any naturally occurring amino acid
  SITE 19456: note = misc_feature - Xaa can be any naturally occurring amino acid
  SITE 19465: note = misc_feature - Xaa can be any naturally occurring amino acid
  SITE 19468: note = misc_feature - Xaa can be any naturally occurring amino acid
  SITE 19471: note = misc_feature - Xaa can be any naturally occurring amino acid
  SITE 19477: note = misc_feature - Xaa can be any naturally occurring amino acid
  SITE 19482: note = misc_feature - Xaa can be any naturally occurring amino acid
  REGION 19484..19486: note = misc_feature - Xaa can be any naturally occurring amino acid
  SITE 19489: note = misc_feature - Xaa can be any naturally occurring amino acid
  REGION 19493..19494: note = misc_feature - Xaa can be any naturally occurring amino acid
  SITE 19496: note = misc_feature - Xaa can be any naturally occurring amino acid
  SITE 19503: note = misc_feature - Xaa can be any naturally occurring amino acid
  REGION 19506..19507: note = misc_feature - Xaa can be any naturally occurring amino acid
  SITE 19509: note = misc_feature - Xaa can be any naturally occurring amino acid
  SITE 19519: note = misc_feature - Xaa can be any naturally occurring amino acid
  REGION 19649..19650: note = misc_feature - Xaa can be any naturally occurring amino acid
  SITE 19652: note = misc_feature - Xaa can be any naturally occurring amino acid
  SITE 19659: note = misc_feature - Xaa can be any naturally occurring amino acid
  REGION 19662..19663: note = misc_feature - Xaa can be any naturally occurring amino acid
  SITE 19665: note = misc_feature - Xaa can be any naturally occurring amino acid
  SITE 19675: note = misc_feature - Xaa can be any naturally occurring amino acid
  REGION 19685..19686: note = misc_feature - Xaa can be any naturally occurring amino acid
  SITE 19688: note = misc_feature - Xaa can be any naturally occurring amino acid
  REGION 19700..19701: note = misc_feature - Xaa can be any naturally occurring amino acid
  SITE 19710: note = misc_feature - Xaa can be any naturally occurring amino acid
  REGION 19712..19713: note = misc_feature - Xaa can be any naturally occurring amino acid
  REGION 19715..19717: note = misc_feature - Xaa can be any naturally occurring amino acid
  SITE 19721: note = misc_feature - Xaa can be any naturally occurring amino acid
  SITE 19725: note = misc_feature - Xaa can be any naturally occurring amino acid
  SITE 19727: note = misc_feature - Xaa can be any naturally occurring amino acid
  REGION 19733..19737: note = misc_feature - Xaa can be any naturally occurring amino acid
  REGION 19739..19740: note = misc_feature - Xaa can be any naturally occurring amino acid
  SITE 19751: note = misc_feature - Xaa can be any naturally occurring amino acid
  SITE 19758: note = misc_feature - Xaa can be any naturally occurring amino acid
  SITE 19760: note = misc_feature - Xaa can be any naturally occurring amino acid
  SITE 19764: note = misc_feature - Xaa can be any naturally occurring amino acid
  SITE 19768: note = misc_feature - Xaa can be any naturally occurring amino acid
  SITE 19777: note = misc_feature - Xaa can be any naturally occurring amino acid
  SITE 19780: note = misc_feature - Xaa can be any naturally occurring amino acid
  SITE 19783: note = misc_feature - Xaa can be any naturally occurring amino acid
  SITE 19789: note = misc_feature - Xaa can be any naturally occurring amino acid
  SITE 19794: note = misc_feature - Xaa can be any naturally occurring amino acid
  REGION 19796..19798: note = misc_feature - Xaa can be any naturally occurring amino acid
  SITE 19801: note = misc_feature - Xaa can be any naturally occurring amino acid
  REGION 19805..19806: note = misc_feature - Xaa can be any naturally occurring amino acid
  SITE 19808: note = misc_feature - Xaa can be any naturally occurring amino acid
  SITE 19815: note = misc_feature - Xaa can be any naturally occurring amino acid
  REGION 19818..19819: note = misc_feature - Xaa can be any naturally occurring amino acid
  SITE 19821: note = misc_feature - Xaa can be any naturally occurring amino acid
  SITE 19831: note = misc_feature - Xaa can be any naturally occurring amino acid
  REGION 19960..19961: note = misc_feature - Xaa can be any naturally occurring amino acid
  SITE 19963: note = misc_feature - Xaa can be any naturally occurring amino acid
  SITE 19970: note = misc_feature - Xaa can be any naturally occurring amino acid
  REGION 19973..19974: note = misc_feature - Xaa can be any naturally occurring amino acid
  SITE 19976: note = misc_feature - Xaa can be any naturally occurring amino acid
  SITE 19986: note = misc_feature - Xaa can be any naturally occurring amino acid
  REGION 19996..19997: note = misc_feature - Xaa can be any naturally occurring amino acid
  SITE 19999: note = misc_feature - Xaa can be any naturally occurring amino acid
  REGION 20011..20012: note = misc_feature - Xaa can be any naturally occurring amino acid
  SITE 20021: note = misc_feature - Xaa can be any naturally occurring amino acid
  REGION 20023..20024: note = misc_feature - Xaa can be any naturally occurring amino acid
  REGION 20026..20028: note = misc_feature - Xaa can be any naturally occurring amino acid
  SITE 20032: note = misc_feature - Xaa can be any naturally occurring amino acid
  SITE 20036: note = misc_feature - Xaa can be any naturally occurring amino acid
  SITE 20038: note = misc_feature - Xaa can be any naturally occurring amino acid
  REGION 20044..20048: note = misc_feature - Xaa can be any naturally occurring amino acid
  REGION 20050..20051: note = misc_feature - Xaa can be any naturally occurring amino acid
  SITE 20062: note = misc_feature - Xaa can be any naturally occurring amino acid
  SITE 20069: note = misc_feature - Xaa can be any naturally occurring amino acid
  SITE 20071: note = misc_feature - Xaa can be any naturally occurring amino acid
  SITE 20075: note = misc_feature - Xaa can be any naturally occurring amino acid
  SITE 20079: note = misc_feature - Xaa can be any naturally occurring amino acid
  SITE 20088: note = misc_feature - Xaa can be any naturally occurring amino acid
  SITE 20091: note = misc_feature - Xaa can be any naturally occurring amino acid
  SITE 20094: note = misc_feature - Xaa can be any naturally occurring amino acid
  SITE 20100: note = misc_feature - Xaa can be any naturally occurring amino acid
  SITE 20105: note = misc_feature - Xaa can be any naturally occurring amino acid
  REGION 20107..20109: note = misc_feature - Xaa can be any naturally occurring amino acid
  SITE 20112: note = misc_feature - Xaa can be any naturally occurring amino acid
  REGION 20116..20117: note = misc_feature - Xaa can be any naturally occurring amino acid
  SITE 20119: note = misc_feature - Xaa can be any naturally occurring amino acid
  SITE 20126: note = misc_feature - Xaa can be any naturally occurring amino acid
  REGION 20129..20130: note = misc_feature - Xaa can be any naturally occurring amino acid
  SITE 20132: note = misc_feature - Xaa can be any naturally occurring amino acid
  SITE 20142: note = misc_feature - Xaa can be any naturally occurring amino acid
  REGION 20272..20273: note = misc_feature - Xaa can be any naturally occurring amino acid
  SITE 20275: note = misc_feature - Xaa can be any naturally occurring amino acid
  SITE 20282: note = misc_feature - Xaa can be any naturally occurring amino acid
  REGION 20285..20286: note = misc_feature - Xaa can be any naturally occurring amino acid
  SITE 20288: note = misc_feature - Xaa can be any naturally occurring amino acid
  SITE 20298: note = misc_feature - Xaa can be any naturally occurring amino acid
  REGION 20308..20309: note = misc_feature - Xaa can be any naturally occurring amino acid
  SITE 20311: note = misc_feature - Xaa can be any naturally occurring amino acid
  REGION 20323..20324: note = misc_feature - Xaa can be any naturally occurring amino acid
  SITE 20333: note = misc_feature - Xaa can be any naturally occurring amino acid
  REGION 20335..20336: note = misc_feature - Xaa can be any naturally occurring amino acid
  REGION 20806..20808: note = misc_feature - Xaa can be any naturally occurring amino acid
  SITE 20812: note = misc_feature - Xaa can be any naturally occurring amino acid
  SITE 20816: note = misc_feature - Xaa can be any naturally occurring amino acid
  SITE 20818: note = misc_feature - Xaa can be any naturally occurring amino acid
  REGION 20824..20828: note = misc_feature - Xaa can be any naturally occurring amino acid
  REGION 20830..20831: note = misc_feature - Xaa can be any naturally occurring amino acid
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLXXEXLYWELSXLTXXIXELGPYTLDRXSLYVNGFTHXXSXPTTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMXXXPLLXPFTXNXTITNLXXXXXMXXPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAXXXPLLXPFTXNXTITNLXXXXXMXXPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGATTGMDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLXXEXLYWELSXLTXXIXELGPYTLDRXSLYVNGFTHXXSXPTTSTPGTSTVXXGTSGTPSSXPXXTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTXXXPLLXPFTXNXTITNLXXXXXMXXPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 44 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 45 - protein, 560 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 46 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 47 - protein, 427 aa, Homo sapiens
MEWPARLCGLWALLLCAGGGGGGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 48 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 49 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 50 - protein, 4130 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFT

SEQ ID NO: 51 - protein, 442 aa, Homo sapiens [sequence omitted]

SEQ ID NO: 52 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 53 - protein, 710 aa, Homo sapiens; identical to SEQ ID NO: 32

SEQ ID NO: 54 - protein, 441 aa, Yersinia pestis
MKERSTELVQGFRHSVPYINAHRGKTFVVMLGGEAIEHENFSSIVNDIGLLHSLGIRLVVVYGARPQIDSNLADHNYEPIYHKHTRVTDARTLEMVKQAAGLLQLDITARLSMSLNNTPLQGAHINVVSGNFIIAQPLGVDDGVDYCHSGRIRRIDEEAIHRQLDNGAIVLLGPVAVSVTGESFNLTSEEVATQLAIKLKAEKMIGFCSSQGVTDSEGNIISELFPNDAQKRIEDLEQDGDYNSGTVRFLRGAVKACRSGVRRSHLLSYQEDGALIQELFSRDGIGTQIVMESAEQVRRATINDIGGILELIRPLEQQGILVRRSREQLEMEIDKFTIIERDNLTIACAALYPFPDEHIGEMACVAVHPDYRSSSRGEMLLNRITNQARQMGLKKLFVLTTRSIHWFQERGFTPAEVDVLPIQKQELYNYQRRSKILLADL

SEQ ID NO: 55 - protein, 1073 aa, Homo sapiens
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 56 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 57 - protein, 393 aa, Homo sapiens [sequence omitted]

SEQ ID NO: 58 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 59 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 60 - protein, 267 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 61 - protein, 462 aa, Homo sapiens
MASNSSSCPTPGGGHLNGYPVPPYAFFFPPMLGGLSPPGALTTLQHQLPVSGYSTPSPATIETQSSSSEEIVPSPPSPPPLPRIYKPCFVCQDKSSGYHYGVSACEGCKGFFRRSIQKNMVYTCHRDKNCIINKVTRNRCQYCRLQKCFEVGMSKESVRNDRNKKKKEVPKPECSESYTLTPEVGELIEKVRKAHQETFPALCQLGKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP

SEQ ID NO: 62 - protein, 455 aa, Homo sapiens
MTTSGHACPVPAVNGHMTHYPATPYPLLFPPVIGGLSLPPLHGLHGHPPPSGCSTPSPATIETQSTSSEELVPSPPSPLPPPRVYKPCFVCQDKSSGYHYGVSACEGCKGFFRRSIQKNMIYTCHRDKNCVINKVTRNRCQYCRLQKCFEVGMSKESVRNDRNKKKKETSKQECTESYEMTAELDDLTEKIRKAHQETFPSLCQLGKYTTNSSADHRVRLDLGLWDKFSELATKCIIKIVEFAKRLPGFTGLTIADQITLLKAACLDILILRICTRYTPEQDTMTFSDGLTLNRTQMHNAGFGPLTDLVFTFANQLLPLEMDDTETGLLSAICLICGDRQDLEEPTKVDKLQEPLLEALKIYIRKRRPSKPHMFPKILMKITDLRSISAKGAERVITLKMEIPGSMPPLIQEMLENSEGHEPLTPSSSGNTAEHSPSISPSSVENSGVSQSPLVQ

SEQ ID NO: 63 - protein, 455 aa, Homo sapiens; identical to SEQ ID NO: 62

SEQ ID NO: 64 - protein, 462 aa, Homo sapiens
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 65 - protein, 533 aa, Homo sapiens
MSWAARPPFLPQRHAAGQCGPVGVRKEMHCGVASRWRRRRPWLDPAAAAAAAVAGGEQQTPEPEPGEAGRDGMGDSGRDSRSPDSSSPNPLPQGVPPPSPPGPPLPPSTAPSLGGSGAPPPPPMPPPPLGSPFPVISSSMGSPGLPPPAPPGFSGPVSSPQINSTVSLPGGGSGPPEDVKPPVLGVRGLHCPPPPGGPGAGKRLCAICGDRSSGKHYGVYSCEGCKGFFKRTIRKDLTYSCRDNKDCTVDKRQRNRCQYCRYQKCLATGMKREAVQEERQRGKDKDGDGEGAGGAPEEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPDAKGLSNPSEVEVLREKVYASLETYCKQKYPEQQGRFAKLLLRLPALRSIGLKCLEHLFFFKLIGDTPIDTFLMEMLEAPHQLA

SEQ ID NO: 66 - protein, 463 aa, Homo sapiens
MYGNYSHFMKFPAGYGGSPGHTGSTSMSPSAALSTGKPMDSHPSYTDTPVSAPRTLSAVGTPLNALGSPYRVITSAMGPPSGALAAPPGINLVAPPSSQLNVVNSVSSSEDIKPLPGLPGIGNMNYPSTSPGSLVKHICAICGDRSSGKHYGVYSCEGCKGFFKRTIRKDLIYTCRDNKDCLIDKRQRNRCQYCRYQKCLVMGMKREAVQEERQRSRERAESEAECATSGHEDMPVERILEAELAVEPKTESYGDMNMENSTNDPVTNICHAADKQLFTLVEWAKRIPHFSDLTLEDQVILLRAGWNELLIASFSHRSVSVQDGILLATGLHVHRSSAHSAGVGSIFDRVLTELVSKMKDMQMDKSELGCLRAIVLFNPDAKGLSNPSEVETLREKVYATLEAYTKQKYPEQPGRFAKLLLRLPALRSIGLKCLEHLFFFKLIGDTPIDTFLMEMLETPLQIT

SEQ ID NO: 67 - protein, 493 aa, Homo sapiens [sequence omitted]

SEQ ID NO: 68 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 69 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 70 - protein, 558 aa, Homo sapiens
MAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQRVRSGDWKGYTGKTITDVINIGIGGSDLGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 71 - protein, 307 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHC

SEQ ID NO: 72 - protein, 247 aa, Homo sapiens
MTILFLTMVISYFGCMKAAPMKEANIRGQGGLAYPGVRTHGTLESVNGPKAGSRGLTSLADTFEHVIEELLDEDHKVRPNEENNKDADLYTSRVMLSSQVPLEPPLLFLLEEYKNYLDAANMSMMVLRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 73 - protein, 1754 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 74 - protein, 1763 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASKMGGSHHHHH

SEQ ID NO: 75 - protein, 1166 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 76 - protein, 193 aa, Homo sapiens [sequence omitted]

SEQ ID NO: 77 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 78 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 79 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 80 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 81 - protein, 454 aa, Homo sapiens
MARPNKFLLWFCCFAWLCFPISLGSQASGGEAQIAASAELESGAMPWSLLQHIDERDRAGLLPALFKVLSVGRGGSPRLQPDSRALHYMKKLYKTYATKEGIPKSNRSHLYNTVRLFTPCTRHKQAPGDQVTGILPSVELLFNLDRITTVEHLLKSVLLYNINNSVSFSSAVKCVCNLMIKEPKSSSRTLGRAPYSFTFNSQFEFGKKHKWIQIDVTSLLQPLVASNKRSIHMSINFTCMKDQLEHPSAQNGLFNMTLVSPSLILYLNDTSAQAYHSWYSLHYKRRPSQGPDQERSLSAYPVGEEAAEDGRSSHHRHRRGQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 82 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 83 - protein, 240 aa, Homo sapiens
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCVEEPEPEPEAAEGDGDKKGNAEGSSDEEGKLVIDEPAKEKNEKGALKRRAGDLLEDSPKRPKEAENPEGEEKEAATLEVERPLPMEVEKNSTPSEPGSGRGPPQEEEEEEDEEEEATKEDAEAPGIRDHESL

SEQ ID NO: 84 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 85 - protein, 657 aa, Homo sapiens [sequence omitted]

SEQ ID NO: 86 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 87 - protein, 257 aa, Homo sapiens
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 88 - protein, 211 aa, Homo sapiens [sequence omitted]

SEQ ID NO: 89 - protein, 353 aa, Homo sapiens [sequence omitted]

SEQ ID NO: 90 - protein, 159 aa, Homo sapiens
MVPSAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 91 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 92 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 93 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 94 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 95 - protein, 270 aa, Ricinus communis
DNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQF

SEQ ID NO: 96 - protein, 262 aa, Ricinus communis
ADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 97 - protein, 560 aa, Corynebacterium diphtheriae
  SITE 9: note = misc_feature - Xaa can be any naturally occurring amino acid
  SITE 139: note = misc_feature - Xaa can be any naturally occurring amino acid
  SITE 149: note = misc_feature - Xaa can be any naturally occurring amino acid
  SITE 510: note = misc_feature - Xaa can be any naturally occurring amino acid
  SITE 525: note = misc_feature - Xaa can be any naturally occurring amino acid
  SITE 558: note = misc_feature - Xaa can be any naturally occurring amino acid
MSRKLFASXLIGALLGIGAPPSAHAGADDVVDSSKSFVMENFSSYHGTKPGYVDSIQKGIQKPKSGTQRNYDDDWKGFYSTDNKYDAAGYSVDNENPLSGKAGDVVKVTYPGLTKVLALKVDNAETIKKELGLSLTEPXMEQVGTEEFXKRFGDGASRVVLSLPFAEGSSSVEYINNWEQAKALSVKLEINFETRGKRGQDAMYEYMAQACAGNRVRRSVGRSLSCINLDWDVIRDKTKTKIESLKEHGPIKNKMSESPNKTVSQEKAKQYLEEFHQTALEHPQLSELKTVTGTNPLFAGANYAAWAVNVAQVIDSETADNLEKTTAALSILPGIGSVMGIADGAVHHNTEEIVAQSIALSSLMVAQAIPLVGELVDIGFAPYNFVESIINLFQVVHNSYNRSAYSPGHKTQPFLHDGYAVSWNTVEDSIIRTGFQGESGHDIKITAENTPLPIASVLLPTIPGKLDVNKSKTHISVNGRKIRMRCRAIDGDVTFCRPKSPVYVGNGVHXNLHVAFHRSSSEKIXSNEISSDSIGVLGYQKTVDHTKVNSKLSLFFEXKS

SEQ ID NO: 98 - protein, 638 aa, Pseudomonas aeruginosa
MHLTPHWIPLVASLGLLAGGSFASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 99 - protein, 316 aa, Gelonium multiflorum
MKGNMKVYWIKIAVATWFCCTTIVLGSTARIFSLPTNDEEETSKTLGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKTSLAAELIIQNYESLVGFD

SEQ ID NO: 100 - protein, 292 aa, Saponaria officinalis
MKIYVVATIAWILLQFSAWTTTDAVTSITLDLVNPTAGQYSSFVDKIRNNVKDPNLKYGGTDIAVIGPPSKDKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKSSNEANSTAYATTVL

SEQ ID NO: 101 - protein, 386 aa, Pseudomonas aeruginosa
LAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 102 - protein, 370 aa, Pseudomonas aeruginosa
LAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLAGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 103 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 104 - protein, 6 aa, synthetic construct
  REGION 1..6: note = PSA derived synthetic polypeptide
HSSKLQ

SEQ ID NO: 105 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 106 - protein, 7 aa, synthetic construct
  REGION 1..7: note = PSA SYNTHETIC POLYPEPTIDE FRAGMENT
HSSKLQK

SEQ ID NO: 107 - protein, 202 aa, Homo sapiens
MCYGKCARCIGHSLVGLALLCIAANILLYFPNGETKYASENHLSRFVWFFSGIVGGGLLMLLPAFVFIGLEQDDCCGCCGHENCGKRCAMLSSVLAALIGIAGSGYCVIVAALGLAEGPLCLDSLGQWNYTFASTEGQYLLDTSTWSECTEPKHIVEWNVSLFSILLALGGIEFILCLIQVINGVLGGICGFCCSHQQQYDC

SEQ ID NO: 108 - protein, 7 aa, synthetic construct
  REGION 1..7: note = PSA SYNTHETIC POLYPEPTIDE FRAGMENT
  SITE 6: note = X - ETHANYL-D-ALANINE
HSSKLXK

SEQ ID NO: 109 - protein, 530 aa, Homo sapiens
MRRLWGAARKPSGAGWEKEWAEAPQEAPGAWSGRLGPGRSGRKGRAVPGWASWPAHLALAARPARHLGGAGQGPRPLHSGTAPFHSRASGERQRRLEPQLQHESRCRSSTPADAWRAEAALPVRAMGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 110 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 111 - protein, 6 aa, synthetic construct
  REGION 1..6: note = PSA SYNTHETIC POLYPEPTIDE FRAGMENT
  SITE 1: note = B - 4Hyp
  SITE 4: note = X - CYCLOHEXYL GLYCINE
BASXQS

SEQ ID NO: 112 - protein, 550 aa, Homo sapiens
MPRIPASPLSCCPPDMLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 113 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 114 - protein, 750 aa, Homo sapiens; identical to SEQ ID NO: 42

SEQ ID NO: 115 - protein, 386 aa, Homo sapiens
MRAAPLLLARAASLNLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALVPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSRATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 116 - protein, 7 aa, synthetic construct
  REGION 1..7: note = PSA SYNTHETIC POLYPEPTIDE FRAGMENT
  SITE 1: note = B - 4Hyp
  SITE 4: note = X - CYCLOHEXYL GLYCINE
BASXQSL

SEQ ID NO: 117 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 118 - protein, 314 aa, Homo sapiens
MPLEQRSQHCKPEEGLEARGEALGLVGAQAPATEEQQTASSSSTLVEVTLGEVPAADSPSPPHSPQGASSFSTTINYTLWRQSDEGSSNQEEEGPRMFPDLESEFQAAISRKMVELVHFLLLKYRAREPVTKAEMLESVLRNCQDFFPVIFSKASEYLQLVFGIEVVEVVPISHLYILVTCLGLSYDGLLGDNQVMPKTGLLIIVLAIIAIEGDCAPEEKIWEELSMLEVFEGREDSVFAHPRKLLMQDLVQENYLEYRQVPGSDPACYEFLWGPRALIETSYVKVLHHTLKIGGEPHISYPPLHERALREGEE

SEQ ID NO: 119 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 120 - protein, 317 aa, Homo sapiens [sequence omitted]

SEQ ID NO: 121 - protein, 760 aa, Homo sapiens; identical to SEQ ID NO: 35

SEQ ID NO: 122 - protein, 1058 aa, Homo sapiens
MDLGPLNICEEMTILHGGFLLAEQLFHPKALAELTKSDWERVGRPIVEALREISSAAAHSQPFAWKKKALIIIWAKVLQPHPVTPSDTETRWQEDLFFSVGNMIPTINHTILFELLKSLEASGLFIQLLMALPTTICHAELERFLEHVTVDTSAEDVAFFLDVWWEVMKHKGHPQDPLLSQFSAMAHKYLPALDEFPHPPKRLRSDPDACPTMPLLAMLLRGLTQIQSRILGPGRKCCALANLADMLTVFALTEDDPQEVSATVYLDKLATVISVWNSDTQNPYHQQALAEKVKEAERDVSLTSLAKLPSETIFVGCEFLHHLLREWGEELQAVLRSSQGTSYDSYRLCDSLTSFSQNATLYLNRTSLSKEDRQVVSELAECVRDFLRKTSTVLKNRALEDITASIAMAVIQQKMDRHMEVCYIFASEKKWAFSDEWVACLGSNRALFRQPDLVLRLLETVIDVSTADRAIPESQIRQVIHLILECYADLSLPGKNKVLAGILRSWGRKGLSEKLLAYVEGFQEDLNTTFNQLTQSASEQGLAKAVASVARLVIVHPEVTVKKMCSLAVVNLGTHKFLAQILTAFPALRFVEEQGPNSSATFMVSCLKETVWMKFSTPKEEKQFLELLNCLMSPVKPQGIPVAALLEPDEVLKEFVLPFLRLDVEEVDLSLRIFIQTLEANACREEYWLQTCSPFPLLFSLCQLLDRFSKYWQLPKEKRCLSLDRKDLAIHILELLCEIVSANAETFSPDVWIKSLSWLHRKLEQLDWTVGLRLKSFFEGHFKCEVPATLFEICKLSEDEWTSQAHPGYGAGTGLLAWMECCCVSSGISERMLSLLVVDVGNPEEVRLFSKGFLVALVQVMPWCSPQEWQRLHQLTRRLLEKQLLHVPYSLEYIQFVPLLNLKPFAQELQLSVLFLRTFQFLCSHSCRDWLPLEGWNHVVKLLCGSLTRLLDSVRAIQAAGPWVQGPEQDLTQEALFVYTQVFCHALHIMAMLHPEVCEPLYVLALETLTCYETLSKTNPSVSSLLQRAHEQRFLKSIAEGIGPEERRQTLLQKMSSF

SEQ ID NO: 123 - protein, 255 aa, Homo sapiens; identical to SEQ ID NO: 44

SEQ ID NO: 124 - protein, 702 aa, Homo sapiens; identical to SEQ ID NO: 40

SEQ ID NO: 125 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 126 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 127 - protein, 8 aa, synthetic construct
  REGION 1..8: note = PSA SYNTHETIC POLYPEPTIDE FRAGMENT
  SITE 1: note = B - 4Hyp
  SITE 4: note = X - CYCLOHEXYL GLYCINE
BSSXQSSP

SEQ ID NO: 128 - protein, 351 aa, Homo sapiens; identical to SEQ ID NO: 1

SEQ ID NO: 129 - protein, 297 aa, Homo sapiens; identical to SEQ ID NO: 6

SEQ ID NO: 130 - protein, 61 aa, Homo sapiens
MKRFLFLLLTISLLVMVQIQTGLSGQNDTSQTSSPSASSSMSGGIFLFFVANAIIHLFCFS

SEQ ID NO: 131 - protein, 8 aa, synthetic construct
  REGION 1..8: note = PSA SYNTHETIC POLYPEPTIDE FRAGMENT
  SITE 1: note = Z - 4-O-acetyl-hydroxyproline
  SITE 4: note = X - CYCLOHEXYL GLYCINE
ZSSXQSSP

SEQ ID NO: 132 - protein, 8 aa, synthetic construct
  REGION 1..8: note = PSA SYNTHETIC POLYPEPTIDE FRAGMENT
RRSSYYSG

SEQ ID NO: 133 - protein, 164 aa, Homo sapiens
EMLQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 134 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 135 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 136 - protein, 6 aa, synthetic construct
  REGION 1..6: note = PSA SYNTHETIC POLYPEPTIDE FRAGMRNT
SSKYQL

SEQ ID NO: 137 - protein, 7 aa, synthetic construct
  REGION 1..7: note = PSA SYNTHGETIC POLYPEPTIDE FRAGMENT
  SITE 1: note = B - N-glutaryl-4-hydroxyproline
  SITE 4: note = X - CYCLOHEXYL GLYCINE
BASXQSL

SEQ ID NO: 138 - protein, 5 aa, synthetic construct
  REGION 1..5: note = CASPASE-3 SYNTHETIC POLYPEPTIDE FRAGMENT
DEVDP

SEQ ID NO: 139 - protein, 8 aa, synthetic construct
  REGION 1..8: note = CASPASE-3 SYNTHETIC POLYPEPTIDE FRAGMENT
KGSGDVEG

SEQ ID NO: 140 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKTELVVEVKSDKLPEEMGLLQGSSGDKRAPGDQGEKYIDLRH

SEQ ID NO: 141 - protein, 4 aa, synthetic construct
  REGION 1..4: note = CATHEPSIN B SYNTHETIC POLYPEPTIDE FRAGMENT
GFLG

SEQ ID NO: 142 - protein, 4 aa, synthetic construct
  REGION 1..4: note = CATHEPSIN B SYNTHETIC POLYPEPTIDE FRAGMENT
KKFA

SEQ ID NO: 143 - protein, 1210 aa, Homo sapiens; identical to SEQ ID NO: 31

SEQ ID NO: 144 - protein, 4 aa, synthetic construct
  REGION 1..4: note = CATHEPSIN B SYNTHETIC POLYPEPTIDE FRAGMENT
AFKK

SEQ ID NO: 145 - protein, 4 aa, synthetic construct
  REGION 1..4: note = CATHEPSIN B SYNTHETIC POLYPEPTIDE FRAGMENT
GFLG

SEQ ID NO: 146 - protein, 9 aa, synthetic construct
  REGION 1..9: note = FAP SYNTHETIC POLYPEPTIDE FRAGMENT
KQEQNPGST

SEQ ID NO: 147 - protein, 11 aa, synthetic construct
  REGION 1..11: note = Synthetic polypeptide
ACDCRGDCFCG

SEQ ID NO: 148 - protein, 13 aa, synthetic construct
  REGION 1..13: note = Synthetic polypeptide
DGARYCRGDCFDG

SEQ ID NO: 149 - protein, 5 aa, synthetic construct
  REGION 1..5: note = Cathepsin B synthetic polypeptide fragment
RGDYK

SEQ ID NO: 150 - protein, 5 aa, Homo sapiens
RGDFK

SEQ ID NO: 151 - protein, 10 aa, Homo sapiens
RGDYKKYDGR

SEQ ID NO: 152 - protein, 14 aa, synthetic construct
  REGION 1..14: note = Cathepsin B synthetic polypeptide fragment
CAGKNFFWKTFTSC

SEQ ID NO: 153 - protein, 5 aa, synthetic construct
  REGION 1..5: note = Cathepsin B synthetic polypeptide fragment
  SITE 5: note = X - N-methyl valine
RGDFX

SEQ ID NO: 154 - protein, 5 aa, Homo sapiens
PHSCN

SEQ ID NO: 155 - protein, 5 aa, Homo sapiens
  SITE 1: note = X - N-acetylproline
  SITE 5: note = Z - 2-aminobutanediamide
XHSCZ

SEQ ID NO: 156 - protein, 25 aa, Homo sapiens
TMPFLFCNVNDVCNFASRNDYSYWL

SEQ ID NO: 157 - protein, 4 aa, synthetic construct
  REGION 1..4: note = Cathepsin B synthetic polypeptide fragment
YSNS

SEQ ID NO: 158 - protein, 5 aa, synthetic construct
  REGION 1..5: note = Cathepsin B synthetic polypeptide fragment
YSNSG

SEQ ID NO: 159 - protein, 20 aa, synthetic construct
  REGION 1..20: note = Cathepsin B synthetic polypeptide fragment
FLSSRLQDLYSIVRRADRAA

SEQ ID NO: 160 - protein, 11 aa, synthetic construct
  REGION 1..11: note = Cathepsin B synthetic polypeptide fragment
IVRRADRAAVP

SEQ ID NO: 161 - protein, 13 aa, synthetic construct
  REGION 1..13: note = Cathepsin B synthetic polyipeptide fragment
RQVFQVAYIIIKA

SEQ ID NO: 162 - protein, 12 aa, synthetic construct
  REGION 1..12: note = Cathepsin B synthetic polypeptide fragment
KAFDITYVRLKF

SEQ ID NO: 163 - protein, 12 aa, synthetic construct
  REGION 1..12: note = Laminin Synthetic Polypeptide C16S
DFKLFAVTIKYR

SEQ ID NO: 164 - protein, 8 aa, synthetic construct
  REGION 1..8: note = Cathepsin B synthetic polypeptide fragment
CPQPRPLC

SEQ ID NO: 165 - protein, 503 aa, Homo sapiens
MLSQLAMLQGSLLLVVATMSVAQQTRQEADRGCETLVVQHGHCSYTFLLPKSEPCPPGPEVSRDSNTLQRESLANPLHLGKLPTQQVKQLEQALQNNTQWLKKLERAIKTILRSKLEQVQQQMAQNQTAPMLELGTSLLNQTTAQIRKLTDMEAQLLNQTSRMDAQMPETFLSTNKLENQLLLQRQKLQQLQGQNSALEKRLQALETKQQEELASILSKKAKLLNTLSRQSAALTNIERGLRGVRHNSSLLQDQQHSLRQLLVLLRHLVQERANASAPAFIMAGEQVFQDCAEIQRSGASASGVYTIQVSNATKPRKVFCDLQSSGGRWTLIQRRENGTVNFQRNWKDYKQGFGDPAGEHWLGNEVVHQLTRRAAYSLRVELQDWEGHEAYAQYEHFHLGSENQLYRLSVVGYSGSAGRQSSLVLQNTSFSTLDSDNDHCLCKCAQVMSGGWWFDACGLSNLNGVYYHAPDNKYKMDGIRWHYFKGPSYSLRASRMMIRPLDI

SEQ ID NO: 166 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 167 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 168 - protein, 226 aa, Homo sapiens
MFIMGLGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 169 - protein, 322 aa, Homo sapiens
MRTSKCFLKTASSQRNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 170 - protein, 364 aa, Homo sapiens
MHRLIFVYTLICANFCSCRDTSATPQSASIKALRNANLRRDDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 171 - protein, 6 aa, synthetic construct
  REGION 1..6: note = KALLIKREIN 2 SYNTHETIC POLYPEPTIDE FRAGMENT
GKAFRR

SEQ ID NO: 172 - protein, 7 aa, synthetic construct
  REGION 1..7: note = MMP-2/-9/ SYNTHETIC POLYPEPTIDE FRAGMENT
  SITE 3: note = Z - citrulline (2-Amino-5-(carbamoylamino)pentanoic acid)
  SITE 5: note = X - homophenylalanine (2-amino-4-phenylbutanoic acid)
EPZGXYL

SEQ ID NO: 173 - protein, 6 aa, synthetic construct
  REGION 1..6: note = MMP-2/-9/ SYNTHETIC POLYPEPTIDE FRAGMENT
GILGVP

SEQ ID NO: 174 - protein, 8 aa, synthetic construct
  REGION 1..8: note = MMP-2/-9/ SYNTHETIC POLYPEPTIDE FRAGMENT
GPLGIAGQ

SEQ ID NO: 175 - protein, 8 aa, synthetic construct
  REGION 1..8: note = MMP-7 SYNTHETIC POLYPEPTIDE FRAGMENT
KRALGLPG

SEQ ID NO: 176 - protein, 8 aa, synthetic construct
  REGION 1..8: note = MMP-7 SYNTHETIC POLYPEPTIDE FRAGMENT
RPLALWRS

SEQ ID NO: 177-178 - skipped (2 entries)

SEQ ID NO: 179 - protein, 4 aa, synthetic construct
  REGION 1..4: note = PLASMIN SYNTHETIC POLYPEPTIDE FRAGMENT
AFKK

SEQ ID NO: 180 - protein, 11 aa, synthetic construct
  REGION 1..11: note = THROMBIN SYNTHETIC POLYPEPTIDE FRAGMENT
  SITE 1: note = X - POLY-L-LYSINE
  SITE 4: note = Z - piperidine-4-carboxylic acid
XGFZRSGGGGG

SEQ ID NO: 181 - protein, 8 aa, synthetic construct
  REGION 1..8: note = TRYPSIN SYNTHETIC POLYPEPTIDE FRAGMENT
  SITE 1: note = X - POLY-L-LYSINE
XGASRFTG